(19) United States
(12) Patent Application Publication
(10) Pub. No.: US 2026/0251657 A1
(43) Pub. Date: Aug. 27, 2026

(54) METHODS FOR ANALYZING LUNG CANCER ASSOCIATED PROTEOMIC MARKERS

(71) Applicant: PrognomiQ, Inc., San Mateo, CA (US)

(72) Inventors: Bruce WILCOX, Keezletown, VA (US); Manway LIU, Burlingame, CA (US); Brian D. KOH, Oakland, CA (US); Philip MA, San Jose, CA (US); Vernon NORVIEL, Evergreen, CO (US)

(21) Appl. No.: 19/367,811

(22) Filed: Oct. 23, 2025

Related U.S. Application Data
(60) Provisional application No. 63/763,177, filed on Feb. 25, 2025.
(60) Provisional application No. 63/867,632, filed on Aug. 20, 2025.
(60) Provisional application No. 63/888,155, filed on Sep. 25, 2025.
(60) Provisional application No. 63/901,412, filed on Oct. 17, 2025.
(60) Provisional application No. 63/904,319, filed on Oct. 23, 2025.

(51) Int. Cl. G01N 33/5752; G01N 33/573; G01N 33/575

(52) U.S. Cl. CPC G01N 33/5752; G01N 33/573; G01N 33/57565; G01N 33/5758; G01N 2333/4716; G01N 2333/988

(57) ABSTRACT

Provided herein are methods of detecting lung cancer associated markers in a biological sample and for using one or more lung cancer associated markers to assess lung cancer in a biological sample. Also provided herein are systems, compositions, and kits for performing the methods disclosed herein.

### CROSS-REFERENCE

This application claims the benefit of U.S. Provisional Application No. 63/867,632, filed Aug. 20, 2025; U.S. Provisional Application No. 63/888,155, filed Sep. 25, 2025; U.S. Provisional Application No. 63/901,412, filed Oct. 17, 2025; and U.S. Provisional Application No. 63/904,319, filed Oct. 23, 2025, all of which are incorporated herein by reference.

### INCORPORATION BY REFERENCE OF SEQUENCE LISTING

The application contains a Sequence Listing, which is submitted herewith in XML format, and is hereby incorporated by reference in its entirety. The XML copy, created Oct. 23, 2025, is named 59521-735.202_SL.xml and is 94,261 bytes in size.

### SUMMARY

Aspects of the present disclosure provide methods, comprising: (a) extracting lung cancer associated proteomic markers from a biofluid sample obtained from a subject or a processed sample therefrom, wherein the lung cancer associated proteomic markers comprise seven or more of: Myoglobin (MB), Complement Component C9 (C9), Cell adhesion molecule (CEA), CA-125 Antigen (CA125), Fragment of Cytokeratin 19 (CYFRA21-1), Glycoprotein 130 (GP130), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, or a proxy proteomic marker thereof as determined using a proteomic association analysis; (b) analyzing the lung cancer associated proteomic markers in (a) by a method comprising: (1) selectively binding at least a subset of the lung cancer associated proteomic markers to one or more detection reagents to form one or more detectable complexes; and (2) detecting one or more signals obtained from the one or more detectable complexes corresponding to a concentration or an amount of the lung cancer associated proteomic markers in the one or more detectable complexes to produce a plurality of proteomic measurements; (c) generating a data set comprising the plurality of proteomic measurements; and (d) analyzing the data set from (c). In some embodiments, the lung cancer associated proteomic markers are predictive of lung cancer when the plurality of proteomic measurements is analyzed with a classifier that is trained to distinguish lung cancer samples from non-cancer samples with a performance characteristic that comprises a sensitivity of at least 80% and a specificity of at least 55%. In some embodiments, the lung cancer associated proteomic markers further comprise ten or more of: Myoglobin (MB), Complement Component C9 (C9), Cell adhesion molecule (CEA), CA-125 Antigen (CA125), Fragment of Cytokeratin 19 (CYFRA21-1), Glycoprotein 130 (GP130), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, or a proxy proteomic marker thereof. In some embodiments, the plurality of proteomic measurements is obtained from fewer than or equal to about 20 of the lung cancer associated proteomic markers. In some embodiments, the plurality of proteomic measurements is obtained from fewer than or equal to about 11 of the lung cancer associated proteomic markers. In some embodiments, the biofluid sample is a blood sample and the processed sample is a plasma sample or a serum sample comprising plasma or serum isolated from the blood sample. In some embodiments, the lung cancer is stage 1 non-small cell lung cancer. In some embodiments, the sensitivity is greater than or equal to about 87%. In some embodiments, the lung cancer is stage 2 non-small cell lung cancer. In some embodiments, the sensitivity is greater than or equal to about 88%. In some embodiments, the lung cancer is stage 3 or 4 non-small cell lung cancer. In some embodiments, the sensitivity is about 100%. In some embodiments, the classifier is trained to distinguish the lung cancer samples from the non-cancer samples using a training cohort comprising no more than 20% of subjects with the lung cancer. In some embodiments, the classifier is trained to distinguish the lung cancer samples from the non-cancer samples with an area under the curve (AUC) that is greater than or equal to about 0.80. In some embodiments, the at least the subset of the one or more lung cancer associated proteomic markers is immobilized to a solid support directly or indirectly. In some embodiments, the solid support is a bead, a welled plate, a lateral flow membrane, a planar surface, a flow cell, or any combination thereof. In some embodiments, the one or more detection reagents comprises an antibody or an antigen-binding fragment coupled directly or indirectly to a detectable label. In some embodiments, the detectable label comprises a fluorescent label, an enzymatic label, a radioactive label, an affinity label, or any combination thereof. In some embodiments, the affinity label comprises streptavidin-biotin. In some embodiments, the fluorescent label comprises a fluorescent molecule or a fluorescence resonance energy transfer pair. In some embodiments, the enzymatic label comprises horse radish peroxidase. In some embodiments, the method for analyzing the lung cancer associated proteomic markers in (b) comprises performing an immunoassay that comprises an enzyme-linked immunosorbent assay (ELISA), a particle-based immunoassay, a proximity extension assay, a lateral flow assay, or any combination thereof. In some embodiments, the immunoassay comprises a fluorescence or bioluminescence readout. In some embodiments, the particle-based immunoassay comprises forming a detectable complex of the one or more detectable complexes, wherein the detectable complex comprises: (i) a receptor immobilized to a particle, wherein the receptor specifically binds to a lung cancer associated proteomic marker of the at least the subset of the lung cancer associated proteomic markers; and (ii) the one or more detection reagents comprising a binding moiety coupled to a detectable label, wherein the binding moiety specifically binds to the lung cancer associated proteomic marker or a molecular tag directly or indirectly coupled thereto. In some embodiments, the receptor comprises an antibody or an antigen-binding fragment. In some embodiments, a detection reagent of the one or more detection reagents comprises an antibody or an antigen-binding fragment coupled to the detectable label. In some embodiments, the detectable label is a fluorescent, enzymatic, radioactive, and affinity label. In some embodiments, the particle is a bead, a nanoparticle, or a surface plasmon resonance (SPR) particle. In some embodiments, the bead comprises a coating layer coupled to a surface of the bead, wherein the coating layer comprises carboxyl (—COOH) groups, streptavidin, or avidin. In some embodiments, the method further comprises administering a therapeutic agent for the treatment of the lung cancer to the subject, wherein the therapeutic agent is provided in Table 2 or Table 40. In some embodiments, the method for analyzing the lung cancer associated proteomic markers in (b) comprises performing at least two different immunoassays. In some embodiments, the at least two different immunoassays comprise a first immunoassay, a second immunoassay, and a third immunoassay, wherein: (i) Myoglobin (MB), Complement Component C9 (C9), Fragment of Cytokeratin 19 (CYFRA21-1), Glycoprotein 130 (GP130), Gamma-enolase (ENO2), Insulin-like growth factor-binding protein 6 (IGFBP-6), Platelet endothelial cell adhesion molecule (PECAM1), Serum amyloid A protein (SAA), or any fragment thereof, or the proxy proteomic marker thereof, is analyzed using the first immunoassay; (ii) Cell adhesion molecule (CEA), CA-125 Antigen (CA125), or any fragment thereof, or the proxy proteomic marker thereof, is analyzed using the second immunoassay; and (iii) Fibrinogen-like protein 1 (FGL1), a fragment thereof, or the proxy proteomic marker thereof, is analyzed using the third immunoassay.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the present inventive concepts will be obtained by reference to the following detailed description that sets forth illustrative embodiments, in which the principles of the inventive concepts are utilized, and the accompanying drawings of which:

FIG. 1 shows a non-limiting example of a computing device; in this case, a device with one or more processors, memory, storage, and a network interface.

FIG. 2 shows a non-limiting example of a web/mobile application provision system; in this case, a system providing browser-based and/or native mobile user interfaces.

FIG. 3 shows a non-limiting example of a cloud-based web/mobile application provision system; in this case, a system comprising an elastically load balanced, auto-scaling web server and application server resources as well synchronously replicated databases.

FIG. 4 shows a map of the clinical sites in the United States where thousands of samples were prospectively collected.

FIG. 5 shows a schematic of the liquid chromatography/mass spectrometry (LC/MS) separation process for the discovery study as described elsewhere herein.

FIG. 6 shows bar graphs of the coefficient of variation (CV) percentage spread of each of four LC/MS instruments in the discovery study.

FIG. 7 shows a bar graph of the CV percentage spread across the four LC/MS instruments in the discovery study.

FIG. 8 shows the CV percentage distribution of precursor quantity for 45 plates for each of the four LC/MS instruments.

FIG. 9 shows the CV percentage distribution of precursor quantity for different numbers of precursors for each of the four LC/MS instruments.

FIG. 10 shows a graph of the number of protein groups identified as a function of the percentage of subjects.

FIG. 11 shows the top 50 gene names (by Open Target cancer association score) of proteins that were detected in the study.

FIG. 12 shows the distribution of protein group counts by subject and nanoparticle.

FIG. 13 shows the number of protein groups vs. platelet contamination index for the five different particles.

FIG. 14 shows the number of protein groups vs. erythrocyte contamination index for the five different particles.

FIG. 15 shows the erythrocyte and platelet contamination indices at different sites.

FIG. 16 shows a comparison of the erythrocyte contamination indices for cancer vs. control using 2,470 subjects.

FIG. 17 shows a comparison of the platelet contamination indices for cancer vs. control using 2,470 subjects.

FIG. 18 shows peptide, mRNA, targeted protein, RNA-intron, and metabolite features of a 682-feature classifier.

FIG. 19 shows that for the discovery study, an AUC of 0.91 was found for proteomics alone, and an AUC of 0.96 was found for multi-omics.

FIG. 20 shows schematics of the methods of in silico ID verification and empirical ID verification.

FIG. 21 shows example MS peaks used in developing an objective ranking criterion.

FIG. 22 shows a schematic of how robust peptide signals were identified.

FIG. 23 shows a comparison of the AUC of the classifier previously trained using 256 peptides and a classifier trained using verified peptides only.

FIG. 24 shows the number of endogenous peptides detected at different amounts of Proteograph plasma on the column.

FIG. 25 shows the endogenous peptide peak area inter-quartile range (IQR) at different amounts of Proteograph plasma.

FIG. 26 shows the peptide quantitative dynamic ranges.

FIG. 27 shows the dwell optimization vs. matrix peptide CV's.

FIG. 28 shows the SIL curve heavy log 10 peak area IQR.

FIG. 29 shows the load on column vs. mean peak area curve for an example peptide.

FIG. 30 shows peptides that fell within the linear range.

FIG. 31 shows marker protein plasma concentration in nanogram/microliter (ng/ml).

FIG. 32 shows a schematic of the process of verification of discovery results in a first IUP study.

FIG. 33 shows full width half maximum (FWHM) distributions for different plates in the first IUP study.

FIG. 34 shows CV's of internal standards across the different plates in the first IUP study.

FIG. 35 shows the process control CV's for 7501 and 7502.

FIG. 36 shows the biological sample CV's for 7501 and 7502.

FIG. 37 shows that approximately 60 proteins were statistically significant at distinguishing between cancer and non-cancer in the first IUP study.

FIG. 38 shows performance comparisons between LC/MS and ELISA for specific proteins in the first IUP study.

FIG. 39 shows the correlation between LC/MS and immunoassay fold changes.

FIG. 40 shows intra-plate concentration % CV for 174 proteins analyzed.

FIG. 41A shows protein group-based subject PI indices by site, sub-group, and class.

FIG. 41B shows protein group-based subject EI indices by site, sub-group, and class.

FIG. 42 shows that 51 proteins were found to be statistically significant at distinguishing between cancer and non-cancer after pre-analytical balancing.

FIG. 43A shows that the second IUP study was balanced by pre-analytical variability.

FIG. 43B shows that the second IUP study was balanced by site.

FIG. 44 shows a schematic of the fully-automated workflow used in the second IUP study.

FIG. 45A shows an overlay of the standard curves of each of the proteins analyzed.

FIG. 45B shows Levey Jennings plots of the process control for 2 assays.

FIG. 46 shows a comparison of the CV's between the first IUP study and second IUP study.

FIG. 47 shows the numbers and types of subjects for the four studies (discovery study, IUP study 1, IUP study 2, and IUP study 3).

FIG. 48 shows the AUROC of the selected lung cancer classifier on the training and hold-out validation data in the lab-developed test described herein.

FIG. 49. shows a screenshot of the summary table with information on each protein analyzed for a subject and the clinical decision.

FIG. 50 shows a schematic of the testing workflow for the lab-developed test described herein.

FIG. 51 shows an example of instructions that may be provided in the sample collection kit.

FIG. 52 shows an example of a test requisition form (TRF) that may be provided with the sample collection kit.

FIG. 53 shows the intra-day CV for CA125 and CEA.

FIG. 54 shows the overall CV for CA125 and CEA.

FIG. 55 shows linear regression plots for Myoglobin in serially diluted plasma samples.

FIGS. 56A-56I shows the Deming regression plots for the proteins meeting the acceptance criteria described herein. FIG. 56A shows the Deming regression plot for gp130. FIG. 56B shows the Deming regression plot for CD31/PECAM-1. FIG. 56C shows the Deming regression plot for Complement Component C9. FIG. 56D shows the Deming regression plot for myoglobin. FIG. 56E shows the Deming regression plot for IGFBP-6. FIG. 56F shows the Deming regression plot for CYFRA21-1. FIG. 56G shows the Deming regression plot for Enolase 2. FIG. 56H shows the Deming regression plot for CEACAM-5/CD66e. FIG. 56I shows the Deming regression plot for FGL1.

FIG. 57 show the Deming regression plot for SAA.

FIG. 58 shows the quality assessed for batch B001.

FIG. 59 shows the quality assessed for batch B002.

FIGS. 60A-I shows the list of proteins run on each Luminex panel. FIG. 60A shows Panel 1 which was a 41-plex assay with a 1:2 dilution using the Luminex Human Discovery Assay.

FIG. 60B shows Panel 2 which was a 38-plex assay with a 1:2 dilution using the Luminex Human Discovery Assay. FIG. 60C shows Panel 3 which was a 16-plex assay with a 1:2 dilution using the Luminex Human Discovery Assay. FIG. 60D shows Panel 8 which was a single assay with a 1:2 dilution using the Luminex Human Discovery Assay. FIG. 60E shows Panel 9 which was a 28-plex assay with a 1:2 dilution using the Luminex Human Tumor Biomarker Luminex Performance Panel. FIG. 60F shows Panel 4 which was a 17-plex assay with a 1:50 dilution using the Luminex Human Discovery Assay. FIG. 60G shows Panel 5 which was a 11-plex assay with a 1:50 dilution using the Luminex Human Discovery Assay. FIG. 60H shows Panel 6 which was a 14-plex assay with a 1:200 dilution using the Luminex Human Discovery Assay. FIG. 60I shows Panel 7 which was a 9-plex assay with a 1:4000 dilution using the Luminex Human Discovery Assay.

FIG. 61 shows the 48 proteins selected from prior studies considered for this study including the accompanying assay.

### DETAILED DESCRIPTION

There is a need for improved methods of lung cancer detection. At least 14 million people in the United States may be at increased risk of lung cancer. The typical standard of care for screening is currently used infrequently due to costs and the need for specialized equipment and facilities such as those that can perform diagnostic imaging. Due to these limitations, about 43% of diagnoses of lung cancer are at a late stage. At a late stage of lung cancer, the 5-year survival rate is only about 9%. The present disclosure provides methods, systems, and kits that may be used for earlier detection of lung cancer in at-risk patients.

Provided herein are methods of detecting lung cancer associated markers, methods of lung cancer screening, and methods of treatment. Also provided herein are systems and kits for performing the methods disclosed herein along with compositions for detecting lung cancer associated markers.

### I. Methods

### 1. Methods of Detecting Lung Cancer Associated Markers

Provided herein are methods of detecting lung cancer associated markers in a biological sample. Also provided herein are methods for using one or more lung cancer associated markers to assess lung cancer in a biological sample.

a. Lung Cancer Associated Markers

In some embodiments, one lung cancer associated marker is used to assess lung cancer in a biological sample. In some embodiments, two lung cancer associated markers are used to assess lung cancer in a biological sample. In some embodiments, three lung cancer associated markers are used to assess lung cancer in a biological sample. In some embodiments, four lung cancer associated markers are used to assess lung cancer in a biological sample. In some embodiments, five lung cancer associated markers are used to assess lung cancer in a biological sample. In some embodiments, six lung cancer associated markers are used to assess lung cancer in a biological sample. In some embodiments, seven lung cancer associated markers are used to assess lung cancer in a biological sample. In some embodiments, eight lung cancer associated markers are used to assess lung cancer in a biological sample. In some embodiments, nine lung cancer associated markers are used to assess lung cancer in a biological sample. In some embodiments, ten lung cancer associated markers are used to assess lung cancer in a biological sample. In some embodiments, eleven lung cancer associated markers are used to assess lung cancer in a biological sample. In some embodiments, twelve lung cancer associated markers are used to assess lung cancer in a biological sample. In some embodiments, thirteen lung cancer associated markers are used to assess lung cancer in a biological sample. In some embodiments, fourteen lung cancer associated markers are used to assess lung cancer in a biological sample. In some embodiments, fifteen lung cancer associated markers are used to assess lung cancer in a biological sample. In some embodiments, sixteen lung cancer associated markers are used to assess lung cancer in a biological sample. In some embodiments, seventeen lung cancer associated markers are used to assess lung cancer in a biological sample. In some embodiments, eighteen lung cancer associated markers are used to assess lung cancer in a biological sample. In some embodiments, nineteen lung cancer associated markers are used to assess lung cancer in a biological sample. In some embodiments, twenty lung cancer associated markers are used to assess lung cancer in a biological sample. In some embodiments, twenty-one lung cancer associated markers are used to assess lung cancer in a biological sample. In some embodiments, twenty-two lung cancer associated markers are used to assess lung cancer in a biological sample. In some embodiments, twenty-three lung cancer associated markers are used to assess lung cancer in a biological sample. In some embodiments, twenty-four lung cancer associated markers are used to assess lung cancer in a biological sample. In some embodiments, about twenty-five lung cancer associated markers are used to assess lung cancer in a biological sample. In some embodiments, about thirty lung cancer associated markers are used to assess lung cancer in a biological sample. In some embodiments, about thirty-five lung cancer associated markers are used to assess lung cancer in a biological sample. In some embodiments, about forty lung cancer associated markers are used to assess lung cancer in a biological sample. In some embodiments, about forty-five lung cancer associated markers are used to assess lung cancer in a biological sample. In some embodiments, about fifty lung cancer associated markers are used to assess lung cancer in a biological sample. In some embodiments, about fifty-five lung cancer associated markers are used to assess lung cancer in a biological sample. In some embodiments, about sixty lung cancer associated markers are used to assess lung cancer in a biological sample. In some embodiments, about sixty-five lung cancer associated markers are used to assess lung cancer in a biological sample. In some embodiments, about seventy lung cancer associated markers are used to assess lung cancer in a biological sample. In some embodiments, about seventy-five lung cancer associated markers are used to assess lung cancer in a biological sample. In some embodiments, about eighty lung cancer associated markers are used to assess lung cancer in a biological sample. In some embodiments, about eighty-five lung cancer associated markers are used to assess lung cancer in a biological sample. In some embodiments, about ninety lung cancer associated markers are used to assess lung cancer in a biological sample. In some embodiments, about ninety-five lung cancer associated markers are used to assess lung cancer in a biological sample. In some embodiments, about one-hundred lung cancer associated markers are used to assess lung cancer in a biological sample.

In some embodiments, fewer than or equal to about five lung cancer associated markers are used to assess lung cancer in a biological sample. In some embodiments, fewer than or equal to about ten lung cancer associated markers are used to assess lung cancer in a biological sample. In some embodiments, fewer than or equal to about eleven lung cancer associated markers are used to assess lung cancer in a biological sample. In some embodiments, fewer than or equal to about twelve lung cancer associated markers are used to assess lung cancer in a biological sample. In some embodiments, fewer than or equal to about thirteen lung cancer associated markers are used to assess lung cancer in a biological sample. In some embodiments, fewer than or equal to about fourteen lung cancer associated markers are used to assess lung cancer in a biological sample. In some embodiments, fewer than or equal to about fifteen lung cancer associated markers are used to assess lung cancer in a biological sample. In some embodiments, fewer than or equal to about sixteen lung cancer associated markers are used to assess lung cancer in a biological sample. In some embodiments, fewer than or equal to about seventeen lung cancer associated markers are used to assess lung cancer in a biological sample. In some embodiments, fewer than or equal to about eighteen lung cancer associated markers are used to assess lung cancer in a biological sample. In some embodiments, fewer than or equal to about nineteen lung cancer associated markers are used to assess lung cancer in a biological sample. In some embodiments, fewer than or equal to about twenty lung cancer associated markers are used to assess lung cancer in a biological sample. In some embodiments, fewer than or equal to about twenty-one lung cancer associated markers are used to assess lung cancer in a biological sample. In some embodiments, fewer than or equal to about twenty-two lung cancer associated markers are used to assess lung cancer in a biological sample. In some embodiments, fewer than or equal to about twenty-three lung cancer associated markers are used to assess lung cancer in a biological sample. In some embodiments, fewer than or equal to about twenty-four lung cancer associated markers are used to assess lung cancer in a biological sample. In some embodiments, fewer than or equal to about twenty-five lung cancer associated markers are used to assess lung cancer in a biological sample. In some embodiments, fewer than or equal to about thirty lung cancer associated markers are used to assess lung cancer in a biological sample. In some embodiments, fewer than or equal to about thirty-five lung cancer associated markers are used to assess lung cancer in a biological sample. In some embodiments, fewer than or equal to about forty lung cancer associated markers are used to assess lung cancer in a biological sample. In some embodiments, fewer than or equal to about forty-five lung cancer associated markers are used to assess lung cancer in a biological sample. In some embodiments, fewer than or equal to about fifty lung cancer associated markers are used to assess lung cancer in a biological sample. In some embodiments, fewer than or equal to about fifty-five lung cancer associated markers are used to assess lung cancer in a biological sample. In some embodiments, fewer than or equal to about sixty lung cancer associated markers are used to assess lung cancer in a biological sample. In some embodiments, fewer than or equal to about sixty-five lung cancer associated markers are used to assess lung cancer in a biological sample. In some embodiments, fewer than or equal to about seventy lung cancer associated markers are used to assess lung cancer in a biological sample. In some embodiments, fewer than or equal to about seventy-five lung cancer associated markers are used to assess lung cancer in a biological sample. In some embodiments, fewer than or equal to about eighty lung cancer associated markers are used to assess lung cancer in a biological sample. In some embodiments, fewer than or equal to about eighty-five lung cancer associated markers are used to assess lung cancer in a biological sample. In some embodiments, fewer than or equal to about ninety lung cancer associated markers are used to assess lung cancer in a biological sample. In some embodiments, fewer than or equal to about ninety-five lung cancer associated markers are used to assess lung cancer in a biological sample. In some embodiments, fewer than or equal to about one-hundred lung cancer associated markers are used to assess lung cancer in a biological sample.

In some embodiments, the lung cancer associated marker is used to assess lung cancer based on the presence, absence, amount, concentration, count, expression, or any combination thereof of the lung cancer associated marker. In some embodiments, the lung cancer associated marker is used to assess lung cancer based on the presence of the lung cancer associated marker. A lung cancer associated marker may be considered present when the lung cancer associated marker is detected above a threshold. In some embodiments, the lung cancer associated marker is used to assess lung cancer based on the absence of the lung cancer associated marker. A lung cancer associated marker may be considered absent when the lung cancer associated marker is detected below a threshold. In some embodiments, the lung cancer associated marker is used to assess lung cancer based on the amount of the lung cancer associated marker. In some embodiments, the lung cancer associated marker is used to assess lung cancer based on the concentration of the lung cancer associated marker. In some embodiments, the lung cancer associated marker is used to assess lung cancer based on the count of the lung cancer associated marker. In some embodiments, the lung cancer associated marker is used to assess lung cancer based on the expression of the lung cancer associated marker. As a non-limiting example one lung cancer associated marker is used to assess lung cancer based on the presence of the lung cancer associated marker and another lung cancer associated marker is used to assess lung cancer based on the concentration of the another lung cancer associated marker.

In some embodiments, the lung cancer associated markers are proteomic markers. In some embodiments, the proteomic marker comprises information about a protein, a polypeptide, a peptide, a proteoform, or any combination thereof. In some embodiments, the proteomic marker comprises a fragment of a protein, a polypeptide, a peptide, a proteoform, or any combination thereof.

In some embodiments, the lung cancer associated proteomic markers comprise Keratin, type I cytoskeletal 19 (KRT19), Glycoprotein 130 (GP130), Complement Component C9 (C9), Cell adhesion molecule (CEA), CA-125 Antigen (CA125), Myoglobin (MB), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, a proxy proteomic marker thereof, or any combination thereof. In some embodiments, Fragment of Cytokeratin 19 (CYFRA21-1) is the lung cancer associated maker from Keratin, type I cytoskeletal 19. In some embodiments, SAA is Serum amyloid A-1 protein (SAA1). In some embodiments, SAA is Serum amyloid A-2 protein (SAA2). In some embodiments, SAA is Serum amyloid A-3 protein (SAA3). In some embodiments, SAA is Serum amyloid A-4 protein (SAA4). In some embodiments, the lung cancer associated proteomic marker comprises SAA, and SAA comprises information on one or more of SAA1, SAA2, SAA3, SAA4, or any combination thereof. In some embodiments, the lung cancer associated proteomic markers comprise information on one or more proteins or fragments thereof provided above. In some embodiments, the lung cancer associated proteomic markers comprise information on one or more protein or fragments thereof provided above by assessing a protein, polypeptide, peptide, or fragment associated with the one or more protein fragments thereof. In some embodiments, the lung cancer associated proteomic markers comprise an amino acid sequence provided in Table 1. In some embodiments, the lung cancer associated proteomic markers comprise an amino acid sequence provided in Table 1 that is truncated. In some embodiments, the truncated sequence is truncated at the C-terminus, the N-terminus, or both. In some embodiments, the lung cancer associated proteomic markers comprise a variant of an amino acid sequence provided in Table 1. In some embodiments, the variant is an insertion, a deletion, or a frameshift mutation. In some embodiments, the variant has a post-translational modification, such as a phosphorylation, glycosylation, ubiquitination, acetylation, methylation, lipidation, or proteolytic cleavage of a portion of the amino acid sequence, or a combination thereof. In some embodiments, the lung cancer associated proteomic markers comprise an isoform of one or more proteomic markers provided in Table 1 resulting, for example, from alternative splicing variant of the gene encoding the proteomic marker. In some embodiments, a proteomic marker has an amino acid sequence that is at least 70%, 80%, 85%, 90%, 91%, 92%, 93%, 94%, 95%, 96%, 97%, 98% or 99% identical to any one of SEQ ID NOs: 1-13. In some embodiments, a proteomic marker has an amino acid sequence that is at least 70%, 80%, 85%, 90%, 91%, 92%, 93%, 94%, 95%, 96%, 97%, 98% or 99% homologous to any one of SEQ ID NOs: 1-13.

TABLE 1
Lung Cancer Associated Proteomic Marker Sequences

| | | SEQ | |
|---|---|---|---|
| Protein | UniProt | ID | |
| Name | Accession | NO: | Sequence |
| Fibrinogen- | Q08830 | 1 | MAKVFSFILVTTALTMGREISALEDCAQEQMRLRAQVR |
| like protein 1 | | | LLETRVKQQQVKIKQLLQENEVQFLDKGDENTVIDLGS |
| (FGL1) | | | KRQYADCSEIFNDGYKLSGFYKIKPLQSPAEFSVYCDM |
| | | | SDGGGWTVIQRRSDGSENFNRGWKDYENGFGNFVQK |
| | | | HGEYWLGNKNLHFLTTQEDYTLKIDLADFEKNSRYAQ |
| | | | YKNFKVGDEKNFYELNIGEYSGTAGDSLAGNFHPEVQ |
| | | | WWASHQRMKFSTWDRDHDNYEGNCAEEDQSGWWFN |
| | | | RCHSANLNGVYYSGPYTAKTDNGIVWYTWHGWWYS |
| | | | LKSVVMKIRPNDFIPNVI |
| Glycoprotein | Q13514 | 2 | TPKFELKNTSGLMFQILQRVILPSGHLTLLQGTILIQKIK |
| 130 (GP130) | | | CIQMAISLM |
| Complement | P02748 | 3 | MSACRSFAVAICILEISILTAQYTTSYDPELTESSGSASHI |
| component | | | DCRMSPWSEWSQCDPCLRQMFRSRSIEVFGQFNGKRC |
| C9 (C9) | | | TDAVGDRRQCVPTEPCEDAEDDCGNDFQCSTGRCIKM |
| | | | RLRCNGDNDCGDFSDEDDCESEPRPPCRDRVVEESELA |
| | | | RTAGYGINILGMDPLSTPFDNEFYNGLCNRDRDGNTLT |
| | | | YYRRPWNVASLIYETKGEKNFRTEHYEEQIEAFKSIIQE |
| | | | KTSNFNAAISLKFTPTETNKAEQCCEETASSISLHGKGSF |
| | | | RFSYSKNETYQLFLSYSSKKEKMFLHVKGEIHLGRFVM |
| | | | RNRDVVLTTTFVDDIKALPTTYEKGEYFAFLETYGTHY |
| | | | SSSGSLGGLYELIYVLDKASMKRKGVELKDIKRCLGYH |
| | | | LDVSLAFSEISVGAEFNKDDCVKRGEGRAVNITSENLID |
| | | | DVVSLIRGGTRKYAFELKEKLLRGTVIDVTDFVNWASS |
| | | | INDAPVLISQKLSPIYNLVPVKMKNAHLKKQNLERAIED |
| | | | YINEFSVRKCHTCQNGGTVILMDGKCLCACPFKFEGIA |
| | | | CEISKQKISEGLPALEFPNEK |
| Platelet | P16284 | 4 | MQPRWAQGATMWLGVLLTLLLCSSLEGQENSFTINSV |
| endothelial | | | DMKSLPDWTVQNGKNLTLQCFADVSTTSHVKPQHQM |
| cell adhesion | | | LFYKDDVLFYNISSMKSTESYFIPEVRIYDSGTYKCTVIV |
| molecule | | | NNKEKTTAEYQVLVEGVPSPRVTLDKKEAIQGGIVRVN |
| (PECAM-1) | | | CSVPEEKAPIHFTIEKLELNEKMVKLKREKNSRDQNFVI |
| | | | LEFPVEEQDRVLSFRCQARIISGIHMQTSESTKSELVTVT |
| | | | ESFSTPKFHISPTGMIMEGAQLHIKCTIQVTHLAQEFPEII |
| | | | IQKDKAIVAHNRHGNKAVYSVMAMVEHSGNYTCKVE |
| | | | SSRISKVSSIVVNITELFSKPELESSFTHLDQGERLNLSCS |
| | | | IPGAPPANFTIQKEDTIVSQTQDFTKIASKSDSGTYICTA |
| | | | GIDKVVKKSNTVQIVVCEMLSQPRISYDAQFEVIKGQTI |
| | | | EVRCESISGTLPISYQLLKTSKVLENSTKNSNDPAVFKD |
| | | | NPTEDVEYQCVADNCHSHAKMLSEVLRVKVIAPVDEV |
| | | | QISILSSKVVESGEDIVLQCAVNEGSGPITYKFYREKEG |
| | | | KPFYQMTSNATQAFWTKQKASKEQEGEYYCTAFNRA |
| | | | NHASSVPRSKILTVRVILAPWKKGLIAVVIIGVIIALLIIA |
| | | | AKCYFLRKAKAKQMPVEMSRPAVPLLNSNNEKMSDP |
| | | | NMEANSHYGHNDDVRNHAMKPINDNKEPLNSDVQYT |
| | | | EVQVSSAESHKDLGKKDTETVYSEVRKAVPDAVESRY |
| | | | SRTEGSLDGT |
| Myoglobin | P02144 | 5 | MGLSDGEWQLVLNVWGKVEADIPGHGQEVLIRLFKGH |
| (MB) | | | PETLEKFDKFKHLKSEDEMKASEDLKKHGATVLTALG |
| | | | GILKKKGHHEAEIKPLAQSHATKHKIPVKYLEFISECIIQ |
| | | | VLQSKHPGDFGADAQGAMNKALELFRKDMASNYKEL |
| | | | GFQG |
| Insulin-like | P24592 | 6 | MTPHRLLPPLLLLLALLLAASPGGALARCPGCGQGVQA |
| growth | | | GCPGGCVEEEDGGSPAEGCAEAEGCLRREGQECGVYT |
| factor- | | | PNCAPGLQCHPPKDDEAPLRALLLGRGRCLPARAPAVA |
| binding | | | EENPKESKPQAGTARPQDVNRRDQQRNPGTSTTPSQPN |
| protein 6 | | | SAGVQDTEMGPCRRHLDSVLQQLQTEVYRGAQTLYVP |
| (IGFBP-6) | | | NCDHRGFYRKRQCRSSQGQRRGPCWCVDRMGKSLPG |
| | | | SPDGNGSSSCPTGSSG |
| Keratin, type | P08727 | 7 | MTSYSYRQSSATSSFGGLGGGSVRFGPGVAFRAPSIHG |
| I cytoskeletal | | | GSGGRGVSVSSARFVSSSSSGAYGGGYGGVLTASDGLL |
| 19 (KRT19) | | | AGNEKLTMQNLNDRLASYLDKVRALEAANGELEVKIR |
| | | | DWYQKQGPGPSRDYSHYYTTIQDLRDKILGATIENSRI |
| | | | VLQIDNARLAADDFRTKFETEQALRMSVEADINGLRRV |
| | | | LDELTLARTDLEMQIEGLKEELAYLKKNHEEEISTLRG |
| | | | QVGGQVSVEVDSAPGTDLAKILSDMRSQYEVMAEQNR |
| | | | KDAEAWFTSRTEELNREVAGHTEQLQMSRSEVTDLRR |
| | | | TLQGLEIELQSQLSMKAALEDTLAETEARFGAQLAHIQ |
| | | | ALISGIEAQLGDVRADSERQNQEYQRLMDIKSRLEQEIA |
| | | | TYRSLLEGQEDHYNNLSASKVL |
| Gamma- | P09104 | 8 | MSIEKIWAREILDSRGNPTVEVDLYTAKGLFRAAVPSG |
| enolase | | | ASTGIYEALELRDGDKQRYLGKGVLKAVDHINSTIAPA |
| (ENO 2) | | | LISSGLSVVEQEKLDNLMLELDGTENKSKFGANAILGV |
| | | | SLAVCKAGAAERELPLYRHIAQLAGNSDLILPVPAFNVI |
| | | | NGGSHAGNKLAMQEFMILPVGAESFRDAMRLGAEVY |
| | | | HTLKGVIKDKYGKDATNVGDEGGFAPNILENSEALELV |
| | | | KEAIDKAGYTEKIVIGMDVAASEFYRDGKYDLDFKSPT |
| | | | DPSRYITGDQLGALYQDFVRDYPVVSIEDPFDQDDWA |
| | | | AWSKFTANVGIQIVGDDLTVTNPKRIERAVEEKACNCL |
| | | | LLKVNQIGSVTEAIQACKLAQENGWGVMVSHRSGETE |
| | | | DTFIADLVVGLCTGQIKTGAPCRSERLAKYNQLMRIEE |
| | | | ELGDEARFAGHNFRNPSVL |
| Cell | P06731 | 9 | MESPSAPPHRWCIPWQRLLLTASLLTFWNPPTTAKLTIE |
| Adhesion | | | STPFNVAEGKEVLLLVHNLPQHLFGYSWYKGERVDGN |
| Molecule | | | RQIIGYVIGTQQATPGPAYSGREIIYPNASLLIQNIIQNDT |
| (CEA) | | | GFYTLHVIKSDLVNEEATGQFRVYPELPKPSISSNNSKP |
| | | | VEDKDAVAFTCEPETQDATYLWWVNNQSLPVSPRLQL |
| | | | SNGNRTLTLFNVTRNDTASYKCETQNPVSARRSDSVIL |
| | | | NVLYGPDAPTISPLNTSYRSGENLNLSCHAASNPPAQYS |
| | | | WFVNGTFQQSTQELFIPNITVNNSGSYTCQAHNSDTGL |
| | | | NRTTVTTITVYAEPPKPFITSNNSNPVEDEDAVALTCEP |
| | | | EIQNTTYLWWVNNQSLPVSPRLQLSNDNRTLTLLSVTR |
| | | | NDVGPYECGIQNELSVDHSDPVILNVLYGPDDPTISPSY |
| | | | TYYRPGVNLSLSCHAASNPPAQYSWLIDGNIQQHTQEL |
| | | | FISNITEKNSGLYTCQANNSASGHSRTTVKTITVSAELP |
| | | | KPSISSNNSKPVEDKDAVAFTCEPEAQNTTYLWWVNG |
| | | | QSLPVSPRLQLSNGNRTLTLFNVTRNDARAYVCGIQNS |
| | | | VSANRSDPVTLDVLYGPDTPIISPPDSSYLSGANLNLSC |
| | | | HSASNPSPQYSWRINGIPQQHTQVLFIAKITPNNNGTYA |
| | | | CFVSNLATGRNNSIVKSITVSASGTSPGLSAGATVGIMI |
| | | | GVLVGVALI |
| Serum | PODJI8 | 10 | MKLLTGLVFCSLVLGVSSRSFFSFLGEAFDGARDMWR |
| amyloid A-1 | | | AYSDMREANYIGSDKYFHARGNYDAAKRGPGGAWAA |
| protein | | | EVITDARENIQRFFGHGAEDSLADQAANEWGRSGKDP |
| (SAA1) | | | NHFRPAGLPEKY |
| Serum | PODJI9 | 11 | MKLLTGLVFCSLVLSVSSRSFFSFLGEAFDGARDMWRA |
| amyloid A-2 | | | YSDMREANYIGSDKYFHARGNYDAAKRGPGGAWAAE |
| protein | | | VISNARENIQRLTGRGAEDSLADQAANKWGRSGRDPN |
| (SAA2) | | | HFRPAGLPEKY |
| Serum | P35542 | 12 | MRLFTGIVFCSLVMGVTSESWRSFFKEALQGVGDMGR |
| amyloid A-4 | | | AYWDIMISNHQNSNRYLYARGNYDAAQRGPGGVWAA |
| protein | | | KLISRSRVYLQGLIDCYLFGNSSTVLEDSKSNEKAEEW |
| (SAA4) | | | GRSGKDPDRFRPDGLPKKY |
| CA-125 | Q8WXI7 | 13 | MLKPSGLPGSSSPTRSLMTGSRSTKATPEMDSGLTGAT |
| Antigen | | | LSPKTSTGAIVVTEHTLPFTSPDKTLASPTSSVVGRTTQS |
| (CA125) | | | LGVMSSALPESTSRGMTHSEQRTSPSLSPQVNGTPSRN |
| | | | YPATSMVSGLSSPRTRTSSTEGNFTKEASTYTLTVETTS |
| | | | GPVTEKYTVPTETSTTEGDSTETPWDTRYIPVKITSPMK |
| | | | TFADSTASKENAPVSMTPAETTVTDSHTPGRTNPSFGTL |
| | | | YSSFLDLSPKGTPNSRGETSLELILSTTGYPFSSPEPGSA |
| | | | GHSRISTSAPLSSSASVLDNKISETSIFSGQSLTSPLSPGV |
| | | | PEARASTMPNSAIPFSMTLSNAETSAERVRSTISSLGTPS |
| | | | ISTKQTAETILTFHAFAETMDIPSTHIAKTLASEWLGSPG |
| | | | TLGGTSTSALTTTSPSTTLVSEETNTHHSTSGKETEGTL |
| | | | NTSMTPLETSAPGEESEMTATLVPTLGFTTLDSKIRSPS |
| | | | QVSSSHPTRELRTTGSTSGRQSSSTAAHGSSDILRATTSS |
| | | | TSKASSWTSESTAQQFSEPQHTQWVETSPSMKTERPPA |
| | | | STSVAAPITTSVPSVVSGFTTLKTSSTKGIWLEETSADTL |
| | | | IGESTAGPTTHQFAVPTGISMTGGSSTRGSQGTTHLLTR |
| | | | ATASSETSADLTLATNGVPVSVSPAVSKTAAGSSPPGG |
| | | | TKPSYTMVSSVIPETSSLQSSAFREGTSLGLTPLNTRHPF |
| | | | SSPEPDSAGHTKISTSIPLLSSASVLEDKVSATSTFSHHK |
| | | | ATSSITTGTPEISTKTKPSSAVLSSMTLSNAATSPERVRN |
| | | | ATSPLTHPSPSGEETAGSVLTLSTSAETTDSPNIHPTGTL |
| | | | TSESSESPSTLSLPSVSGVKTTFSSSTPSTHLFTSGEETEE |
| | | | TSNPSVSQPETSVSRVRTTLASTSVPTPVFPTMDTWPTR |
| | | | SAQFSSSHLVSELRATSSTSVINSTGSALPKISHLTGTAT |
| | | | MSQTNRDTFNDSAAPQSTTWPETSPRFKTGLPSATTTV |
| | | | STSATSLSATVMVSKFTSPATSSMEATSIREPSTTILTTE |
| | | | TTNGPGSMAVASTNIPIGKGYITEGRLDTSHLPIGTTASS |
| | | | ETSMDFTMAKESVSMSVSPSQSMDAAGSSTPGRTSQFV |
| | | | DTFSDDVYHLTSREITIPRDGTSSALTPQMTATHPPSPDP |
| | | | GSARSTWLGILSSSPSSPTPKVTMSSTFSTQRVTTSMIM |
| | | | DTVETSRWNMPNLPSTTSLTPSNIPTSGAIGKSTLVPLD |
| | | | TPSPATSLEASEGGLPTLSTYPESTNTPSIHLGAHASSES |
| | | | PSTIKLTMASVVKPGSYTPLTFPSIETHIHVSTARMAYSS |
| | | | GSSPEMTAPGETNTGSTWDPTTYITTTDPKDTSSAQVST |
| | | | PHSVRTLRTTENHPKTESATPAAYSGSPKISSSPNLTSPA |
| | | | TKAWTITDTTEHSTQLHYTKLAEKSSGFETQSAPGPVS |
| | | | VVIPTSPTIGSSTLELTSDVPGEPLVLAPSEQTTITLPMAT |
| | | | WLSTSLTEEMASTDLDISSPSSPMSTFAIFPPMSTPSHEL |
| | | | SKSEADTSAIRNTDSTTLDQHLGIRSLGRTGDLTTVPITP |
| | | | LTTTWTSVIEHSTQAQDTLSATMSPTHVTQSLKDQTSIP |
| | | | ASASPSHLTEVYPELGTQGRSSSEATTFWKPSTDTLSRE |
| | | | IETGPTNIQSTPPMDNTTTGSSSSGVTLGIAHLPIGTSSPA |
| | | | ETSTNMALERRSSTATVSMAGTMGLLVTSAPGRSISQS |
| | | | LGRVSSVLSESTTEGVTDSSKGSSPRLNTQGNTALSSSL |
| | | | EPSYAEGSQMSTSIPLTSSPTTPDVEFIGGSTFWTKEVTT |
| | | | VMTSDISKSSARTESSSATLMSTALGSTENTGKEKLRTA |
| | | | SMDLPSPTPSMEVTPWISLTLSNAPNTTDSLDLSHGVHT |
| | | | SSAGTLATDRSLNTGVTRASRLENGSDTSSKSLSMGNS |
| | | | THTSMTYTEKSEVSSSIHPRPETSAPGAETTLTSTPGNR |
| | | | AISLTLPFSSIPVEEVISTGITSGPDINSAPMTHSPITPPTIV |
| | | | WTSTGTIEQSTQPLHAVSSEKVSVQTQSTPYVNSVAVS |
| | | | ASPTHENSVSSGSSTSSPYSSASLESLDSTISRRNAITSWL |
| | | | WDLTTSLPTTTWPSTSLSEALSSGHSGVSNPSSTTTEFPL |
| | | | FSAASTSAAKQRNPETETHGPQNTAASTLNTDASSVTG |
| | | | LSETPVGASISSEVPLPMAITSRSDVSGLTSESTANPSLG |
| | | | TASSAGTKLTRTISLPTSESLVSFRMNKDPWTVSIPLGS |
| | | | HPTTNTETSIPVNSAGPPGLSTVASDVIDTPSDGAESIPT |
| | | | VSFSPSPDTEVTTISHFPEKTTHSFRTISSLTHELTSRVTPI |
| | | | PGDWMSSAMSTKPTGASPSITLGERRTITSAAPTTSPIVL |
| | | | TASFTETSTVSLDNETTVKTSDILDARKTNELPSDSSSSS |
| | | | DLINTSIASSTMDVTKTASISPTSISGMTASSSPSLFSSDR |
| | | | PQVPTSTTETNTATSPSVSSNTYSLDGGSNVGGTPSTLP |
| | | | PFTITHPVETSSALLAWSRPVRTFSTMVSTDTASGENPT |
| | | | SSNSVVTSVPAPGTWTSVGSTTDLPAMGFLKTSPAGEA |
| | | | HSLLASTIEPATAFTPHLSAAVVTGSSATSEASLLTTSES |
| | | | KAIHSSPQTPTTPTSGANWETSATPESLLVVTETSDTTL |
| | | | TSKILVTDTILFSTVSTPPSKFPSTGTLSGASFPTLLPDTP |
| | | | AIPLTATEPTSSLATSFDSTPLVTIASDSLGTVPETTLTMS |
| | | | ETSNGDALVLKTVSNPDRSIPGITIQGVTESPLHPSSTSPS |
| | | | KIVAPRNTTYEGSITVALSTLPAGTTGSLVFSQSSENSET |
| | | | TALVDSSAGLERASVMPLTTGSQGMASSGGIRSGSTHS |
| | | | TGTKTFSSLPLTMNPGEVTAMSEITTNRLTATQSTAPKG |
| | | | IPVKPTSAESGLLTPVSASSSPSKAFASLTTAPPTWGIPQ |
| | | | STLTFEFSEVPSLDTKSASLPTPGQSLNTIPDSDASTASSS |
| | | | LSKSPEKNPRARMMTSTKAISASSFQSTGFTETPEGSAS |
| | | | PSMAGHEPRVPTSGTGDPRYASESMSYPDPSKASSAMT |
| | | | STSLASKLTTLFSTGQAARSGSSSSPISLSTEKETSFLSPT |
| | | | ASTSRKTSLFLGPSMARQPNILVHLQTSALTLSPTSTLN |
| | | | MSQEEPPELTSSQTIAEEEGTTAETQTLTFTPSETPTSLLP |
| | | | VSSPTEPTARRKSSPETWASSISVPAKTSLVETTDGTLV |
| | | | TTIKMSSQAAQGNSTWPAPAEETGSSPAGTSPGSPEMS |
| | | | TTLKIMSSKEPSISPEIRSTVRNSPWKTPETTVPMETTVE |
| | | | PVTLQSTALGSGSTSISHLPTGTTSPTKSPTENMLATERV |
| | | | SLSPSPPEAWTNLYSGTPGGTRQSLATMSSVSLESPTAR |
| | | | SITGTGQQSSPELVSKTTGMEFSMWHGSTGGTTGDTHV |
| | | | SLSTSSNILEDPVTSPNSVSSLTDKSKHKTETWVSTTAIP |
| | | | STVLNNKIMAAEQQTSRSVDEAYSSTSSWSDQTSGSDI |
| | | | TLGASPDVTNTLYITSTAQTTSLVSLPSGDQGITSLTNPS |
| | | | GGKTSSASSVTSPSIGLETLRANVSAVKSDIAPTAGHLS |
| | | | QTSSPAEVSILDVTTAPTPGISTTITTMGTNSISTTTPNPE |
| | | | VGMSTMDSTPATERRTTSTEHPSTWSSTAASDSWTVTD |
| | | | MTSNLKVARSPGTISTMHTTSFLASSTELDSMSTPHGRI |
| | | | TVIGTSLVTPSSDASAVKTETSTSERTLSPSDTTASTPIST |
| | | | FSRVQRMSISVPDILSTSWTPSSTEAEDVPVSMVSTDHA |
| | | | STKTDPNTPLSTFLFDSLSTLDWDTGRSLSSATATTSAP |
| | | | QGATTPQELTLETMISPATSQLPFSIGHITSAVTPAAMA |
| | | | RSSGVTFSRPDPTSKKAEQTSTQLPTTTSAHPGQVPRSA |
| | | | ATTLDVIPHTAKTPDATFQRQGQTALTTEARATSDSWN |
| | | | EKEKSTPSAPWITEMMNSVSEDTIKEVTSSSSVLRTLNT |
| | | | LDINLESGTTSSPSWKSSPYERIAPSESTTDKEAIHPSTNT |
| | | | VETTGWVTSSEHASHSTIPAHSASSKLTSPVVTTSTREQ |
| | | | AIVSMSTTTWPESTRARTEPNSFLTIELRDVSPYMDTSS |
| | | | TTQTSIISSPGSTAITKGPRTEITSSKRISSSFLAQSMRSSD |
| | | | SPSEAITRLSNFPAMTESGGMILAMQTSPPGATSLSAPT |
| | | | LDTSATASWTGTPLATTQRFTYSEKTTLFSKGPEDTSQP |
| | | | SPPSVEETSSSSSLVPIHATTSPSNILLTSQGHSPSSTPPVT |
| | | | SVFLSETSGLGKTTDMSRISLEPGTSLPPNLSSTAGEALS |
| | | | TYEASRDTKAIHHSADTAVTNMEATSSEYSPIPGHTKPS |
| | | | KATSPLVTSHIMGDITSSTSVFGSSETTEIETVSSVNQGL |
| | | | QERSTSQVASSATETSTVITHVSSGDATTHVTKTQATFS |
| | | | SGTSISSPHQFITSTNTFTDVSTNPSTSLIMTESSGVTITT |
| | | | QTGPTGAATQGPYLLDTSTMPYLTETPLAVTPDFMQSE |
| | | | KTTLISKGPKDVSWTSPPSVAETSYPSSLTPFLVTTIPPA |
| | | | TSTLQGQHTSSPVSATSVLTSGLVKTTDMLNTSMEPVT |
| | | | NSPQNLNNPSNEILATLAATTDIETIHPSINKAVTNMGT |
| | | | ASSAHVLHSTLPVSSEPSTATSPMVPASSMGDALASISIP |
| | | | GSETTDIEGEPTSSLTAGRKENSTLQEMNSTTESNIILSN |
| | | | VSVGAITEATKMEVPSFDATFIPTPAQSTKFPDIFSVASS |
| | | | RLSNSPPMTISTHMTTTQTGSSGATSKIPLALDTSTLETS |
| | | | AGTPSVVTEGFAHSKITTAMNNDVKDVSQTNPPFQDEA |
| | | | SSPSSQAPVLVTTLPSSVAFTPQWHSTSSPVSMSSVLTSS |
| | | | LVKTAGKVDTSLETVTSSPQSMSNTLDDISVTSAATTDI |
| | | | ETTHPSINTVVTNVGTTGSAFESHSTVSAYPEPSKVTSP |
| | | | NVTTSTMEDTTISRSIPKSSKTTRTETETTSSLTPKLRETS |
| | | | ISQEITSSTETSTVPYKELTGATTEVSRTDVTSSSSTSFPG |
| | | | PDQSTVSLDISTETNTRLSTSPIMTESAEITITTQTGPHGA |
| | | | TSQDTFTMDPSNTTPQAGIHSAMTHGFSQLDVTTLMSR |
| | | | IPQDVSWTSPPSVDKTSSPSSFLSSPAMTTPSLISSTLPED |
| | | | KLSSPMTSLLTSGLVKITDILRTRLEPVTSSLPNFSSTSD |
| | | | KILATSKDSKDTKEIFPSINTEETNVKANNSGHESHSPAL |
| | | | ADSETPKATTQMVITTTVGDPAPSTSMPVHGSSETTNIK |
| | | | REPTYFLTPRLRETSTSQESSFPTDTSFLLSKVPTGTITEV |
| | | | SSTGVNSSSKISTPDHDKSTVPPDTFTGEIPRVFTSSIKTK |
| | | | SAEMTITTQASPPESASHSTLPLDTSTTLSQGGTHSTVT |
| | | | QGFPYSEVTTLMGMGPGNVSWMTTPPVEETSSVSSLM |
| | | | SSPAMTSPSPVSSTSPQSIPSSPLPVTALPTSVLVTTTDVL |
| | | | GTTSPESVTSSPPNLSSITHERPATYKDTAHTEAAMHHS |
| | | | TNTAVTNVGTSGSGHKSQSSVLADSETSKATPLMSTTS |
| | | | TLGDTSVSTSTPNISQTNQIQTEPTASLSPRLRESSTSEKT |
| | | | SSTTETNTAFSYVPTGAITQASRTEISSSRTSISDLDRPTI |
| | | | APDISTGMITRLFTSPIMTKSAEMTVTTQTTTPGATSQGI |
| | | | LPWDTSTTLFQGGTHSTVSQGFPHSEITTLRSRTPGDVS |
| | | | WMTTPPVEETSSGFSLMSPSMTSPSPVSSTSPESIPSSPLP |
| | | | VTALLTSVLVTTTNVLGTTSPEPVTSSPPNLSSPTQERLT |
| | | | TYKDTAHTEAMHASMHTNTAVANVGTSISGHESQSSV |
| | | | PADSHTSKATSPMGITFAMGDTSVSTSTPAFFETRIQTES |
| | | | TSSLIPGLRDTRTSEEINTVTETSTVLSEVPTTTTTEVSRT |
| | | | EVITSSRTTISGPDHSKMSPYISTETITRLSTFPFVTGSTE |
| | | | MAITNQTGPIGTISQATLTLDTSSTASWEGTHSPVTQRF |
| | | | PHSEETTTMSRSTKGVSWQSPPSVEETSSPSSPVPLPAIT |
| | | | SHSSLYSAVSGSSPTSALPVTSLLTSGRRKTIDMLDTHS |
| | | | ELVTSSLPSASSFSGEILTSEASTNTETIHFSENTAETNM |
| | | | GTTNSMHKLHSSVSIHSQPSGHTPPKVTGSMMEDAIVS |
| | | | TSTPGSPETKNVDRDSTSPLTPELKEDSTALVMNSTTES |
| | | | NTVFSSVSLDAATEVSRAEVTYYDPTFMPASAQSTKSP |
| | | | DISPEASSSHSNSPPLTISTHKTIATQTGPSGVTSLGQLTL |
| | | | DTSTIATSAGTPSARTQDFVDSETTSVMNNDLNDVLKT |
| | | | SPFSAEEANSLSSQAPLLVTTSPSPVTSTLQEHSTSSLVS |
| | | | VTSVPTPTLAKITDMDTNLEPVTRSPQNLRNTLATSEAT |
| | | | TDTHTMHPSINTAVANVGTTSSPNEFYFTVSPDSDPYK |
| | | | ATSAVVITSTSGDSIVSTSMPRSSAMKKIESETTFSLIFRL |
| | | | RETSTSQKIGSSSDTSTVFDKAFTAATTEVSRTELTSSSR |
| | | | TSIQGTEKPTMSPDTSTRSVTMLSTFAGLTKSEERTIAT |
| | | | QTGPHRATSQGTLTWDTSITTSQAGTHSAMTHGFSQLD |
| | | | LSTLTSRVPEYISGTSPPSVEKTSSSSSLLSLPAITSPSPVP |
| | | | TTLPESRPSSPVHLTSLPTSGLVKTTDMLASVASLPPNL |
| | | | GSTSHKIPTTSEDIKDTEKMYPSTNIAVTNVGTTTSEKES |
| | | | YSSVPAYSEPPKVTSPMVTSFNIRDTIVSTSMPGSSEITRI |
| | | | EMESTFSLAHGLKGTSTSQDPIVSTEKSAVLHKLTTGAT |
| | | | ETSRTEVASSRRTSIPGPDHSTESPDISTEVIPSLPISLGIT |
| | | | ESSNMTIITRTGPPLGSTSQGTFTLDTPTTSSRAGTHSMA |
| | | | TQEFPHSEMTTVMNKDPEILSWTIPPSIEKTSFSSSLMPS |
| | | | PAMTSPPVSSTLPKTIHTTPSPMTSLLTPSLVMTTDTLGT |
| | | | SPEPTTSSPPNLSSTSHEILTTDEDTTAIEAMHPSTSTAAT |
| | | | NVETTSSGHGSQSSVLADSEKTKATAPMDTTSTMGHTT |
| | | | VSTSMSVSSETTKIKRESTYSLTPGLRETSISQNASFSTD |
| | | | TSIVLSEVPTGTTAEVSRTEVTSSGRTSIPGPSQSTVLPEI |
| | | | STRTMTRLFASPTMTESAEMTIPTQTGPSGSTSQDTLTL |
| | | | DTSTTKSQAKTHSTLTQRFPHSEMTTLMSRGPGDMSW |
| | | | QSSPSLENPSSLPSLLSLPATTSPPPISSTLPVTISSSPLPVT |
| | | | SLLTSSPVTTTDMLHTSPELVTSSPPKLSHTSDERLTTGK |
| | | | DTTNTEAVHPSTNTAASNVEIPSSGHESPSSALADSETS |
| | | | KATSPMFITSTQEDTTVAISTPHFLETSRIQKESISSLSPK |
| | | | LRETGSSVETSSAIETSAVLSEVSIGATTEISRTEVTSSSR |
| | | | TSISGSAESTMLPEISTTRKIIKFPTSPILAESSEMTIKTQT |
| | | | SPPGSTSESTFTLDTSTTPSLVITHSTMTQRLPHSEITTLV |
| | | | SRGAGDVPRPSSLPVEETSPPSSQLSLSAMISPSPVSSTLP |
| | | | ASSHSSSASVTSLLTPGQVKTTEVLDASAEPETSSPPSLS |
| | | | STSVEILATSEVTTDTEKIHPFSNTAVTKVGTSSSGHESP |
| | | | SSVLPDSETTKATSAMGTISIMGDTSVSTLTPALSNTRKI |
| | | | QSEPASSLTTRLRETSTSEETSLATEANTVLSKVSTGAT |
| | | | TEVSRTEAISFSRTSMSGPEQSTMSQDISIGTIPRISASSV |
| | | | LTESAKMTITTQTGPSESTLESTLNLNTATTPSWVETHSI |
| | | | VIQGFPHPEMTTSMGRGPGGVSWPSPPFVKETSPPSSPL |
| | | | SLPAVTSPHPVSTTFLAHIPPSPLPVTSLLTSGPATTTDIL |
| | | | GTSTEPGTSSSSSLSTTSHERLTTYKDTAHTEAVHPSTN |
| | | | TGGTNVATTSSGYKSQSSVLADSSPMCTTSTMGDTSVL |
| | | | TSTPAFLETRRIQTELASSLTPGLRESSGSEGTSSGTKMS |
| | | | TVLSKVPTGATTEISKEDVTSIPGPAQSTISPDISTRTVS |
| | | | WFSTSPVMTESAEITMNTHTSPLGATTQGTSTLDTSSTT |
| | | | SLTMTHSTISQGFSHSQMSTLMRRGPEDVSWMSPPLLE |
| | | | KTRPSFSLMSSPATTSPSPVSSTLPESISSSPLPVTSLLTSG |
| | | | LAKTTDMLHKSSEPVTNSPANLSSTSVEILATSEVTTDT |
| | | | EKTHPSSNRTVTDVGTSSSGHESTSFVLADSQTSKVTSP |
| | | | MVITSTMEDTSVSTSTPGFFETSRIQTEPTSSLTLGLRKT |
| | | | SSSEGTSLATEMSTVLSGVPTGATAEVSRTEVTSSSRTSI |
| | | | SGFAQLTVSPETSTETITRLPTSSIMTESAEMMIKTQTDP |
| | | | PGSTPESTHTVDISTTPNWVETHSTVTQRFSHSEMTTLV |
| | | | SRSPGDMLWPSQSSVEETSSASSLLSLPATTSPSPVSSTL |
| | | | VEDFPSASLPVTSLLNPGLVITTDRMGISREPGTSSTSNL |
| | | | SSTSHERLTTLEDTVDTEDMQPSTHTAVTNVRTSISGHE |
| | | | SQSSVLSDSETPKATSPMGTTYTMGETSVSISTSDFFETS |
| | | | RIQIEPTSSLTSGLRETSSSERISSATEGSTVLSEVPSGAT |
| | | | TEVSRTEVISSRGTSMSGPDQFTISPDISTEAITRLSTSPI |
| | | | MTESAESAITIETGSPGATSEGTLTLDTSTTTFWSGTHST |
| | | | ASPGFSHSEMTTLMSRTPGDVPWPSLPSVEEASSVSSSL |
| | | | SSPAMTSTSFFSTLPESISSSPHPVTALLTLGPVKTTDML |
| | | | RTSSEPETSSPPNLSSTSAEILATSEVTKDREKIHPSSNTP |
| | | | VVNVGTVIYKHLSPSSVLADLVTTKPTSPMATTSTLGN |
| | | | TSVSTSTPAFPETMMTQPTSSLTSGLREISTSQETSSATE |
| | | | RSASLSGMPTGATTKVSRTEALSLGRTSTPGPAQSTISP |
| | | | EISTETITRISTPLTTTGSAEMTITPKTGHSGASSQGTFTL |
| | | | DTSSRASWPGTHSAATHRSPHSGMTTPMSRGPEDVSW |
| | | | PSRPSVEKTSPPSSLVSLSAVTSPSPLYSTPSESSHSSPLR |
| | | | VTSLFTPVMMKTTDMLDTSLEPVTTSPPSMNITSDESLA |
| | | | TSKATMETEAIQLSENTAVTQMGTISARQEFYSSYPGLP |
| | | | EPSKVTSPVVTSSTIKDIVSTTIPASSEITRIEMESTSTLTP |
| | | | TPRETSTSQEIHSATKPSTVPYKALTSATIEDSMTQVMS |
| | | | SSRGPSPDQSTMSQDISTEVITRLSTSPIKTESTEMTITTQ |
| | | | TGSPGATSRGTLTLDTSTTFMSGTHSTASQGFSHSQMT |
| | | | ALMSRTPGDVPWLSHPSVEEASSASFSLSSPVMTSSSPV |
| | | | SSTLPDSIHSSSLPVTSLLTSGLVKTTELLGTSSEPETSSP |
| | | | PNLSSTSAEILAITEVTTDTEKLEMTNVVTSGYTHESPSS |
| | | | VLADSVTTKATSSMGITYPTGDTNVLTSTPAFSDTSRIQ |
| | | | TKSKLSLTPGLMETSISEETSSATEKSTVLSSVPTGATTE |
| | | | VSRTEAISSSRTSIPGPAQSTMSSDTSMETITRISTPLTRK |
| | | | ESTDMAITPKTGPSGATSQGTFTLDSSSTASWPGTHSAT |
| | | | TQRFPQSVVTTPMSRGPEDVSWPSPLSVEKNSPPSSLVS |
| | | | SSSVTSPSPLYSTPSGSSHSSPVPVTSLFTSIMMKATDML |
| | | | DASLEPETTSAPNMNITSDESLAASKATTETEAIHVFEN |
| | | | TAASHVETTSATEELYSSSPGFSEPTKVISPVVTSSSIRD |
| | | | NMVSTTMPGSSGITRIEIESMSSLTPGLRETRTSQDITSST |
| | | | ETSTVLYKMPSGATPEVSRTEVMPSSRTSIPGPAQSTMS |
| | | | LDISDEVVTRLSTSPIMTESAEITITTQTGYSLATSQVTLP |
| | | | LGTSMTFLSGTHSTMSQGLSHSEMTNLMSRGPESLSWT |
| | | | SPRFVETTRSSSSLTSLPLTTSLSPVSSTLLDSSPSSPLPVT |
| | | | SLILPGLVKTTEVLDTSSEPKTSSSPNLSSTSVEIPATSEI |
| | | | MTDTEKIHPSSNTAVAKVRTSSSVHESHSSVLADSETTI |
| | | | TIPSMGITSAVDDTTVFTSNPAFSETRRIPTEPTFSLTPGF |
| | | | RETSTSEETTSITETSAVLYGVPTSATTEVSMTEIMSSNR |
| | | | IHIPDSDQSTMSPDIITEVITRLSSSSMMSESTQMTITTQK |
| | | | SSPGATAQSTLTLATTTAPLARTHSTVPPRFLHSEMTTL |
| | | | MSRSPENPSWKSSLFVEKTSSSSSLLSLPVTTSPSVSSTL |
| | | | PQSIPSSSFSVTSLLTPGMVKTTDTSTEPGTSLSPNLSGTS |
| | | | VEILAASEVTTDTEKIHPSSSMAVTNVGTTSSGHELYSS |
| | | | VSIHSEPSKATYPVGTPSSMAETSISTSMPANFETTGFEA |
| | | | EPFSHLTSGFRKTNMSLDTSSVTPTNTPSSPGSTHLLQSS |
| | | | KTDFTSSAKTSSPDWPPASQYTEIPVDIITPFNASPSITES |
| | | | TGITSFPESRFTMSVTESTHHLSTDLLPSAETISTGTVMP |
| | | | SLSEAMTSFATTGVPRAISGSGSPFSRTESGPGDATLSTI |
| | | | AESLPSSTPVPFSSSTFTTTDSSTIPALHEITSSSATPYRV |
| | | | DTSLGTESSTTEGRLVMVSTLDTSSQPGRTSSSPILDTR |
| | | | MTESVELGTVTSAYQVPSLSTRLTRTDGIMEHITKIPNE |
| | | | AAHRGTIRPVKGPQTSTSPASPKGLHTGGTKRMETTTT |
| | | | ALKTTTTALKTTSRATLTTSVYTPTLGTLTPLNASMQM |
| | | | ASTIPTEMMITTPYVFPDVPETTSSLATSLGAETSTALPR |
| | | | TTPSVFNRESETTASLVSRSGAERSPVIQTLDVSSSEPDT |
| | | | TASWVIHPAETIPTVSKTTPNFFHSELDTVSSTATSHGA |
| | | | DVSSAIPTNISPSELDALTPLVTISGTDTSTTFPTLTKSPH |
| | | | ETETRTTWLTHPAETSSTIPRTIPNFSHHESDATPSIATSP |
| | | | GAETSSAIPIMTVSPGAEDLVTSQVTSSGTDRNMTIPTL |
| | | | TLSPGEPKTIASLVTHPEAQTSSAIPTSTISPAVSRLVTSM |
| | | | VTSLAAKTSTTNRALTNSPGEPATTVSLVTHPAQTSPTV |
| | | | PWTTSIFFHSKSDTTPSMTTSHGAESSSAVPTPTVSTEVP |
| | | | GVVTPLVTSSRAVISTTIPILTLSPGEPETTPSMATSHGEE |
| | | | ASSAIPTPTVSPGVPGVVTSLVTSSRAVTSTTIPILTFSLG |
| | | | EPETTPSMATSHGTEAGSAVPTVLPEVPGMVTSLVASS |
| | | | RAVTSTTLPTLTLSPGEPETTPSMATSHGAEASSTVPTV |
| | | | SPEVPGVVTSLVTSSSGVNSTSIPTLILSPGELETTPSMAT |
| | | | SHGAEASSAVPTPTVSPGVSGVVTPLVTSSRAVTSTTIPI |
| | | | LTLSSSEPETTPSMATSHGVEASSAVLTVSPEVPGMVTS |
| | | | LVTSSRAVTSTTIPTLTISSDEPETTTSLVTHSEAKMISAI |
| | | | PTLAVSPTVQGLVTSLVTSSGSETSAFSNLTVASSQPETI |
| | | | DSWVAHPGTEASSVVPTLTVSTGEPFTNISLVTHPAESS |
| | | | STLPRTTSRFSHSELDTMPSTVTSPEAESSSAISTTISPGIP |
| | | | GVLTSLVTSSGRDISATFPTVPESPHESEATASWVTHPA |
| | | | VTSTTVPRTTPNYSHSEPDTTPSIATSPGAEATSDFPTIT |
| | | | VSPDVPDMVTSQVTSSGTDTSITIPTLTLSSGEPETTTSFI |
| | | | TYSETHTSSAIPTLPVSPGASKMLTSLVISSGTDSTTTFPT |
| | | | LTETPYEPETTAIQLIHPAETNTMVPRTTPKFSHSKSDTT |
| | | | LPVAITSPGPEASSAVSTTTISPDMSDLVTSLVPSSGTDT |
| | | | STTFPTLSETPYEPETTATWLTHPAETSTTVSGTIPNFSH |
| | | | RGSDTAPSMVTSPGVDTRSGVPTTTIPPSIPGVVTSQVTS |
| | | | SATDTSTAIPTLTPSPGEPETTASSATHPGTQTGFTVPIRT |
| | | | VPSSEPDTMASWVTHPPQTSTPVSRTTSSFSHSSPDATP |
| | | | VMATSPRTEASSAVLTTISPGAPEMVTSQITSSGAATST |
| | | | TVPTLTHSPGMPETTALLSTHPRTETSKTFPASTVFPQV |
| | | | SETTASLTIRPGAETSTALPTQTTSSLFTLLVTGTSRVDL |
| | | | SPTASPGVSAKTAPLSTHPGTETSTMIPTSTLSLGLLETT |
| | | | GLLATSSSAETSTSTLTLTVSPAVSGLSSASITTDKPQTV |
| | | | TSWNTETSPSVTSVGPPEFSRTVTGTTMTLIPSEMPTPPK |
| | | | TSHGEGVSPTTILRTTMVEATNLATTGSSPTVAKTTTTF |
| | | | NTLAGSLFTPLTTPGMSTLASESVTSRTSYNHRSWISTT |
| | | | SSYNRRYWTPATSTPVTSTFSPGISTSSIPSSTAATVPFM |
| | | | VPFTLNFTITNLQYEEDMRHPGSRKFNATERELQGLLK |
| | | | PLFRNSSLEYLYSGCRLASLRPEKDSSATAVDAICTHRP |
| | | | DPEDLGLDRERLYWELSNLTNGIQELGPYTLDRNSLYV |
| | | | NGFTHRSSMPTTSTPGTSTVDVGTSGTPSSSPSPTTAGPL |
| | | | LMPFTLNFTITNLQYEEDMRRTGSRKFNTMESVLQGLL |
| | | | KPLFKNTSVGPLYSGCRLTLLRPEKDGAATGVDAICTH |
| | | | RLDPKSPGLNREQLYWELSKLINDIEELGPYTLDRNSL |
| | | | YVNGFTHQSSVSTTSTPGTSTVDLRTSGTPSSLSSPTIMA |
| | | | AGPLLVPFTLNFTITNLQYGEDMGHPGSRKFNTTERVL |
| | | | QGLLGPIFKNTSVGPLYSGCRLTSLRSEKDGAATGVDAI |
| | | | CIHHLDPKSPGLNRERLYWELSQLTNGIKELGPYTLDR |
| | | | NSLYVNGFTHRTSVPTSSTPGTSTVDLGTSGTPFSLPSPA |
| | | | TAGPLLVLFTLNFTITNLKYEEDMHRPGSRKENTTERVL |
| | | | QTLLGPMFKNTSVGLLYSGCRLTLLRSEKDGAATGVD |
| | | | AICTHRLDPKSPGVDREQLYWELSQLTNGIKELGPYTL |
| | | | DRNSLYVNGFTHWIPVPTSSTPGTSTVDLGSGTPSSLPS |
| | | | PTTAGPLLVPFTLNFTITNLKYEEDMHCPGSRKENTTER |
| | | | VLQSLLGPMFKNTSVGPLYSGCRLTLLRSEKDGAATGV |
| | | | DAICTHRLDPKSPGVDREQLYWELSQLTNGIKELGPYT |
| | | | LDRNSLYVNGFTHQTSAPNTSTPGTSTVDLGTSGTPSSL |
| | | | PSPTSAGPLLVPFTLNFTITNLQYEEDMHHPGSRKFNTT |
| | | | ERVLQGLLGPMFKNTSVGLLYSGCRLTLLRPEKNGAAT |
| | | | GMDAICSHRLDPKSPGLNREQLYWELSQLTHGIKELGP |
| | | | YTLDRNSLYVNGFTHRSSVAPTSTPGTSTVDLGTSGTPS |
| | | | SLPSPTTAVPLLVPFTLNFTITNLQYGEDMRHPGSRKFN |
| | | | TTERVLQGLLGPLFKNSSVGPLYSGCRLISLRSEKDGAA |
| | | | TGVDAICTHHLNPQSPGLDREQLYWQLSQMTNGIKEL |
| | | | GPYTLDRNSLYVNGFTHRSSGLTTSTPWTSTVDLGTSG |
| | | | TPSPVPSPTTTGPLLVPFTLNFTITNLQYEENMGHPGSR |
| | | | KFNITESVLQGLLKPLFKSTSVGPLYSGCRLTLLRPEKD |
| | | | GVATRVDAICTHRPDPKIPGLDRQQLYWELSQLTHSITE |
| | | | LGPYTLDRDSLYVNGFTQRSSVPTTSTPGTFTVQPETSE |
| | | | TPSSLPGPTATGPVLLPFTLNFTITNLQYEEDMRRPGSR |
| | | | KFNTTERVLQGLLMPLFKNTSVSSLYSGCRLTLLRPEK |
| | | | DGAATRVDAVCTHRPDPKSPGLDRERLYWKLSQLTHG |
| | | | ITELGPYTLDRHSLYVNGFTHQSSMTTTRTPDTSTMHL |
| | | | ATSRTPASLSGPMTASPLLVLFTINFTITNLRYEENMHH |
| | | | PGSRKFNTTERVLQGLLRPVFKNTSVGPLYSGCRLTLL |
| | | | RPKKDGAATKVDAICTYRPDPKSPGLDREQLYWELSQ |
| | | | LTHSITELGPYTLDRDSLYVNGFTQRSSVPTTSIPGTPTV |
| | | | DLGTSGTPVSKPGPSAASPLLVLFTLNFTITNLRYEENM |
| | | | QHPGSRKFNTTERVLQGLLRSLFKSTSVGPLYSGCRLTL |
| | | | LRPEKDGTATGVDAICTHHPDPKSPRLDREQLYWELSQ |
| | | | LTHNITELGPYALDNDSLFVNGFTHRSSVSTTSTPGTPT |
| | | | VYLGASKTPASIFGPSAASHLLILFTLNFTITNLRYEENM |
| | | | WPGSRKFNTTERVLQGLLRPLFKNTSVGPLYSGCRLTL |
| | | | LRPEKDGEATGVDAICTHRPDPTGPGLDREQLYLELSQ |
| | | | LTHSITELGPYTLDRDSLYVNGFTHRSSVPTTSTGVVSE |
| | | | EPFTLNFTINNLRYMADMGQPGSLKFNITDNVMQHLLS |
| | | | PLFQRSSLGARYTGCRVIALRSVKNGAETRVDLLCTYL |
| | | | QPLSGPGLPIKQVFHELSQQTHGITRLGPYSLDKDSLYL |
| | | | NGYNEPGPDEPPTTPKPATTFLPPLSEATTAMGYHLKTL |
| | | | TLNFTISNLQYSPDMGKGSATFNSTEGVLQHLLRPLFQ |
| | | | KSSMGPFYLGCQLISLRPEKDGAATGVDTTCTYHPDPV |
| | | | GPGLDIQQLYWELSQLTHGVTQLGFYVLDRDSLFINGY |
| | | | APQNLSIRGEYQINFHIVNWNLSNPDPTSSEYITLLRDIQ |
| | | | DKVTTLYKGSQLHDTFRFCLVTNLTMDSVLVTVKALF |
| | | | SSNLDPSLVEQVFLDKTLNASFHWLGSTYQLVDIHVTE |
| | | | MESSVYQPTSSSSTQHFYLNFTITNLPYSQDKAQPGTTN |
| | | | YQRNKRNIEDALNQLFRNSSIKSYFSDCQVSTFRSVPNR |
| | | | HHTGVDSLCNFSPLARRVDRVAIYEEFLRMTRNGTQLQ |
| | | | NFTLDRSSVLVDGYSPNRNEPLTGNSDLPFWAVILIGLA |
| | | | GLLGVITCLICGVLVTTRRRKKEGEYNVQQQCPGYYQS |
| | | | HLDLEDLQ |

In some embodiments, the lung cancer associated proteomic marker is Fragment of Cytokeratin 19 (CYFRA21-1) or a fragment thereof. In some embodiments, the lung cancer associated proteomic marker is Keratin, type I cytoskeletal 19 (KRT19) or a fragment thereof. In some embodiments, the lung cancer associated proteomic marker is Glycoprotein 130 (GP130) or a fragment thereof. In some embodiments, the lung cancer associated proteomic marker is Complement Component C9 (C9) or a fragment thereof. In some embodiments, the lung cancer associated proteomic marker is Cell adhesion molecule (CEA) or a fragment thereof. In some embodiments, the lung cancer associated proteomic marker is CEACAM5 or a fragment thereof. In some embodiments, the lung cancer associated proteomic marker is CA-125 Antigen (CA125) or a fragment thereof. In some embodiments, the lung cancer associated proteomic marker is Myoglobin (MB) or a fragment thereof. In some embodiments, the lung cancer associated proteomic marker is Gamma-enolase (ENO2) or a fragment thereof. In some embodiments, the lung cancer associated proteomic marker is Fibrinogen-like protein 1 (FGL1) or a fragment thereof. In some embodiments, the lung cancer associated proteomic marker is Insulin-like growth factor-binding protein 6 (IGFBP-6) or a fragment thereof. In some embodiments, the lung cancer associated proteomic marker is Platelet endothelial cell adhesion molecule (PECAM1) or a fragment thereof. In some embodiments, the lung cancer associated proteomic marker is Serum amyloid A protein (SAA) or a fragment thereof. In some embodiments, the lung cancer associated proteomic marker is Serum amyloid A-1 protein (SAA1) or a fragment thereof. In some embodiments, the lung cancer associated proteomic marker is Serum amyloid A-2 protein (SAA2) or a fragment thereof. In some embodiments, the lung cancer associated proteomic marker is Serum amyloid A-3 protein (SAA3) or a fragment thereof. In some embodiments, the lung cancer associated proteomic marker is Serum amyloid A-4 protein (SAA4) or a fragment thereof. In some embodiments, the lung cancer associated proteomic marker is SAA or a fragment thereof and SAA or the fragment thereof comprises information on one or more of SAA1, SAA2, SAA3, SAA4, or any combination thereof.

In some embodiments, Fibrinogen-like protein 1 (FGL1) is referred to by one or more alternative names. Non-limiting examples of alternative names for FGL1 include HP-041, Hepassocin (HPS), Hepatocyte-derived fibrinogen-related protein 1 (HFREP-1 or HFREP1), or Liver fibrinogen-related protein 1 (LFIRE-1). In some embodiments, Platelet endothelial cell adhesion molecule (PECAM-1) is referred to by one or more alternative names. Non-limiting examples of alternative names for PECAM-1 include EndoCAM, GPIIA′, PECA1, or CD31. In some embodiments, Myoglobin (MB) is referred to by one or more alternative names. Non-limiting examples of alternative names for MB include Nitrite reductase MB or Pseudoperoxidase MB. In some embodiments, Insulin-like growth factor-binding protein 6 (IGFBP-6) is referred to by one or more alternative names. Non-limiting examples of alternative names for IGFBP-6 include IBP-6, IBP6, or IGF-binding protein 6. In some embodiments, Keratin, type I cytoskeletal 19 (KRT19) is referred to by one or more alternative names. Non-limiting examples of alternative names for KRT19 include cytokeratin-19 (CK-19) or Keratin-19 (K19). In some embodiments, Gamma-enolase (ENO 2) is referred to by one or more alternative names. Non-limiting examples of alternative names for ENO 2 include 2-phospho-D-glycerate hydro-lyase, Enolase 2, Neural enolase, or Neuron-specific enolase (NSE). In some embodiments, Cell Adhesion Molecule (CEA) is referred to by one or more alternative names. Non-limiting examples of alternative names for CEA include Cell adhesion molecule CEACAM5, Carcinoembryonic antigen (CEA), Carcinoembryonic antigen-related cell adhesion molecule 5 (CEA cell adhesion molecule 5), Meconium antigen 100, or CD66e. In some embodiments, Serum amyloid A-1 protein (SAA1) is referred to by one or more alternative names. Non-limiting examples of alternative names for SAA1 include Amyloid protein A, Amyloid fibril protein AA. In some embodiments, Serum amyloid A-2 protein (SAA2) is referred to by one or more alternative names. Non-limiting examples of alternative names for SAA2 include Amyloid A2 protein. In some embodiments, Serum amyloid A-4 protein (SAA4) is referred to by one or more alternative names. Non-limiting examples of alternative names for SAA4 include Constitutively expressed serum amyloid A protein (C-SAA) or (CSAA). In some embodiments, CA-125 Antigen (CA125) is referred to by one or more alternative names. Non-limiting examples of alternative names for CA125 include MUC-16 or MUC 16, Mucin-16, Ovarian cancer-related tumor marker CA125, (CA-125) or Ovarian carcinoma antigen CA125.

Methods of the present disclosure, in some embodiments, comprise obtaining biomarker measurements corresponding to one or more lung cancer associated proteomic markers. In some embodiments, methods comprise obtaining biomarker measurements corresponding to a plurality of lung cancer associated proteomic markers. In some embodiments, the biomarker measurements comprise information on Keratin, type I cytoskeletal 19 (KRT19). In some embodiments, the information on Keratin, type I cytoskeletal 19 (KRT19) comprises information on Fragment of Cytokeratin 19 (CYFRA21-1). In some embodiments, a biomarker measurement comprises information on Glycoprotein 130 (GP130) or a fragment thereof. In some embodiments, a biomarker measurement comprises information on Complement Component C9 (C9) or a fragment thereof. In some embodiments, a biomarker measurement comprises information on Cell adhesion molecule (CEA) or a fragment thereof. In some embodiments, a biomarker measurement comprises information on CA-125 Antigen (CA125) or a fragment thereof. In some embodiments, a biomarker measurement comprises information on Myoglobin (MB) or a fragment thereof. In some embodiments, a biomarker measurement comprises information on Gamma-enolase (ENO2) or a fragment thereof. In some embodiments, a biomarker measurement comprises information on Fibrinogen-like protein 1 (FGL1) or a fragment thereof. In some embodiments, a biomarker measurement comprises information on Insulin-like growth factor-binding protein 6 (IGFBP-6) or a fragment thereof. In some embodiments, a biomarker measurement comprises information on Platelet endothelial cell adhesion molecule (PECAM1) or a fragment thereof. In some embodiments, a biomarker measurement comprises information on Serum amyloid A protein (SAA) or a fragment thereof. In some embodiments, a biomarker measurement comprises information on Serum amyloid A-1 protein (SAA1) or a fragment thereof. In some embodiments, a biomarker measurement comprises information on Serum amyloid A-2 protein (SAA2) or a fragment thereof. In some embodiments, a biomarker measurement comprises information on Serum amyloid A-3 protein (SAA3) or a fragment thereof. In some embodiments, a biomarker measurement comprises information on Serum amyloid A-4 protein (SAA4) or a fragment thereof. In some embodiments, a biomarker measurement comprises information on Serum amyloid A protein (SAA) or a fragment thereof and the information for SAA comprises information on one or more of SAA1, SAA2, SAA3, SAA4, or any combination thereof. In some embodiments, biomarker measurements comprise a concentration or an amount of the lung cancer associated proteomic marker detected in a biological sample obtained from the subject.

In some embodiments, two or more biomarker measurements are obtained for a single lung cancer associated proteomic marker, as a non-limiting example, information on CEA and a fragment of CEA may be detected. As another non-limiting example, information on a fragment of CEA and information on another fragment of CEA may be detected in separate assays. In some embodiments, biomarker measurements corresponding to CEA and the fragment of CEA may be detected in combination with one or more of KRT19, GP130, C9, CA125, MB, ENO2, FGL1, IGFBP-6, PECAM1, SAA, or any fragment thereof to assess lung cancer in a biological sample. As another non-limiting example, information for SAA may include information for two or more proteomic markers of the SAA family of proteins. In some embodiments, SAA-1, SAA-2, or both are detected in combination with one or more of KRT19, GP130, C9, CA125, MB, ENO2, FGL1, IGFBP-6, PECAM1, CEA, or any fragment thereof to assess lung cancer in a biological sample. In some embodiments, SAA1, SAA2, SAA4, or any combination thereof are detected in combination with one or more of KRT19, GP130, C9, CA125, MB, ENO2, FGL1, IGFBP-6, PECAM1, CEA, or any fragment thereof to assess lung cancer in a biological sample.

In some embodiments, the lung cancer associated markers may be associated with one or more biological processes. Examples of biological processes may include, but are not limited to, immune evasion (e.g., immune checkpoint resistance), inflammation (e.g., cytokine signaling), tumor microenvironment modulation (e.g., innate immunity), angiogenesis, oxygen transport (e.g., hypoxia), cytoskeletal remodeling, altered glycolytic metabolism, cell adhesion, acute phase response and growth regulation (e.g., cell proliferation). In some embodiments, the lung cancer associated biomarkers may comprise one or more proxy biomarkers that can be used in lieu of one or more of the lung cancer associated markers provided in Table 1. Proxy biomarkers may be related to a characteristic while not being directly involved in a biological process, disease, or condition, but may reflect or be correlated with the biological process, disease or condition. As a non-limiting example, FGL1 is involved in immune evasion but may be difficult to detect. A proxy biomarker that is involved in immune evasion that may be used in lieu of FGL1 comprises STAT3 or PD-L1. Use of proxy biomarkers may provide a way to approximate a biomarker that is directly related with a biological process but is difficult to detect or whose detection levels may be below a level of detection. In some embodiments, the detection level may be below a level of detection due to noise. Proxy biomarkers may be selected due to their ability to be detected in samples acquired through less invasive or non-invasive methods. In some embodiments, proxy biomarkers for immune invasion may comprise STAT3 (Signal Transducer and Activator of Transcription 3), PD-L1 (CD274), or LAG-3. In some embodiments, proxy biomarkers for inflammation (e.g., cytokine signaling) may comprise IL-6, STAT3, and/or C/EBPβ. In some embodiments, proxy biomarkers for tumor microenvironment modulation (e.g., innate immunity) may comprise C3, C5, and clusterin. In some embodiments, the proxy biomarkers for angiogenesis may comprise EGF, VEGFR-2/3, von Willebrand Factor (vWF), and CD105. In some embodiments, the proxy biomarkers for oxygen transport (e.g., hypoxia) may comprise HIF-1a, Carbonic Anhydrase IX (CAIX), and LDHA. In some embodiments, the proxy biomarkers for growth regulation (e.g., cell proliferation) may comprise IGFBP-2, IGFBP-3, IGFBP-5, and IGF-1R. In some embodiments, the proxy biomarkers for cytoskeletal remodeling may comprise CK7, or CK5/6. In some embodiments, the proxy biomarkers for altered glycolytic metabolism may comprise LDH, PKM2, ENO1, and GAPDH. In some embodiments, the proxy biomarkers for cell adhesion may comprise CEACAM-1, CEACAM-6, SCC antigen, and ProGRP. In some embodiments, the proxy biomarkers for acute phase response may comprise CRP, fibrinogen, haptoglobin, serum amyloid P, and IL-6.

In some embodiments, proxy biomarkers for the lung cancer associated markers disclosed herein are identified using statistical tests of association. In some embodiments, proxy biomarkers for the lung cancer associated markers disclosed herein are identified using mathematical models of the desirable relationship between the proxy biomarker and the lung cancer associated marker (e.g., regression). In some embodiments, proxy biomarkers for the lung cancer associated markers disclosed herein are identified based on a Pearson Correlation Coefficient. In some embodiments, proxy biomarkers for the lung cancer associated markers disclosed herein are identified based on a Deming Regression slope. In some embodiments, proxy biomarkers for the lung cancer associated markers disclosed herein are identified based on a Pearson Correlation Coefficient and a Deming Regression slope.

In some embodiments, the Pearson Correlation Coefficient is greater than or equal to 0.80. In some embodiments, the Pearson Correlation Coefficient is greater than or equal to 0.81. In some embodiments, the Pearson Correlation Coefficient is greater than or equal to 0.82. In some embodiments, the Pearson Correlation Coefficient is greater than or equal to 0.83. In some embodiments, the Pearson Correlation Coefficient is greater than or equal to 0.84. In some embodiments, the Pearson Correlation Coefficient is greater than or equal to 0.85. In some embodiments, the Pearson Correlation Coefficient is greater than or equal to 0.86. In some embodiments, the Pearson Correlation Coefficient is greater than or equal to 0.87. In some embodiments, the Pearson Correlation Coefficient is greater than or equal to 0.88. In some embodiments, the Pearson Correlation Coefficient is greater than or equal to 0.89. In some embodiments, the Pearson Correlation Coefficient is greater than or equal to 0.90. In some embodiments, the Pearson Correlation Coefficient is greater than or equal to 0.91. In some embodiments, the Pearson Correlation Coefficient is greater than or equal to 0.92. In some embodiments, the Pearson Correlation Coefficient is greater than or equal to 0.93. In some embodiments, the Pearson Correlation Coefficient is greater than or equal to 0.94. In some embodiments, the Pearson Correlation Coefficient is greater than or equal to 0.95. In some embodiments, the Pearson Correlation Coefficient is greater than or equal to 0.96. In some embodiments, the Pearson Correlation Coefficient is greater than or equal to 0.97. In some embodiments, the Pearson Correlation Coefficient is greater than or equal to 0.98. In some embodiments, the Pearson Correlation Coefficient is greater than or equal to 0.99. In some embodiments, the Pearson Correlation Coefficient is greater than or equal to 1.0. In some embodiments, the Pearson Correlation Coefficient is between 0.80 and 1.0. In some embodiments, the Pearson Correlation Coefficient is between 0.85 and 0.99. In some embodiments, the Pearson Correlation Coefficient is between 0.90 and 0.98. In some embodiments, the Pearson Correlation Coefficient is between 0.92 and 0.96. In some embodiments, the Pearson Correlation Coefficient is between 0.93 and 0.95. In some embodiments, the Pearson Correlation Coefficient is 0.94.

In some embodiments, the Deming Regression slopes fall within [0.50, 1.50]. In some embodiments, the Deming Regression slopes fall within [0.65, 1.45]. In some embodiments, the Deming Regression slopes fall within [0.60, 1.40]. In some embodiments, the Deming Regression slopes fall within [0.65, 1.35]. In some embodiments, the Deming Regression slopes fall within [0.70, 1.30]. In some embodiments, the Deming Regression slopes fall within [0.75, 1.25]. In some embodiments, the Deming Regression slopes fall within [0.80, 1.20]. In some embodiments, the Deming Regression slopes fall within [0.85, 1.15]. In some embodiments, the Deming Regression slopes fall within [0.87, 1.13]. In some embodiments, the Deming Regression slopes fall within [0.90, 1.10].

As a non-limiting example, if the lung cancer associated marker is difficult to detect or has detection levels below a level of detection, a proxy biomarker may be identified by calculating the Pearson Correlation Coefficient and the Deming Regression slope between the measurements of each candidate proxy biomarker and the measurements of the lung cancer associated marker. The proxy biomarker may then be selected from among the candidates based on having a Pearson Correlation Coefficient and a Deming Regression slope that are both sufficiently large. As another non-limiting example, if the lung cancer associated marker is directly related to a biological process but is also sensitive to non-biological processes (e.g., pre-analytical variability), a proxy biomarker may be identified by the method described in the previous non-limiting example after accounting for the effects of the non-biological processes on the lung cancer associated marker. The effects of the non-biological processes on the lung cancer associated marker may be accounted for by a regression model on measurements of the non-biological processes to measurements of the lung cancer associated marker and retaining the residual.

b. Biological Sample

In some embodiments, the biological sample is a biofluid sample. Non-limiting examples of a biofluid sample include cerebral spinal fluid (CSF), synovial fluid (SF), urine, blood, plasma, serum, tears, semen, whole blood, milk, nipple aspirate, ductal lavage, vaginal fluid, nasal fluid, ear fluid, gastric fluid, pancreatic fluid, trabecular fluid, lung lavage, prostatic fluid, sputum, fecal matter, bronchial lavage, fluid from swabs, bronchial aspirants, sweat, or saliva. In some embodiments, the biofluid sample is blood. In some embodiments, the biofluid sample is whole blood. In some embodiments, the biofluid sample is plasma. In some embodiments, the biofluid sample is serum. In some embodiments, the biological sample is fluidized solids of biological material. A non-limiting example of fluidized solids of biological material is a tissue homogenate. In some embodiments, the biological sample is derived from cell culture. In some embodiments, the biological sample may be lyophilized. In some embodiments, the lyophilized biological sample is reconstituted. In some embodiments, the lyophilized biological sample comprises proteins.

As a non-limiting example, a blood sample may be collected from a subject and processed according to the methods described herein. In this non-limiting example, the blood sample is collected from a subject using a needle and blood collection tube. In some embodiments, the needle is a 21-gauge needle. In some embodiments, the blood collection tube is a K₂EDTA blood collection tube. In this non-limiting embodiment, the K₂EDTA blood collection tube with the subject's blood is inverted. In some embodiments, the K₂EDTA blood collection tube with the subject's blood is inverted 5-15 times. In some embodiments, the K₂EDTA blood collection tube with the subject's blood is inverted 8-10 times. In this non-limiting example, after inverting the K₂EDTA blood collection tube with the subject's blood the K₂EDTA blood collection tube is centrifuged. In some embodiments, the K₂EDTA blood collection tube is centrifuged for about 10-20 minutes. In some embodiments, the K₂EDTA blood collection tube is centrifuged for about 15 minutes. In some embodiments, the K₂EDTA blood collection tube is centrifuged at about 1000-1500×g. In some embodiments, the K₂EDTA blood collection tube is centrifuged at about 1300×g. In some embodiments, the K₂EDTA blood collection tube with the subject's blood is centrifuged within 1 hour of collection. In some embodiments, the K₂EDTA blood collection tube with the subject's blood is centrifuged within 45 minutes of collection. In some embodiments, the K₂EDTA blood collection tube with the subject's blood is centrifuged within 30 minutes of collection. In some embodiments, the K₂EDTA blood collection tube with the subject's blood is centrifuged within 15 minutes of collection. In this non-limiting example, the plasma from the centrifuged K₂EDTA blood collection tube is transferred to a blood storage tube. In some embodiments, the blood storage tube is a FluidX™ tube. In some embodiments, the blood storage tube comprising the subject's plasma may be shipped to a testing facility. In some embodiments, the plasma may be shipped on wet ice. In some embodiments, the plasma may be shipped on dry ice. In some embodiments, the plasma may be shipped with ice packs. In some embodiments, the plasma sample may be stored at about −20° C. until ready to ship. In some embodiments, the plasma sample may be stored at about −80° C. until ready to ship.

As another non-limiting example, a plasma sample of a patient may be processed for the detection of one or more lung cancer associated markers. In this non-limiting example, the plasma sample is stored frozen and may be thawed before processing. In some embodiments, the plasma sample is lyophilized and thawed and reconstituted before processing. In some embodiments, the plasma sample is cryopreserved and thawed before processing. In some embodiments, the plasma sample is stored at about −20° C. In some embodiments, the plasma sample is stored at about −80° C. In some embodiments, the plasma sample is thawed on ice. In some embodiments, the plasma sample is thawed in a cold tray. In some embodiments, the plasma sample is thawed at about 4° C. In some embodiments, the plasma sample is thawed at room temperature. In some embodiments, the plasma sample thaws for about 15 minutes. In some embodiments, the plasma sample thaws for about 30 minutes. In some embodiments, the plasma sample thaws for about 45 minutes. In some embodiments, the plasma sample thaws for about 60 minutes. In some embodiments, the plasma sample thaws for about 75 minutes. In some embodiments, the plasma sample thaws for about 90 minutes. In this non-limiting embodiment, once the plasma sample is thawed, the plasma sample is centrifuged. In some embodiments, the centrifuge is a cold centrifuge. In some embodiments, the centrifuge is about 4° C. In some embodiments, the plasma sample is centrifuged for 5-20 minutes. In some embodiments, the plasma sample is centrifuged for about 10 minutes. In some embodiments, the samples are centrifuged at 3000-5000 rpm. In some embodiments, the samples are centrifuged at 3500-4500 rpm. In some embodiments, the samples are centrifuged at 3800-4100 rpm. In some embodiments, the samples are centrifuged at 3900 rpm. In some embodiments, after centrifuging, the plasma sample is ready for use with the methods disclosed herein.

c. Assays

Disclosed herein are methods comprising contacting a lung cancer associated marker with a detectable reagent sufficient to measure the lung cancer associated marker. In some embodiments, the lung cancer associated marker is a protein, a polypeptide, a peptide, or a fragment thereof. In some embodiments, the detection of a protein, a polypeptide, a peptide, or a fragment thereof generates proteomic data.

Proteomic data may be generated by any of a variety of methods. Generating proteomic data may include using a detection reagent that binds to a peptide or protein and yields a detectable signal. After use of a detection reagent that binds to a peptide or protein and yields a detectable signal, a readout may be obtained that is indicative of the presence, absence, amount, concentration of the protein or peptide. Generating proteomic data may include concentrating, filtering, or centrifuging a sample.

Lung cancer associated markers may be enriched prior to assaying or measuring them. The enrichment may enrich one set of lung cancer associated markers and not another set, or may enrich a single lung cancer associated marker and not another lung cancer associated marker. For example, the enrichment may enrich one set of proteins and not another set, or may enrich a single protein and not another protein. Enrichment may be obtained through the use of an affinity reagent, for example by incubating the affinity reagent with a sample prior to measuring the lung cancer associated marker in the sample. The affinity reagent may include an antibody. The affinity reagent may include a particle such as a nanoparticle. Lung cancer associated markers, such as proteins, may be adsorbed to the affinity reagent, separated from the rest of the sample, and then assayed by using an assay described herein.

In some embodiments, the lung cancer associated markers are detected with an immunoassay. In some embodiments, the lung cancer associated markers are detected with a proximity extension assay. In some embodiments, an amount of the lung cancer associated markers are measured. In some embodiments, a concentration of the lung cancer associated markers are measured. In some embodiments, a presence of the lung cancer associated marker is measured.

In some embodiments, a single lung cancer associated marker is detected in an immunoassay. In some embodiments, one or more lung cancer associated markers are detected in a single immunoassay. In some embodiments, a lung cancer associated marker is detected in two or more immunoassays. In some embodiments, two or more lung cancer associated markers are detected using different immunoassays. In some embodiments, three or more lung cancer associated markers are detected using three different immunoassays.

In some embodiments, the immunoassay is an enzyme-linked immunosorbent assay (ELISA). In some embodiments the immunoassay is direct ELISA. In some embodiments, the immunoassay is indirect ELISA. In some embodiments, the immunoassay is a sandwich ELISA. In some embodiments, the immunoassay is competitive ELISA. In some embodiments, a lung cancer associated marker is detected with a labeled ligand. In some embodiments, a lung cancer associated marker is detected by first binding to a primary ligand and then binding to a labeled secondary ligand. In some embodiments, a lung cancer associated marker is detected by first binding to a capture ligand and then binding to a labeled ligand. In some embodiments, the capture ligand and the labeled ligand bind to different regions or sites of the lung cancer associated marker. In some embodiments, the lung cancer associated marker is detected by competing with a labeled control marker for binding to a ligand. In some embodiments, the ligand is an antibody. In some embodiments, the ligand is an antigen binding fragment. Non-limiting examples of ligands include antibodies, antigens, antigen-binding fragments, and nucleic acids (e.g., aptamers).

In some embodiments, the label is an enzyme label. In some embodiments, the enzyme label is detected by a colorimetric/chromogenic or chemiluminescence assay. Non-limiting examples of enzyme labels include horseradish peroxidase (HRP) or alkaline phosphatase (AP). In some embodiments, the label is a fluorescent label. Non-limiting examples of fluorescent labels include Alexa fluor dyes, such as, for example Alexa fluor 488 or Alexa fluor 647. Other non-limiting examples of fluorescent labels includes a fluorescent protein and fluorescence resonance energy transfer pairs. In some embodiments, the label is a fluorescent dye. A non-limiting example of a fluorescent dye includes fluorescein isothiocyanate (FITC). In some embodiments, the label is a chemiluminescent substrate. A non-limiting example of a chemiluminescent substrate includes luminol. In some embodiments, the label is an affinity tag. A non-limiting example of an infinity tag is a streptavidin-biotin label. In some embodiments, the label is a radioactive label. In some embodiments, the label is a nucleic acid sequence. In some embodiments, the signal produced by the label correlates to the amount, concentration, or presence of a lung cancer associated marker. In some embodiments, the signal produced by the label inversely correlates to the amount, concentration, or presence of a lung cancer associated marker.

In some embodiments, the immunoassay is a lateral flow assay. In some embodiments, the immunoassay is a sandwich format lateral flow assay. In some embodiments, the immunoassay is a competitive format lateral flow assay. In some embodiments, the immunoassay is a multiplex lateral flow assay. In some embodiments, the immunoassay comprises detection ligands conjugated to a visible label. As a non-limiting example, a labeled ligand may bind to a lung cancer associated marker and when the bound ligand and lung cancer associated marker continue to flow through the membrane they bind to immobilized capture ligands. In some embodiments, the ligand comprises a label. In some embodiments, the label comprises gold nanoparticles. In some embodiments, the label comprises colored latex beads. In some embodiments, the label comprises fluorescent dyes. In some embodiments, the label comprises magnetic particles. In some embodiments, the label comprises carbon nanoparticles. In some embodiments, the label comprises up-converting phosphor. In some embodiments, the ligand is an antibody. In some embodiments, the ligand is an antigen binding fragment. Non-limiting examples of ligands include antibodies, antigens, antigen-binding fragments, and nucleic acids.

In some embodiments, the immunoassay is a particle-based assay. In some embodiments, the particle-based assay uses beads. In some embodiments, the beads comprise microspheres. In some embodiments, the beads comprise nanoparticles. In some embodiments, the beads are polystyrene beads. In some embodiments, the beads are magnetic beads. In some embodiments, the beads are latex beads. In some embodiments, the beads are silica beads. In some embodiments, the beads are surface plasmon resonance (SPR) particles. In some embodiments, the bead comprises a coating layer coupled to the surface of the bead. In some embodiments, the coating layer comprises carboxyl (—COOH) groups. In some embodiments, the coating layer comprises streptavidin. In some embodiments, the coating layer comprises avidin. In some embodiments, the beads are coated with ligands. Non-limiting examples of ligands include antibodies, antigens, antigen-binding fragments, and nucleic acids. In some embodiments, the beads comprise a label. In some embodiments, the label is a fluorescent label. In some embodiments, a bead comprises two labels. In this embodiment, the two labels may comprise different fluorophores. In some embodiments, beads with different ligands are labeled with different fluorophores. In some embodiments, beads with different ligands are labeled with two different fluorophores in unique ratios. In some embodiments, the beads are color coded to enable identification of the target proteomic marker in a multiplexed fashion.

In some embodiments, the immunoassay is a planar immunoassay. In some embodiments, the planar surface comprises a coating layer coupled to the planar surface. In some embodiments, the coating layer comprises carboxyl (—COOH) groups. In some embodiments, the coating layer comprises streptavidin. In some embodiments, the coating layer comprises avidin. In some embodiments, the planar surface is coated with ligands. Non-limiting examples of ligands include antibodies, antigens, antigen-binding fragments, and nucleic acids. In some embodiments, the planar surface comprises a label. In some embodiments, the label is a fluorescent label. In some embodiments, a planar surface comprises two labels. In this embodiment, the two labels may comprise different fluorophores. In some embodiments, planar surfaces with different ligands are labeled with different fluorophores. In some embodiments, planar surfaces with different ligands are labeled with two different fluorophores in unique ratios. In some embodiments, the planar surface is color coded to enable identification of the target proteomic marker in a multiplexed fashion.

In some embodiments, the immunoassay is a proximity extension assay. In some embodiments, the proximity extension assay comprises a ligand for binding to a protein. In some embodiments, the proximity extension assay comprises two ligands for binding to a protein. Non-limiting examples of ligands include antibodies, antigens, antigen-binding fragments, and nucleic acids. In some embodiments, the label is a nucleic acid. As a non-limiting example, a first antibody and a second antibody may bind to different parts of a marker, such a protein, polypeptide, peptide, or fragment thereof (e.g., the lung cancer associated markers disclosed herein). When the first and second antibody bind to the marker, nucleic acids conjugated to the first and second antibody are brought into proximity allowing for an extension reaction of the nucleic acids resulting in a new nucleic acid that can be detected. In this non-limiting example, the amount of nucleic acid detected using quantitative polymerase chain reaction (qPCR), sequencing, or digital droplet polymerase chain reaction (ddPCR) reflects the amount of protein present in the sample.

In some embodiments, the lung cancer associated marker is detected based on a readout from the label, such as a label disclosed herein. In some embodiments, the readout is a fluorescent readout. In some embodiments, the fluorescent readout is obtained from one or more fluorescent proteins. In some embodiments, the fluorescent readout is obtained from a fluorescence resonance energy transfer. In some embodiments, the instrument for analysis uses lasers to identify the magnitude of signal from the label. For example, the Luminex™ instrument uses lasers to identify the color of each bead (identifying the target) and the magnitude of the fluorescent signal (quantifying the amount of the target) from the fluorescent label of the antibody specific bound to the target. In some embodiments, the instrument for analysis is a plate reader. As an example, the instrument may be a plate reader configured to expose the ligand-bound target proteomic marker(s) with light through each well of a microplate and measure the amount of light transmitted or emitted by the sample. In this example, the signal is quantified, providing an optical density (OD) value corresponding to the concentration of the target proteomic marker in the well. In some embodiments, the instrument comprises optics for one or more detection modes, such as absorbance, fluorescence, luminescence, time-resolved fluorescence, and fluorescence polarization. In some embodiments, the signal produced is analyzed by measuring absorbance. In some embodiments, the signal produced is analyzed by measuring fluorescence. In some embodiments, the signal produced is analyzed by measuring luminescence. In some embodiments, the signal produced is analyzed using quantitative polymerase chain reaction (qPCR). In some embodiments, the signal is analyzed using sequencing. Non-limiting examples of sequencing include Sanger sequencing, amplicon sequencing, and Next-Generation sequencing. In some embodiments, the signal is analyzed using digital droplet polymerase chain reaction (ddPCR). In some embodiments, the immunoassay comprises mass spectrometry and the readout is a mass spectrum conveying information about an abundance of a target proteomic marker.

In some embodiments, a first immunoassay is used to analyze a first subset of the lung cancer associated proteomic markers. The first subset of the lung cancer proteomic markers can be Myoglobin (MB), Complement Component C9 (C9), Fragment of Cytokeratin 19 (CYFRA21-1), Glycoprotein 130 (GP130), Gamma-enolase (ENO2), Insulin-like growth factor-binding protein 6 (IGFBP-6), Platelet endothelial cell adhesion molecule (PECAM1), Serum amyloid A protein (SAA), or any fragment thereof, or the proxy proteomic marker thereof. In some embodiments, the first subset is Myoglobin (MB), Complement Component C9 (C9), Fragment of Cytokeratin 19 (CYFRA21-1), Glycoprotein 130 (GP130), Gamma-enolase (ENO2), Insulin-like growth factor-binding protein 6 (IGFBP-6), Platelet endothelial cell adhesion molecule (PECAM1), and Serum amyloid A protein (SAA), wherein one or more of the proteomic markers may be substituted with a proxy proteomic marker if needed. In some embodiments, the first immunoassay is a sandwich immunoassay. In some embodiments, the first immunoassay comprises contacting the target proteomic marker with a capture receptor (e.g., antibody) immobilized (directly or indirectly) to a solid support (e.g., particle) under conditions sufficient to specifically bind a target proteomic marker to the capture receptor to form a first binding complex; and contacting a detection reagent to the first binding complex. In some embodiments, the detection reagent comprises a binding moiety specific to the first binding complex (e.g., capture receptor or tag coupled thereto, or target proteomic marker or tag coupled thereto) and a label that conveys one or more signals that can be detected. In some embodiments, a magnitude of the one or more signals is proportionate to the abundance (amount concentration) of the proteomic marker in the biofluid sample. In some embodiments, solid support is a bead that is color-coded to enable multiplexed analysis in a single sample. In some embodiments, a second immunoassay is used to analyze a second subset of the proteomic markers. In some embodiments, the second subset is or comprises Cell adhesion molecule (CEA), CA-125 Antigen (CA125), or any fragment thereof, or the proxy proteomic marker thereof. In some embodiments, the second subset is or comprises Cell adhesion molecule (CEA) and CA-125 Antigen (CA125), wherein one or more of the proteomic markers may be substituted with a proxy proteomic marker if needed. In some embodiments, lung cancer associated proteomic marker is contacted simultaneously to a biotinylated receptor (e.g., mouse monoclonal antibody) and a horseradish peroxidase (HRP)-labeled detection reagent (e.g., mouse monoclonal antibody) under conditions sufficient to form a complex that is captured by streptavidin-coated wells and detected with a luminescent reaction triggered by HRP activity. In some embodiments, a third immunoassay to analyze a third subset of the lung cancer associated proteomic markers. In some embodiments, the third subset of the lung cancer comprises Fibrinogen-like protein 1 (FGL1), a fragment thereof, or the proxy proteomic marker thereof. In some embodiments, the third immunoassay is an ELISA. In some embodiments, the ELISA is a sandwich ELISA.

### 2. Methods of Lung Cancer Screening

Provided herein are methods of screening a subject for the lung cancer based, at least in part, on an analysis of lung cancer associated markers detected in a biological sample obtained from the subject. In some embodiments, the subject is at risk of having the lung cancer.

a. Lung Cancer

In some embodiments, the lung cancer is non-small cell lung cancer. In some embodiments, the non-small cell lung cancer is adenocarcinoma. In some embodiments, the non-small cell lung cancer is squamous cell carcinoma. In some embodiments, the non-small cell carcinoma is epidermoid carcinoma. In some embodiments, the non-small cell lung cancer is large cell carcinoma. In some embodiments, the lung cancer is small cell lung cancer. In some embodiments, the lung cancer originates in the lung tissue. In some embodiments, the lung cancer metastasizes to other tissues of the subject. In some embodiments, the lung cancer is an early-stage lung cancer. In some embodiments, the early-stage lung cancer is stage I. In some embodiments, the early-stage lung cancer is stage II. In some embodiments, the lung cancer is late-stage lung cancer. In some embodiments, the late-stage cancer is stage IV. In some embodiments, the lung cancer is stage I lung cancer. In some embodiments, the lung cancer is stage II lung cancer. In some embodiments, the lung cancer is stage III lung cancer. In some embodiments, the lung cancer is stage IV lung cancer. In some embodiments, the lung cancer originated in the lung. In some embodiments, the lung cancer metastasized to the lungs.

b. Subject

In some embodiments, the subject is at risk of having the lung cancer. In some embodiments, the subject is not at risk of having the lung cancer. In some embodiments, the subject is asymptomatic (e.g., no signs or symptoms of lung cancer). In some embodiments, the subject is at risk of having the lung cancer based on the subject's age. In some embodiments, the subject is 50 years of age or older. In some embodiments, the subject is 55 years of age or older. In some embodiments, the subject is 60 years of age or older. In some embodiments, the subject is 65 years of age or older. In some embodiments, the subject is 70 years of age or older. In some embodiments, the subject is 75 years of age or older. In some embodiments, the subject is 80 years of age or older. In some embodiments, the subject is at risk for having the lung cancer based, at least in part, on the subject being between 50 and 80 years old. In some embodiments, the subject is at risk for having the lung cancer based, at least in part, on the subject being between 50 and 77 years old. In some embodiments, the subject is at risk for having the lung cancer based, at least in part, on the subject being between 50 and 75 years old.

In some embodiments, the subject is at risk of having the lung cancer based, at least in part, on the subject's smoking history. In some embodiments, the subject's smoking history comprises being a current smoker. In some embodiments, the subject's smoking history comprises being a past smoker. In some embodiments, the subject's smoking history comprises being a social smoker. In some embodiments, the subject's smoking history comprises what they smoked.

In some embodiments, the subject's smoking history comprises the number of packs the subject smoked a day. In some embodiments, the subject's smoking history comprises the number of packs the subject smoked a year. In some embodiments, the smoking history of the subject comprises smoking greater than or equal to 5 packs of cigarettes per year. In some embodiments, the smoking history of the subject comprises smoking greater than or equal to 10 packs of cigarettes per year. In some embodiments, the smoking history of the subject comprises smoking greater than or equal to 15 packs of cigarettes per year. In some embodiments, the smoking history of the subject comprises smoking greater than or equal to 20 packs of cigarettes per year. In some embodiments, the smoking history of the subject comprises smoking greater than or equal to 25 packs of cigarettes per year. In some embodiments, the smoking history of the subject comprises smoking greater than or equal to 30 packs of cigarettes per year. In some embodiments, the smoking history of the subject comprises smoking greater than or equal to 35 packs of cigarettes per year. In some embodiments, the smoking history of the subject comprises smoking greater than or equal to 40 packs of cigarettes per year. In some embodiments, the smoking history of the subject comprises smoking greater than or equal to 45 packs of cigarettes per year. In some embodiments, the smoking history of the subject comprises smoking greater than or equal to 50 packs of cigarettes per year.

In some embodiments, the subject's smoking history comprises the number of cigarettes the subject smoked a day. In some embodiments, the smoking history of the subject comprises smoking greater than or equal to 1 cigarette per day. In some embodiments, the smoking history of the subject comprises smoking greater than or equal to 2 cigarettes per day. In some embodiments, the smoking history of the subject comprises smoking greater than or equal to 3 cigarettes per day. In some embodiments, the smoking history of the subject comprises smoking greater than or equal to 4 cigarettes per day. In some embodiments, the smoking history of the subject comprises smoking greater than or equal to 5 cigarettes per day. In some embodiments, the smoking history of the subject comprises smoking greater than or equal to 10 cigarette per day. In some embodiments, the smoking history of the subject comprises smoking greater than or equal to 15 cigarettes per day. In some embodiments, the smoking history of the subject comprises smoking greater than or equal to 20 cigarettes per day. In some embodiments, the smoking history of the subject comprises smoking greater than or equal to 25 cigarettes per day. In some embodiments, the smoking history of the subject comprises smoking greater than or equal to 30 cigarettes per day. In some embodiments, the smoking history of the subject comprises smoking greater than or equal to 35 cigarettes per day. In some embodiments, the smoking history of the subject comprises smoking greater than or equal to 40 cigarettes per day. In some embodiments, the smoking history of the subject comprises smoking greater than or equal to 45 cigarettes per day. In some embodiments, the smoking history of the subject comprises smoking greater than or equal to 50 cigarettes per day.

In some embodiments, the subject's smoking history comprises the duration of time the subject smoked. In some embodiments, the subject's smoking history comprises the length of time they smoked. In some embodiments, the smoking history of the subject comprises smoking for greater than or equal to one year. In some embodiments, the smoking history of the subject comprises smoking for greater than or equal to two years. In some embodiments, the smoking history of the subject comprises smoking for greater than or equal to three years. In some embodiments, the smoking history of the subject comprises smoking for greater than or equal to four years. In some embodiments, the smoking history of the subject comprises smoking for greater than or equal to five years. In some embodiments, the smoking history of the subject comprises smoking for greater than or equal to ten years. In some embodiments, the smoking history of the subject comprises smoking for greater than or equal to fifteen years. In some embodiments, the smoking history of the subject comprises smoking for greater than or equal to twenty years. In some embodiments, the smoking history of the subject comprises smoking for greater than or equal to twenty-five years. In some embodiments, the smoking history of the subject comprises smoking for greater than or equal to thirty years. In some embodiments, the smoking history of the subject comprises smoking for greater than or equal to thirty-five years. In some embodiments, the smoking history of the subject comprises smoking for greater than or equal to forty years. In some embodiments, the smoking history of the subject comprises smoking for greater than or equal to forty-five years. In some embodiments, the smoking history of the subject comprises smoking for greater than or equal to fifty years.

In some embodiments, the subject's smoking history comprises the amount of time since they quit smoking. In some embodiments, the smoking history of the subject comprises having quit smoking for greater than or equal to one year. In some embodiments, the smoking history of the subject comprises having quit smoking for greater than or equal to two years. In some embodiments, the smoking history of the subject comprises having quit smoking for greater than or equal to three years. In some embodiments, the smoking history of the subject comprises having quit smoking for greater than or equal to four years. In some embodiments, the smoking history of the subject comprises having quit smoking for greater than or equal to five years. In some embodiments, the smoking history of the subject comprises having quit smoking for greater than or equal to ten years. In some embodiments, the smoking history of the subject comprises having quit smoking for greater than or equal to fifteen years. In some embodiments, the smoking history of the subject comprises having quit smoking for greater than or equal to twenty years. In some embodiments, the smoking history of the subject comprises having quit smoking for greater than or equal to twenty-five years. In some embodiments, the smoking history of the subject comprises having quit smoking for greater than or equal to thirty years. In some embodiments, the smoking history of the subject comprises having quit smoking for greater than or equal to thirty-five years. In some embodiments, the smoking history of the subject comprises having quit smoking for greater than or equal to forty years. In some embodiments, the smoking history of the subject comprises having quit smoking for greater than or equal to forty-five years. In some embodiments, the smoking history of the subject comprises having quit smoking for greater than or equal to fifty years.

In some embodiments, the subject has a greater than or equal to 5 pack-year history. In some embodiments, the subject has a greater than or equal 10 pack-year history. In some embodiments, the subject has a greater than or equal 15 pack-year history. In some embodiments, the subject has a greater than or equal 20 pack-year history. In some embodiments, the subject has a greater than or equal 25 pack-year history. In some embodiments, the subject has a greater than or equal 30 pack-year history. In these embodiments, a pack-year history is determined as a multiple of the number of packs a day and the number of years of smoking history. As a non-limiting example, a subject that smokes two packs a day for 10 years would have a 20-pack year history; however, a subject that smokes one pack a day for 20 years would also have a 20-pack year history.

In some embodiments, the subject meets the criteria of the U.S. Preventative Services Task Force (USPSTF) for lung cancer screening. In some embodiments, the criteria from the USPSTF have a grade of ‘B’ or higher. In some embodiments, the criteria from the USPSTF have a grade of ‘A’ or higher. In some embodiments, the subject meets the criteria of the Centers for Medicare and Medicaid Services (CMS) for lung cancer screening. In some embodiments, the subject meets the criteria of an ex-US agency that is equivalent to the USPSTF or CMS for lung cancer screening.

A non-limiting example of a subject for use of the test disclosed herein for assessing lung cancer includes a subject that is at least 50 years old with a greater than or equal to 20 pack-year history. In some embodiments, this subject is a current smoker. In some embodiments, this subject has quit smoking in fewer than or equal to 15 years.

In some embodiments, the subject is at risk of having the lung cancer based on the subject's medical history. In some embodiments, the subject's medical history includes diagnostic imaging of a mass in the lungs of the subject. Non-limiting examples of diagnostic imaging include computed tomography (CT), magnetic resonance imaging (MRI), an ultrasound, a chest X-ray, a positron emission tomography (PET), a PET-CT. In some embodiments, the subject has not previously been diagnosed with cancer. In some embodiments, the subject has not been previously diagnosed with lung cancer. In some embodiments, the subject has been previously diagnosed with cancer. In some embodiments, the subject has previously been diagnosed with lung cancer. In some embodiments, the subject is human.

c. Classifier

In some embodiments, a classifier may be applied to the proteomic measurements. In some embodiments, the classifier may be applied to a combination of the proteomic measurements and clinical history (e.g., age, smoking history, medical history, race, ethnicity, any clinical indicators for lung cancer, or any combination thereof). The classifier may provide a quantitative result, qualitative result, or both. The result may be for a biofluid sample. The classifier may distinguish lung cancer from non-lung cancer. The quantitative result may be a degree of risk that the subject has the lung cancer. The degree of risk may be a probability, a likelihood, a time-to-event, or any combination thereof. The qualitative result may be a determination that the subject has the lung cancer or not. The qualitative result may be an odds ratio that a subject is likely to develop the lung cancer or not.

The classifier may comprise a trained machine learning model. The classifier may comprise an aggregation of the input to the machine learning model or a portion thereof (such as a layer in a neural network, or a decision tree in a gradient boosted model). The input to the machine learning may comprise proteomic measurements. The input to the machine learning model may comprise features (such as measurements of individual proteins in the proteomic measurements).

The aggregation may comprise applying a weight or array of weights to one or more of the proteomic measurements. The aggregation may comprise applying a transformation (such as a linear or non-linear transformation) to the proteomic measurements (either after applying the weights or before). A transformation may be a linear transformation, an affine transformation, a polynomial expansion, a logarithmic transformation, an exponential transformation, a power transformation, a square root transformation, a reciprocal transformation, a standardization (e.g., z-score), a min-max scaling, a max-abs scaling, a robust scaling (e.g., median and IQR), a normalization (e.g., L1, L2), a one-hot encoding, a frequency encoding, a binary encoding, a mean encoding, an embedding (learned or pre-trained), a binning (e.g., equal width or equal frequency), a discretization, a clipping, a thresholding, a principal component analysis (PCA), an independent component analysis (ICA), a kernel PCA, a Fourier transform, a wavelet transform, a random projection, a batch normalization, a layer normalization, noise injection, quantile transformation, rank transformation, Box-Cox transformation, Yeo-Johnson transformation, sigmoid function, tanh function, ReLU (Rectified Linear Unit), a variant of ReLU (e.g., Leaky ReLU, ELU, GELU, SELU), softmax, softplus, swish, attention-based transformations, convolution, pooling (max, average, global), positional encoding, residual connections, feature hashing, or any combination thereof. The aggregation may comprise an accumulation (such as a summation). The aggregation may comprise a summation. The aggregation may comprise applying a transformation to the summation. The machine learning model may comprise multiple aggregations. The multiple aggregations may comprise the same aggregation (such as comprising the same accumulation, and transformation). The multiple aggregations may comprise different aggregations (such as having a different accumulation, transformation, or both). The aggregation may comprise the addition of a bias.

The accumulation may comprise a summation of values (such as a summation of weighted inputs). The accumulation may comprise multiplication of values (such as the weighted inputs). The accumulation may comprise a polynomial (such as in polynomial regression or to generate interaction features) which may comprise term, such as interaction terms or squared terms. The polynomial accumulation may generate interaction features. An interaction feature may represent the combined effect of two or more features on the output (e.g., distinguishing lung cancer from non-cancer), which may capture relationships between the two or more input variables. The accumulation may comprise a min/max aggregation which may find the minimum and/or maximum value(s) among a plurality of features. The accumulation may comprise an attention-weighted summation (such as in transformers) where the features may be aggregated according to a learned attention score. For example, input features may be given a weight depending on their relevance to a query, and the final aggregation may be a weighted sum, where weights vary per instance (such as across different regions of the input features or across inputs which are temporally dependent). This allows the model to dynamically focus on different inputs for each prediction. The accumulation may comprise a geometric (e.g., log-sum) accumulation. The accumulation may comprise a graph-based accumulation (such as those used in a graph neural network or a graph convolution network) where a machine learning model may comprise a plurality of nodes connected by a plurality of edges where nodes are connected by edges and which may aggregate features through a message passing algorithm and accumulate (such as by summing) the features of connected nodes.

The classifier may comprise linear regression, logistic regression, ridge regression, lasso regression, elastic net, polynomial regression, Bayesian linear regression, stepwise regression, support vector machine (SVM), support vector regression (SVR), kernel SVM, decision tree, random forest, gradient boosting machines (GBM), XGBoost, LightGBM, CatBoost, AdaBoost, histogram-based gradient boosting, k-nearest neighbors (KNN), naive Bayes (e.g., Gaussian, multinomial, Bernoulli), linear discriminant analysis (LDA), quadratic discriminant analysis (QDA), perceptron, multilayer perceptron (MLP), feedforward neural network, convolutional neural network (CNN), recurrent neural network (RNN), long short-term memory (LSTM), gated recurrent unit (GRU), transformer, vision transformer (ViT), BERT, GPT, autoencoder, variational autoencoder (VAE), sparse autoencoder, denoising autoencoder, generative adversarial network (GAN), deep belief network (DBN), restricted Boltzmann machine (RBM), self-organizing map (SOM), extreme learning machine (ELM), probabilistic graphical models, Markov decision process (MDP), hidden Markov model (HMM), conditional random field (CRF), Kalman filter, Gaussian process regression, Gaussian mixture model (GMM), k-means clustering, hierarchical clustering, DBSCAN, OPTICS, spectral clustering, mean shift clustering, agglomerative clustering, affinity propagation, principal component analysis (PCA), independent component analysis (ICA), factor analysis, non-negative matrix factorization (NMF), matrix factorization, collaborative filtering (user-based, item-based), singular value decomposition (SVD), neural collaborative filtering, reinforcement learning (e.g., Q-learning, SARSA, DQN, actor-critic, PPO, A3C, DDPG, TD3, SAC), evolutionary algorithms (e.g., genetic algorithms, genetic programming), swarm optimization (e.g., particle swarm, ant colony), simulated annealing, Monte Carlo methods, ensemble models (e.g., bagging, boosting, stacking, voting), rule-based models (e.g., rule fit, decision rules), fuzzy logic systems, symbolic regression, neuroevolution models, or any combination thereof. The classifier may comprise a linear regression algorithm. The classifier may comprise a logistic regression algorithm. The classifier may comprise a gradient boosted model. In some embodiments, measurements from the lung cancer associated markers detected in a biological sample of a subject are input into a classifier, which provides a quantitative or qualitative result related to the lung cancer.

### 1) Performance Characteristics

In some embodiments, the classifier distinguishes the lung cancer from non-cancer with a performance characteristic comprising a sensitivity of at least 85%, a specificity of at least 55%, an area under the curve (AUC) that is greater than or equal to about 0.80, or any combination thereof. In some embodiments the classifiers performance characteristic may be measured using accuracy, balanced accuracy, precision, recall (sensitivity), specificity, F1 score, F0.5 score, F2 score, ROC AUC (area under the ROC curve), PR AUC (area under the precision-recall curve), top-k accuracy, label ranking average precision (LRAP, average precision, macro-averaged metrics, micro-averaged metrics, weighted-averaged metrics, confusion matrix values (e.g., TP, FP, TN, FN), mean absolute error (MAE), mean squared error (MSE), root mean squared error (RMSE), mean absolute percentage error (MAPE), symmetric mean absolute percentage error (SMAPE), mean squared log error (MSLE), root mean squared log error (RMSLE), median absolute error, mean average precision (MAP), perplexity, out-of-bag error (OOB error), or any combination thereof.

The performance characteristic may comprise sensitivity. Sensitivity is a measure of a tests ability to correctly identify individuals who have a condition (e.g., lung cancer). The sensitivity may be greater than or equal to about 50%, 55%, 60%, 65%, 70%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. In some embodiments, the sensitivity is about 50% to 99%, 60% to 98%, 65% to 97%, 70% to 96%, 75% to 95%, 76% to 94%, 77% to 93%, 78% to 92%, 79% to 91%, 80% to 90%, 81% to 89%, 82% to 88%, or 83% to 87%. The sensitivity may be greater than or equal to about 87%. The sensitivity may be greater than or equal to about 88%. The sensitivity may be 100%. In some embodiments, the sensitivity of the classifier may differ depending on the stage and/or type of the lung cancer detected. In some embodiments, the sensitivity is greater than or equal to about 87% for stage 1 non-small cell lung cancer. In some embodiments, the sensitivity is greater than or equal to about 88% for stage 2 non-small cell lung cancer. In some embodiments, the sensitivity is equal to about 100% for stage 3 or stage 4 non-small cell lung cancer.

The performance characteristic may comprise an area under the curve (AUC). The AUC is a measure of the test's overall ability to discriminate between non-lung cancer and lung cancer individuals, with a value ranging from 0.5 (random chance) to 1.0 (perfect accuracy). The AUC may be greater than or equal to about 0.80. The AUC may be greater than or equal to about 0.82. The AUC may be greater than or equal to about 0.50, 0.55, 0.60, 0.65, 0.70, 0.75, 0.80, 0.81, 0.82, 0.83, 0.84, 0.85, 0.86, 0.87, 0.88, 0.89, 0.90, 0.91, 0.92, 0.93, 0.94, 0.95, 0.96, 0.97, 0.98, or 0.99. AUC may be equal to about 1.0. The AUC may be 0.50 to 0.99, 0.55 to 0.98, 0.60 to 0.97, 0.65 to 0.96, 0.70 to 0.95, 0.75 to 0.94, 0.80 to 0.93, 0.81 to 0.92, 0.82 to 0.91, 0.83 to 0.90, 0.84 to 0.89, or 0.85 to 0.88.

The performance may comprise specificity. Specificity is a measure of a test's ability to correctly identify individuals that do not have the condition (e.g., lung cancer). The specificity may be greater than or equal to about 40%, 50%, 51%, 52%, 53% 54%, 55%, 56%, 57%, 58%, 59%, 60%, 61%, 62%, 63%, 64%, 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%. The specificity may be 51% to 99%, 52% to 98%, 53% to 97%, 54% to 96%, 55% to 95%, 56% to 94%, 57% to 93%, 58% to 92%, 59% to 91%, 60% to 90%, 61% to 89%, 62% to 88%, 63% to 87%, 64% to 86%, 65% to 85%, 66% to 84%, 67%, to 83%, 68%, to 82%, 69% to 81%, 70% to 80%, 71% to 79%, 72% to 78%, 73% to 77%, or 74% to 76%. The specificity may be 100%.

The performance may be a negative predictive value (NPV). NPV measures the likelihood that a person who tests negative for a condition truly does not have the condition (e.g., lung cancer). The NPV may be greater than or equal to about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%. The NPV may be 50% to 99%, 60% to 98%, 65% to 97%, 70% to 96%, 75% to 95%, 76% to 94%, 77% to 93%, 78% to 92%, 79% to 91%, 80% to 90%, 81% to 89%, 82% to 88%, or 83% to 87%. In some embodiments, the NPV of the classifier may differ depending on the stage and/or type of the lung cancer detected. In some embodiments, the NPV is greater than or equal to 99% (e.g., 99.8%) for stage 1 non-small cell lung cancer.

The performance may be a positive predictive value (PPV). PPV measures the likelihood that a person who tests positive for a condition truly has the condition (e.g., lung cancer). The PPV may be greater than or equal to about 50%, 55%, 60%, 65%, 70%, 75%, 80%, 85%, 86%, 87%, 88%, 89%, 90%, 91%, 92%, 93%, 94%, 95%, 96%, 97%, 98%, 99%, or 100%. The PPV may be 50% to 99%, 60% to 98%, 65% to 97%, 70% to 96%, 75% to 95%, 76% to 94%, 77% to 93%, 78% to 92%, 79% to 91%, 80% to 90%, 81% to 89%, 82% to 88%, or 83% to 87%.

### 2) Training the Classifier

In some embodiments, the classifier is trained. A machine learning model (such as a classifier) may be trained to make predictions or detect patterns. Training may comprise a training algorithm which may comprise a loss and an optimizer. Training may comprise providing a training cohort (e.g., a training dataset) as input data (such as a protein measurement data) to the machine learning model. The machine learning model may learn by applying the training algorithm to the model such that the training algorithm calculates a loss (e.g., an error) of the models output over at least a portion of the training data and updates the machine learning model to improve the loss of the machine learning model. Training may be performed in an iterative fashion where iterations comprise the model generating output for at least a portion of the training data, a loss being calculated, and the optimizer updating the model. Training may continue until a criterion has been met such as a number of iterations with improvement in the loss under a minimum value, a set number of iteration, the updates to the model are below a given threshold (e.g., a gradient becoming too small). The loss may be calculated based on a target outcome. The target outcome may may comprise labels (e.g., a ground truth), such as in supervised learning. In supervised learning, the model may be trained on using labels and input data that correspond to the labels, the model then may learn to map inputs to the labels. For example, the input data comprises two samples and two labels and sample 1 is labeled with label 1, sample 2 is labeled with label 2.

The target outcome may be based the distribution of the training dataset, such as in unsupervised learning unsupervised learning, or self-supervised learning. In unsupervised learning, the model may receive inputs and learn structures and/or groupings in the training data. In self-supervised learning, a model may take as input the training dataset and automatically generate labels from the data itself, for example a portion of the input may be hidden (e.g., masked) and the machine learning model may predict them. Another example of self-supervised learning may have the machine learning model compare two inputs and minimize or maximize the difference between the two inputs.

During training the parameters of the machine learning model may be adjusted to reduce the loss. This adjustment may be guided by the optimizer which comprises an optimization algorithm. Optimization algorithms, incrementally update the parameters to reduce (e.g., minimize) the loss.

A training dataset may be balanced where the samples are distributed approximately uniformly across the possible observable samples or labels. A training dataset may be unbalanced where the samples distribution is biased across the possible observable samples or labels. For example, a lung cancer training dataset is composed of 1% lung cancer sample and 99% non-lung cancer samples. In some cases, an unbalanced dataset may be enriched (e.g., modified to comprise a higher proportion of a sample type or of a portion of the distribution of samples). Enrichment may comprise removing samples. Enrichment may comprise selectively generating more samples of a given sample type (such as lung cancer or not-lung cancer). A dataset may be enriched by a percentage such that the dataset comprises the percentage of a given sample type.

In some embodiments, the classifier of the present disclosure is training with a training cohort that is minimally enriched. In some embodiments, the one or more performance characteristics of the classifier is obtained using a training cohort that is minimally enriched. In some embodiments, the training cohort is enriched no more than 1%, no more than 2%, no more than 3%, no more than 4%, no more than 5%, no more than 6%, no more than 7%, no more than 8%, no more than 9%, no more than 10%, no more than 11%, no more than 12%, no more than 13%, no more than 14%, no more than 15%, no more than 20%, no more than 25%, no more than 30%, no more than 35%, no more than 40%, no more than 45%, no more than 50%, no more than 60%, no more than 70%, no more than 80%, no more than 90%, no more than 99%. In some embodiments, the training cohort is enriched no more than 12%. In some embodiments, the training cohort is enriched from 1% to 15%, 2% to 14%, 3% to 13%, 4% to 12%, 5% to 11%, 6% to 10%, or 7% to 9%. In some embodiments, the training cohort is enriched no more than 12%.

### 3) Thresholding

In some embodiments, the classifier distinguishes the lung cancer from non-cancer by applying a threshold to one or more measurements from the lung cancer associated markers. In some embodiments, a threshold is applied to the output of a machine learning model. In some embodiments, the distinguishing comprises outputting a probability score. In some embodiments, the threshold is determined using an analysis of a precision-recall curve. The precision recall score may be based on the probability scores of a dataset. In some embodiments, the threshold is determined using an analysis of a receiver operating curve. The receiver operation curve may be based on the probability scores of a dataset. The machine learning model may output probability scores for a plurality of samples in the dataset. The receiver operation curve may be generated based on the output probability scores for the plurality of samples in the dataset. The precision-recall curve may be generated based on the output probability scores for the plurality of samples in the dataset.

A threshold may be determined which corresponds to a given sensitivity and/or specificity. The threshold may be determined by analyzing a curve (such as a precision-recall cure or a receiver operating curve) The analyzing may comprise scanning a curve (such as a precision recall curve or a receiver operating curve) and identifying the position on the curve that corresponds to a sensitivity and specificity. The threshold may be recorded to be applied to future samples. The threshold may be applied to the output of the machine learning model for a sample to determine a classification of the sample. For example, a sample may be input to a machine learning model which outputs a value (such as a probability) the threshold may be applied to the output and if the output is above the threshold the sample is classified as a first class (such as lung cancer), if the output is below the threshold the sample is classified as a second class (such as non-lung cancer). In some embodiments, the analysis determines a threshold corresponding to the sensitivity of greater than or equal to about 85% and the specificity of greater than or equal to about 55%. In some embodiments, the analysis determines a threshold corresponding to any combination of sensitivity and specificity provide above in Section (I)(2)(c)(1)(“Performance Characteristics”).

d. Test Report

In some embodiments, the result of the test is displayed in a test report. In some embodiments, the test report indicates the subject is positive for lung cancer. In some embodiments, the test report indicates the subject is negative for lung cancer. In some embodiments, the test report indicates the test was inconclusive. In some embodiments, an inconclusive test result recommends redoing the test. In some embodiments, an inconclusive test result recommends additional testing. In some embodiments, the additional testing comprises diagnostic imaging. Non-limiting examples of diagnostic imaging include computed tomography (CT), magnetic resonance imaging (MRI), an ultrasound, a chest X-ray, a positron emission tomography (PET), a PET-CT.

In some embodiments, the test report indicates a likelihood of cancer in the subject. In some embodiments, the test report indicates a high likelihood of lung cancer in the subject. In some embodiments, the test report indicates a low likelihood of lung cancer in the subject. In some embodiments, the test report indicates a moderate likelihood of lung cancer in the subject. In some embodiments, the test report indicates a percentage of the likelihood of cancer in the subject.

In some embodiments, the test report indicates the subject has an elevated test result. In some embodiments, an elevated test result indicates a subject has an elevated risk of lung cancer. In some embodiments, an elevated risk of lung cancer is elevated relative to subjects at risk for lung cancer meeting inclusion criteria. In some embodiments, an elevated test result indicates a subject has a higher risk for lung cancer than the baseline. In some embodiments, a test report indicating a subject has an elevated test result recommends additional diagnostic testing. In some embodiments, additional diagnostic testing comprises diagnostic imaging. In some embodiments, the additional diagnostic testing comprises a biopsy. In some embodiments, the test report indicates the subject has a non-elevated test result. In some embodiments, a non-elevated test result indicates a subject does not have an elevated risk of lung cancer. In some embodiments, a non-elevated result is not elevated relative to subjects at risk for lung cancer meeting inclusion criteria. In some embodiments, a non-elevated test result indicates a subject has a similar risk of lung cancer as the baseline. In some embodiments, a test report indicating a subject has a non-elevated test result recommends continuing to participate in lung cancer screening. In some embodiments, a baseline is determined from a subject eligible for lung cancer screening. In some embodiments, the baseline is generated from a subject that meets the inclusion criteria. In some embodiments, the baseline is generated from subjects meeting the inclusion criteria with and without lung cancer. As a non-limiting example, the baseline is generated from a subject meeting the inclusion criteria including that they are 50 years or older and have a 20 pack-year smoking history. In some embodiments, the inclusion criteria also include having a current smoking history or having quit smoking in the last 15 years (e.g., 15 years or less). In some embodiments, the inclusion criteria are based on the U.S. preventative services task force (USPSTF) recommendations for lung cancer screenings. In some embodiments, the inclusion criteria are included in Mazzone et al. (ATS Assembly on Thoracic Oncology. Evaluating Molecular Biomarkers for the Early Detection of Lung Cancer: When Is a Biomarker Ready for Clinical Use? An Official American Thoracic Society Policy Statement. Am J Respir Crit Care Med. 2017 Oct. 1; 196(7):e15-e29) or Cotton et al. (Improving the Efficiency of Lung Cancer Screening Through a Blood-based Lung Cancer Screening Test Prior to Low-Dose CT. (P4.04C.07). World Conference on Lung Cancer. 2024 Sep. 9 San Diego, CA, United States.) which are hereby incorporated in their entirety by reference. In some embodiments, the baseline is based on the classifier or model output for assessing a subject's risk of lung cancer.

In some embodiments, the test report indicates the subject's test has been canceled. In some embodiments, the test report indicates the subjects test had no results obtained. In some embodiments, the test report indicates the subject needs to repeat the test. In some embodiments, the test report indicates the subject should follow up with the standard of care recommended by their prescribing physician. In some embodiments, the test report indicates the subject should follow up with diagnostic imaging. Non-limiting examples of diagnostic imaging include computed tomography (CT), magnetic resonance imaging (MRI), an ultrasound, a chest X-ray, a positron emission tomography (PET), a PET-CT.

### 3. Methods of Treatment

Provided herein are methods of treating lung cancer in a subject. In some embodiments, the methods comprises administering a therapeutic agent for the treatment of the lung cancer to the subject. In some embodiments, a method of treating lung cancer in a subject comprises surgery. In some embodiments, a method of treating lung cancer in a subject comprises radiation therapy. In some embodiments, a method for treating lung cancer in a subject uses two or more a therapeutic agent, surgery, and radiation therapy. In some embodiments, a method for treating lung cancer in a subject comprises two or more therapeutic agents. In some embodiments, a method for treating lung cancer in a subject comprises two or more therapeutic agents in combination with surgery, radiation therapy, or both. In some embodiments, the subject is or has previously been classified as having the lung cancer based, at least in part, on an analysis of measurements from the lung cancer associated markers detected in a biological sample of a subject.

a. Therapeutic Agents for Treatment of Lung Cancer

In some embodiments, the therapeutic agent for treatment of the lung cancer is a targeted therapy. In some embodiments, the therapeutic agent for treatment of the lung cancer is a chemotherapy. In some embodiments, the therapeutic agent for treatment of the lung cancer is an immunotherapy. In some embodiments, the therapeutic agent for treatment of the lung cancer is a non-targeted therapy. In some embodiments, the non-targeted therapy for treatment of lung cancer is a chemotherapy.

In some embodiments, the therapeutic agent for treatment of the lung cancer is an inhibitor. In some embodiments, the therapeutic agent for treatment of the lung cancer is an angiogenesis inhibitor. In some embodiments, the therapeutic agent for treatment of the lung cancer is an agonist. In some embodiments, the therapeutic agent for treatment of the lung cancer is a small molecule. In some embodiments, the therapeutic agent for treatment of the lung cancer is an antibody. In some embodiments, an antibody includes intact polyclonal antibodies, intact monoclonal antibodies, antibody fragments (such as Fab, Fab′, F(ab′)2, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity. In some embodiments, the therapeutic agent for treatment of the lung cancer is a monoclonal antibody. In some embodiments, the therapeutic agent for treatment of the lung cancer is a modulator. In some embodiments, the therapeutic agent for treatment of the lung cancer is an allosteric modulator. In some embodiments, the lung cancer originated in the lung. In some embodiments, the lung cancer metastasized to the lungs.

In some embodiments, the therapeutic agent for treatment of the lung cancer interacts with one or more proteins. In some embodiments, the therapeutic agent for treatment of the lung cancer targets one or more proteins. In some embodiments, the therapeutic agent for treatment of the lung cancer interacts with the one or more proteins by inhibiting the one or more proteins. In some embodiments, the therapeutic agent for treatment of the lung cancer interacts with the one or more proteins by binding the one or more proteins. In some embodiments, the therapeutic agent for treatment of the lung cancer interacts with the one or more proteins by agonizing the one or more proteins. In some embodiments, the therapeutic agent for treatment of the lung cancer interacts with the one or more proteins by modulating the one or more proteins. In some embodiments, the one or more proteins is one or more of the proteins disclosed herein. In some embodiments, the one or more proteins is one or more of the proteins disclosed in Table 1. In some embodiments, the one or more proteins is a proxy protein or one or more proxy proteins of the one or more proteins disclosed herein. In some embodiments, the one or more proteins is a proxy protein or one or more proxy proteins of the one or more proteins disclosed in Table 1. As a non-limiting example, the therapeutic agent for treatment of the lung cancer may include one or more of the therapeutic agents listed in Table 40 which targets the one or more proteins disclosed in Table 40. Each of the references recited in Table 40 are herein incorporated in their entirety.

TABLE 40
Therapeutic agents targeting FGL1, gp130, or PECAM-1

| | Therapeutic | |
|---|---|---|
| Protein | Agent | Reference |
| Fibrinogen-like protein | Anti-FGL1 | Tian T, Xie X, Yi W, Zhou Y, Xu Y, Wang Z, |
| 1 (FGL1) | antibody | Zhang J, Lin M, Zhang R, Lv Z, Li X, Lv L, |
| | | Xu Y. FBXO38 mediates FGL1 ubiquitination |
| | | and degradation to enhance cancer immunity |
| | | and suppress inflammation. Cell Rep. 2023 |
| | | Nov. 28; 42(11): 113362. doi: |
| | | 10.1016/j.celrep.2023.113362. Epub 2023 Nov. 7. |
| | | PMID: 37938970. |
| | | Wang J, Sanmamed MF, Datar I, Su TT, Ji L, |
| | | Sun J, Chen L, Chen Y, Zhu G, Yin W, Zheng |
| | | L, Zhou T, Badri T, Yao S, Zhu S, Boto A, |
| | | Sznol M, Melero I, Vignali DAA, Schalper K, |
| | | Chen L. Fibrinogen-like Protein 1 Is a Major |
| | | Immune Inhibitory Ligand of LAG-3. Cell. |
| | | 2019 Jan. 10; 176(1-2): 334-347.e12. doi: |
| | | 10.1016/j.cell.2018.11.010. Epub 2018 Dec. 20. |
| | | PMID: 30580966; PMCID: PMC6365968. |
| | | Liu J, Liu Q, Qian W, Zong C, Wang R. IL-6 |
| | | promotes metastasis and EMT of non-small |
| | | cell lung cancer cells by up-regulating FGL1 |
| | | via STAT3 pathway. Transl Cancer Res. 2025 |
| | | Jul. 30; 14(7): 3973-3990. doi: 10.21037/tcr- |
| | | 2025-119. Epub 2025 Jul. 27. PMID: |
| | | 40792131; PMCID: PMC12335700. |
| | AAV9 | Tang XY, Zhang RZ, Feng ZB, Zhou YL, Du |
| | AAV6 | WG, Shu C, Shen Y, Li MC, Cai JC, Yan XL, |
| | | Ma N, Zhao JB. FGL1-mediated lymph node |
| | | metastasis in stage T1 non-small cell lung |
| | | cancer: therapeutic targeting. Exp Hematol |
| | | Oncol. 2025 Sep. 29; 14(1): 117. doi: |
| | | 10.1186/s40164-025-00709-5. PMID: |
| | | 41024301; PMCID: PMC12481761. |
| Glycoprotein 130 | Receptor | McIntosh BJ, Hartmann GG, Yamada-Hunter |
| (GP130) | antagonist | SA, Liu P, Williams CF, Sage J, Cochran JR. |
| | | An engineered interleukin-11 decoy cytokine |
| | | inhibits receptor signaling and proliferation in |
| | | lung adenocarcinoma. Bioeng Transl Med. |
| | | 2023 Jul. 18; 8(6): e10573. doi: |
| | Acovenosigenin A | 10.1002/btm2.10573. PMID: 38023717; |
| | β-glucoside | PMCID: PMC10658506. |
| | | Liu Z, Wang Q, Chi Y, Chen R, Zhao L, Liu |
| | | Z, Zhai J, Li S, Han N, Yin J. Acovenosigenin |
| | | A β-glucoside mediates JAK2-STAT3 |
| | | signaling pathway by targeting GP130 in A549 |
| | | and H460 cells based on integrative analysis of |
| | | transcriptome and proteome and biological |
| | | verification. Bioorg Chem. 2024 |
| | | October; 151: 107633. doi: |
| | | 10.1016/j.bioorg.2024.107633. Epub 2024 Jul. 11. |
| | | PMID: 39003941. |
| | Bazedoxifene | Huang Y, Lin J, Fu X, Li L, Fu S. |
| | | Bazedoxifene Inhibits Cell Viability, Colony- |
| | | Forming Activity, and Cell Migration in |
| | | Human Non-Small Cell Lung Cancer Cells and |
| | | Improves the Treatment Efficacy of Paclitaxel |
| | | and Gemcitabine. Clin Respir J. 2024 |
| | | August; 18(8): e13822. doi: 10.1111/crj.13822. |
| | | PMID: 39152779; PMCID: PMC11329873. |
| | Epirubicin | Liu F, Jin H, Shen J, Wu D, Tian Y, Huang C. |
| | | Gp130 degradation induced by epirubicin |
| | | contributes to chemotherapy efficacy. |
| | | Biochem Biophys Res Commun. 2019 Nov. 12; |
| | | 519(3): 572-578. doi: |
| | | 10.1016/j.bbrc.2019.09.055. Epub 2019 Sep. 16. |
| | | PMID: 31537377. |
| Platelet endothelial | anti-PECAM-1 | DeLisser H, Liu Y, Desprez PY, Thor A, |
| cell adhesion molecule | antibody | Briasouli P, Handumrongkul C, Wilfong J, |
| (PECAM-1) | | Yount G, Nosrati M, Fong S, Shtivelman E, |
| | | Fehrenbach M, Cao G, Moore DH, Nayak S, |
| | | Liggitt D, Kashani-Sabet M, Debs R. Vascular |
| | | endothelial platelet endothelial cell adhesion |
| | | molecule 1 (PECAM-1) regulates advanced |
| | | metastatic progression. Proc Natl Acad Sci U S A. |
| | | 2010 Oct. 26; 107(43): 18616-21. doi: |
| | | 10.1073/pnas. 1004654107. Epub 2010 Oct. 6. |
| | | Erratum in: Proc Natl Acad Sci U S A. 2011 |
| | | Mar. 1; 108(9): 3821. Nyack, Shruti [corrected |
| | | to Nayak, Shruti]. PMID: 20926749; PMCID: |
| | | PMC2973011. |

In some embodiments, treatment for lung cancer comprises reverses the lung cancer. In some embodiments, treatment for lung cancer comprises curing the lung cancer. In some embodiments, treatment for lung cancer comprises slowing progression of the lung cancer. In some embodiments, treatment for lung cancer comprises reversing progression of the lung cancer. In some embodiments, treatment for lung cancer comprises preventing progression of the lung cancer.

In some embodiments, the therapeutic agent for treatment of the lung cancer is a KRAS inhibitor. In some embodiments, the therapeutic agent for treatment of the lung cancer is an EGFR inhibitor. In some embodiments, the therapeutic agent for treatment of the lung cancer is an ALK inhibitor. In some embodiments, the therapeutic agent for treatment of the lung cancer is a ROS1 inhibitor. In some embodiments, the therapeutic agent for treatment of the lung cancer is a BRAF inhibitor. In some embodiments, the therapeutic agent for treatment of the lung cancer is a RET inhibitor. In some embodiments, the therapeutic agent for treatment of the lung cancer is a MET inhibitor. In some embodiments, the therapeutic agent for treatment of the lung cancer is a HER2-directed therapeutic. In some embodiments, the therapeutic agent for treatment of the lung cancer is a TRK inhibitor. In some embodiments, the therapeutic agent for treatment of the lung cancer is an antibody-drug conjugate. In some embodiments, the therapeutic agent for treatment of the lung cancer is an immunotherapy. In some embodiments, the therapeutic agent is selected from Table 2.

TABLE 2
Therapeutic Agents for Treatment of Lung Cancer

Abraxane (Paclitaxel Albumin-stabilized Nanoparticle Formulation)
Adagrasib
Ado-Trastuzumab Emtansine
Afatinib Dimaleate
Afinitor (Everolimus)
Afinitor Disperz (Everolimus)
Alecensa (Alectinib)
Alectinib
Alimta (Pemetrexed Disodium)
Alunbrig (Brigatinib)
Alymsys (Bevacizumab)
Amivantamab-vmjw
Atezolizumab
Atezolizumab and Hyaluronidase-tqjs
Augtyro (Repotrectinib)
Avastin (Bevacizumab)
Bevacizumab
Binimetinib
Bizengri (Zenocutuzumab-zbco)
Braftovi (Encorafenib)
Brigatinib
Cabozantinib
Capmatinib Hydrochloride
Carboplatin
Cemiplimab-rwlc
Ceritinib
Cisplatin
Crizotinib
Cyramza (Ramucirumab)
Dabrafenib Mesylate
Dacomitinib
Datopotamab
Datopotamab Deruxtecan-dlnk
Docetaxel
Doxorubicin Hydrochloride
Durvalumab
Encorafenib
Enhertu (Fam-Trastuzumab Deruxtecan-nxki)
Ensacove (Ensartinib Hydrochloride)
Ensartinib Hydrochloride
Entrectinib
Erdafitinib
Erlotinib Hydrochloride
Etoposide
Everolimus
Fam-Trastuzumab Deruxtecan-nxki
Gavreto (Pralsetinib)
Gefitinib
Gilotrif (Afatinib Dimaleate)
Gemcitabine Hydrochloride
Gemzar (Gemcitabine Hydrochloride)
Imatinib (Gleevec)
Imfinzi (Durvalumab)
Imjudo (Tremelimumab-actl)
Infugem (Gemcitabine Hydrochloride)
Ipilimumab
Iressa (Gefitinib)
Keytruda (Pembrolizumab)
Krazati (Adagrasib)
Lazcluze (Lazertinib Mesylate Hydrate)
Lazertinib Mesylate Hydrate
Libtayo (Cemiplimab-rwlc)
Lorbrena (Lorlatinib)
Lorlatinib
Lumakras (Sotorasib)
Lurbinectedin
Mekinist (Trametinib Dimethyl Sulfoxide)
Mektovi (Binimetinib)
Methotrexate Sodium
Mvasi (Bevacizumab)
Necitumumab
Nivolumab
Nivolumab and Hyaluronidase-nvhy
Opdivo (Nivolumab)
Opdivo Qvantig (Nivolumab and Hyaluronidase-nvhy)
Osimertinib Mesylate
Paclitaxel
Paclitaxel Albumin-stabilized Nanoparticle Formulation
Pembrolizumab
Pemetrexed Disodium
Portrazza (Necitumumab)
Pralsetinib
Ramucirumab
Repotrectinib
Retevmo (Selpercatinib)
Rozlytrek (Entrectinib)
Rybrevant (Amivantamab-vmjw)
Selpercatinib
Sotorasib
Sunvozertinib
Tabrecta (Capmatinib Hydrochloride)
Tafinlar (Dabrafenib Mesylate)
Tagrisso (Osimertinib Mesylate)
Taletrectinib
Tarceva (Erlotinib Hydrochloride)
Taxotere (Docetaxel)
Tecentriq (Atezolizumab)
Tecentriq Hybreza (Atezolizumab and Hyaluronidase-tqjs)
Telisotuzumab Vedotin-tllv
Tepmetko (Tepotinib Hydrochloride)
Tepotinib Hydrochloride
Trametinib Dimethyl Sulfoxide
Tremelimumab-actl
Vemurafenib
Vizimpro (Dacomitinib)
Vinorelbine Tartrate
Xalkori (Crizotinib)
Yervoy (Ipilimumab)
Zenocutuzumab-zbco
Zirabev (Bevacizumab)
Zongertinib
Zykadia (Ceritinib)
Carboplatin-Taxol
Gemcitabine-Cisplatin
Etopophos (Etoposide Phosphate)
Etoposide
Etoposide Phosphate
Hycamtin (Topotecan Hydrochloride)
Imdelltra (Tarlatamab-dlle)
Lurbinectedin
Tarlatamab-dlle
Topotecan Hydrochloride
Zepzelca (Lurbinectedin)

Also provided herein are methods of treating a disease or a condition other than lung cancer in a subject comprising administering a therapeutic agent for the treatment of the disease or the condition other than the lung cancer to the subject. In some embodiments, the disease or the condition is a comorbidity of the lung cancer. In some embodiments, the comorbidity is chronic obstructive pulmonary disease (COPD), peripheral vascular disease (PVD), diabetes, congestive heart failure, cerebrovascular disease, renal disease, or a combination thereof.

b. Dosages and Routes of Administration

In general, methods disclosed herein comprise administering a therapeutic agent by intravenous (“i.v.”) administration. However, in some instances, methods comprise administering a therapeutic agent by oral administration. In some instances, methods comprise administering a therapeutic agent by intramuscular injection. It is conceivable that one may also administer therapeutic agents disclosed herein by other routes, such as subcutaneous injection, intraperitoneal injection, intradermal injection, transdermal injection, percutaneous administration, intranasal administration, intralymphatic injection, or any other suitable administration. Routes, dosage, time points, and duration of administrating therapeutics may be adjusted.

An effective dose and dosage of therapeutics to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. In some embodiments, the beneficial response may be measured by detecting a measurable improvement in the size of a tumor (e.g., the tumor stops growing or the tumor shrinks). An “improvement,” as used herein refers to shift in the presence, level, or activity towards a presence, level, or activity, observed in normal individuals (e.g., individuals who do not suffer from the disease or condition). In instances wherein the therapeutic agent is not therapeutically effective or is not providing a sufficient alleviation of the disease or condition, or symptom of the disease or condition, then the dosage amount and/or route of administration may be changed, an additional agent may be administered to the subject, along with the therapeutic agent, or the therapeutic agent may be changed to a different therapeutic agent. In some embodiments, the additional agent is another therapeutic agent.

Suitable dose and dosage administrated to a subject is determined by factors including, but no limited to, the particular therapeutic agent, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated. In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously (or over a short period of time) or at appropriate intervals, for example as two, three, four or more sub-doses per week or per month. Non-limiting examples of effective dosages of for oral delivery of a therapeutic agent include between about 0.1 mg/kg and about 100 mg/kg of body weight per day, and preferably between about 0.5 mg/kg and about 50 mg/kg of body weight per day. In other instances, the oral delivery dosage of effective amount is about 1 mg/kg and about 10 mg/kg of body weight per day of active material. Non-limiting examples of effective dosages for intravenous administration of the therapeutic agent include at a rate between about 0.01 to 100 μmol/kg body weight/min. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the therapeutic agent used, the disease or condition to be treated, the mode of administration, the requirements of the individual subject, the severity of the disease or condition being treated, and the judgment of the practitioner. The effective dosage ranges may be adjusted based on subject's response to the treatment. Some routes of administration may require higher concentrations of effective amount of therapeutics than other routes.

The dose and administration schedule may be selected and adjusted based on the level of disease, or tolerability in the subject, which may be monitored during the course of treatment. The therapeutic agent may be administered once per day, twice a day, once per week, multiple times per week, but less than once per day, multiple times per month but less than once per day, multiple times per month but less than once per week, once per month, once per five weeks, once per six weeks, once per seven weeks, once per eight weeks, once per nine weeks, once per ten weeks, or intermittently to relieve or alleviate symptoms of the disease. Administration may continue at any of the disclosed intervals until remission of the tumor or symptoms of the cancer being treated. Administration may continue after remission or relief of symptoms is achieved where such remission or relief is prolonged by such continued administration.

In certain embodiments wherein the patient's condition does not improve, upon the doctor's discretion the administration of therapeutic agent is administered chronically, that is, for an extended period of time, including throughout the duration of the patient's life in order to ameliorate or otherwise control or limit the symptoms of the patient's disease or condition. In certain embodiments wherein a patient's status does improve, the dose of therapeutic agent being administered may be temporarily reduced or temporarily suspended for a certain length of time (e.g., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. In certain embodiments, the dose of drug being administered may be temporarily reduced or temporarily suspended for a certain length of time (e.g., a “drug diversion”). In specific embodiments, the length of the drug diversion is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug diversion is, by way of example only, by 10%-100%, including by way of example only 10%, 15%, 20%, 25%, 30%, 35%, 40%, 45%, 50%, 55%, 60%, 65%, 70%, 75%, 80%, 85%, 90%, 95%, and 100%. After a suitable length of time, the normal dosing schedule is optionally reinstated.

In some embodiments, once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis.

Toxicity and therapeutic efficacy of such therapeutic regimens are determined by standard pharmaceutical procedures in cell cultures or experimental animals, including, but not limited to, the determination of the LD50 and the ED50. The dose ratio between the toxic and therapeutic effects is the therapeutic index and it is expressed as the ratio between LD50 and ED50. In certain embodiments, the data obtained from cell culture assays and animal studies are used in formulating the therapeutically effective daily dosage range and/or the therapeutically effective unit dosage amount for use in mammals, including humans. In some embodiments, the daily, weekly, monthly dosage amount of the therapeutic agent described herein lies within a range of circulating concentrations that include the ED50 with minimal toxicity. In certain embodiments, the daily, weekly, monthly dosage range and/or the unit dosage amount varies within this range depending upon the dosage form employed and the route of administration utilized.

### II. Systems

Provided here systems comprising compositions, computer systems, and/or kits for screening for lung cancer. In some embodiments, the systems or kits of the present disclosure comprise compositions capable of detecting one or more lung cancer associated markers.

### 1. Compositions for Detecting Lung Cancer Associated Markers

Provided herein are compositions for detecting one or more lung cancer associated markers. In some embodiments, the composition comprises a ligand. In some embodiments, the ligand is an antibody. In some embodiments, the ligand is an antigen-binding fragment. In some embodiments, the ligand is an antigen. In some embodiments, the ligand is a nucleic acid. In one aspect, provided herein are antibodies and antigen-binding fragments. In some embodiments, an antibody comprises an antigen-binding fragment that refers to a portion of an antibody having antigenic determining variable regions of an antibody. Examples of antigen-binding fragments include, but are not limited to, Fab, Fab′, F(ab′)₂, and Fv fragments, linear antibodies, single chain antibodies, and multispecific antibodies formed from antibody fragments.

In some embodiments, an antibody refers to a molecule that recognizes and specifically binds to a target, such as a protein, a polypeptide, a peptide, a fragment thereof, or combinations of the foregoing through at least one antigen recognition site within a variable region of the molecule. In some embodiments, an antibody includes intact polyclonal antibodies, intact monoclonal antibodies, antibody fragments (such as Fab, Fab′, F(ab′)₂, and Fv fragments), single chain Fv (scFv) mutants, a CDR-grafted antibody, multispecific antibodies, chimeric antibodies, humanized antibodies, human antibodies, fusion proteins comprising an antigen determination portion of an antibody, and any other modified molecule comprising an antigen recognition site so long as the antibodies exhibit the desired biological activity. In some embodiments, the antibody or antigen-binding fragment used in methods of detecting in Section (I)(1)(c) specifically bind to at least a portion of the lung cancer associated proteomic markers in Table 1.

An antibody can be of any the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. The antibodies disclosed herein can be used in an immunoassay, such as those described elsewhere herein. Antibodies can be naked or conjugated to other molecules such as labels. The label can be any label capable of detecting a target or lung cancer associated marker, such as those described elsewhere herein.

In some embodiments, the composition comprises a solid support. Non-limiting examples of solid supports include a well, a welled plate, a bead, a membrane, a lateral flow membrane, and a flow cell. In some embodiments, the composition comprises a ligand affixed to a solid support. The ligand may be a ligand as disclosed herein. For example, a ligand may be an antibody, an antigen, an antigen-binding fragment, or a nucleic acid.

### 2. Computer Systems for Lung Cancer Screening

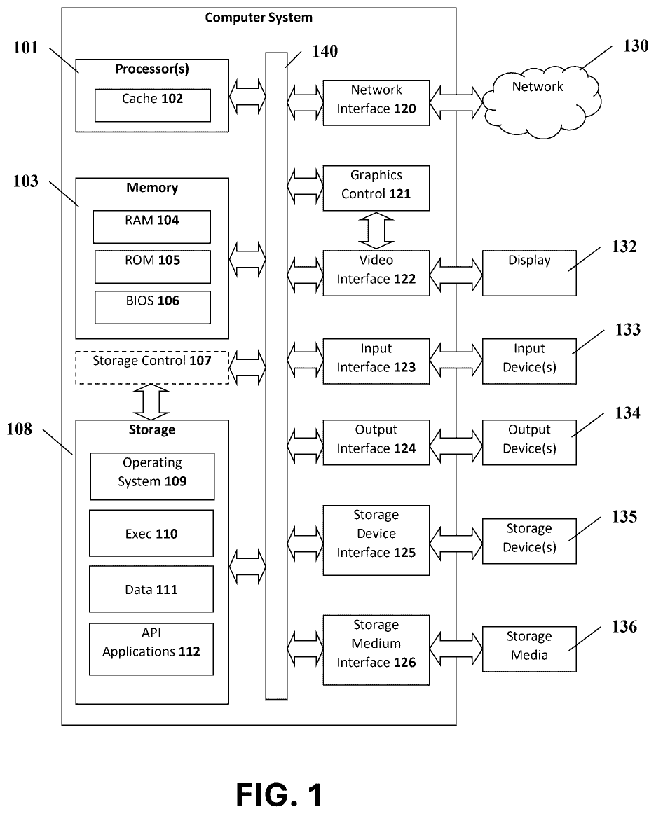

Disclosed herein, in some embodiments, are methods and systems of the present disclosure utilizing one or more computer systems for screening lung cancer. Referring to FIG. 1, a block diagram is shown depicting an example machine that includes a computer system 100 (e.g., a processing or computing system) within which a set of instructions can execute for causing a device to perform or execute any one or more of the aspects and/or methodologies for static code scheduling of the present disclosure. The components in FIG. 1 are examples only and do not limit the scope of use or functionality of any hardware, software, embedded logic component, or a combination of two or more such components implementing particular embodiments.

Computer system 100 may include one or more processors 101, a memory 103, and a storage 108 that communicate with each other, and with other components, via a bus 140. The bus 140 may also link a display 132, one or more input devices 133 (which may, for example, include a keypad, a keyboard, a mouse, a stylus, etc.), one or more output devices 134, one or more storage devices 135, and various tangible storage media 136. All of these elements may interface directly or via one or more interfaces or adaptors to the bus 140. For instance, the various tangible storage media 136 can interface with the bus 140 via storage medium interface 126. Computer system 100 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 100 includes one or more processor(s) 101 (e.g., central processing units (CPUs), general purpose graphics processing units (GPGPUs), or quantum processing units (QPUs)) that carry out functions. Processor(s) 101 optionally contains a cache memory unit 102 for temporary local storage of instructions, data, or computer addresses. Processor(s) 101 are configured to assist in execution of computer readable instructions. Computer system 100 may provide functionality for the components depicted in FIG. 1 as a result of the processor(s) 101 executing non-transitory, processor-executable instructions embodied in one or more tangible computer-readable storage media, such as memory 103, storage 108, storage devices 135, and/or storage medium 136. The computer-readable media may store software that implements particular embodiments, and processor(s) 101 may execute the software. Memory 103 may read the software from one or more other computer-readable media (such as mass storage device(s) 135, 136) or from one or more other sources through a suitable interface, such as network interface 120. The software may cause processor(s) 101 to carry out one or more processes or one or more operations of one or more processes described or illustrated herein. Carrying out such processes or operations may include defining data structures stored in memory 103 and modifying the data structures as directed by the software.

The memory 103 may include various components (e.g., machine readable media) including, but not limited to, a random access memory component (e.g., RAM 104) (e.g., static RAM (SRAM), dynamic RAM (DRAM), ferroelectric random access memory (FRAM), phase-change random access memory (PRAM), etc.), a read-only memory component (e.g., ROM 105), and any combinations thereof. ROM 105 may act to communicate data and instructions unidirectionally to processor(s) 101, and RAM 104 may act to communicate data and instructions bidirectionally with processor(s) 101. ROM 105 and RAM 104 may include any suitable tangible computer-readable media described below. In one example, a basic input/output system 106 (BIOS), including basic routines that help to transfer information between elements within computer system 100, such as during start-up, may be stored in the memory 103.

Fixed storage 108 is connected bidirectionally to processor(s) 101, optionally through storage control unit 107. Fixed storage 108 provides additional data storage capacity and may also include any suitable tangible computer-readable media described herein. Storage 108 may be used to store operating system 109, executable(s) 110, data 111, applications 112 (application programs), and the like. Storage 108 can also include an optical disk drive, a solid-state memory device (e.g., flash-based systems), or a combination of any of the above. Information in storage 108 may, in appropriate cases, be incorporated as virtual memory in memory 103.

In one example, storage device(s) 135 may be removably interfaced with computer system 100 (e.g., via an external port connector (not shown)) via a storage device interface 125. Particularly, storage device(s) 135 and an associated machine-readable medium may provide non-volatile and/or volatile storage of machine-readable instructions, data structures, program modules, and/or other data for the computer system 100. In one example, software may reside, completely or partially, within a machine-readable medium on storage device(s) 135. In another example, software may reside, completely or partially, within processor(s) 101.

Bus 140 connects a wide variety of subsystems. Herein, reference to a bus may encompass one or more digital signal lines serving a common function, where appropriate. Bus 140 may be any of several types of bus structures including, but not limited to, a memory bus, a memory controller, a peripheral bus, a local bus, and any combinations thereof, using any of a variety of bus architectures. As an example and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 100 may also include an input device 133. In one example, a user of computer system 100 may enter commands and/or other information into computer system 100 via input device(s) 133. Examples of an input device(s) 133 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 133 may be interfaced to bus 140 via any of a variety of input interfaces 123 (e.g., input interface 123) including, but not limited to, serial, parallel, game port, USB, FIREWIRE, THUNDERBOLT, or any combination of the above.

In particular embodiments, when computer system 100 is connected to network 130, computer system 100 may communicate with other devices, specifically mobile devices and enterprise systems, distributed computing systems, cloud storage systems, cloud computing systems, and the like, connected to network 130. Communications to and from computer system 100 may be sent through network interface 120. For example, network interface 120 may receive incoming communications (such as requests or responses from other devices) in the form of one or more packets (such as Internet Protocol (IP) packets) from network 130, and computer system 100 may store the incoming communications in memory 103 for processing. Computer system 100 may similarly store outgoing communications (such as requests or responses to other devices) in the form of one or more packets in memory 103 and communicated to network 130 from network interface 120. Processor(s) 101 may access these communication packets stored in memory 103 for processing.

Examples of the network interface 120 include, but are not limited to, a network interface card, a modem, and any combination thereof. Examples of a network 130 or network segment 130 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 130, may employ a wired and/or a wireless mode of communication. In general, any network topology may be used.

Information and data can be displayed through a display 132. Examples of a display 132 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 132 can interface to the processor(s) 101, memory 103, and fixed storage 108, as well as other devices, such as input device(s) 133, via the bus 140. The display 132 is linked to the bus 140 via a video interface 122, and transport of data between the display 132 and the bus 140 can be controlled via the graphics control 121. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 132, computer system 100 may include one or more other peripheral output devices 134 including, but not limited to, an audio speaker, a printer, a storage device, and any combinations thereof. Such peripheral output devices may be connected to the bus 140 via an output interface 124. Examples of an output interface 124 include, but are not limited to, a serial port, a parallel connection, a USB port, a FIREWIRE port, a THUNDERBOLT port, and any combinations thereof.

In addition or as an alternative, computer system 100 may provide functionality as a result of logic hardwired or otherwise embodied in a circuit, which may operate in place of or together with software to execute one or more processes or one or more operations of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm operations described in connection with the embodiments disclosed herein may be implemented as electronic hardware, computer software, or combinations of both. To clearly illustrate this interchangeability of hardware and software, various illustrative components, blocks, modules, circuits, and operations have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The operations of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An example storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetBSD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® PS3®, Sony® PS4®, Microsoft® Xbox 360®, Microsoft Xbox One, Nintendo® Wii®, Nintendo® Wii U®, and Ouya®.

a. Non-Transitory Computer Readable Storage Medium

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

b. Computer Program

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

c. Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, XML, and document-oriented database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or extensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® ActionScript, JavaScript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

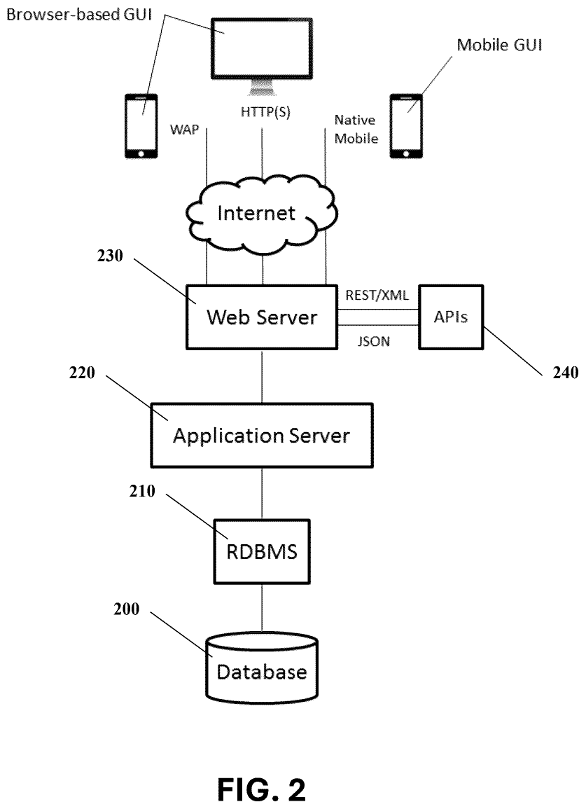

Referring to FIG. 2, in a particular embodiment, an application provision system comprises one or more databases 200 accessed by a relational database management system (RDBMS) 210. Suitable RDBMSs include Firebird, MySQL, PostgreSQL, SQLite, Oracle Database, Microsoft SQL Server, IBM DB2, IBM Informix, SAP Sybase, Teradata, and the like. In this embodiment, the application provision system further comprises one or more application severs 220 (such as Java servers, .NET servers, PHP servers, and the like) and one or more web servers 230 (such as Apache, IIS, GWS and the like). The web server(s) optionally expose one or more web services via app application programming interfaces (APIs) 240. Via a network, such as the Internet, the system provides browser-based and/or mobile native user interfaces.

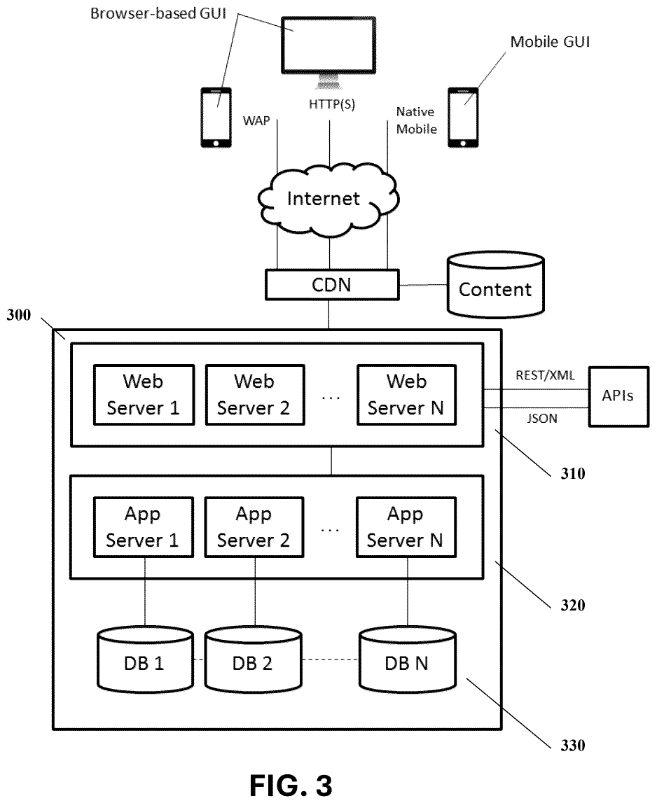

Referring to FIG. 3, in a particular embodiment, an application provision system alternatively has a distributed, cloud-based architecture 300 and comprises elastically load balanced, auto-scaling web server resources 310 and application server resources 320 as well synchronously replicated databases 330.

d. Mobile Application

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, JavaScript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

e. Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

f. Web Browser Plug-In

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM Blackberry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

g. Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, a distributed computing resource, a cloud computing resource, or combinations thereof. In further various embodiments, a software module comprises a plurality of files, a plurality of sections of code, a plurality of programming objects, a plurality of programming structures, a plurality of distributed computing resources, a plurality of cloud computing resources, or combinations thereof. In various embodiments, the one or more software modules comprise, by way of non-limiting examples, a web application, a mobile application, a standalone application, and a distributed or cloud computing application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

h. Classifier

In some embodiments, the platforms, systems, media, and methods disclosed herein include a classifier. In some embodiments, a system may be configured to load, run, and/or store a software module, script or codebase that comprises a classifier. In some embodiments, the system may comprise a computer system as described elsewhere herein, in various embodiments. For example, a system may comprise one or more of a processor, a memory, and a storage. The one or more processors may comprise a specialized processor with an architecture capable of performing operations on array-based information (such as an array of parameters in a machine learning model). The specialized processor may comprise a TPU. The specialized processor may comprise a grid-like structure of interconnected processing elements (e.g., a systolic array). The system may run, store or load a software module comprising a machine learning method. The machine learning method may comprise a classifier. For example, the machine learning method may be one of the various embodiments described herein comprising a machine learning method.

i. Databases

In some embodiments, the platforms, systems, media, and methods disclosed herein include one or more databases, or use of the same. In view of the disclosure provided herein, those of skill in the art will recognize that many databases are suitable for storage and retrieval of proteomic information. In various embodiments, suitable databases include, by way of non-limiting examples, relational databases, non-relational databases, object-oriented databases, object databases, entity-relationship model databases, associative databases, XML databases, document oriented databases, and graph databases. Further non-limiting examples include SQL, PostgreSQL, MySQL, Oracle, DB2, Sybase, and MongoDB. In some embodiments, a database is Internet-based. In further embodiments, a database is web-based. In still further embodiments, a database is cloud computing based. In a particular embodiment, a database is a distributed database. In other embodiments, a database is based on one or more local computer storage devices.

### 3. Kits

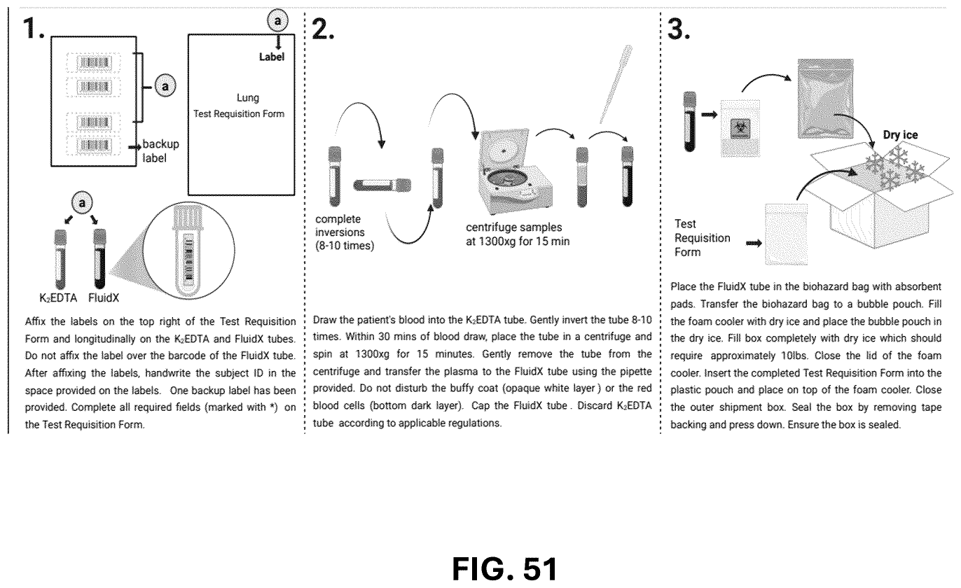

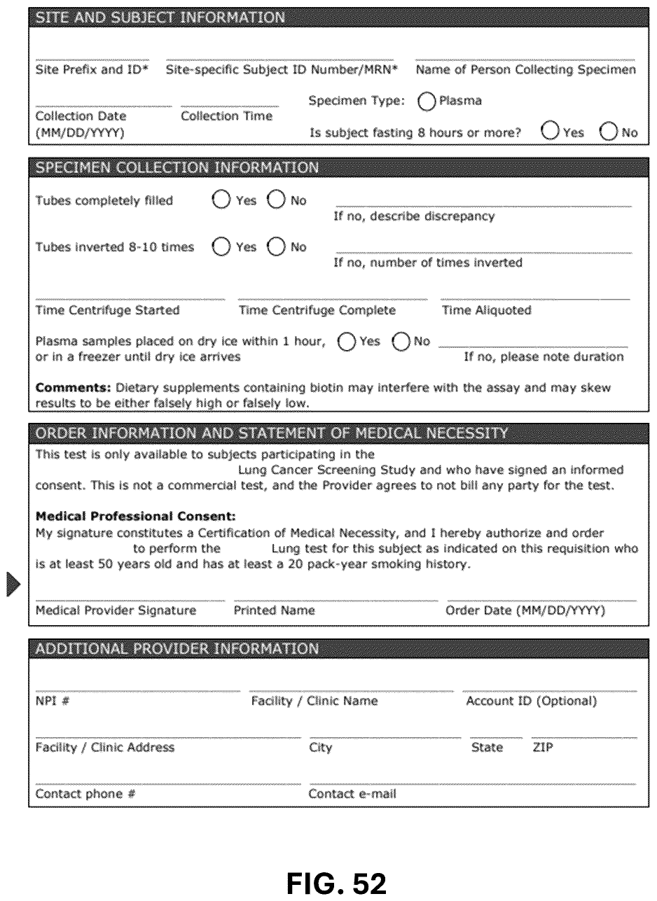

Provided herein are sample collection kits. In some embodiments, a sample collection kit comprises a test requisition form (TRF), instructions, barcoded labels, a biohazard bag with an absorbent pad, a bubble pouch, a box, and a return label. In some embodiments, the sample collection kit further comprises one or more of a blood collection tube, a needle, an alcohol prep pad, a tourniquet, a bandage, a pipette, a resealable plastic bag for the TRF, a foam cooler, and sealing tape. In some embodiments, the blood collection tube comprises a K2 EDTA tube. In some embodiments the blood collection tube comprises a storage tube. In some embodiments the storage tube is a FluidX tube. In some embodiments, the needle is a 21-gauge needle. An example of instructions that may be provided in the sample collection kit are shown in FIG. 51. An example of a test requisition form (TRF) that may be provided with the sample collection kit is shown in FIG. 52.

The materials or components assembled in the kit can be provided to a practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components.

Provided herein are lung cancer associated marker detection kits. The kit may comprise ligands such as antibodies as described herein, which can be used to perform the methods described herein. The kit may comprise solid supports. Non-limiting examples of solid supports comprise wells, a welled plate, beads, a lateral flow membrane, a flow cell or membranes. In some embodiments, the kits disclosed herein may be used to diagnose and/or treat a disease or condition in a subject; or select a patient for treatment and/or monitor a treatment disclosed herein. In some embodiments, the kit comprises the compositions described herein, which can be used to perform the methods described herein. Kits comprise an assemblage of materials or components, including at least one of the compositions. In some embodiments, the kit comprises all of the components necessary and/or sufficient to perform an assay for detecting and measuring one or more lung cancer associated markers, including all controls, directions for performing assays, and any necessary software for analysis and presentation of results.

The materials or components assembled in the kit can be provided to a practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. The packaging materials employed in the kit are those customarily utilized in gene expression assays and in the administration of treatments. As used herein, the term “package” refers to a suitable solid matrix or material such as glass, plastic, paper, foil, and the like, capable of holding the individual kit components.

In some instances, the kits described herein comprise components for detecting the presence, absence, amount, and/or concentration of a lung cancer associated marker described herein. In some embodiments, the kit comprises the compositions (e.g., probes, antibodies) described herein. The disclosure provides kits suitable for assays such as immunoassays (e.g., enzyme-linked immunosorbent assay (ELISA), lateral flow, and particle-based assays).

### III. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

The term “in vivo” is used to describe an event that takes place in a subject's body.

The term “ex vivo” is used to describe an event that takes place outside of a subject's body. An ex vivo assay is not performed on a subject. Rather, it is performed upon a sample separate from a subject. An example of an ex vivo assay performed on a sample is an “in vitro” assay.

The term “in vitro” is used to describe an event that takes places contained in a container for holding laboratory reagent such that it is separated from the biological source from which the material is obtained. In vitro assays can encompass cell-based assays in which living or dead cells are employed. In vitro assays can also encompass a cell-free assay in which no intact cells are employed.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The terms “increased”, “increasing”, or “increase” are used herein to generally mean an increase by a statically significant amount. In some aspects, the terms “increased,” or “increase,” mean an increase of at least 10% as compared to a reference level, for example an increase of at least about 10%, at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, standard, or control. Other examples of “increase” include an increase of at least 2-fold, at least 5-fold, at least 10-fold, at least 20-fold, at least 50-fold, at least 100-fold, at least 1000-fold or more as compared to a reference level.

The terms “decreased”, “decreasing”, or “decrease” are used herein generally to mean a decrease by a statistically significant amount. In some aspects, “decreased” or “decrease” means a reduction by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (e.g., absent level or non-detectable level as compared to a reference level), or any decrease between 10-100% as compared to a reference level. In the context of a marker or symptom, by these terms is meant a statistically significant decrease in such level. The decrease can be, for example, at least 10%, at least 20%, at least 30%, at least 40% or more, and is preferably down to a level accepted as within the range of normal for an individual without a given disease. Other examples of “decrease” include a decrease of at least 2-fold, at least 5-fold, at least 10-fold, at least 20-fold, at least 50-fold, at least 100-fold, at least 1000-fold or more as compared to a reference level.

As used herein, the terms “homologous,” “homology,” or “percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993). Such a formula is incorporated into the basic local alignment search tool (BLAST) programs of Altschul et al. (J Mol Biol. 1990 Oct. 5; 215(3):403-10; Nucleic Acids Res. 1997 Sep. 1; 25(17):3389-402). Percent homology of sequences can be determined using the most recent version of BLAST, as of the filing date of this application. Percent identity of sequences can be determined using the most recent version of BLAST, as of the filing date of this application.

As used herein, the term “percent (%) identity”, or “percent sequence identity,” with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. As used herein, the term “percent (%) identity”, or “percent sequence identity,” with respect to a reference nucleic acid sequence is the percentage of nucleotides in a candidate sequence that are identical with the nucleotides in the reference nucleic acid sequence, after aligning the sequences and introducing gaps, if necessary, to achieve the maximum percent sequence identity. Alignment for purposes of determining percent sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

As used herein, as it relates to machine learning and pattern recognition, the term “feature” generally refers to an individual measurable property or characteristic of a phenomenon being observed. The concept of “feature” is related to that of an explanatory variable used in statistical techniques such as for example, but not limited to, linear regression and logistic regression. Features may be numeric or categorical (e.g., structural features such as strings and graphs are used in syntactic pattern recognition). As used herein, the term “input features” (or “features”) generally refers to variables that are used by the trained algorithm (e.g., machine learning model or classifier) to predict an output classification (label) of a sample, e.g., a condition, sequence content (e.g., mutations), suggested data collection operations, or suggested treatments. Values of the variables may be determined for a sample and used to determine a classification.

For a plurality of assays, the system identifies feature sets to input into a trained algorithm (e.g., machine learning model or classifier). The system performs an assay on each biological sample and forms a feature vector from the measured values. The system inputs the feature vector into the machine learning model and obtains an output classification of whether the biological sample has a specified property. In some embodiments, the machine learning model outputs a classifier capable of distinguishing between two or more groups or classes of subjects or features in a population of subjects or features of the population. In some embodiments, the classifier is a trained machine learning classifier.

In some embodiments, the informative loci or features of biomarkers in a cancer tissue are assayed to form a profile. Receiver-operating characteristic (ROC) curves may be generated by plotting the performance of a particular feature (e.g., any of the biomarkers described herein and/or any item of additional biomedical information) in distinguishing between two populations (e.g., subjects responding and not responding to a therapeutic agent). In some embodiments, the feature data across the entire population (e.g., the cases and controls) are sorted in ascending order based on the value of a single feature.

Where a UniProt accession number is referred to, a feature or biomarker may include a protein. Where an Ensembl accession number is referred to, a feature or biomarker may include an RNA such as an mRNA. Ensembl and UniProt references are current as of the effective priority date of their disclosure in this application, as provided found at useast.ensembl.org and www.uniprot.org, respectively.

The section headings used herein are for organizational purposes only and are not to be construed as limiting the subject matter described.

### IV. Exemplary Embodiments

Among the exemplary embodiments are:

Embodiment 1. A method for the quantitative detection of lung cancer associated proteomic markers in a biofluid sample, the method comprising: (a) obtaining a biofluid sample from a subject, wherein the subject is an age of 50 or older and is at risk for developing lung cancer as determined by, at least in part, a smoking history and the age of the subject; (b) measuring an amount or a concentration of the lung cancer associated proteomic markers in the biofluid sample or a processed sample therefrom with an immunoassay to obtain proteomic measurements; and (c) applying a classifier to the proteomic measurements to provide a quantitative or qualitative result for the biofluid sample of the lung cancer, wherein the classifier distinguishes the lung cancer from a non-cancer with a performance characteristic comprising a sensitivity of at least 80% and a specificity of at least 55%.

Embodiment 2. The method of embodiment 1, wherein the biofluid sample is a blood sample and the processed sample therefrom is a plasma sample or a serum sample comprising plasma or serum isolated from the blood sample.

Embodiment 3. The method of embodiment 1, wherein the lung cancer is stage 1 non-small cell lung cancer.

Embodiment 4. The method of embodiment 3, wherein the sensitivity is greater than or equal to about 81%.

Embodiment 5. The method of embodiment 1, wherein the lung cancer is stage 2 non-small cell lung cancer.

Embodiment 6. The method of embodiment 5, wherein the sensitivity is about 100%.

Embodiment 7. The method of embodiment 1, wherein the lung cancer is stage 3 or stage 4 non-small cell lung cancer.

Embodiment 8. The method of embodiment 7, wherein the sensitivity is greater than or equal to about 88%.

Embodiment 9. The method of embodiment 1, wherein the performance characteristic of the classifier is obtained using a training cohort that is enriched no more than 12%.

Embodiment 10. The method of embodiment 1, wherein the performance characteristic of the classifier comprises an area under the curve (AUC) that is greater than or equal to about 0.80.

Embodiment 11. The method of embodiment 10, wherein the AUC is greater than or equal to about 0.82.

Embodiment 12. The method of embodiment 1, wherein the proteomic measurements are obtained from fewer than or equal to about 50 lung cancer associated proteomic markers.

Embodiment 13. The method of embodiment 1, wherein the proteomic measurements are obtained from fewer than or equal to about 20 lung cancer associated proteomic markers.

Embodiment 14. The method of embodiment 1, wherein the proteomic measurements are obtained from fewer than or equal to about 11 lung cancer associated proteomic markers.

Embodiment 15. The method of embodiment 1, wherein the age of the subject is 50 to 75 years old.

Embodiment 16. The method of embodiment 1, wherein the smoking history of the subject comprises smoking greater than or equal to about 20 packs of cigarettes per year.

Embodiment 17. The method of embodiment 1, wherein the measuring the amount of the concentration of the lung cancer associated proteomic markers with the immunoassay comprises: (i) contacting the lung cancer associated proteomic markers with one or more detection reagents under conditions sufficient to couple the lung cancer associated proteomic markers to the one or more detection reagents; and (ii) detecting a signal associated with the one or more detection reagents coupled to the lung cancer associated proteomic markers.

Embodiment 18. The method of embodiment 17, wherein the lung cancer associated proteomic markers are immobilized to a solid support directly or indirectly.

Embodiment 19. The method of embodiment 1, wherein the immunoassay is a sandwich immunoassay.

Embodiment 20. The method of embodiment 1, wherein the measuring the amount of the concentration of the lung cancer associated proteomic markers with the immunoassay comprises: (i) contacting the lung cancer associated proteomic markers with one or more detection reagents and one or more receptors immobilized to a solid support under conditions sufficient to form a detectable binding complex, wherein the detectable binding complex comprises the one or more detection reagents coupled to the one or more receptors and the lung cancer associated proteomic markers; and (ii) detecting a signal associated with the one or more detection reagents in the detectable binding complex.

Embodiment 21. The method of embodiment 20, wherein the solid support is a bead, a welled plate, a lateral flow membrane, a planar surface, or a flow cell, or any combination thereof.

Embodiment 22. The method of embodiment 1, wherein the immunoassay comprises an enzyme-linked immunosorbent assay (ELISA), a particle-based immunoassay, a lateral flow assay, a proximity extension assay, or any combination thereof.

Embodiment 23. The method of embodiment 22, wherein the immunoassay comprises a fluorescent readout.

Embodiment 24. The method of embodiment 23, wherein the fluorescent readout is obtained from one or more fluorescent proteins or a fluorescence resonance energy transfer.

Embodiment 25. The method of embodiment 22, wherein the ELISA is a sandwich ELISA.

Embodiment 26. The method of embodiment 22, wherein the solid surface-based immunoassay utilizes: (i) a receptor immobilized to a solid surface, wherein the receptor specifically binds to a lung cancer associated proteomic marker of the lung cancer associated proteomic markers; and (ii) a detection reagent comprising a binding moiety coupled to a detectable label, wherein the binding moiety specifically binds to the lung cancer associated proteomic marker or a molecular tag coupled thereto.

Embodiment 27. The method of embodiment 26, wherein the receptor comprises an antibody or an antigen-binding fragment.

Embodiment 28. The method of embodiment 26, wherein the detection reagent comprises an antibody or an antigen-binding fragment coupled to a detectable label.

Embodiment 29. The method of embodiment 28, wherein the detectable label is a fluorescent, an enzymatic, a radioactive, or an affinity label.

Embodiment 30. The method of embodiment 29, wherein the affinity label is streptavidin-biotin.

Embodiment 31. The method of embodiment 29, wherein the fluorescent label comprises a fluorescent protein or fluorescence resonance energy transfer pairs.

Embodiment 32. The method of embodiment 29, wherein the enzymatic label comprises horseradish peroxidase (HRP) or alkaline phosphatase (AP).

Embodiment 33. The method of embodiment 26, wherein the solid surface is a bead, a nanoparticle, a planar surface, or a surface plasmon resonance (SPR) particle.

Embodiment 34. The method of embodiment 26, wherein the solid surface is a magnetic bead or a polystyrene bead.

Embodiment 35. The method of embodiment 26, wherein the solid surface comprises a coating layer coupled to a surface of the bead, wherein the coating layer comprises carboxyl (—COOH) groups, streptavidin or avidin.

Embodiment 36. The method of embodiment 1, wherein the classifier distinguishes the lung cancer from the non-cancer by applying a threshold to an aggregation of the proteomic measurements for all the lung cancer associated proteomic markers.

Embodiment 37. The method of embodiment 36, wherein the threshold is determined using an analysis of a precision-recall curve.

Embodiment 38. The method of embodiment 37, wherein, the analysis determines a threshold corresponding to the sensitivity of at least 80% and the specificity of at least 55%.

Embodiment 39. The method of embodiment 36, wherein the aggregation comprises a summation.

Embodiment 40. The method of embodiment 39, wherein the aggregation comprises applying a transformation to the summation.

Embodiment 41. The method of embodiment 1, wherein the classifier comprises a linear regression algorithm.

Embodiment 42. The method of embodiment 1, wherein the classifier comprises a logistic regression algorithm.

Embodiment 43. The method of embodiment 1, wherein the classifier comprises a gradient boosted model.

Embodiment 44. The method of embodiment 1, wherein the quantitative result is a degree of risk that the subject has the lung cancer.

Embodiment 45. The method of embodiment 1, wherein the qualitative result is a determination that the subject has the lung cancer or not.

Embodiment 46. The method of embodiment 1, wherein the qualitative result is an odds ratio that a subject is likely to develop the lung cancer or not.

Embodiment 47. A method comprising: (a) obtaining a biofluid sample from a subject at risk of having lung cancer, wherein the biofluid sample comprises one or more lung cancer associated proteomic markers; (b) extracting the one or more lung cancer associated proteomic markers from the biofluid sample or a processed sample therefrom, wherein the one or more lung cancer associated proteomic markers comprise Myoglobin (MB) or any fragment thereof, or a combination thereof; (c) analyzing the one or more lung cancer associated proteomic markers in (b) by a method comprising: (1) selectively detecting at least, a subset of the lung cancer associated proteomic markers by binding one or more detection reagents directly or indirectly to the at least the subset of the one or more lung cancer associated proteomic markers to form one or more detectable complexes; (2) detecting one or more signals obtained from the one or more detectable complexes; and (3) measuring a concentration or an amount of the one or more lung cancer associated proteomic markers in the one or more detectable complexes to produce a plurality of proteomic measurements; (d) generating a data set comprising the plurality of proteomic measurements; and (e) analyzing the data set from (d).

Embodiment 48. The method of embodiment 47, wherein the one or more lung cancer associated proteomic markers further comprises Complement Component C9 (C9), Cell adhesion molecule (CEA), CA-125 Antigen (CA125), Fragment of Cytokeratin 19 (CYFRA21-1), Glycoprotein 130 (GP130), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, or any combination thereof.

Embodiment 49. The method of embodiment 47, wherein the biofluid sample is a blood sample and the processed sample is a plasma sample or a serum sample comprising plasma or serum isolated from the blood sample.

Embodiment 50. The method of embodiment 47, wherein the one or more lung cancer associated proteomic markers are predictive of the lung cancer when the plurality of proteomic measurements are analyzed with a classifier that is trained to distinguish the lung cancer from a non-cancer and has a performance characteristic comprising a sensitivity of at least 80% and a specificity of at least 55%.

Embodiment 51. The method of embodiment 50, wherein the lung cancer is stage 1 non-small cell lung cancer.

Embodiment 52. The method of embodiment 51, wherein the sensitivity is greater than or equal to about 81%.

Embodiment 53. The method of embodiment 50, wherein the lung cancer is stage 2 non-small cell lung cancer.

Embodiment 54. The method of embodiment 53, wherein the sensitivity is about 100%.

Embodiment 55. The method of embodiment 50, wherein the lung cancer is stage 3 or 4 non-small cell lung cancer.

Embodiment 56. The method of embodiment 55, wherein the sensitivity is greater than or equal to about 88%.

Embodiment 57. The method of embodiment 50, wherein the performance characteristic of the classifier is obtained using a training cohort that is enriched no more than 12%.

Embodiment 58. The method of embodiment 50, wherein the performance characteristic of the classifier comprises an area under the curve (AUC) that is greater than or equal to about 0.80.

Embodiment 59. The method of embodiment 58, wherein the AUC is greater than or equal to about 0.82.

Embodiment 60. The method of embodiment 47, wherein the proteomic measurements are obtained from fewer than or equal to about 50 lung cancer associated proteomic markers.

Embodiment 61. The method of embodiment 47, wherein the proteomic measurements are obtained from fewer than or equal to about 20 lung cancer associated proteomic markers.

Embodiment 62. The method of embodiment 47, wherein the proteomic measurements are obtained from fewer than or equal to about 11 lung cancer associated proteomic markers.

Embodiment 63. The method of embodiment 47, wherein the subject is at risk of having the lung cancer based, at least in part, on a smoking history of the subject that comprises smoking greater than or equal to about 20 packs of cigarettes per year.

Embodiment 64. The method of embodiment 47, wherein the subject is at risk of having the lung cancer based, at least in part, on an age of the subject being 50 years or older.

Embodiment 65. The method of embodiment 64, wherein the age of the subject is 50 to 75 years old.

Embodiment 66. The method of embodiment 47, wherein the at least the subset of the one or more lung cancer associated proteomic markers is immobilized to a solid support directly or indirectly.

Embodiment 67. The method of embodiment 66, wherein the solid support is a bead, a welled plate, a lateral flow membrane, a planar surface, or a flow cell, or any combination thereof.

Embodiment 68. The method of embodiment 47, wherein one or more detection reagents comprise an antibody or an antigen-binding fragment coupled to a detectable label.

Embodiment 69. The method of embodiment 68, wherein the detectable label is a fluorescent, enzymatic, radioactive, and affinity label.

Embodiment 70. The method of embodiment 69, wherein the affinity label is streptavidin-biotin.

Embodiment 71. The method of embodiment 69, wherein the fluorescent label comprises a fluorescent protein or fluorescence resonance energy transfer pairs.

Embodiment 72. The method of embodiment 69, wherein the enzymatic label comprises horseradish peroxidase (HRP) or alkaline phosphatase (AP).

Embodiment 73. The method of embodiment 47, wherein the method for analyzing the one or more lung cancer associated proteomic markers in (c) comprises performing an immunoassay that comprises an enzyme-linked immunosorbent assay (ELISA), a particle-based immunoassay, a proximity extension assay, or a lateral flow assay, or any combination thereof.

Embodiment 74. The method of embodiment 73, wherein the immunoassay comprises a fluorescent readout.

Embodiment 75. The method of embodiment 74, wherein the fluorescent readout is obtained from one or more fluorescent proteins or a fluorescence resonance energy transfer.

Embodiment 76. The method of embodiment 73, wherein the ELISA is a sandwich ELISA.

Embodiment 77. The method of embodiment 73, wherein the particle-based immunoassay utilizes: (i) a receptor immobilized to a particle, wherein the receptor specifically binds to a lung cancer associated proteomic marker of the at least the subset of the lung cancer associated proteomic markers; and (ii) the one or more detection reagents comprises a binding moiety coupled to a detectable label, wherein the binding moiety specifically binds to the lung cancer associated proteomic marker or a molecular tag coupled thereto.

Embodiment 78. The method of embodiment 77, wherein the receptor comprises an antibody or an antigen-binding fragment.

Embodiment 79. The method of embodiment 77, wherein the detection reagent of the one or more detection reagents comprises an antibody or an antigen-binding fragment coupled to a detectable label.

Embodiment 80. The method of embodiment 79, wherein the detectable label is a fluorescent, enzymatic, radioactive, and affinity label.

Embodiment 81. The method of embodiment 80, wherein the affinity label is streptavidin-biotin.

Embodiment 82. The method of embodiment 80, wherein the fluorescent label comprises a fluorescent protein or fluorescence resonance energy transfer pairs.

Embodiment 83. The method of embodiment 80, wherein the enzymatic label comprises horseradish peroxidase (HRP) or alkaline phosphatase (AP).

Embodiment 84. The method of embodiment 77, wherein the particle is a bead, a nanoparticle, or a surface plasmon resonance (SPR) particle.

Embodiment 85. The method of embodiment 84, wherein the bead is a magnetic bead or a polystyrene bead.

Embodiment 86. The method of embodiment 85, wherein the bead comprises a coating layer coupled to a surface of the bead, wherein the coating layer comprises carboxyl (—COOH) groups, streptavidin or avidin.

Embodiment 87. A method of treating lung cancer in a subject, the method comprising: administering a therapeutic agent for the treatment of the lung cancer to the subject, wherein the subject is classified as having the lung cancer based, at least in part, on an analysis of proteomic measurements for lung cancer associated proteomic markers obtained from a biofluid sample from the subject by a classifier trained to distinguish the lung cancer from a non-cancer with a performance characteristic comprising a sensitivity of at least 80% and a specificity of at least 55%.

Embodiment 88. The method of embodiment 87, wherein the proteomic measurements are obtained with an immunoassay that comprises an enzyme-linked immunosorbent assay (ELISA), a bead-based immunoassay, a proximity extension assay, or a lateral flow assay, or any combination thereof.

Embodiment 89. The method of embodiment 88, wherein the immunoassay comprises a fluorescent readout.

Embodiment 90. The method of embodiment 89, wherein the fluorescent readout is obtained from one or more fluorescent proteins or a fluorescence resonance energy transfer.

Embodiment 91. The method of embodiment 87, wherein the analysis of proteomic measurements provides a quantitative result of the lung cancer, wherein the quantitative result is a degree of risk that the subject has the lung cancer.

Embodiment 92. The method of embodiment 87, wherein the analysis of proteomic measurements provides a qualitative result of the lung cancer, wherein the qualitative result is a determination that the subject has the lung cancer or not.

Embodiment 93. The method of embodiment 92, wherein the qualitative result is an odds ratio that the subject is likely to develop the lung cancer or not.

Embodiment 94. A method of treating lung cancer in a subject, the method comprising: (a) determining whether the subject is classified as having the lung cancer or not having the lung cancer, wherein the determining comprises: (i) obtaining or having obtained a biofluid sample from the subject; and (ii) performing or having performed an immunoassay on the biofluid sample or a processed sample therefrom to obtain proteomic measurements associated with an amount or a concentration of lung cancer associated proteomic markers in the biofluid sample; and (b) applying a classifier to the proteomic measurements to provide a quantitative or qualitative result for the biofluid sample of the lung cancer, wherein the classifier distinguishes the lung cancer from a non-cancer with a performance characteristic comprising a sensitivity of at least 80% and a specificity of at least 55%; (c) if the subject is classified as having the lung cancer, then administering a therapeutic agent for treatment of the lung cancer to the subject; and (d) if the subject is classified as not having the lung cancer, then administering a therapeutic agent for treatment of a condition other than the lung cancer.

Embodiment 95. The method of embodiment 94, wherein the condition other than the lung cancer is a comorbidity of the lung cancer.

Embodiment 96. The method of embodiment 95, wherein the comorbidity is chronic obstructive pulmonary disease (COPD), peripheral vascular disease (PVD), diabetes, congestive heart failure, cerebrovascular disease, or renal disease.

Embodiment 97. The method of embodiment 94, wherein the quantitative result is a degree of risk that the subject has the lung cancer.

Embodiment 98. The method of embodiment 94, wherein the qualitative result is a determination that the subject has the lung cancer or not.

Embodiment 99. The method of embodiment 94, wherein the qualitative result is an odds ratio that the subject is likely to develop the lung cancer or not.

Embodiment 100. The method of any one of embodiments 94-99, wherein the lung cancer is stage 1 non-small cell lung cancer.

Embodiment 101. The method of embodiment 100, wherein the sensitivity is greater than or equal to about 81%.

Embodiment 102. The method of any one of embodiments 94-99, wherein the lung cancer is stage 2 non-small cell lung cancer.

Embodiment 103. The method of embodiment 102, wherein the sensitivity is about 100%.

Embodiment 104. The method of any one of embodiments 94-99, wherein the lung cancer is stage 3 or stage 4 non-small cell lung cancer.

Embodiment 105. The method of embodiment 104, wherein the sensitivity is greater than or equal to about 88%.

Embodiment 106. The method of any one of embodiments 94-99, wherein the performance characteristic of the classifier is obtained using a training cohort that is enriched no more than 12%.

Embodiment 107. The method of any one of embodiments 94-99, wherein the performance characteristic of the classifier comprises an area under the curve (AUC) that is greater than or equal to about 0.80.

Embodiment 108. The method of embodiment 107, wherein the AUC is greater than or equal to about 0.82.

Embodiment 109. The method of any one of embodiments 94-108, wherein the proteomic measurements are obtained from fewer than or equal to about 50 lung cancer associated proteomic markers.

Embodiment 110. The method of any one of embodiments 94-108, wherein the proteomic measurements are obtained from fewer than or equal to about 20 lung cancer associated proteomic markers.

Embodiment 111. The method of any one of embodiments 94-108, wherein the proteomic measurements are obtained from fewer than or equal to about 11 lung cancer associated proteomic markers.

Embodiment 112. The method of any one of embodiments 94-111, wherein the subject is at risk of having the lung cancer based, at least in part, on a smoking history of the subject that comprises smoking greater than or equal to about 20 packs of cigarettes per year.

Embodiment 113. The method of any one of embodiments 94-112, wherein the subject is at risk of having the lung cancer based, at least in part, on an age of the subject being 50 years or older.

Embodiment 114. The method of embodiment 113, wherein the age of the subject is 50 to 75 years old.

Embodiment 115. The method of any one of embodiments 94-114, wherein the therapeutic agent for the treatment of the lung cancer is provided in Table 2.

Embodiment 116. The method of any one of embodiments 94-115, wherein the therapeutic agent for the treatment of the lung cancer is administered to the subject intravenously or subcutaneously.

Embodiment 117. The method of any one of embodiments 94-116, wherein the classifier distinguishes the lung cancer from the non-cancer by applying a threshold to an aggregation of the proteomic measurements for all the lung cancer associated proteomic markers.

Embodiment 118. The method of embodiment 117, wherein the threshold is determined using an analysis of a precision-recall curve.

Embodiment 119. The method of embodiment 118, wherein the analysis determines a threshold corresponding to the sensitivity of at least 80% and the specificity of at least 55%.

Embodiment 120. The method of embodiment 117, wherein the aggregation comprises a summation.

Embodiment 121. The method of embodiment 120, wherein the aggregation comprises applying a transformation to the summation.

Embodiment 122. The method of any one of embodiments 94-121, wherein the classifier comprises a linear regression algorithm.

Embodiment 123. The method of any one of embodiments 94-121, wherein the classifier comprises a logistic regression algorithm.

Embodiment 124. The method of any one of embodiments 94-121, wherein the classifier comprises a gradient boosted model.

Embodiment 125. A computer-implemented system comprising a computing device comprising at least one processor, an operating system configured to perform executable instructions, a memory, and a computer program including the executable instructions comprising:
(a) receiving proteomic measurements associated with an amount or a concentration of the lung cancer associated proteomic markers detected in the biofluid sample or a processed sample therefrom;
(b) inputting the proteomic measurements into a classifier that distinguishes the lung cancer from non-cancer with a performance characteristic comprising a sensitivity of at least 85% and a specificity of at least 55%; and
(c) providing a quantitative or qualitative result for the biofluid sample of the lung cancer.

Embodiment 126. Non-transitory computer-readable storage media encoded with a computer program including instructions executable by one or more processors to create a lung cancer screening application, comprising:
(a) a database, in a computer memory, comprising proteomic measurements associated with an amount or a concentration of the lung cancer associated proteomic markers detected in the biofluid sample or a processed sample therefrom from a subject; and
(b) a software module configured to:
   (i) receive the proteomic measurements;
   (ii) input the proteomic measurements into a classifier that distinguishes the lung cancer from non-cancer with a performance characteristic comprising a sensitivity of at least 85% and a specificity of at least 55%; and
   (iii) provide a quantitative or qualitative result for the biofluid sample of the lung cancer.

Embodiment 127. A computer-implemented method for quantitative detection of lung cancer associated proteomic markers in a biofluid sample, comprising:
(a) receiving proteomic measurements associated with an amount or a concentration of the lung cancer associated proteomic markers detected in the biofluid sample or a processed sample therefrom;
(b) inputting the proteomic measurements into a classifier that distinguishes the lung cancer from non-cancer with a performance characteristic comprising a sensitivity of at least 85% and a specificity of at least 55%; and
(c) providing a quantitative or qualitative result for the biofluid sample of the lung cancer.

### V. Examples

The following examples are included for illustrative purposes only and are not intended to limit the scope of the inventive concepts.

### Example 1: Case-Controlled Discovery and Verification Study of Liquid Biopsy Lung Cancer Biomarkers

In the United States there exists an estimated 14 million plus people with a high-risk of developing lung cancer. Of the at-risk population, approximately 5-10% receive standard of care screening. Due to this low adoption of screening, lung cancer is diagnosed in later stages where the 5-year survival rate is about 9%. If screening adoption and efficacy improved, more patients could be screened and potentially diagnosed earlier in the cancer progression when survivability is higher.

Here, a highly accurate and reliable screening method was developed, verified, and validated using rigorous approaches to feature selection with a focus of generating high quality data to produce a high-quality model.

### Discovery

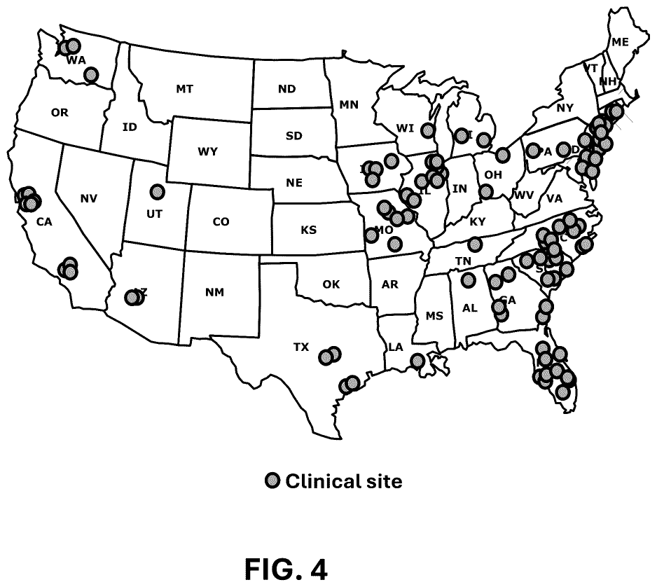

Samples were prospectively collected from 98 clinical sites distributed across the United States to increase patient accessibility and minimize site biases. All clinical sites used identical sample collection protocols to minimize pre-analytical variability. FIG. 4 shows a map of the clinical sites in the United States. Over 7,000 subject samples were prospectively collected along with over 1,000 cancer subject samples. The samples were collected for analysis using various ‘omics approaches.

Noise may arise from various sources in the data collection process. For example, a significant source of noise may be introduced to data by the machine the sample is processed on. If samples in a training set comprise noise that is specific to the machine they were processed on, the model may learn the machine's noise signature. This may cause the model to perform well on test data using the same machine as the training set but may perform worse on data from other machines.

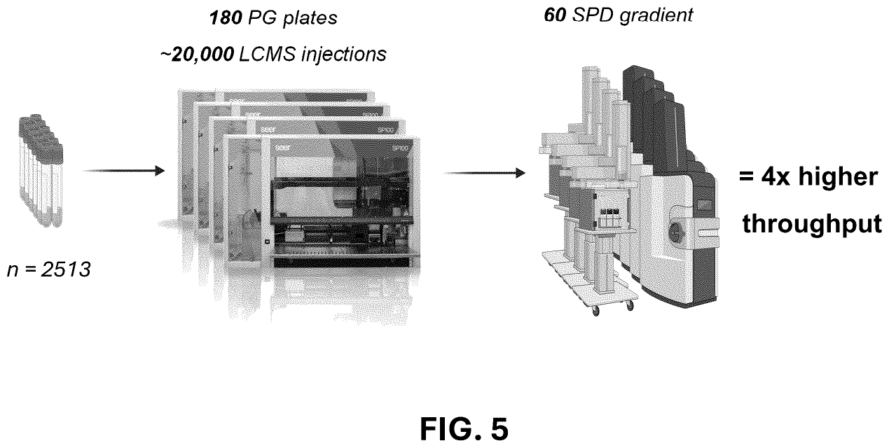

If multiple machines are used to generate training data, variations in the number of samples from different classes (such as cancer vs non-cancer) that are run on different machines (e.g., mostly cancer samples being run on machine A and mostly non-cancer samples being run on machine B) may introduce machine specific noise that the model may associate with cancer or non-cancer. This may improve the model's performance during training due to the model learning the specific noise signatures from the machines (e.g., machine A vs. machine B) rather than signals from the input features (such as biomarkers). To minimize the machine specific noise present in the dataset, four liquid chromatography/mass spectrometry (LC/MS) machines were tested for variations in noise. As shown in FIG. 5, 2,513 samples were run on four LC/MS machines using 180 Proteograph (PG) plates resulting in about 20,000 LC/MS injections.

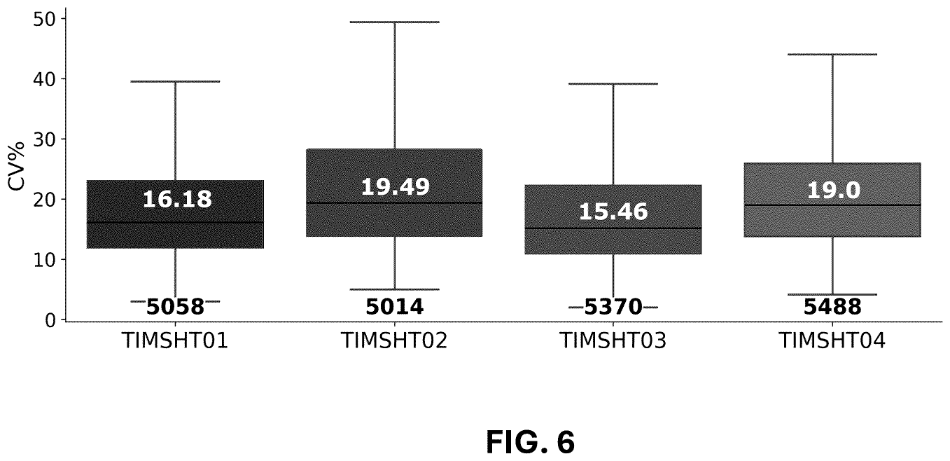

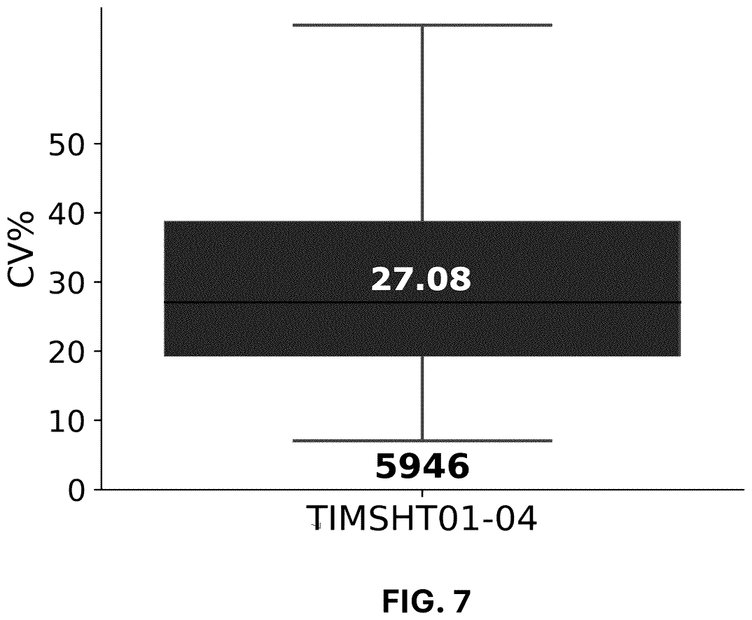

High reproducibility was demonstrated across the four instruments using a standard three-plate reproducibility study. From the three-plate reproducibility study the intra-instrument coefficient of variation percentage (% CV) for the precursors were all below 20% (FIG. 6). Further, the inter-batch and inter-instrument % CV was within acceptable performance at less than 30% (FIG. 7).

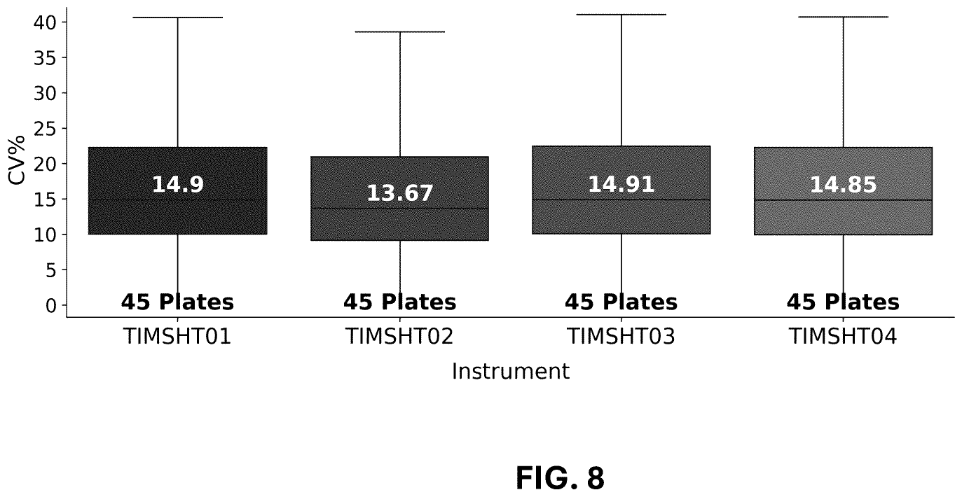

Another source of noise may come from within the batches of samples. Intra-batch variability of measured values may arise due to differences in sample processing (such as environmental differences like humidity or temperature, or operator variability like fluctuations in volumes of reagents). As shown in FIG. 8, the intra-batch CV % was low (less than 15%) for each of the 4 instruments each running 45 plates (totaling over 180 batches). This demonstrates that the intra-batch variability on the machines was low and produced reproducible measurements.

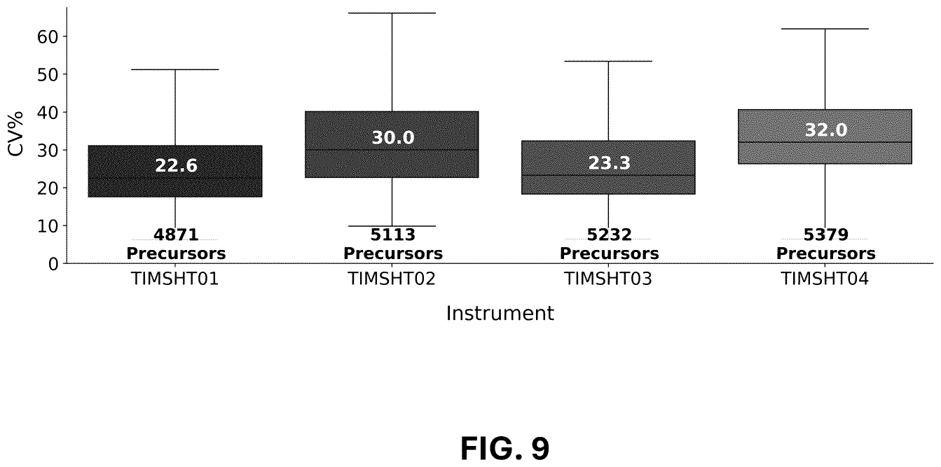

Inter-batch variability can arise from differences in the processing of samples in different batches. This can occur due to variations in the concentration of reagents or variations in equipment (such as calibrations of pipettes). When intra-batch variability is low, inter-batch variability may still be high. Such noise introduced across batches may lead to the model learning the noise introduced by different variations across batches which may be repeatable in a batch. Therefore, if one batch contains predominantly cancer samples and another batch contains predominantly non-cancer samples, the model may learn to associate what is actually inter-batch variation with cancer or non-cancer. This may result in a model that is brittle when the variation of the two batches is not present, leading to poor performance. As shown in FIG. 9, the inter-batch % CV across all 45 plates on each of the four machines was far lower than the full stack biological variability.

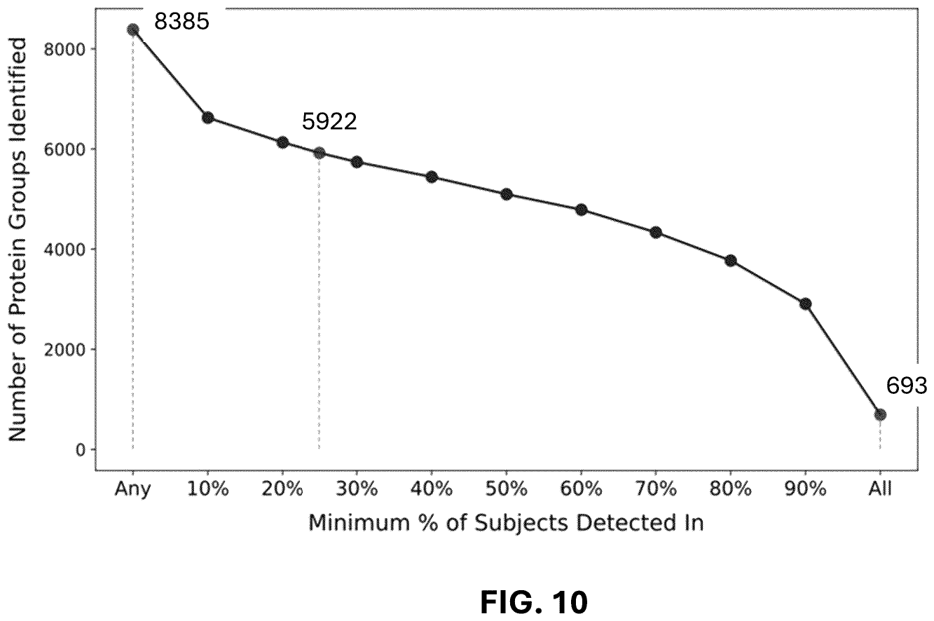

A graph of the minimum percentage of subjects in which a protein group is identified (x-axis) versus the number of protein groups thus identified (y-axis) is shown in FIG. 10. Eight-thousand three-hundred and eighty-five protein groups were identified overall (e.g., found in at least one subject) while 693 protein groups were identified in 100% of subjects (e.g., found in all subjects). To be considered as input into the machine learning algorithm, protein groups were required to be identified in at least 25% of subjects. Five-thousand and nine-hundred protein groups met this requirement. An alternate threshold could have been selected instead, 25% was selected because it was presumed to give a good balance between requiring consistent identification of the same protein groups across different subjects (which reduces spurious identifications) and prioritizing those protein groups that are identified in cancer but wholly absent in non-cancer subjects or vice versa (which increases cancer discriminative power).

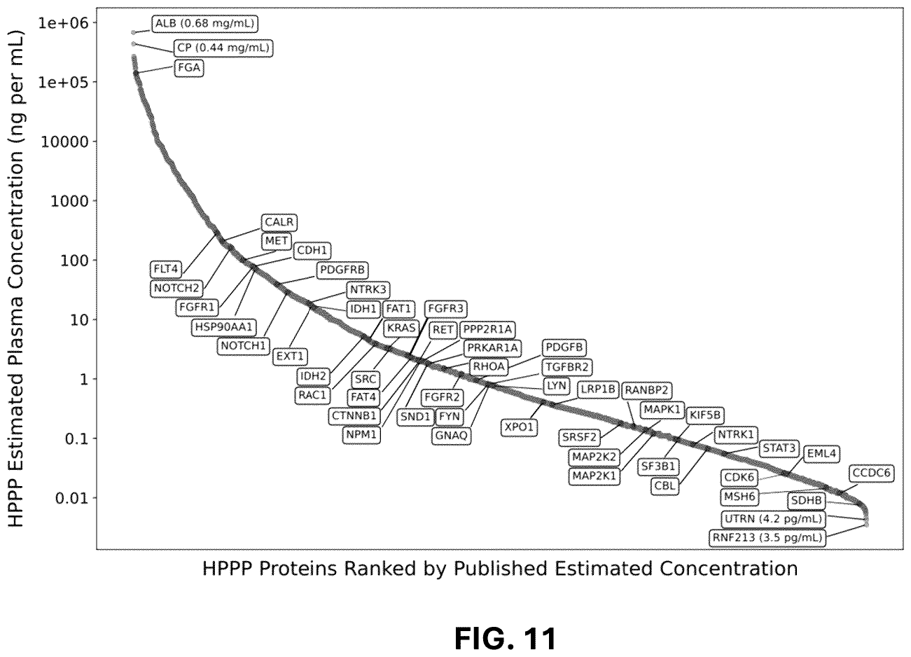

The 8,300 plus protein groups were assessed using the Human Plasma Proteome Project (HPPP) database. Approximately 83% of the proteins in the HPPP database were detected among the 8,300 plus protein groups. FIG. 11 shows the top 50 gene names (by Open Target cancer association score) of proteins, shown in black. Proteins not listed in Open Targets that were detected in the study are shown in grey. These proteins provided opportunities for discovery.

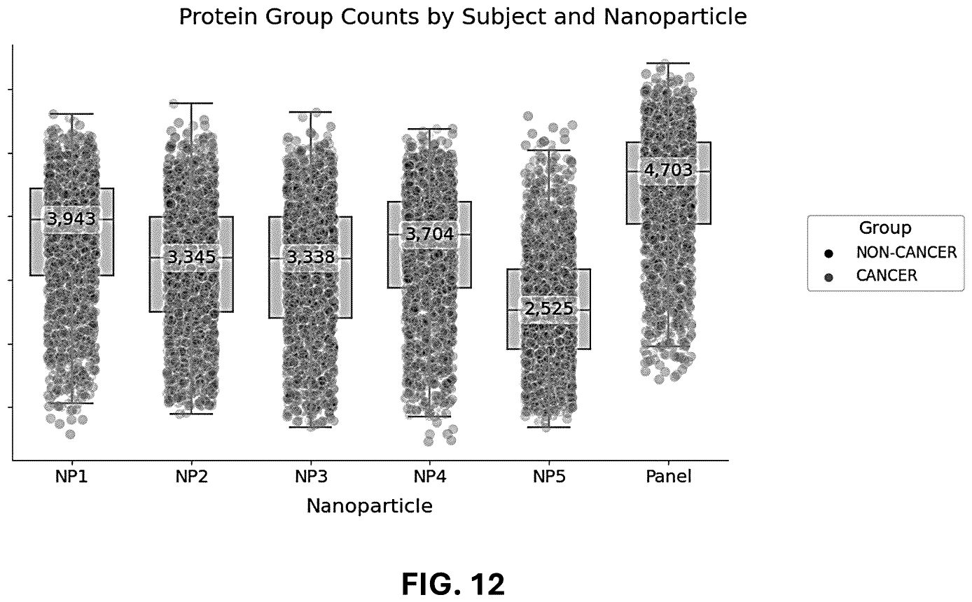

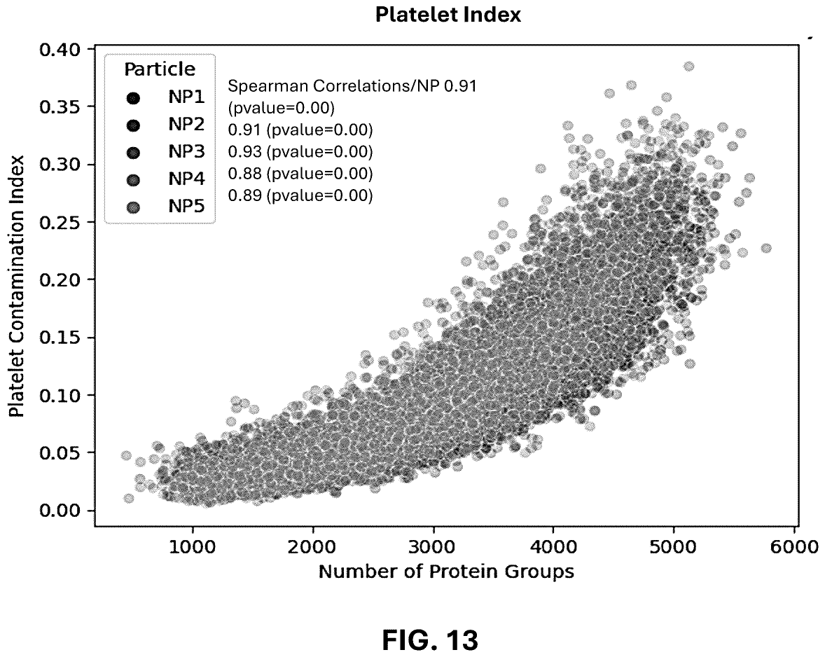

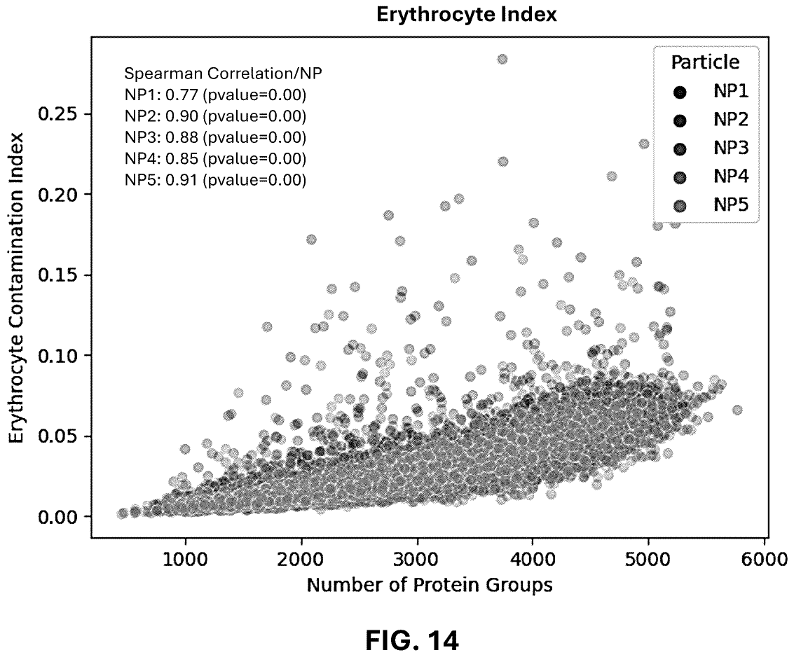

For the purposes of biomarker discovery, nanoparticles were used to extract proteins from samples. To understand how much variability there is in the protein groups detected across a single nanoparticle, protein group counts were established by subject for each nanoparticle (see FIG. 12). The differences in reported protein groups on each nanoparticle raised concerns that it was related to pre-analytical variability. To address this concern, the erythrocyte and platelet indices were calculated for each sample. As shown in FIG. 13 and FIG. 14, there is a clear correlation in protein group counts and contamination. This analysis ensured the proteins included in the classifier were disease specific rather than pre-analytical factors.

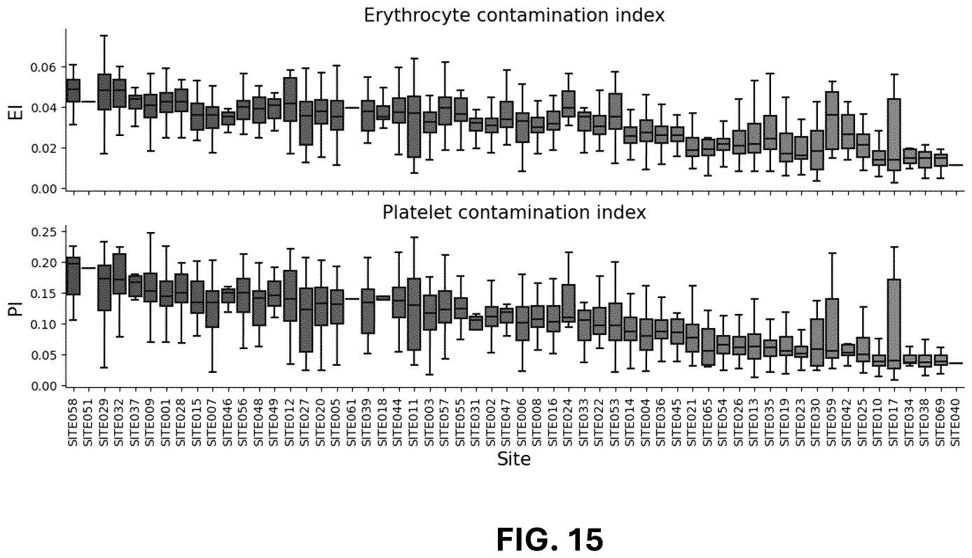

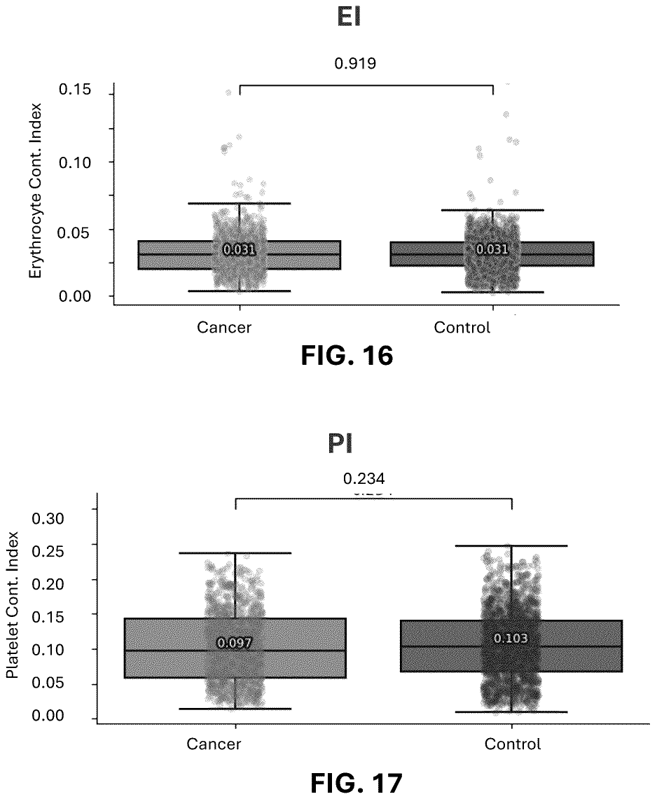

Additionally, platelet and erythrocyte contamination across collection sites was assessed (see FIG. 15). The variability in erythrocyte and platelet contamination indices across sites highlighted the importance of doing large studies across many sites—the noise averages out. It was also important that there was no statistically significant or meaningful bias between erythrocyte contamination between cancer and non-cancer samples or platelet contamination between cancer and non-cancer samples. As shown in FIG. 16, the erythrocyte contamination indices for cancer and non-cancer (control) indicated no bias between the two groups. Similarly, as shown in FIG. 17, the platelet contamination indices for cancer and non-cancer (control) indicated no bias between the two groups. These analyses were run on 2,470 subjects using Wilcoxon test comparisons annotated.

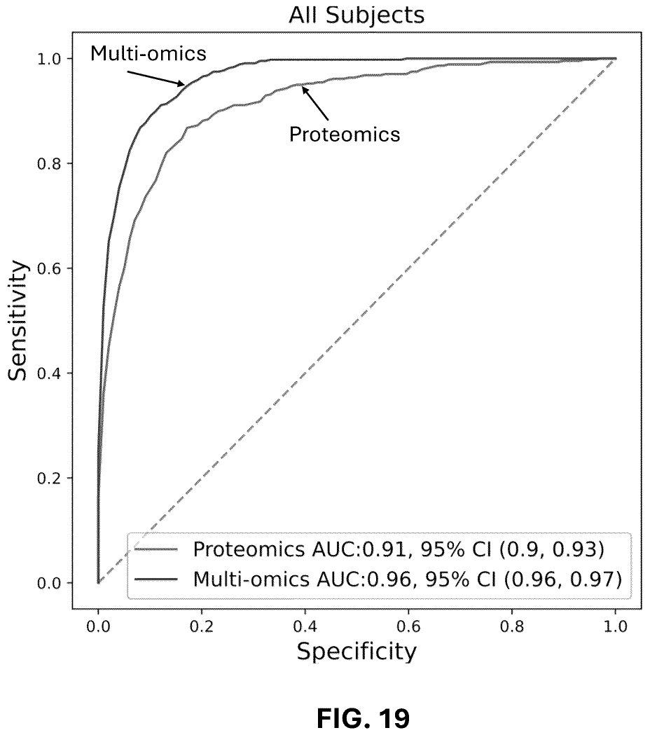

A validated classifier using 682 features covering multiple ‘omics types was assessed. The classifier had an overall sensitivity of 89% in the validation cohort comprising 398 subjects. The classifier had a specificity of 89%. When the performance was broken out by stage, the classifier demonstrated an 80% sensitivity for Stage I lung cancer, an 88% sensitivity for Stage II lung cancer, and a 98-100% sensitivity for Stage III-IV lung cancer. Overall, the classifier had an area under the curve (AUC) of 0.96 (FIG. 19).

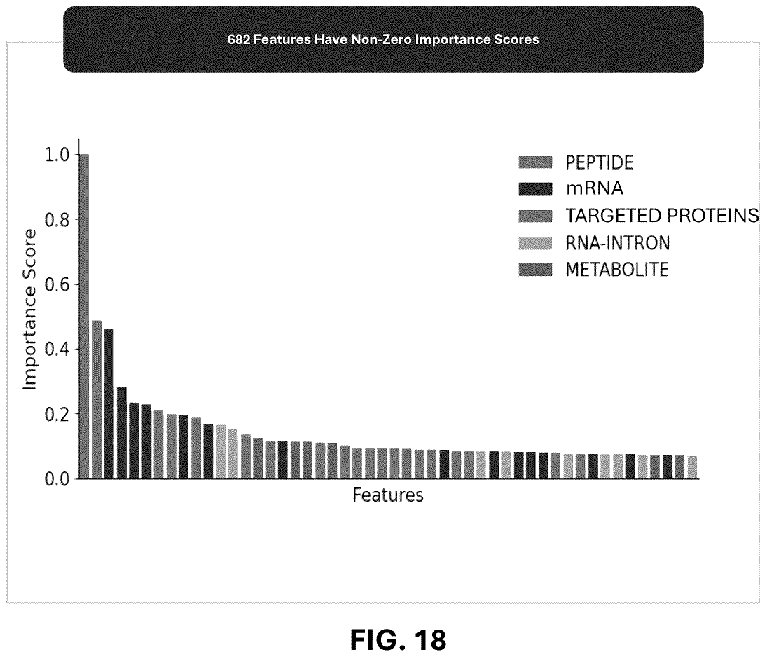

To gain a broader understanding of the relative contributions of the different ‘omics types to the validation model, the 682 features that comprised the model were ranked based on the mean-information-gain criterion. FIG. 18 shows the ‘omics type distribution among the top 50 features ranked by importance score. The multiple ‘omics classifier had an AUC of 0.96 (FIG. 19)

The most notable features in the multiple ‘omics classifier was proteins. A proteomics only classifier was assessed. The proteomics only classifier had an AUC of 0.91 (FIG. 19). Further, the proteomics only classifier had an overall sensitivity of 88% in the validation cohort comprising 398 subjects. The proteomics only classifier had a specificity of 68%. When the performance was broken out by stage, the proteomics only classifier demonstrated an 86% sensitivity for Stage I lung cancer, a 92% sensitivity for Stage II lung cancer, and a 92-96% sensitivity for Stage III-IV lung cancer.

### Verification

Verification of the discovery results was performed in an intended use population (IUP) cohort (n≈1,968). The goal of this portion of the study was to (1) verify the proteins identified in the discovery portion and demonstrate similar performance in an IUP cohort, (2) detect cancer with strong sensitivity at Stage I, and (3) differentiate cancer signal from background of non-cancer signals that are known confounders.

The verification portion of the study had broad geographic diversity across the U.S. (more than 90 clinical sites). There was biological diversity in the non-cancer controls, including inflammatory conditions such as COPD. There was an optimized uniform blood collection protocol. The study was 12% enriched.

There were 1,968 subjects enrolled in this portion of the study. The major exclusion criterion was no prior history of cancer. The key inclusion criterion was age 50-75 with greater than or equal to 25 pack years of smoking history. Early-stage cancers were well-represented, with 28% being Stage I.

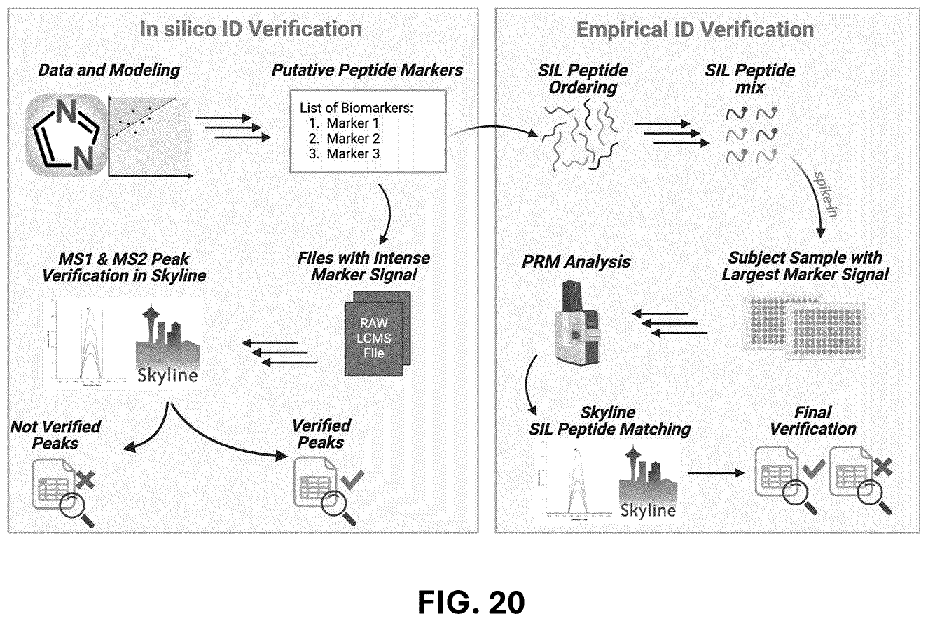

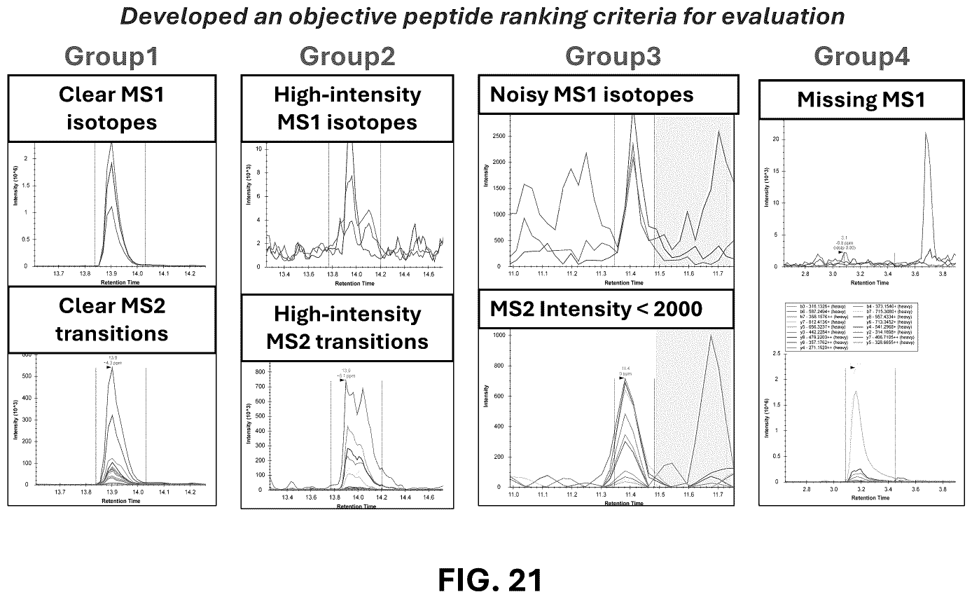

Prior to performing the verification, the output from DIA-NN (data-independent acquisition neural network) was verified. The output from DIA-NN was verified using in silico and empirical verification methods (FIG. 20). Three-hundred and fifty-seven peptide markers were empirically verified. The empirical verification used stable isotope labeled (SIL) peptides spiked into select samples. The spiked samples were processed using mass spectrometry and the SIL peptides were matched using Skyline before final verification. The in silico verification used files with intense marker signals and verified the MS1 and MS2 peaks in Skyline before final verification. Four qualitative criteria were developed for evaluation of peptides (FIG. 21). The four qualitative criteria included Group 1 representing clear MS1 isotopes and clear MS2 transitions, Group 2 representing high-intensity MS1 isotopes and High-intensity MS2 transitions, Group 3 representing noisy MS1 isotopes and MS2 intensities <2000, and Group 4 representing missing MS1.

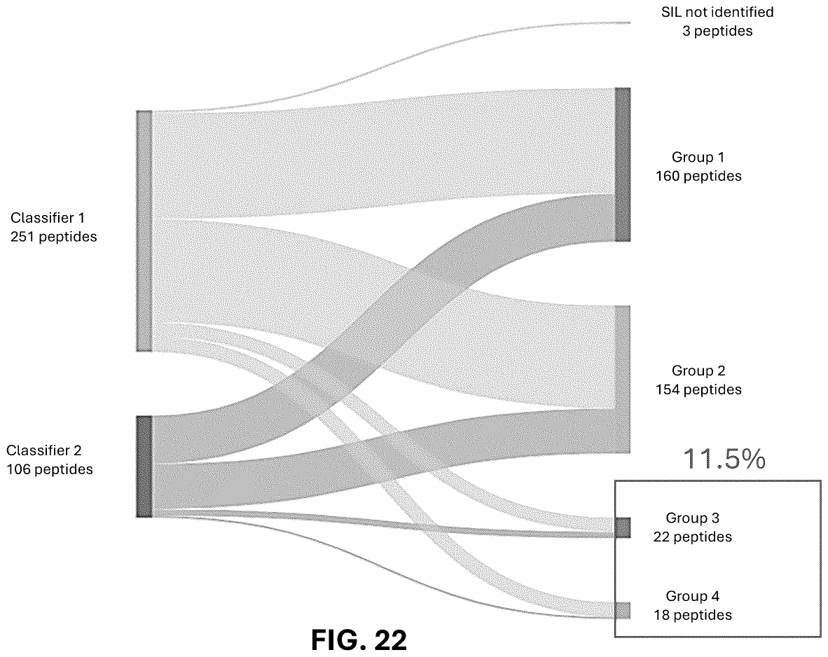

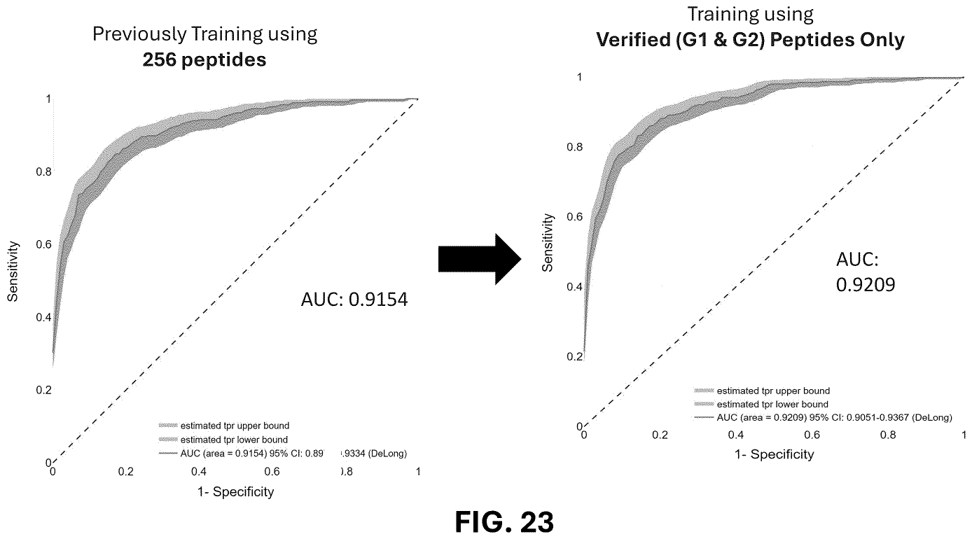

A “fail fast” strategy was used to identify robust peptide signals (FIG. 22). Classifier 1 comprised 251 peptides and Classifier 2 comprised 106 peptides. From Classifier 1 and Classifier 2, four groups of peptides were identified: Group 1 (160 peptides), Group 2 (154 peptides), Group 3 (22 peptides), and Group 4 (18 peptides). The SIL was not identified for three peptides. Group 3 and 4 comprised 11.5% of the total identified peptides. These peptides in Group 3 and 4 were determined to be false positive peptides identified with DIA-NN. When the analysis with DIA-NN was run, a 1% false discovery rate was used. The performance of the classifier trained on 256 peptides and on verified peptides (group 1 and group 2 only) is shown in FIG. 23. The removal of the false positives from the classifier did not reduce the performance as shown from the AUC of 0.9154 for the 256 peptides and the AUC of 0.9209 for the verified peptides from group 1 and group 2.

### Example 2: Development of Fully Quantitative Targeted Mass Spectrometry Verification Assay

Three tiers of targeted MS assays/measurements were developed based on the intended purpose of the measurements (“fit for purpose” concept) and then worked to define the extent of analytical validation required in each Tier as shown in Table 3.

TABLE 3
Comparisons of different types of assays

| | | | | Present |
|---|---|---|---|---|
| | | | | Disclosure - |
| | Tier 1 Assay | Tier 2 Assay | Tier 3 Assay | Tier 2 Assay |
| Areas of | Clinical | Research use | Exploratory/Discovery | Research use |
| Application | Bioanalysis/ | assays for | Studies | assays for |
| | Diagnostic | quantifying | | quantifying |
| | Laboratory | proteins, | | proteins, |
| | Test | peptides and | | peptides and |
| | | PTM's | | PTM's |
| Number of | Single or | 10's to 100's | 10's to 100's | 351 analytes |
| Analytes | small number | of analytes | of analytes | |
| | of analytes | | | |
| Degree of | High, | Moderate-to- | Low-to-moderate | High, |
| Analytical | including | high | | including |
| Validation | batch-to-batch | | | batch-to-batch |
| | QC | | | QC |
| Labeled | Yes, for every | Yes, for every | None-to-limited | Yes, for every |
| Internal | analyte | analyte | | analyte |
| Standards | | | | |
| Reference | Yes | Limited use | No | 5 point |
| Standards | | | | standard curve |
| Specificity | High | High | Moderate-to-high | High |
| Precision | High (<20- | Moderate-to- | Low-to-moderate; | Moderate-to- |
| | 25% CV) | high (<20- | similar to label free | high (<25%) |
| | | 35% CV) | discovery | |
| Quantitative | Important | Not applicable | Not applicable | Not applicable |
| Accuracy | | | | |
| Repeatability | High | High | Moderate-to-high | High |

A systematic approach was used for assay development. The operations for assay development comprised transition selection, CE optimization, RE assessment, an SIL curve, loading mass and carryover, Tier 2 full method validation, baselining between systems, Proteograph reproducibility, LC-MS reproducibility, and a verification study. In parallel, peptide stability and ion monitoring were also performed and used for the verification study.

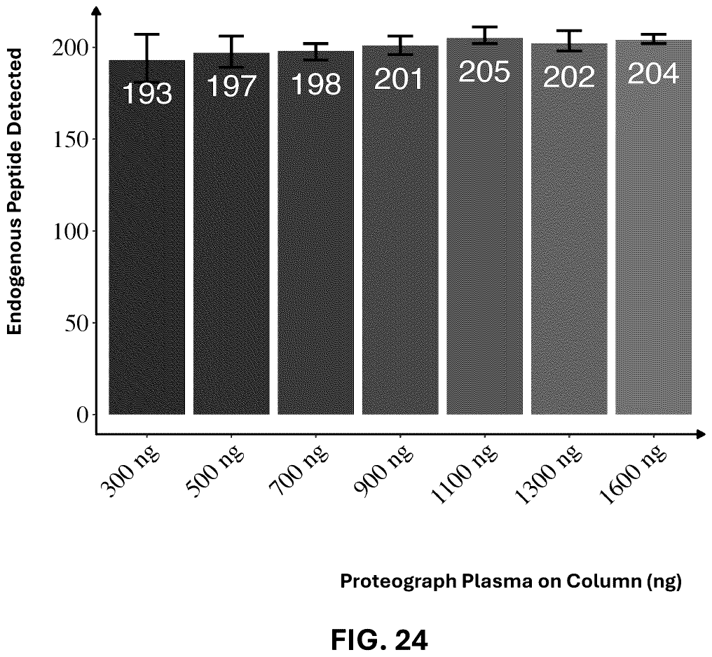

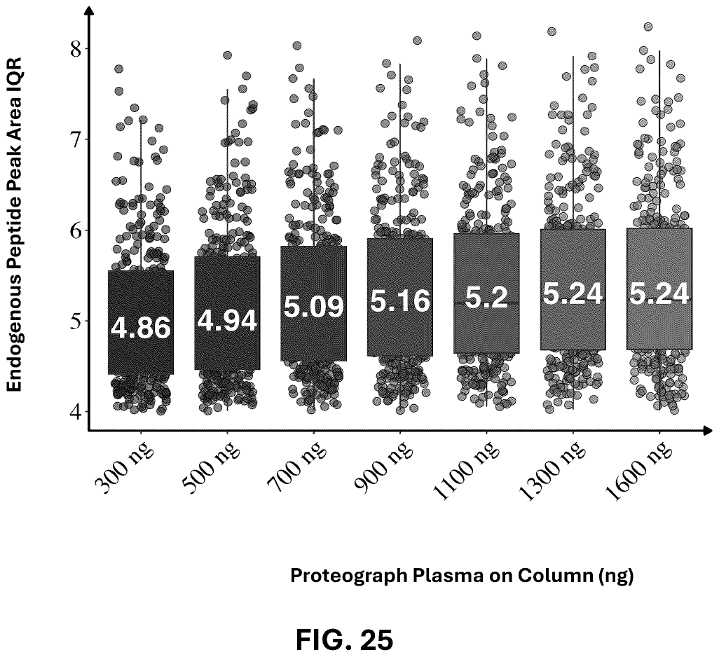

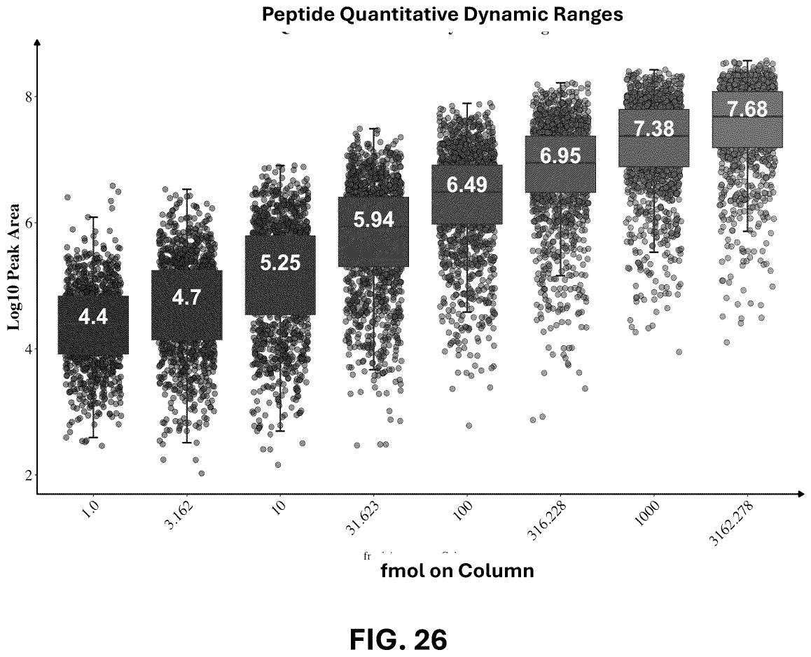

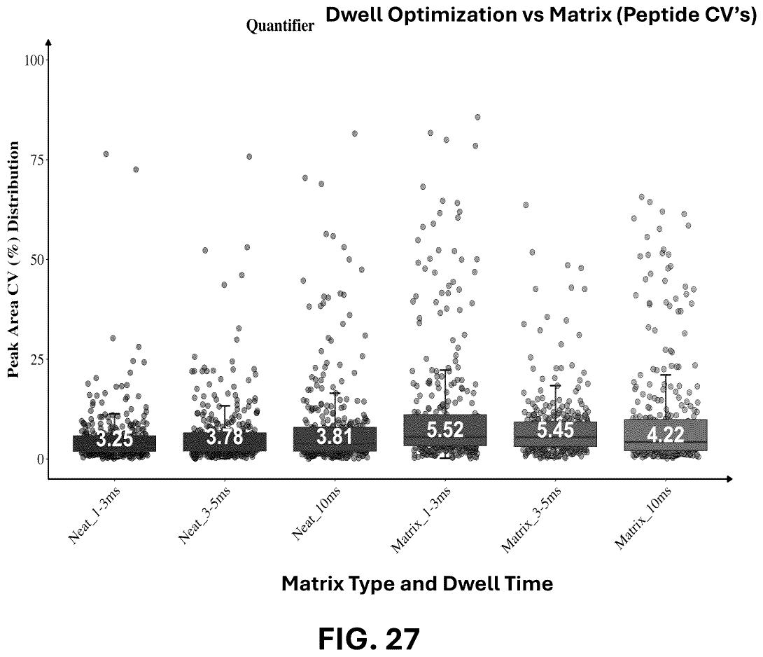

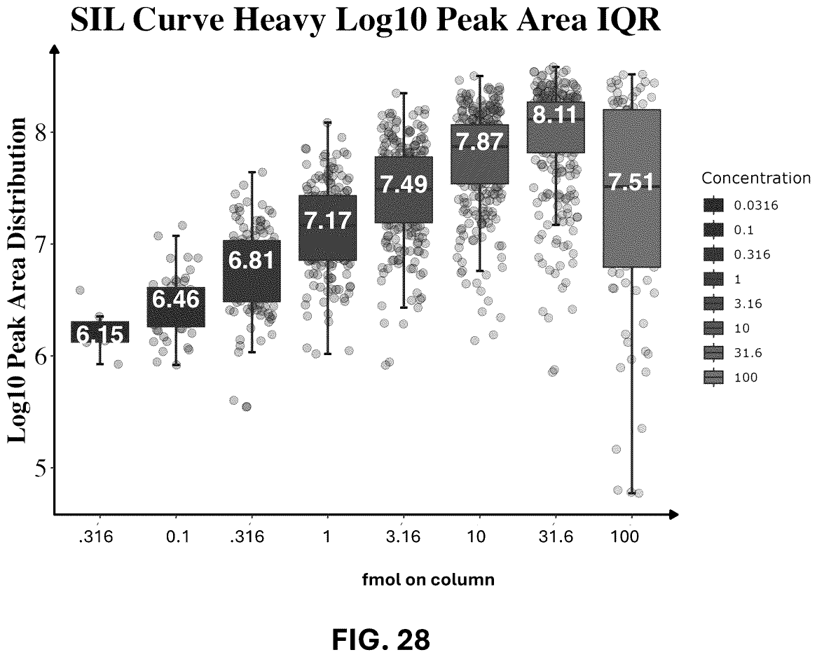

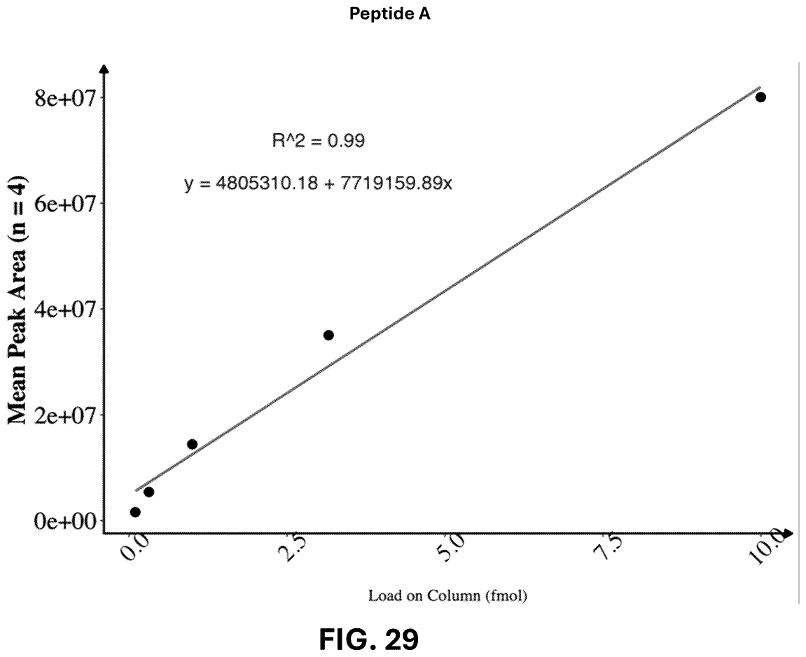

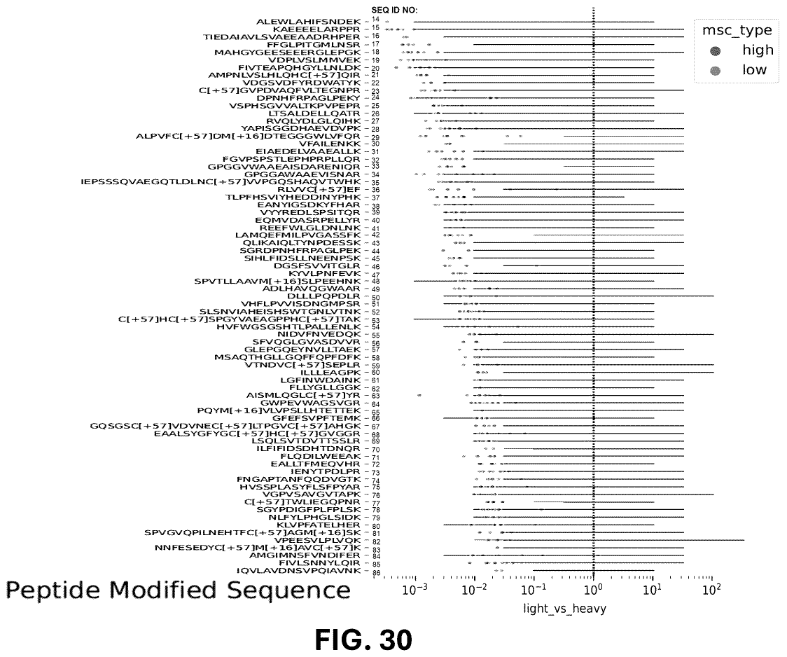

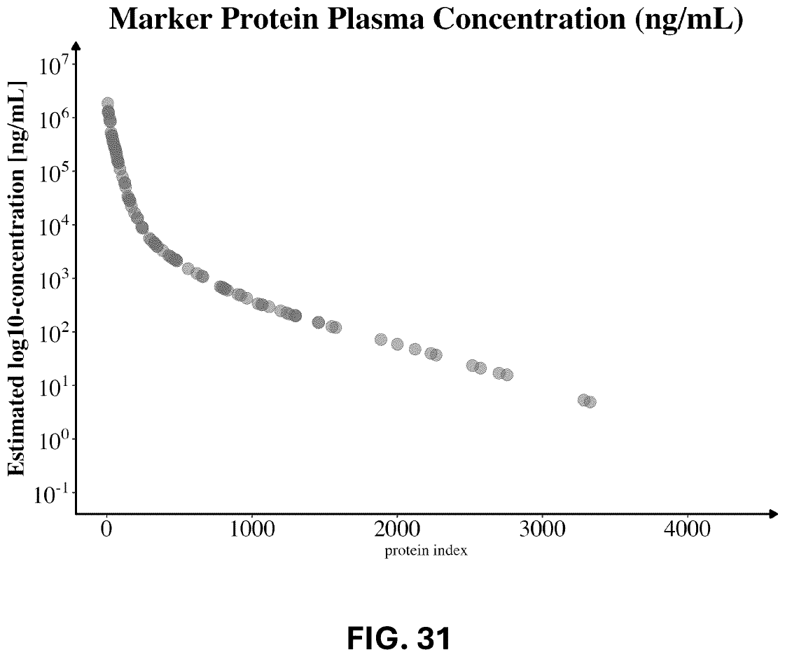

Optimization of an approximately 300 peptide (2,892 transitions) quantitative assay was performed. The matrix load was optimized prior to testing samples for the verification study. FIG. 24 shows the number of endogenous peptides detected at different amounts of Proteograph plasma on the column. Nine hundred nanograms (ng) was used for these assays. The liquid chromatography conditions included a 300 micrometer (μm)×150 millimeter (mm) column with a 10 microliter (μL) per minute flow rate. FIG. 25 shows the endogenous peptide peak area interquartile range (IQR) at different amounts of Proteograph plasma. FIG. 26 shows the peptide quantitative dynamic ranges (Log 10 Peak Area vs. femtomole (fmol) on column). FIG. 27 shows the dwell optimization vs. matrix peptide CV's (peak area coefficient of variation distribution vs. matrix type and dwell time). The dwell time was compared between neat and Proteograph plasma. The quantitation of 300 peptides was balanced in each sample injection. FIG. 28 shows the SIL Curve Heavy Log 10 Peak Area IQR. This information was used to determine how much SIL needs to be loaded on the column for all peptides. FIG. 29 shows a typical calibration curve for a peptide selected at random. Eighty percent of endogenous peptides that were potentially detected in the representative assays fell within the linear range defined in the SIL Curves experiment (FIG. 29). FIG. 30 shows the linear dynamic range for select peptides (line through graph). Verification of the approximately 300 peptides was also performed using a deep targeted assay (FIG. 31).

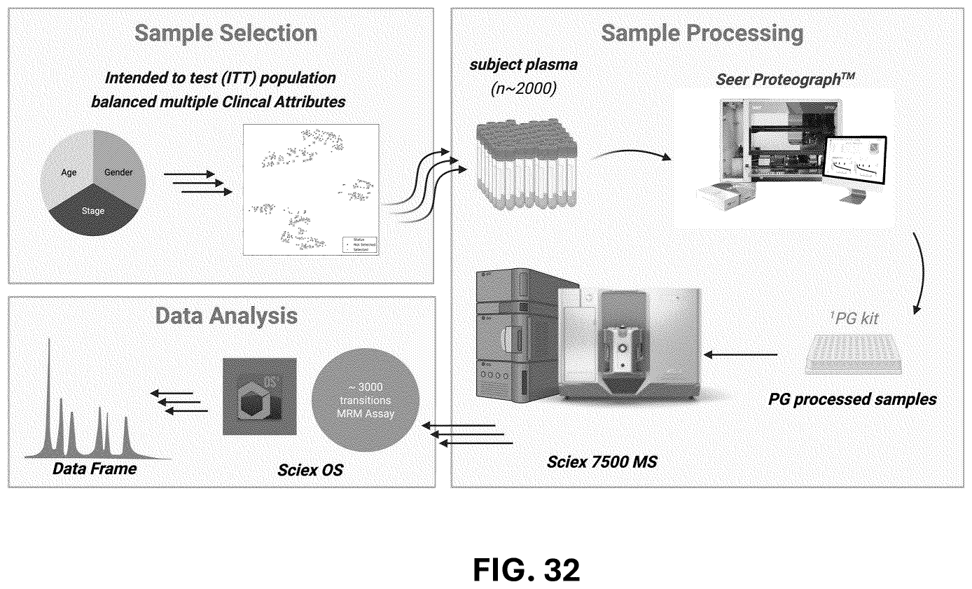

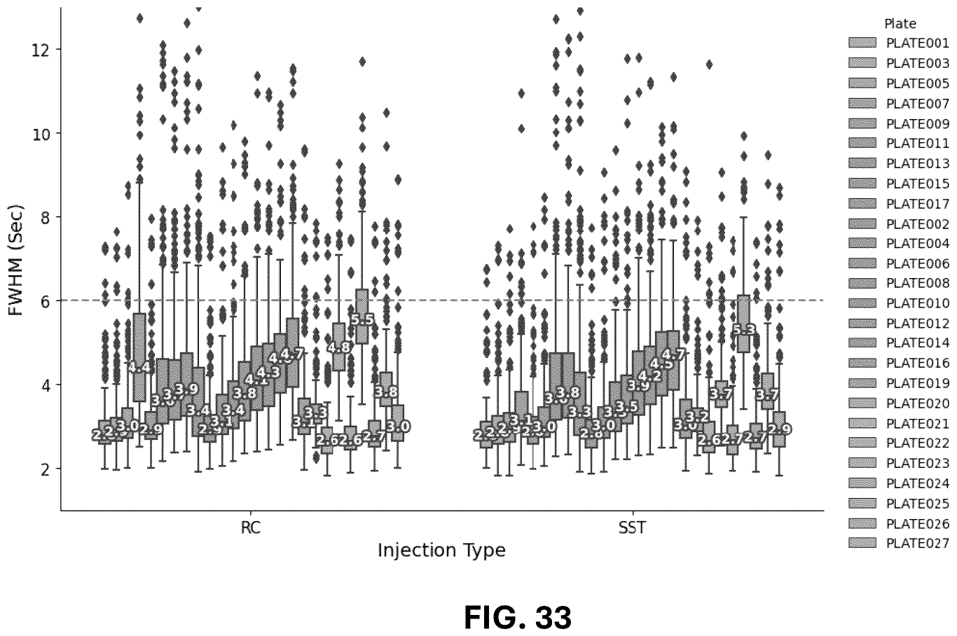

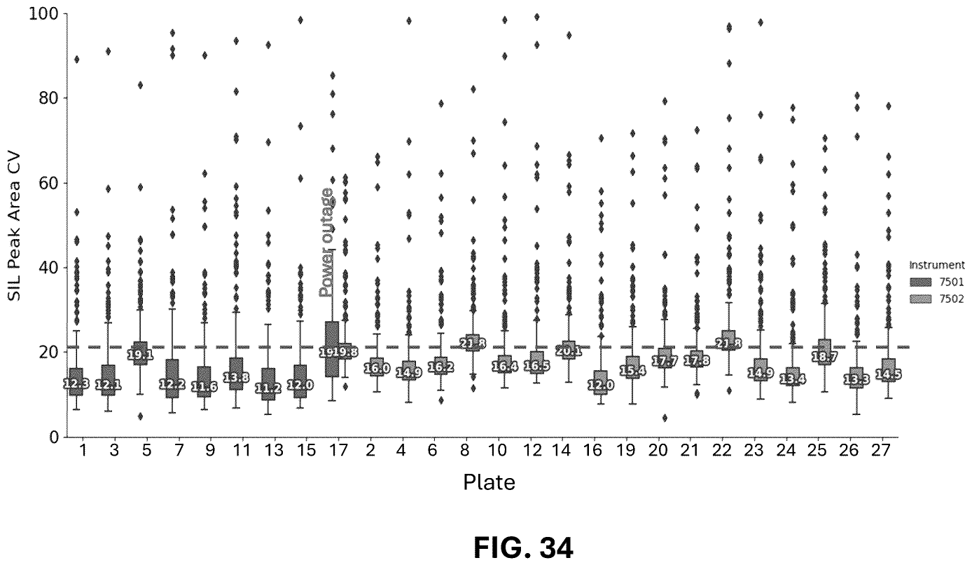

Following optimization, the verification cohort of 1,820 subjects was run using a Proteograph kit across two Sciex 7500 MS machines. FIG. 32 shows a schematic of the verification process used. The liquid chromatography column performance was closely monitored throughout the study to ensure data quality. One metric monitored was the full width at half maximum (FWHM) of the peaks to determine column quality. FIG. 33 shows FWHM distributions in seconds for different plates. For both the process controls (PC) or system suitability test (SST), the FWHM increased and then dropped correlating to a column change. The targeted LC/MS assay demonstrated low CV's across the biological samples in each batch, with most below 20%. Additionally, high technical reproducibility across the study was maintained. FIG. 34 shows CV's of internal standards across the different plates.

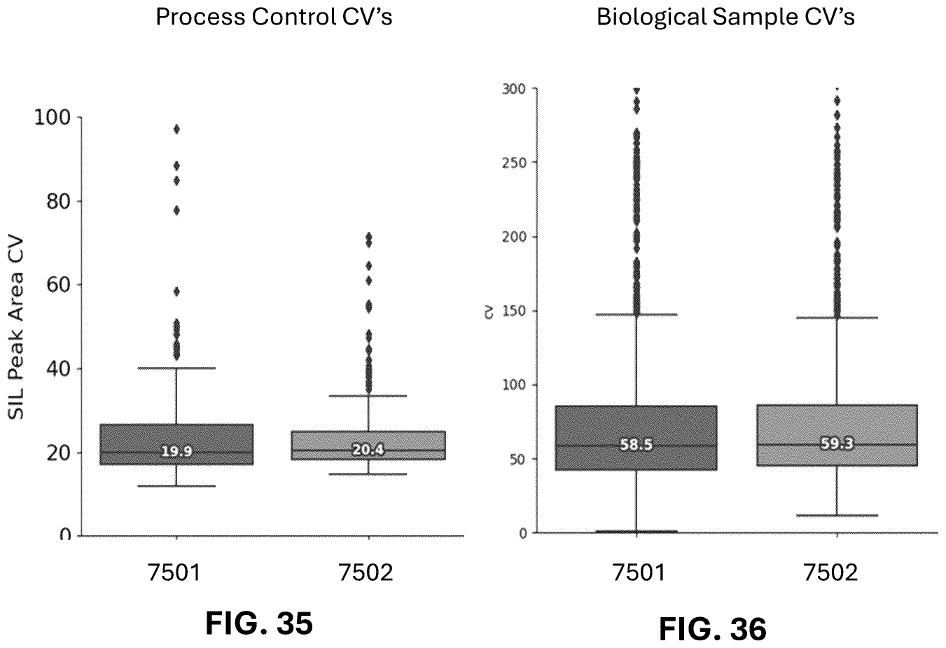

A strong biological signal was suggested by differences in technical and biological variances. Despite numerous technical challenges, the technical CV's across this study were lower than in the previous study and this study's reproducibility was higher than in the previous study. The process control % CVs were around 20% (FIG. 35) and the biological sample % CVs were approximately three times higher than the process control % CVs (FIG. 36). These results demonstrated there was real biological significance over the noise generated from the process controls.

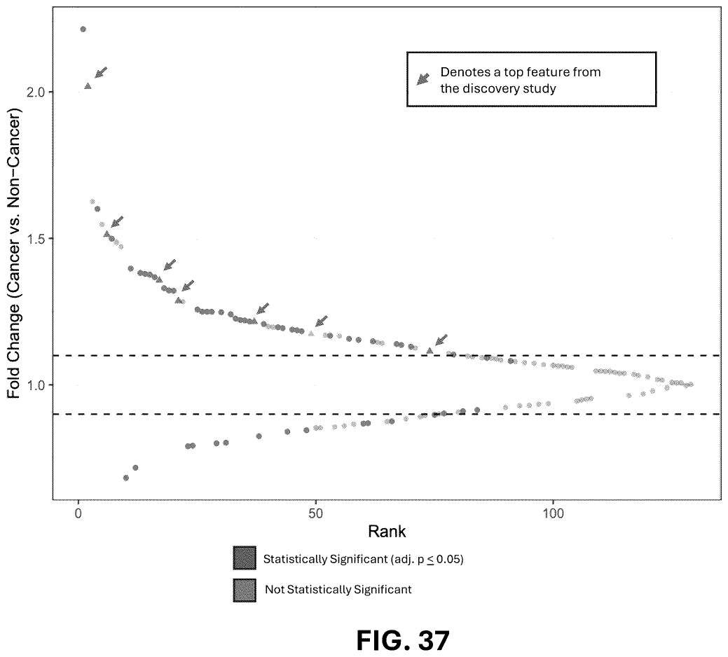

Ultimately, approximately 60 proteins were found to be statistically significant in the new IUP cohort (FIG. 37). Six out of the 7 top proteins (denoted by arrows) from the discovery portion of the study were statistically significant with targeted LC/MS. Fifty-three additional proteins were found to be statistically significant on targeted LC/MS between cancer and non-cancer subjects. After verifying these proteins identified in the discovery cohort, analysis was performed to translate the test to another platform.

### Example 3: Translation of Unbiased LC/MS Discoveries to Immunoassay Platforms

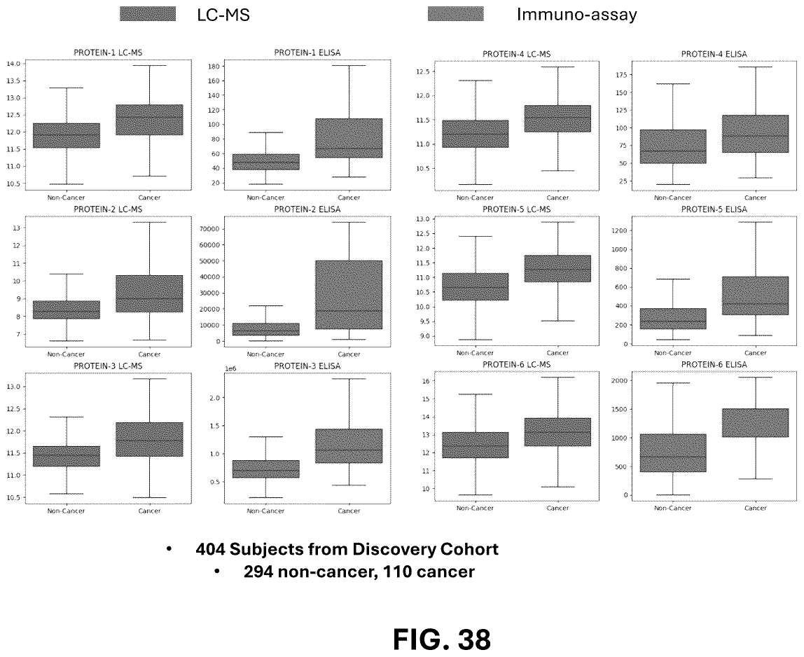

The translation of the top 178 proteins from the discovery study from targeted mass spectrometry to a multi-plexed immunoassay was assessed. First, a pilot study was conducted to assess six key proteins from the classifier on LC/MS and ELISA platforms with 404 subject samples from the discovery cohort. The comparison of LC/MS and ELISA analysis for each of the six proteins showed similar performance across platforms and samples (FIG. 38).

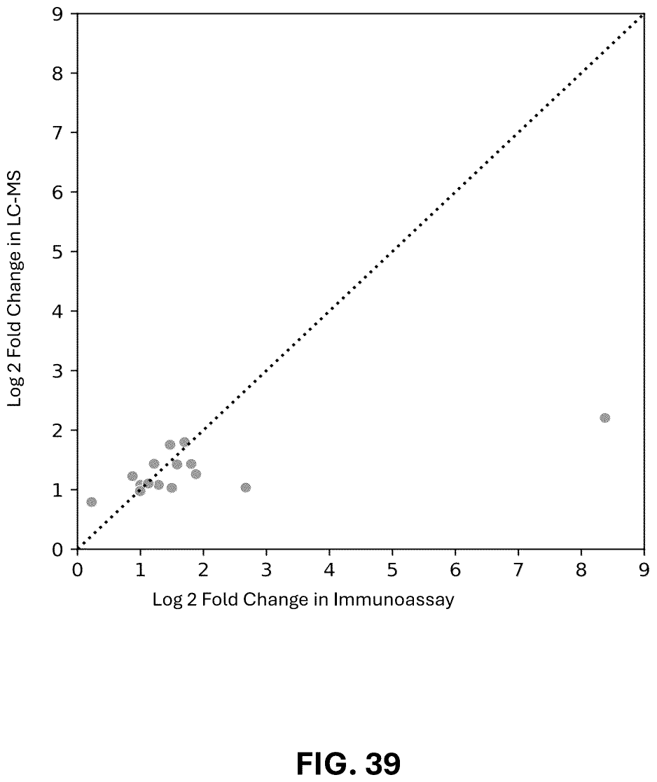

Additionally, a strong correlation between Log 2 fold changes on LC/MS and immunoassay was observed and indicated good concordance between the platforms (FIG. 39). However, one protein had an 8-fold change on immunoassay as compared to LC/MS. This is thought to be due to issues with specificity for this protein in the immunoassay format.

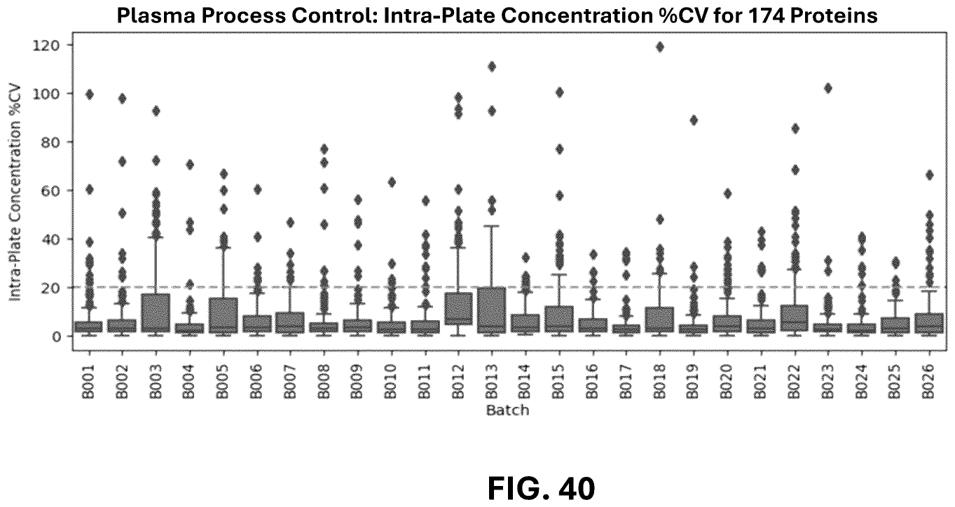

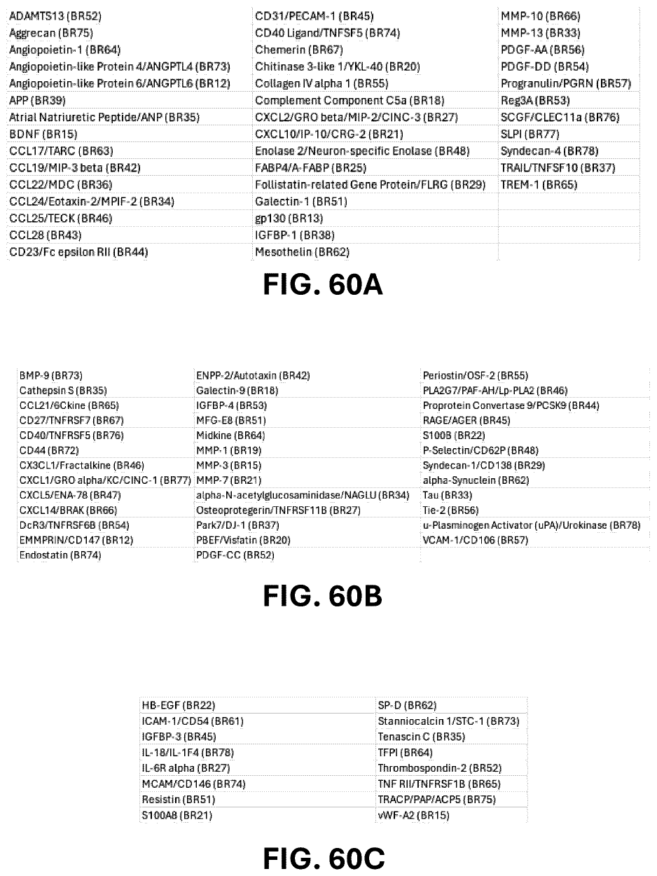

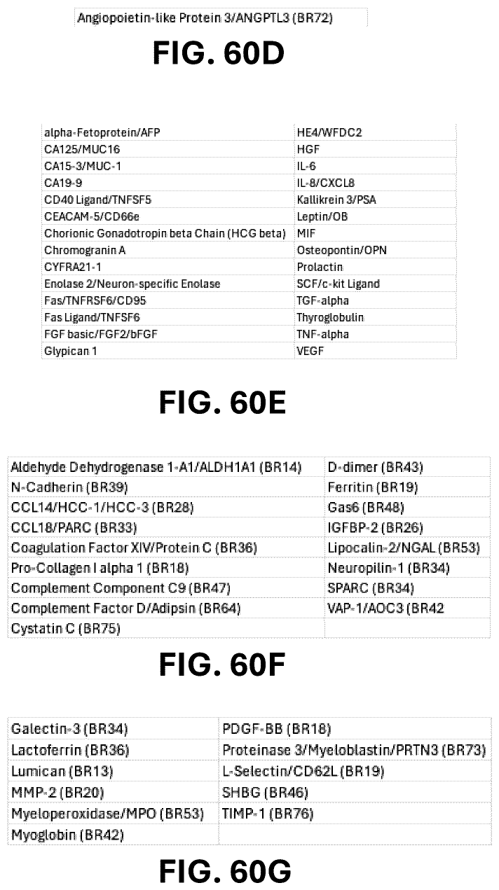

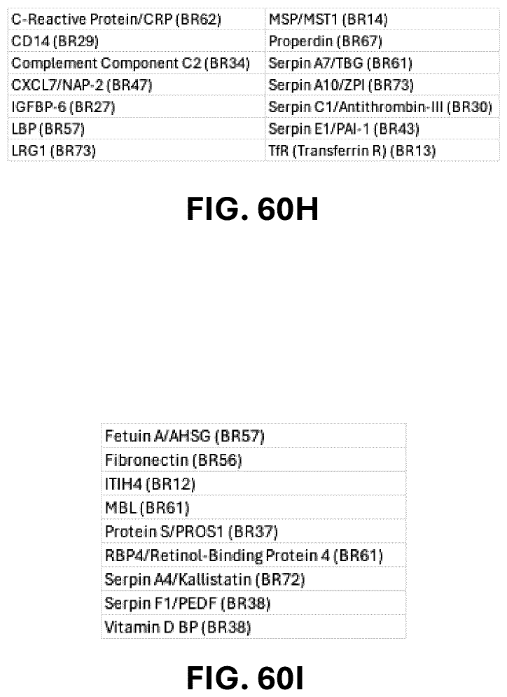

The immunoassays for the top 178 proteins were run in a semi-automated platform including the use of automation for plasma dilutions, generating standard curves, and plating controls. One hundred and seventy-five proteins were run across 9 panels on the Luminex platform using either the Luminex Discovery or Luminex High-Performance Panels. Panel 1 (FIG. 60A), Panel 2 (FIG. 60B), Panel 3 (FIG. 60C), Panel 8 (FIG. 60D), and Panel 9 (FIG. 60E) were run with a 1:2 dilution. Panel 4 (FIG. 60F) and Panel 5 (FIG. 60G) were run with a 1:50 dilution. Panel 6 (FIG. 60H) was run with a 1:200 dilution. Panel 7 (FIG. 60I) was run with a 1:4000 dilution. FIGS. 60A-I shows the proteins run in each panel with the bead regions in parenthesis where applicable. Three proteins (FGL1, ORM2, and SAA) were run on single ELISAs with Human Fibrinogen-like protein 1/FLG1 ELISA kit (Abcam), Human alpha 1-acid Glycoprotein ELISA kit/ORM1 (R&D Systems), and Human Serum Amyloid A1/SAA1 ELISA kit (R&D Systems), respectively. In addition, CA125 and CEA were run using the Ortho/VITROS assay. LTBP2 was also assessed for assay development, but could not be established on the Luminex assay. FIG. 40 shows the intra-plate concentration % CV for process controls of 174 proteins. There were multiple process controls on each plate/batch. The median CV % were all below 20% with most below 5%. This indicated good reproducibility.

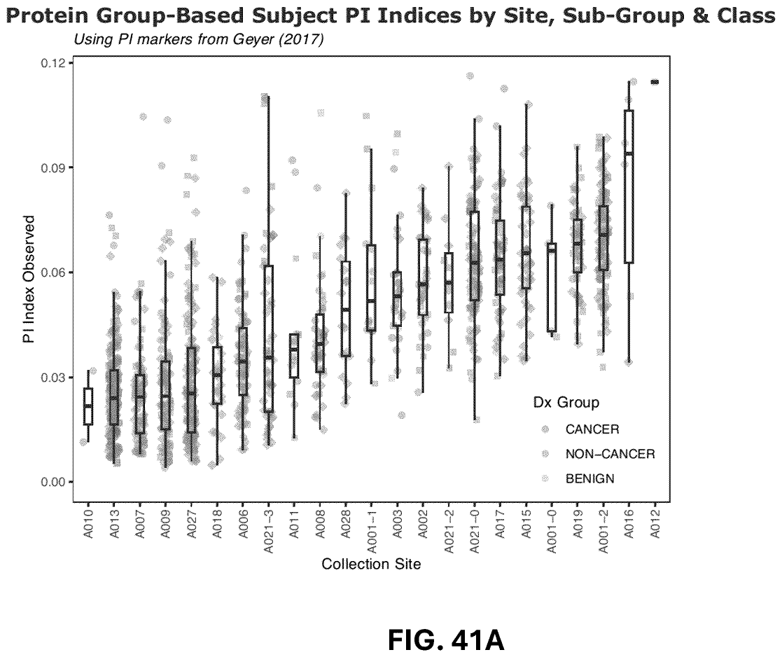

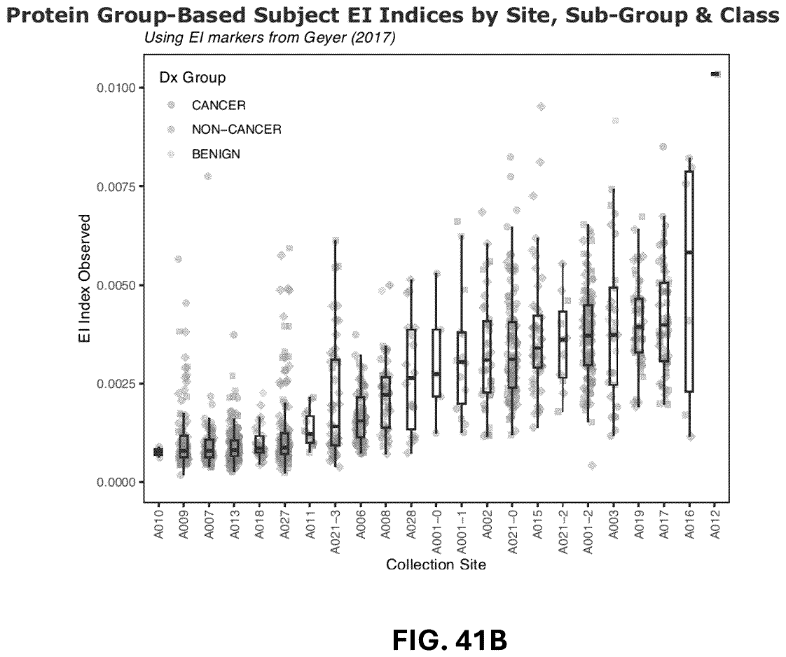

In conjunction with the translation work, samples were run on an Orbitrap Astral Mass Spectrometer to generate unbiased data that looked at pre-analytical variability. Pre-analytical variability was a consideration by site. Variability in the number of cancer, non-cancer, and benign samples appeared across sites with some sites having a much higher number of one type of sample over the others. This may cause a classifier trained on such data to learn site specific noise signatures, in such a case the classifier may not perform as well when given samples without the site-specific noise. Additionally, the PI and EI index for each site may vary introducing site specific EI and PI based noise that may have a similar effect on a classifier's performance as the site specific noise. Here, some of the highest recruiting sites primarily recruited only non-cancer samples; these sites also happened to be the cleanest sites. Sites that recruited more cancer samples tended to have more contamination. FIG. 41A shows protein group-based subject PI indices by site, sub-group, and class. FIG. 41B shows protein group-based subject EI indices by site, sub-group, and class. To minimize these issues, sites were balanced and capped. Additionally, some of the immunoassays had some correlation with the EI and PI in the panel creating a balance.

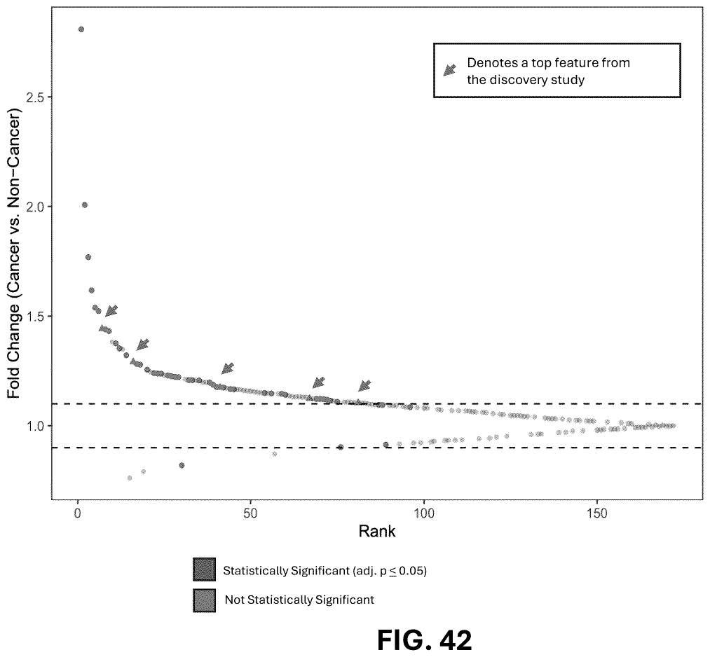

After pre-analytical balancing, 51 proteins were statistically significant. Most of the key proteins of interest were translated and verified in this experiment. FIG. 42 shows the rank and significance of proteins analyzed. Arrows in FIG. 42 indicate top features from the discovery study.

Performance of <20 features of a single analyte (proteins) had a 90% overall sensitivity (n=82) and 69% specificity (n=853) in the training set cohort (n=935). Performance of the classifier by stage of disease was 87% sensitivity (n=26) for Stage I, 88% sensitivity (n=12) for Stage II, and 100% sensitivity (n=29) for Stage III and IV. Again, there was a drop in specificity over the initial classifier; however, this is likely due to the harder IUP cohort of 12% since the performance on theses samples was similar between immunoassay and LC-MS platforms.

### Example 4: Validation of the Liquid Biopsy Lung Cancer Test Using Immunoassays

Some goals of this study were to (1) validate proteins verified in the previous study in a third and final IUP cohort, (2) detect lung cancer with strong sensitivity at Stage I, and (3) differentiate cancer signal from background of non-cancer signals that may be known confounders.

The study was designed to have broad geographic diversity across the U.S. (more than 46 clinical sites). The study was balanced to minimize potential confounders from sites and pre-analytical variability. The study used case control prospectively collected samples. The study was 8% enriched.

There were 2,048 subjects enrolled with molecular data collected. The major exclusion criterion was no prior history of cancer. The key inclusion criterion was age 50-75 with greater than or equal to 25 pack years of smoking history. Early-stage cancers were well-represented with approximately 42% Stage I lung cancer.

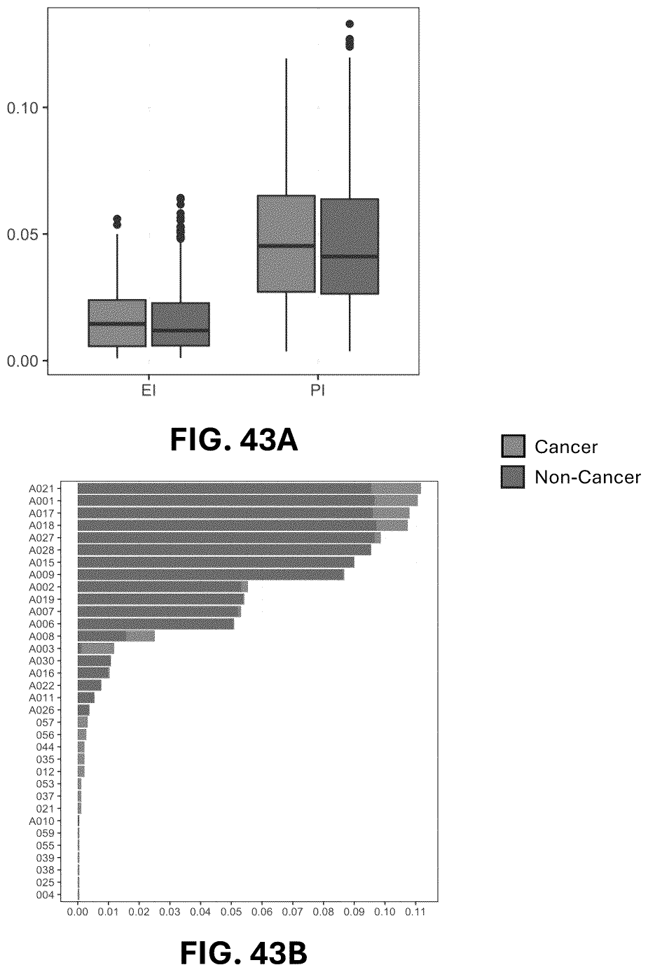

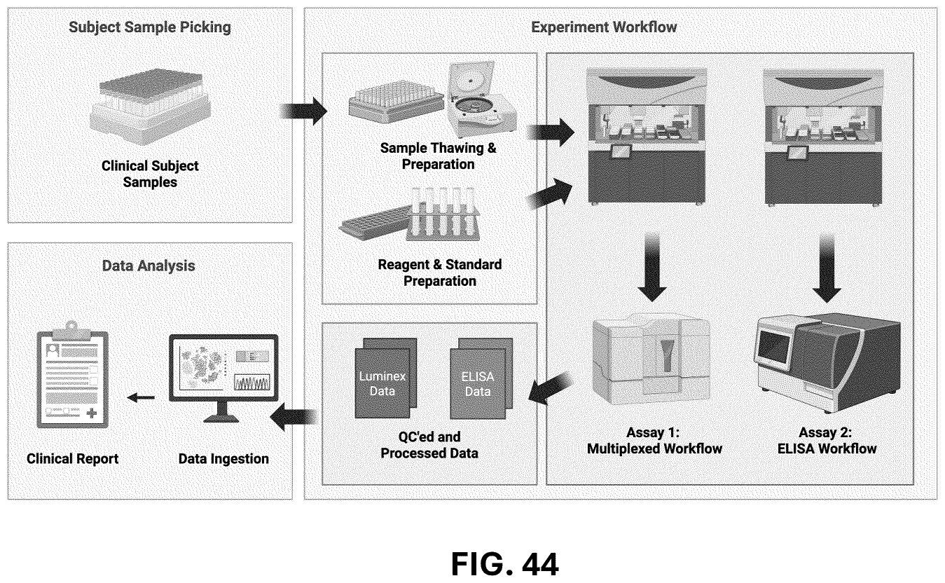

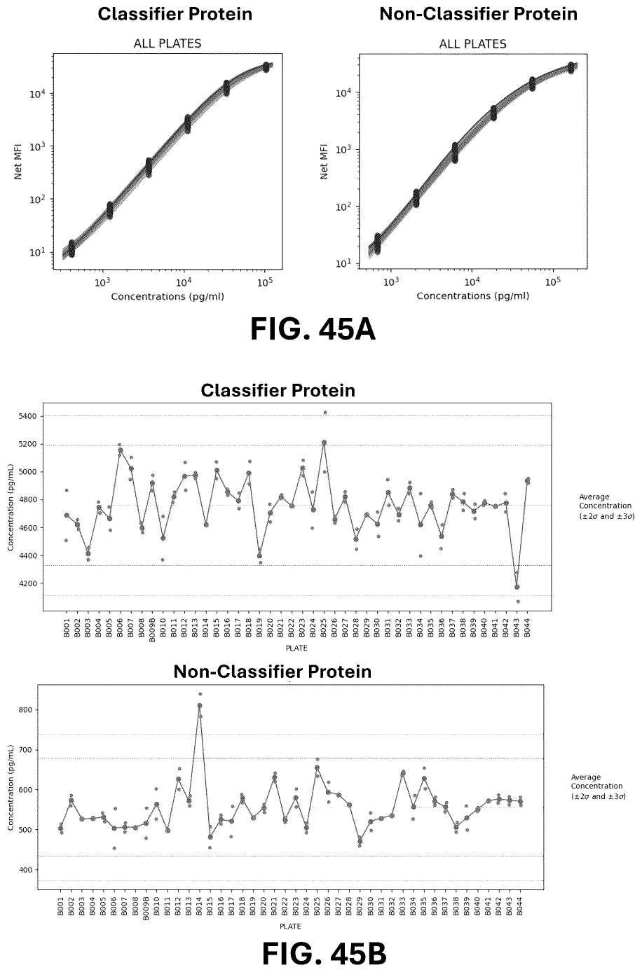

Based on what was learned in the translation study, an Orbitrap Astral Mass Spectrometer was again used to balance the study in advance of data collection. The study was balanced to ensure there was no statistically significant bias between cancer and non-cancer samples for EI and PI (FIG. 43A). The study was also balanced by limiting the number of cancer samples that could come from any one site (FIG. 43B). Following balancing, samples were processed in the fully automated workflow for this study (FIG. 44). Assay performance was shown to be highly reproducible for the 47 protein immunoassays. For each of these proteins standard curves were overlaid showing good reproducibility across the study (FIG. 45A). The Levey Jennings plots of the process controls for two assays are also shown (FIG. 45B).

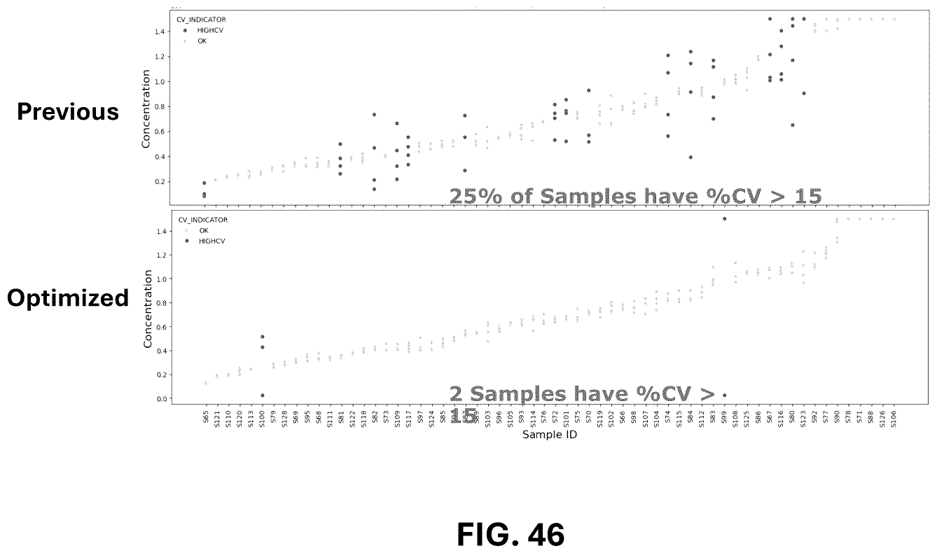

Variability among samples was observed prior to analytical validation. This variability was assessed to ensure robust data collection across samples taken at the same time (which may be expected to have low variability); however, as shown in FIG. 46 the previous data (e.g., non-optimized data) demonstrated a high CV (% CV of >15) for about 25% of the samples. After optimizing sample handling procedures (such as freezing and thawing practices, described elsewhere herein) only two samples had a % CV of >15 (low variability) which were subsequently found to have had no sample in one well contributing to the CV significantly. Thus, variability in the handling of the samples further reduced noise in the sample collection and processing which may contribute to improving classifier performance.

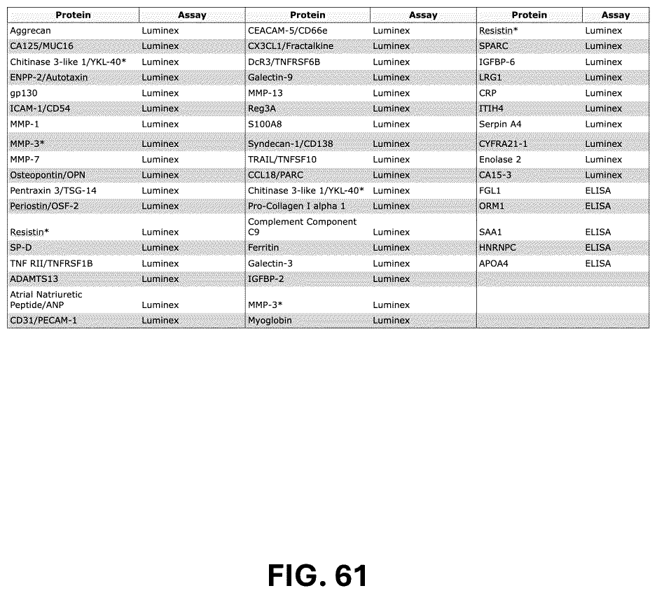

Study subjects were assigned to training and validation partitions using methods designed to balance covariate distributions across cohorts, while applying limits to reduce overrepresentation from individual clinical sites in accordance with study inclusion and exclusion criteria. FIG. 61 shows the 48 proteins and the corresponding assays considered for this study including HNRNPC (UniProt ID: P07910) and SerpinA4 (UniProt ID: P29622) which were included for their promise as candidates for use in the classifier to distinguish between lung cancer. The lung cancer classifier was trained using protein measurements from eight Luminex-based multiplex panels, four ELISA-based assays, and two Ortho-based assays targeting 46 proteins identified in prior studies. Models were selected for validation through training cross-validation and implementation of defined cross validation strategies. The validated models were evaluated using statistical metrics, including additional evaluation on subjects excluded from the data set.

Forty-six proteins, identified in earlier studies for product development, were measured using three immunoassay platforms: eight Luminex-based multiplex panels (Panels 15-22), four ELISA-based assays (ORM1, FGL1, APOA4, S100A8/A9) and two Ortho/VITROS based assays (CEA, CA125). The clinical test under development was distinct from the one evaluated in prior studies necessitating a new round of machine learning training utilizing protein measurements from the Luminex, ELISA and Ortho/VITROS based immunoassays. Details of proteins analyzed in the eight Luminex-based multiplex panels, four ELISA assays, and two Ortho/VITROS assays are shown in Table 41.

TABLE 41
List of Proteins analyzed on Luminex Panels (panel
15-22), ELISA, and/or Ortho/VIRTOS Assays

| Panel | Protein | Uniprot |
|---|---|---|
| PL15 | Aggrecan | P16112 |
| 1:2 dilutions | CA125/MUC16 | Q8WXI7 |
| Luminex Human Discovery Assay | Chitinase 3-like 1/YKL-40 | P36222 |
| (15-Plex) LXSAHM-15 | ENPP-2/Autotaxin | Q13822 |
| | gp130 | P40189 |
| | ICAM-1/CD54 | P05362 |
| | MMP-1 | P03956 |
| | MMP-3 | P08254 |
| | MMP-7 | P09237 |
| | Osteopontin/OPN | P10451 |
| | Pentraxin 3/TSG-14 | P26022 |
| | Periostin/OSF-2 | Q15063 |
| | Resistin | Q9HD89 |
| | SP-D | P35247 |
| | TNF RII/TNFRSF1B | P20333 |
| PL16 | ADAMTS13 | Q76LX8 |
| 1:2 dilutions | Atrial Natriuretic Peptide/ANP | P01160 |
| Luminex Human Discovery Assay | CD31/PECAM-1 | P16284 |
| (12-Plex) LXSAHM-12 | CEACAM-5/CD66e | P06731 |
| | CX3CL1/Fractalkine | P78423 |
| | DcR3/TNFRSF6B | O95407 |
| | Galectin-9 | O00182 |
| | MMP-13 | P45452 |
| | Reg3A | Q06141 |
| | S100A8 | P05109 |
| | Syndecan-1/CD138 | P18827 |
| | TRAIL/TNFSF10 | P50591 |
| PL17 | CCL18/PARC | P55774 |
| 1:50 dilution | Complement Component C9 | P02748 |
| Luminex Human Discovery Assay | Ferritin | P02792 |
| (12-Plex) Custom-LxSA-H-12 | Galectin-3 | P17931 |
| | IGFBP-2 | P18065 |
| | MMP-3 | P08254 |
| | Myoglobin | P02144 |
| | Pro-Collagen I alpha 1 | P02452 |
| | Resistin | Q9HD89 |
| | SPARC | P09486 |
| | TNF RII/TNFRSF1B | P20333 |
| | Chitinase 3-like 1/YKL-40 | P36222 |
| PL18 | IGFBP-6 | P24592 |
| 1:200 dilution | Complement Component C9 | P02748 |
| Luminex Human Discovery Assay | CRP | P02741 |
| (4-Plex) LXSAHM-04 | LRG1 | P02750 |
| PL19 | ITIH4 | Q14624 |
| 1:4000 dilution | Complement Component C9 | P02748 |
| Luminex Human Discovery Assay | CRP | P02741 |
| (4-Plex) LXSAHM-04 | LRG1 | P02750 |
| PL20 | CA125/MUC16 | Q8WXI7 |
| 1:2 dilutions | CA15-3/MUC1 | P15941 |
| Luminex Performance Human | CYFRA21-1 | P08727 |
| (4-Plex) LXSAHM-04 | Enolase 2/Neuron-specific Enolase | P09104 |
| PL21 | CECAM-5/CD66e | P06731 |
| 1:8 dilutions | Osteopontin/OPN | P10451 |
| Luminex Performance Human | | |
| Tumor Biomarker Panel (2-Plex) | | |
| FCSTM25-02 | | |
| PL22 | SAA | P0DJI8 |
| Luminex Thermo Fisher | SAA | P0DJI9 |
| ProcartaPlex Assay (1-Plex) | | |
| EPX01A-12136-901 | | |
| ELISA | FGL1 | Q08830 |
| | ORM1 | P02763 |
| | APOA4 | P06727 |
| | SAA | P0DJI8 |
| | SAA | P0DJI9 |
| | S100A8 | P05109 |
| Ortho/VITROS Immunodiagnostic | CA125 | Q8WXI7 |
| System markers | CEA | P06731 |

A subset of the 2,068 study subjects were selected and evenly divided into training and testing partitions for machine learning of lung cancer classification models. Lung cancer classification models were trained on the training partition study data and evaluated by cross-validation. The top lung cancer classifier model based on the highest average specificity at 85% sensitivity across 200 repeats of 2-fold cross-validation was selected for validation on the testing partition of the study data.

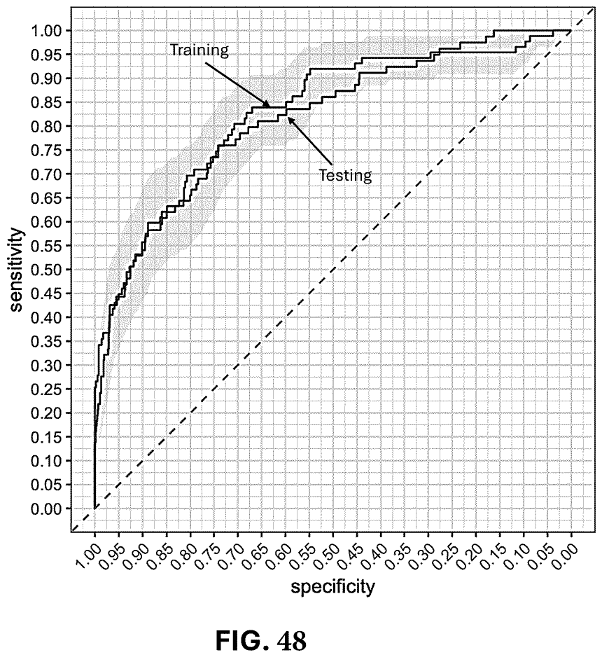

The area under the receiver operating characteristic curve (AUROC) of the selected lung cancer classifier on the training and test data is shown in FIG. 48. This classifier had an AUROC of 0.83 with the training data and an AUROC of 0.82 with the test data (FIG. 48). The bootstrap estimate of the 95% confidence interval around the AUROC in the training partition is shown in the light grey shading (FIG. 48). The AUROC on test data fell within the 95% confidence interval established from the training data. The specificity at 85% sensitivity also fell within the 95% confidence interval established from training with a specificity of 61% (95% CI 53% to 74%) for the training data and a specificity of 56% (95% CI 42% to 74%) for the testing data.

Using the probability threshold at 85% sensitivity, the bootstrap estimates of the sensitivity per cancer stage on the testing partition were 81% (95% CI 50% to 100%) for Stage I, 100% (95% CI 56% to 100%) for Stage II, 88% (95% CI 50% to 100%) for Stage III, and 89% (95% CI 50% to 100%) for Stage IV.

The final classifier included the analysis of eleven proteins and twelve measurements. The eleven proteins were C9, CEACAM-5/CEA, CYFRA21-1, CA125, Myoglobin, Enolase 2, gp130, FGL1, IGFBP-6, PECAM-1, and SAA. Two measurements used in the final classifier were generated from CEACAM-5/CEA. Classifier details on the feature, immunoassay, and logistic regression coefficients of the features in the validated model are shown in Table 42. The logistic regression coefficients refer to the increase (positive) or decrease (negative) in log-odds of a non-cancer diagnosis (e.g., not elevated) per unit increase of the corresponding protein feature.

TABLE 42
Logistic Regression Feature Coefficients

| Feature | Feature Type | Coeff (Non-Cancer) |
|---|---|---|
| (Intercept) | (Intercept) | 26.47774721 |
| (Alpha) | (Alpha) | 0 |
| (Lambda) | (Lambda) | 0 |
| PL17\|Complement Component C9 | Luminex | −1.03823597 |
| PL21\|CEACAM-5/CD66e | Luminex | 0.56525527 |
| ORTHO\|CEA | ORTHO | −0.54238374 |
| PL20\|CYFRA21-1 | Luminex | −0.5053308 |
| ORTHO\|CA125 | ORTHO | −0.40066858 |
| PL17\|Myoglobin | Luminex | 0.24862465 |
| PL20\|Enolase 2/Neuron-specific Enolase | Luminex | −0.24624901 |
| PL15\|gp130 | Luminex | −0.18477899 |
| ELISA\|FGL1_500X | ELISA | −0.1241148 |
| PL18\|IGFBP-6 | Luminex | 0.09950091 |
| PL16\|CD31/PECAM-1 | Luminex | 0.09303025 |
| PL22\|SAA | Luminex | −0.06480559 |

Additional data was collected on 587 subjects that were not part of the study. These subjects were used to estimate the general performance of the cancer classifier at a fixed cancer probability threshold. The cancer probability threshold was fixed to 0.3781735, corresponding to the largest probability value that would correctly call 67 of the 79 cancer subjects in the testing partition (e.g., 84.8% sensitivity or 85% sensitivity when rounding to two significant figures). On the 587 subjects, the cancer classifier with that fixed cancer probability threshold achieved an overall sensitivity of 87% and specificity of 52%.

Table 43 shows the breakdown of the number of correctly and incorrectly called subjects on all 587 subjects that were not a part of the study with Table 44 showing the breakdown specifically for cancer subjects across different cancer stages. Performance of the validated lung cancer classification model on the subset of the 587 subjects with cancer stages I-IV, using the same probability threshold as in Table 43, resulted 80%, 80%, 89%, and 100% sensitivities for Stages I, II, III, and IV, respectively.

TABLE 43
Overview of Correct and Incorrect Calls:
Total Subjects and Cancer Stage Subset

| Truth | Prediction | Number |
|---|---|---|
| Cancer | Cancer | 88 |
| | Non-Cancer | 13 |
| Non-Cancer | Cancer | 231 |
| | Non-Cancer | 255 |

TABLE 44
Cancer Subject Classification Results by Stage

| Truth | Prediction | Number |
|---|---|---|
| Stage I | Cancer | 28 |
| | Non-Cancer | 7 |
| Stage II | Cancer | 8 |
| | Non-Cancer | 2 |
| Stage III | Cancer | 25 |
| | Non-Cancer | 3 |
| Stage IV | Cancer | 10 |
| | Non-Cancer | 0 |

In conclusion, a lung cancer classification model on 11 proteins was trained on the training partition subjects and validated within pre-specified requirements on the testing partition. On a held-out set of subjects that were excluded from the study and using the decision threshold set to 85% sensitivity on the testing partition, the model achieved comparable performance as on the testing partition. This finding affirmed that the model performance is likely to generalize to subjects outside of those specifically selected for the study. The outcome from the study is a lung cancer classification model on 11 proteins with anticipated performance of 85% sensitivity and 55% specificity in the intended use population (IUP).

In summary, across the multiple stages of the study (e.g., discovery, verification, translation, and validation), >6,500 subject samples across 3 distinct cohorts were analyzed to discover, verify, and validate protein markers for the early detection of lung cancer. Rigorous study design and quality samples may be necessary for successful discovery and diagnostic development. Each consecutive cohort throughout the development process was increasingly similar to an Intended Use Population and incorporated less enrichment. High Stage I detection performance was retained across all cohorts. The initial classifier was 682 multi ‘omics features. The final validated assay was less than 20 proteins. The final set of proteins demonstrated high performance across all cohorts, demonstrating the generalizability of the proteins in the validated assay. These studies validated the capabilities of deep unbiased proteomics to discover novel markers for real world clinical tests.

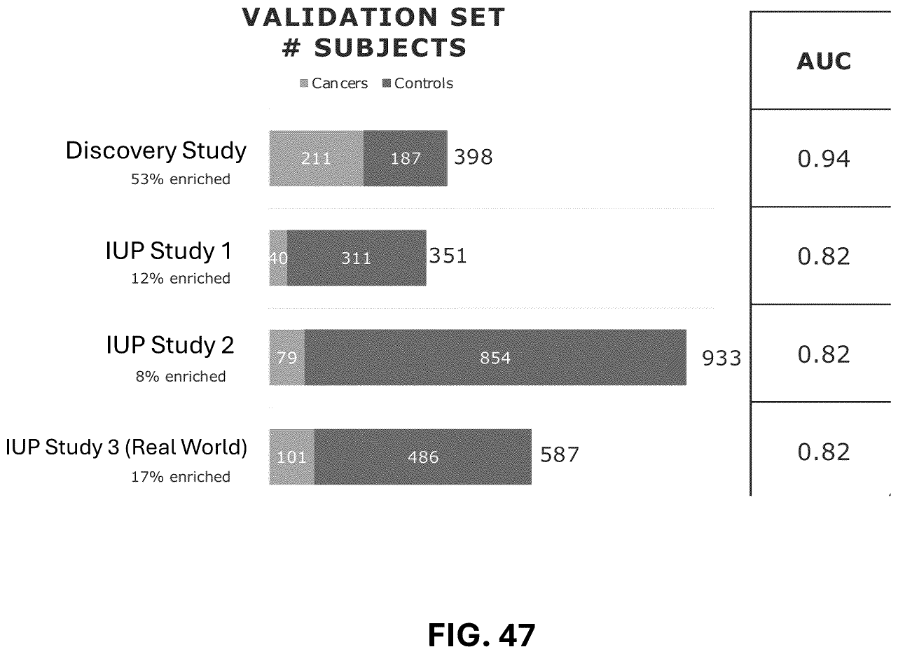

Across the various iterations of the classifiers, the classifiers remained highly generalizable. Decreasing enrichment and lower numbers of features is a much more difficult task to deploy a classifier on. Here, a comparison of four classifiers with differing enrichment and differing numbers of feature is shown for the four studies (FIG. 47). The AUC for the Discovery Study was 0.94. The AUC for IUP Study 1 was 0.82. The AUC for IUP Study 2 was 0.82. The AUC for IUP Study 3 (the validation set) was 0.82. A high level of performance was maintained throughout development and with the primary drop in performance (between the mosaic and the ITS/LDT/IUP classifiers) is primarily attributable to the significantly lower enrichment.

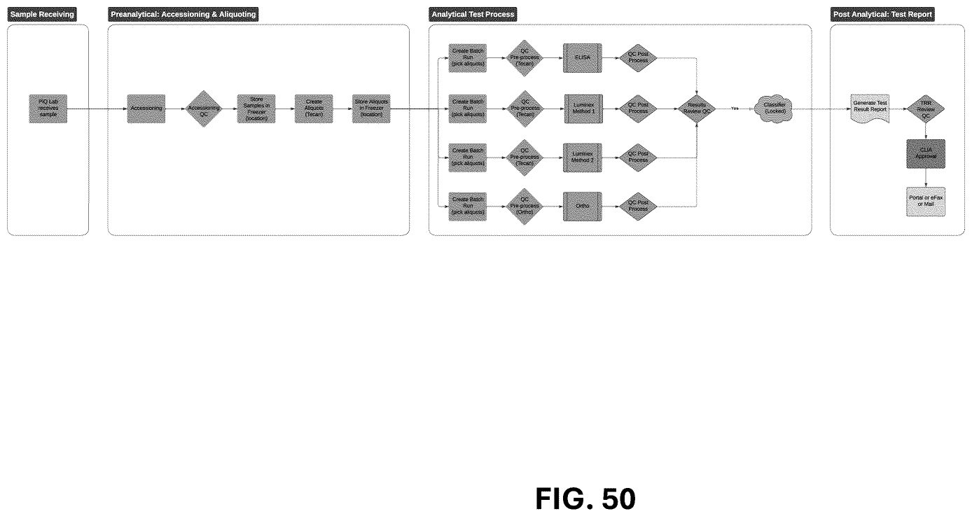

The following examples detail the workflow for the liquid biopsy lung cancer test developed herein and tested/validated in the studies above. FIG. 50 shows a schematic of the testing workflow.

### Example 5: Sample Collection

A provider used a sample collection kit to collect blood from a subject that is at least 50 years old and has at least a 20 pack-year smoking history following the instructions as provide in the sample collection kit (FIG. 51).

First, sample labels were attached to a K₂EDTA tube, a FluidX™ tube (sample storage tube), and a test requisition form. The sample label attached to the FluidX™ tube was attached in a way as to avoid covering the barcode on the FluidX™ tube. On each sample label, in the space provided, a subject ID was written. The test requisition form for the subject was completed. Information included on the test requisition form includes information on the site and subject, the specimen collection, the order from the physician, and additional provider information as provided in FIG. 52.

Second, whole blood was collected via standard venipuncture into the labeled K₂EDTA tube using a 21-gauge needle and inverted 8-10 times. Within 30 minutes of the blood collection the sample was processed as follows. The blood was spun down in a centrifuge at approximately 1300×g for approximately 15 minutes. The plasma was transferred from the K₂EDTA tube to the FluidX tube using a transfer pipette. The buffy coat and red blood cells were avoided when pipetting the plasma out of the K₂EDTA tube. Finally, the plasma sample was placed on dry ice for shipping within 1 hour of processing. Alternatively, the plasma sample could have been placed in the freezer until ready for shipment on dry ice. The sample was shipped overnight to a testing facility.

Once received at the testing facility, the plasma samples were assessed for meeting the criteria for sample acceptance or rejection. The accepted plasma sample was stored in a −80° C. freezer and the sample information was entered into the systems at the testing facility.

### Example 6: Sample Aliquoting

### Sample Thawing

Plasma samples stored in the original FluidX tube received from a provider were retrieved from a −80° C. freezer and evenly distributed with spacing between tubes into a cold 96-well aluminum block equilibrated to 3-5° C. The 96-well aluminum block was equilibrated by having previously been placed in a refrigerator at 3-5° C. for at least 2 hours. The cold 96-well aluminum block with the samples was placed back in the refrigerator at 3-5° C. for 75 minutes. At the 75-minute mark, the original FluidX sample tubes were visually inspected to ensure the samples were fully thawed. Any deviations to the 75-minute thawing time was recorded, if present.

### Sample Centrifugation:

After thawing was complete for all samples, the samples were centrifuged at 3900 revolutions per minute (RPM) for approximately 10 minutes at approximately 4° C. After centrifugation the samples were ready for analysis. If the samples were to be analyzed immediately, the samples were processed as outlined in the assays (Luminex Method 1, Luminex Method 2, ELISA Method, and VITROS/Ortho Method) described herein. If the samples were not ready to be analyzed immediately, the capped tubes were maintained at 3-5° C. for short term storage (2-8 hours) or stored at −80° C. or lower for long-term storage (>8 hours).

### Example 7: Luminex Methods

Analyte-specific antibodies were pre-coated onto magnetic microparticles embedded with fluorophores at set ratios for each unique microparticle region. Microparticles, standards and samples were pipetted into wells and the immobilized antibodies bound to the analytes of interest. After washing away any unbound substances, a biotinylated antibody cocktail specific to the analytes of interest was added to each well. Following a wash to remove any unbound biotinylated antibody, streptavidin-phycoerythrin conjugate (Streptavidin-PE), which binds to the biotinylated antibody, was added to each well. Final washes remove unbound Streptavidin-PE, the microparticles are resuspended in buffer and read using the MAGPIX®. A magnet in the analyzer captures and holds the superparamagnetic microparticles in a monolayer. Two spectrally distinct Light Emitting Diodes (LEDs) illuminate the microparticles. One LED excites the dyes inside each microparticle to identify the region and the second LED excites the PE to measure the amount of analyte bound to the microparticle. A sample from each well was imaged with a CCD camera with a set of filters to differentiate excitation levels.

Analysis with the Luminex® FLEXMAP 3D® used one laser to excite the dyes inside each microparticle to identify the microparticle region and the second laser to excite the PE to measure the amount of analyte bound to the microparticle. All excitation emitted as each microparticle passed through the flow cell was then analyzed to differentiate excitation levels using a Photomultiplier Tube (PMT) and an Avalanche Photodiode.

### Luminex Method 1

The following procedure outlines the experimental protocol performed on Luminex Assay Panels 15, 17, 18, and 20 for use on the Luminex Flexmap 3D Reader. Panel 15 was used to generate protein information on gp130. Panel 17 was used to generate protein information on C9 and Myoglobin. Panel 18 was used to generate protein information on IGFBP-6. Panel 20 was used to generate protein information on KRT19, specifically CYFRA21-1, and ENO2. The following procedure may be performed manually or using an automated system such as the Tecan Fluent 780 platform as was done here. Some of the materials and reagents used in this procedure are included in Table 4.

TABLE 4
Select Materials and Reagents for use in Luminex Method 1.

| Material/Reagent Name | Manufacturer |
|---|---|
| 1.5 mL Custom Tube Carrier | MDR Precision |
| 100 mL disposable trough, transparent PP, Tecan Sterile | Tecan |
| 1000 μL Disposable Tips Filtered, Pure, Combi, Single Stack, | Tecan |
| Wide-Bore | |
| 25 mL disposable trough, PP gray, Tecan Pure | Tecan |
| 287 mL 96-Well Reservoir, Pyramid-bottom | Agilent |
| 7.6 mL Tri-coded Tube, 24-format, External Thread | Azenta |
| Screw cap micro tube, 2 ml, PCR Performance Tested, Low | Sarstedt |
| protein-binding | |
| 96 Deep Well PP plate | Thermo Scientific |
| Biohazard Waste bag | Medegen Medical Products |
| Black Plate Lid | Agilent |
| Custom Tube Carrier | MDR Precision |
| Microplate 96 well, PS F-Bottom (Chimney Well) | Greiner |
| 96 Well Microplate, V-Bottom | Eppendorf |
| Tecan 1000 μL LiHa Disposable Tips, with filter | Tecan |
| Tecan SBS Box empty for refill 1000 μL Disposable Tips | Tecan |
| Tecan SBS Box empty for refill 10 μL, 50 μL and 200 μL LiHa | Tecan |
| Disposable Tips | |
| μClear ™ Bottom 96-well Polystyrene Microplates | Greiner Bio-One |
| Tecan 200 μL LiHa Disposable Tips, with filter, Combi | Tecan |
| Tecan 200 μL LiHa Disposable Tips, with filter | Tecan |
| Panel 20: Luminex Performance Assay 1:2 dilution in RD6-65 | R&D Systems |
| Panel 15: Luminex Human Discovery Assay 1:2 dilution in | R&D Systems |
| RD6-52 | |
| Panel 17: Luminex Human Discovery Assay 1:50 dilution in | R&D Systems |
| RD6-52 | |
| Panel 18: Luminex Human Discovery Assay 1:200 dilution in | R&D Systems |
| RD6-52 | |
| 1.5 × 0.5″ Desk Direct Thermal Label | ULINE |
| 1X Wash buffer - 20x diluted Teknova 20x PBS with 1% Tween | In-house |
| 20 | |
| RD6-65 Calibrator Diluent | R&D Systems |
| RD6-52 Calibrator Diluent | R&D Systems |
| De-ionized water | In-house |
| 70% Isopropyl Alcohol | Fisher scientific |

Following thawing and centrifugation, 200 μL of plasma from the original sample tube from the provider was transferred to 1.0 mL FluidX tubes. The 200 μL of plasma were then transferred to a plate labeled “Neat Plasma”. The standards for panel 15 and panel 20 were serially diluted into a plate labeled “Standard”. The standards for panel 15 included gp130 with a concentration of 73,330.0 pg/mL in standard 1 and a concentration of 301.7695 pg/mL for standard 6. The standards for panel 20 included KRT19, specifically CYFRA21-1, and ENO2 with a concentration of 8,800.00 pg/mL and 54,000.0 pg/mL, respectively, for standard 1 and a concentration of 3621.40 pg/mL and 222.2222 pg/mL, respectively, for standard 6. “Neat Plasma” and “Standard” plates were sealed and centrifuged for 2 minutes at 4000 RPM. After centrifugation, the seals were removed, and the plates were checked for bubbles. The samples from the “Neat Plasma” plate were then transferred and diluted for use with panel 20 and panel 15 plates. Samples for use in panel 20 had a 1:2 dilution in RD6-65 Calibrator Diluent. Samples for use in panel 15 had a 1:2 dilution in RD6-52 Calibrator Diluent.

Panel 20 beads were prepared and diluted. The diluted panel 20 beads were vortexed for 15 second. Plate 1 (Panel 20) was loaded with 50 μL of the diluted panel 20 beads. Then 50 μL of the standard curve, blanks, and controls were added to Plate 1. Finally, 50 μL of the diluted samples were added to Plate 1 in duplicate. Table 5 shows an example layout of Plate 1. Once everything was added to plate 1, the plate was covered with a black lid and incubated for 2 hours with shaking at 800 RPM on a BioShake.

Upon completion of the 2-hour incubation, the plate was washed with 1× Wash Buffer (1×PBS with tween-20) on a Hydrospeed plate washer. Then 50 μL of previously prepared panel 20 detection antibodies for detection of KRT19, specifically CYFRA21-1, and ENO2 was added to all wells of the plate. The plate was covered and incubated for 1 hour with shaking.

TABLE 5
Example layout of plate for processing with Luminex Method 1 and 2

| | 1 | 2 | 3 | 4 | 5 | 6 | 7 | 8 | 9 | 10 | 11 | 12 |
|---|---|---|---|---|---|---|---|---|---|---|---|---|
| A | Std 1 | Std 1 | High | PC | Sample | Sample | Sample | Sample | Sample | Sample | Sample | Blank |
| | | | QC | | 7 | 15 | 23 | 31 | 3 | 11 | 19 | |
| B | Std 2 | Std 2 | High | PC | Sample | Sample | Sample | Sample | Sample | Sample | Sample | Blank |
| | | | QC | | 8 | 16 | 24 | 32 | 4 | 12 | 20 | |
| C | Std 3 | Std 3 | Med | Sample | Sample | Sample | Sample | Sample | Sample | Sample | Sample | Blank |
| | | | QC | 1 | 9 | 17 | 25 | 33 | 5 | 13 | 21 | |
| D | Std 4 | Std 4 | Med | Sample | Sample | Sample | Sample | Sample | Sample | Sample | Sample | Blank |
| | | | QC | 2 | 10 | 18 | 26 | 34 | 6 | 14 | 22 | |
| E | Std 5 | Std 5 | Low | Sample | Sample | Sample | Sample | Sample | Sample | Sample | Sample | Blank |
| | | | QC | 3 | 11 | 19 | 27 | 35 | 7 | 15 | 23 | |
| F | Std 6 | Std 6 | Low | Sample | Sample | Sample | Sample | Sample | Sample | Sample | Sample | Blank |
| | | | QC | 4 | 12 | 20 | 28 | 36 | 8 | 16 | 34 | |
| G | Blank | Blank | Blank | Sample | Sample | Sample | Sample | Sample | Sample | Sample | Sample | Blank |
| | | | | 5 | 13 | 21 | 29 | 1 | 9 | 17 | 35 | |
| H | Blank | Blank | Blank | Sample | Sample | Sample | Sample | Sample | Sample | Sample | Sample | Blank |
| | | | | 6 | 14 | 22 | 30 | 2 | 10 | 18 | 36 | |

Upon completion of the 1-hour incubation, the plate was washed with 1× wash buffer on a Hydrospeed plate washer. Then 50 μL of previously prepared Panel 20 Streptavidin-PE was added to all wells of the plate. The plate was covered and incubated for 30 minutes with shaking. After the 30-minute incubation, the plate was washed, and the beads resuspended with 100 μL pf 1× wash buffer. The plate was covered and incubated for 5 minutes. The lid of the plate was then removed and immediately placed on the FlexMap 3D Luninex reader. Data on KRT19, specifically CYFRA21-1, and ENO2 were analyzed.

Panel 15 beads were prepared and diluted. The diluted panel 15 beads were vortexed for 15 second. Plate 2 (Panel 15) was loaded with 50 μL of the diluted panel 15 beads. Then 50 μL of the standard curve, blanks and controls were added to Plate 2. Finally, 50 μL of the diluted samples were added to Plate 2 in duplicate. Table 5 shows an example layout of Plate 2. Once everything was added to plate 2, the plate was covered with a black lid and incubated for 2 hours with shaking at 800 RPM on a BioShake.

Upon completion of the 2-hour incubation, the plate was washed with 1× Wash Buffer comprising 1×PBS with tween-20 on a hydrospeed plate washer. Then 50 μL of previously prepared panel 15 detection antibody for detection of gp130 was added to all wells of the plate. The plate was covered and incubated for 1 hour with shaking.

Upon completion of the 1-hour incubation, the plate was washed with 1× wash buffer on a Hydrospeed plate washer. Then 50 μL of previously prepared Panel 15 Streptavidin-PE was added to all wells of the plate. The plate was covered and incubated for 30 minutes with shaking. After the 30-minute incubation, the plate was washed, and the beads resuspended with 100 μL pf 1× wash buffer. The plate was covered and incubated for 5 minutes. The lid of the plate was then removed and immediately placed on the FlexMap 3D Luninex reader. Data on gp130 was analyzed.

The standards for panel 17 and panel 18 were serially diluted into a plate labeled “Standard”. The standards for panel 17 included C9 and myoglobin with a concentration of 7,413,650.0 pg/mL and 2,840.0 pg/mL, respectively, for standard 1 and a concentration of 30,508.8477 pg/mL and 11.6872 pg/mL, respectively, for standard 6. The standards for panel 18 included IGFBP-6 with a concentration of 9,400.0 pg/mL for standard 1 and a concentration of 38.6831 pg/mL for standard 6. The “Standard” plate was sealed and centrifuged for 2 minutes at 4000 RPM. After centrifugation, the seal was removed, and the plate was checked for bubbles. The samples from the previous “Neat Plasma” plate were transferred and diluted for use with panel 17 and panel 18 plates. Samples for use in panel 17 had a 1:50 dilution in RD6-52 Calibrator Diluent. Samples for use in panel 18 had a 1:200 dilution in RD6-52 Calibrator Diluent.

Panel 17 beads were prepared and diluted. The diluted panel 17 beads were vortexed for 15 second. Plate 3 (Panel 17) was loaded with 50 μL of the diluted panel 17 beads. Then 50 μL of the standard curve, blanks and controls were added to Plate 3. Finally, 50 μL of the diluted samples were added to Plate 3 in duplicate. Table 5 shows an example layout of Plate 3. Once everything was added to plate 3, the plate was covered with a black lid and incubated for 2 hours with shaking at 800 RPM on a BioShake.

Upon completion of the 2-hour incubation, the plate was washed with 1× Wash Buffer comprising 1×PBS with tween-20 on a hydrospeed plate washer. Then 50 μL of previously prepared panel 17 detection antibodies used for the detection of C9 and myoglobin was added to all wells of the plate. The plate was covered and incubated for 1 hour with shaking.

Upon completion of the 1-hour incubation, the plate was washed with 1× wash buffer on a Hydrospeed plate washer. Then 50 μL of previously prepared Panel 17 Streptavidin-PE was added to all wells of the plate. The plate was covered and incubated for 30 minutes with shaking. After the 30-minute incubation, the plate was washed, and the beads resuspended with 100 μL pf 1× wash buffer. The plate was covered and incubated for 5 minutes. The lid of the plate was then removed and immediately placed on the FlexMap 3D Luninex reader. Data on C9 and myoglobin were analyzed.

Panel 18 beads were prepared and diluted. The diluted panel 18 beads were vortexed for 15 second. Plate 4 (Panel 18) was loaded with 50 μL of the diluted panel 18 beads. Then 50 μL of the standard curve, blanks and controls were added to Plate 4. Finally, 50 μL of the diluted samples were added to Plate 4 in duplicate. Table 5 shows an example layout of Plate 4. Once everything was added to plate 4, the plate was covered with a black lid and incubated for 2 hours with shaking at 800 RPM on a BioShake.

Upon completion of the 2-hour incubation, the plate was washed with 1× Wash Buffer comprising 1×PBS with tween-20 on a hydrospeed plate washer. Then 50 μL of previously prepared panel 18 detection antibody used for the detection of IGFBP-6 was added to all wells of the plate. The plate was covered and incubated for 1 hour with shaking.

Upon completion of the 1-hour incubation, the plate was washed with 1× wash buffer on a Hydrospeed plate washer. Then 50 μL of previously prepared Panel 18 Streptavidin-PE was added to all wells of the plate. The plate was covered and incubated for 30 minutes with shaking. After the 30-minute incubation, the plate was washed, and the beads resuspended with 100 μL pf 1× wash buffer. The plate was covered and incubated for 5 minutes. The lid of the plate was then removed and immediately placed on the FlexMap 3D Luminex reader. Data on IGFBP-6 was analyzed.

### Luminex Method 2

The following procedure outlines the experimental protocol performed on Luminex Assay Panels 21, 16, and 22 for use on the Luminex Flexmap 3D Reader. Panel 21 was used to generate protein information on CEA, specifically CEACAM-5. Panel 16 was used to generate protein information on PECAM-1. Panel 22 was used to generate protein information on SAA. The following procedure may be performed manually or using an automated system such as the Tecan Fluent 780 platform as was done here. Some of the materials and reagents used in this procedure are included in Table 6.

TABLE 6
Materials and Reagents Used in Luminex Method 2

| Material Name | Manufacturer |
|---|---|
| 96-well flat-bottom microplate | Greiner |
| 2-20 μL LTS Pipette Tips | Mettler-Toledo |
| 20-200 μL LTS Pipette Tips | Mettler-Toledo |
| 100-1200 μL LTS Pipette Tips | Mettler-Toledo |
| Microcentrifuge tube rack | N/A |
| 2.0 mL cryo screw-cap tubes | Sarstedt |
| 7.6 mL FluidX tube | Azenta |
| 15 mL amber conical tube | Greiner |
| 25 mL disposable reagent reservoirs, with divider | N/A |
| 5 mL disposable reagent reservoir | Fisherbrand |
| 96-well Plate Sealers | Thermofisher |
| Black plate lids | Agilent |
| 1.5 mL Custom Tube Carrier | MDR Precision |
| 100 mL disposable trough, transparent PP, Tecan Sterile | Tecan |
| 1000 μL Disposable Tips Filtered, Pure, Combi, Single Stack, | Tecan |
| Wide-Bore | |
| 25 mL disposable trough, PP gray, Tecan Pure | Tecan |
| 287 mL 96-Well Reservoir, Pyramid-bottom | Agilent |
| 7.6 mL Tri-coded Tube, 24-format, External Thread | Azenta |
| 96 Deep Well PP plate | Thermo Scientific |
| 96 Well Microplate, V-Bottom | Eppendorf |
| Biohazard Waste bag | Medegen Medical Products |
| Black Plate Lid | Agilent |
| Custom Tube Carrier | MDR Precision |
| Microplate 96 well, PS F-Bottom (Chimney Well) | Greiner |
| Panel 16: Luminex Human Discovery Assay | R&D Systems |
| 1:2 dilution in RD6-52 | |
| Panel 21: Luminex Performance Assay | R&D Systems |
| 1:8 dilution in RD6-65 | |
| Panel 22: ProcartaPlex Invitrogen Assay | ThermoFisher Scientific |
| 1:4000 dilution in UAB | |
| Tecan 1000 μL LiHa Disposable Tips, with filter | Tecan |
| Tecan 200 μL LiHa Disposable Tips, with filter | Tecan |
| Tecan 200 μL LiHa Disposable Tips, with filter, Combi | Tecan |
| Tecan SBS Box empty for refill 10 μL, 50 μL, and 200 μL | Tecan |
| LiHa Disposable Tips | |
| Tecan SBS Box empty for refill 1000 μL Disposable Tips | Tecan |
| μClear ™ Bottom 96-well Polystyrene Microplates | Greiner Bio-One |
| 1.5 × 0.5″ Desk Direct Thermal Label | ULINE |
| ProcartaPlex ™ Human Simplex and Combinable Panels | ThermoFisher |
| Streptavidin-PE concentrate | Bio-Techne |
| xMAP ® Sheath Fluid PLUS | Invitrogen ™ |
| 20X PBS with 0.5% Tween 20 | Teknova |
| 1X Wash buffer - 20x diluted Teknova 20x PBS with 1% | In-house |
| Tween 20 | |
| RD6-65 Calibrator Diluent | R&D Systems |
| RD6-52 Calibrator Diluent | R&D Systems |
| 1X Universal Assay Buffer (UAB) | ThermoFisher Scientific |
| De-ionized water | In-house |
| 70% Isopropyl Alcohol | Fisher scientific |

Following thawing and centrifugation, 200 μL of plasma from the original sample tube from the provider was transferred to 1.0 mL FluidX tubes. The 200 μL of plasma were then transferred to a plate labeled “Neat Plasma”. The standards for panel 16 and panel 21 were serially diluted into a plate labeled “Standard”. The standards for panel 16 included PECAM-1 with a concentration of 183,390.0 pg/mL for standard 1 and a concentration of 754.6914 pg/mL for standard 6. The standards for panel 21 included CEA, specifically CEACAM5, with a concentration of 14,000.0 pg/mL for standard 1 and a concentration of 57.6132 pg/mL for standard 6. The “Neat Plasma” and “Standard” plates were sealed and centrifuged for 2 minutes at 4000 RPM. After centrifugation, the seals were removed, and the plates were checked for bubbles. The samples from the “Neat Plasma” plate were then transferred and diluted for use with panel 16 and panel 21 plates. Samples for use in panel 16 had a 1:2 dilution in RD6-52 Calibrator Diluent. Samples for use in panel 21 had a 1:8 dilution in RD6-65 Calibrator Diluent.

Panel 21 beads were prepared and diluted. The diluted panel 21 beads were vortexed for 15 second. Plate 1 (Panel 21) was loaded with 50 μL of the diluted panel 21 beads. Then 50 μL of the standard curve, blanks and controls were added to Plate 1. Finally, 50 μL of the diluted samples were added to Plate 1 in duplicate. Table 5 shows an example layout of Plate 1. Once everything was added to plate 1, the plate was covered with a black lid and incubated for 2 hours with shaking at 800 RPM on a BioShake.

Upon completion of the 2-hour incubation, the plate was washed with 1× Wash Buffer (1×PBS with tween-20) on a Hydrospeed plate washer. Then 50 μL of previously prepared panel 21 detection antibody used for detection of CEA, specifically CEACAM-5 was added to all wells of the plate. The plate was covered and incubated for 1 hour with shaking.

Upon completion of the 1-hour incubation, the plate was washed with 1× wash buffer on a Hydrospeed plate washer. Then 50 μL of previously prepared Panel 18 Streptavidin-PE was added to all wells of the plate. The plate was covered and incubated for 30 minutes with shaking. After the 30-minute incubation, the plate was washed, and the beads resuspended with 100 μL pf 1× wash buffer. The plate was covered and incubated for 5 minutes. The lid of the plate was then removed and immediately placed on the FlexMap 3D Luminex reader. Data on CEA, specifically CEACAM-5, was analyzed.

Panel 16 beads were prepared and diluted. The diluted panel 16 beads were vortexed for 15 second. Plate 2 (Panel 16) was loaded with 50 μL of the diluted panel 16 beads. Then 50 μL of the standard curve, blanks and controls were added to Plate 2. Finally, 50 μL of the diluted samples were added to Plate 2 in duplicate. Table 5 shows an example layout of Plate 2. Once everything was added to plate 2, the plate was covered with a black lid and incubated for 2 hours with shaking at 800 RPM on a BioShake.

Upon completion of the 2-hour incubation, the plate was washed with 1× Wash Buffer (1×PBS with tween-20) on a Hydrospeed plate washer. Then 50 μL of previously prepared panel 16 detection antibody used for detection of PECAM-1 was added to all wells of the plate. The plate was covered and incubated for 1 hour with shaking.

Upon completion of the 1-hour incubation, the plate was washed with 1× wash buffer on a Hydrospeed plate washer. Then 50 μL of previously prepared Panel 16 Streptavidin-PE was added to all wells of the plate. The plate was covered and incubated for 30 minutes with shaking. After the 30-minute incubation, the plate was washed, and the beads resuspended with 100 μL of 1× wash buffer. The plate was covered and incubated for 5 minutes. The lid of the plate was then removed and immediately placed on the FlexMap 3D Luminex reader. Data on PECAM-1 was analyzed.

Standard 1 comprising SAA was prepared by reconstituting the lyophilizate with 500 μL of 1× Universal Assay Buffer (UAB). Alternatively, when Standard 1 comprising SAA is already prepared, the standard is removed from the −80° C. freezer, thawed at room temperature, vortexed, and centrifuged for 5 seconds. Four hundred μL of prepared Standard 1 comprising SAA was transferred to new tube labeled STD-P022 and the stock tube of prepared Standard 1 is placed (or returned) to the −80° C. freezer.

The high-level control or QC (HQC) was prepared by mixing 120 μL of 1×UAB with 480 μL of STD-P022 comprising SAA. The mid-level control or QC (MQC) was prepared by mixing 150 μL 1×UAB with 150 μL HQC. The low-level control or QC (LQC) was prepared by mixing 225 μL of 1×UAB with 75 μL HQC. Alternatively, when HQC, MQC, and LQC are already prepared, HQC, MQC, and LQC are removed from the −80° C. freezer, thawed at room temperature, vortexed, and centrifuged for 5 seconds.

The standards for panel 22 were serially diluted into a plate labeled “Standard”. The standards for panel 22 included SAA with a concentration of 110,900.0 pg/mL for standard 1 and a concentration of 456.3786 pg/mL for standard 6. The “Standard” plate was sealed and centrifuged for 2 minutes at 4000 RPM. After centrifugation, the seal was removed, and the plate was checked for bubbles. The samples from the previous “Neat Plasma” plate were transferred and diluted for use with panel 17 and panel 18 plates. Samples for use in panel 22 had a 1:4,000 dilution in UAB.

Beads for panel 22 were prepared by bringing one vial of 50× Simplex beads to room temperature, vortexing the vial for 30 seconds (but not inverting), and mixing 110 μL of 50× Simplex beads with 5390 50× Simplex beads 1× Wash Buffer in a new tube (Beads-P022). The Beads-P022 tube was vortexed for 30 seconds and covered in foil until ready for use.

The Beads-P022 tube was vortexed for 15 seconds. Plate 3 (Panel 22) was loaded with 50 μL of the Beads-P022. Then 50 μL of the standard curve, blanks and controls were added to Plate 3. Finally, 50 μL of the diluted samples were added to Plate 3 in duplicate. Table 5 shows an example layout of Plate 3. Once everything was added to plate 3, the plate was covered with a black lid and incubated for 2 hours with shaking at 800 RPM on a BioShake.

The detection antibody mixture specific for SAA was prepared by centrifuging the detection antibody stock vial for 30 seconds, combining 60 μL of the detection antibody specific for SAA stock with 5940 μL 1× Wash Buffer into an amber tube, and vortexing the amber tube for 15 seconds.

Upon completion of the 2-hour incubation, the plate was washed with 1× Wash Buffer (1×PBS with tween-20) on a Hydrospeed plate washer. Then 50 μL of previously prepared panel 22 detection antibody used for detection of SAA was added to all wells of the plate. The plate was covered and incubated for 1 hour with shaking.

Streptavidin-PE was prepared by centrifuging the streptavidin-PE concentrate vial for 30 seconds, vortexing the streptavidin-PE concentrate vial (but not inverting), combining 220 μL of streptavidin-PE concentrate with 5780 μL 1× Wash Buffer in an amber tube, and vortexing the amber tube for 15 seconds.

Upon completion of the 1-hour incubation, the plate was washed with 1× wash buffer on a Hydrospeed plate washer. Then 50 μL of previously prepared Panel 22 Streptavidin-PE was added to all wells of the plate. The plate was covered and incubated for 30 minutes with shaking. After the 30-minute incubation, the plate was washed and the beads resuspended with 100 μL of 1× wash buffer. The plate was covered and incubated for 5 minutes. The lid of the plate was then removed and immediately placed on the FlexMap 3D Luminex reader. Data on PECAM-1 was analyzed.

### Example 8: ELISA Method

Human FGL1 lyophilized recombinant protein was reconstituted to 10,000 pg/mL with Sample Diluent NS generating an FGL1 stock. Four Intermediate Solutions, each with a concentration of 6,000 pg/mL, was prepared from the FGL1 stock and the Sample Diluent NS. Standard 1 with a concentration of 3,000 pg/mL was prepared from the first Intermediate Solution and Diluent NS. The High-Quality Control (HQC) with a concentration of 1,500 pg/mL was prepared from the second Intermediate Solution and Sample Diluent NS. The Medium-Quality Control (MQC) with a concentration of 500 pg/mL was prepared from the third Intermediate Solution and Sample Diluent NS. The Low-Quality Control (LQC) with a concentration of 150 μg/mL was prepared from the fourth Intermediate Solution and Sample Diluent NG. Alternatively, if the FGL1 stock, standard 1, HQC, MQC, or LQC were made previously they can be removed from the −80° C. freezer for use in the Human FGL1 ELISA Assay (Ab284622).

The plate washer was primed, and a standard curve was prepared by making serial dilutions of Standard 1 using Sample Diluent NS in a 96-well plate labeled “Standards”. The concentrations of the dilutions following the 3,000 pg/mL Standard 1 was 1,500 pg/mL, 750 μg/mL, 375 pg/mL, 187.5 pg/mL, 93.75 pg/mL, and 46.88 pg/mL (ST1-ST7, respectively). Following thawing and centrifugation as described above, 100 μL of plasma from the original sample tube from the provider was transferred to 1.0 mL FluidX tubes. The 100 μL of plasma were then transferred to a plate labeled “Neat Plasma”. The “Neat Plasma” and “Standard” plates were sealed and centrifuged for 2 minutes at 4000 RPM. After centrifugation, the seals were removed, and the plates were checked for bubbles.

Fifty μL of the standard curve (ST1-ST7) and blanks (Blank) in duplicate were added to the first two columns of the Pre-Coated 96-Well Microplate (FGL1 ELISA plate). Fifty μL of the high (HQC), medium (MQC), and low (LQC) controls (CTR1_1, CTR1_2, CTR1_3 on plate map) and blanks (BL1) in duplicate were added to the third column of the FLG1 ELISA plate. Fifty μL of 1:500 diluted samples were added into the top-half of columns 4-12 of the FGL1 ELISA plate (first replicate). Fifty μL of 1:500 diluted samples were added into the bottom-half of columns 4-12 of the FGL1 ELISA plate (second replicate). Table 7 provides a layout of the samples in the FGL1 ELISA plate.

TABLE 7
Example layout for running samples on FGL1 ELISA plate.

| | 1 | 2 | 3 | 4 | 5 | 6 | 7 | 8 | 9 | 10 | 11 | 12 |
|---|---|---|---|---|---|---|---|---|---|---|---|---|
| A | Std 1 | Std 1 | High | Sample | Sample | Sample | Sample | Sample | Sample | Sample | Sample | Sample |
| | | | QC | 1 | 9 | 17 | 25 | 33 | 41 | 49 | 57 | 65 |
| B | Std 2 | Std 2 | High | Sample | Sample | Sample | Sample | Sample | Sample | Sample | Sample | Sample |
| | | | QC | 2 | 10 | 18 | 26 | 34 | 42 | 50 | 58 | 66 |
| C | Std 3 | Std 3 | Med | Sample | Sample | Sample | Sample | Sample | Sample | Sample | Sample | Sample |
| | | | QC | 3 | 11 | 19 | 27 | 35 | 43 | 51 | 59 | 67 |
| D | Std 4 | Std 4 | Med | Sample | Sample | Sample | Sample | Sample | Sample | Sample | Sample | Sample |
| | | | QC | 4 | 12 | 20 | 28 | 36 | 44 | 52 | 60 | 68 |
| E | Std 5 | Std 5 | Low | Sample | Sample | Sample | Sample | Sample | Sample | Sample | Sample | Sample |
| | | | QC | 5 | 13 | 21 | 29 | 37 | 45 | 53 | 61 | 69 |
| F | Std 6 | Std 6 | Low | Sample | Sample | Sample | Sample | Sample | Sample | Sample | Sample | Sample |
| | | | QC | 6 | 14 | 22 | 30 | 38 | 46 | 54 | 62 | 70 |
| G | Std 7 | Std 7 | Blank | Sample | Sample | Sample | Sample | Sample | Sample | Sample | Sample | Sample |
| | | | | 7 | 15 | 23 | 31 | 39 | 47 | 55 | 63 | 71 |
| H | Blank | Blank | Blank | Sample | Sample | Sample | Sample | Sample | Sample | Sample | Sample | Sample |
| | | | | 8 | 16 | 24 | 32 | 40 | 48 | 56 | 64 | 72 |

The FGL1 ELISA plate was covered with a black lid was incubated on a shaker (Bioshaker) for 1 hour at 400 rpm shaking speed. Following incubation, the plate was washed with 1× Wash Buffer PT (Hydrospeed plate washer). Then 100 μL of TMB substrate was added to the FGL1 ELISA plate. The FGL1 ELISA plate was again incubated on a shaker (Bioshaker) for 9 minutes at 400 rpm shaking speed. Then 100 μL of stop solution was added to the FGL1 ELISA plate and the plate analyzed using Tecan plate reader (Tecan Infinite 200 Pro) at 450 nm endpoint readings. The reagents used in this example are described in Table 8. This procedure may be performed manually or through the use of an automated machine such as the Tecan Fluent 780 as was used here.

TABLE 8
Reagents used in FGL1 ELISA Assay

| Reagent Name | Manufacturer |
|---|---|
| Huma Fibrinogen-like protein 1/FGL1 ELISA kit | Abcam (Ab284622) |
| Human FGL-1 Recombinant Protein | Abcam (Supplied in ab284622 kit) |
| Sample Diluent NS | Abcam (Supplied in ab284622 kit) |
| Custom Assay control set (High, Mid, Low) for FGL1 | In-house |
| ELISA | |
| De-ionized water | In-house |
| Bulk TMB substrate 1X | Abcam (ab210902) |
| Bulk 10X Wash buffer | Abcam (ab206977) |
| Stop solution | R&D Systems (895032) |
| 70% Isopropyl Alcohol | Fisher scientific (04-355-71) |

### Example 9: VITROS (Ortho) Method

The VITROS® ECiQ Immunodiagnostic Analyzer is designed to detect and quantify specific analytes in patient plasma using immunoassay technology. The system employs capture antibodies that selectively bind to target proteins—such as carcinoembryonic antigen (CEA) and cancer antigen 125 II (CA125 II (CA125a))—to facilitate accurate measurement of their concentrations. The CEA assay is based on a sandwich immunometric immunoassay performed on the VITROS ECi/ECiQ Immunodiagnostic systems. The CA125 assay uses a sandwich immunometric immunoassay format on the same VITROS platforms.

Test tubes were labeled with barcodes to identify the sample and placed in a Universal Sample Tray with the barcodes facing out. Micro Sample Cups were placed in each test tube. Approximately 200 μL of plasma was transferred from the aliquoted FluidX tube to the sample's corresponding Micro Sample Cup in the labeled test tube. The Micro Sample Cups were checked for bubbles.

The Universal Sample Tray comprising the samples was loaded onto the VITROS ECiQ machine. The sample barcodes were entered into the VITROS ECiQ System and were categorized as “Patient” and “Plasma”. The proteins to be run with the sample were selected.

The machine was checked to confirm that the appropriate VITROS Signal Reagent, VITROS Universal Wash Reagent, and calibrated reagent lots were loaded. Once everything was confirmed, the samples were run on the machine. Reagents used with the VITROS® ECiQ Immunodiagnostic Analyzer are included in Table 9.

The carcinoembryonic antigen (CEA) in the sample bound simultaneously to a biotinylated mouse monoclonal anti-CEA antibody and a horseradish peroxidase (HRP)-labeled mouse monoclonal anti-CEA antibody. The immune complex was captured by streptavidin-coated wells, and unbound materials were removed by washing. A luminescent reaction was triggered by HRP activity, and the emitted light was directly proportional to the CEA concentration. In this procedure the reaction may also include information on one or more of BGP1 (CEACAMI), NCA (CEACAM6), NCA-2, or any combination thereof.

The CA-125 (OC125) defined antigen in the sample reacted with a biotinylated M11 mouse monoclonal antibody and an HRP-labeled CA-125 (OC125) mouse monoclonal antibody. The complex was captured by streptavidin-coated wells, followed by washing. A luminescent signal was generated via HRP-catalyzed oxidation of luminol derivatives, and the signal intensity was proportional to the CA125 concentration.

TABLE 9
Reagents used in VITROS Method

| Reagent name | Manufacturer | Catalog # |
|---|---|---|
| CA125 Calibrator | Ortho Clinical Diagnostics | 617 7751 |
| CA125 Reagent Pack | Ortho Clinical Diagnostics | 617 7746 |
| CEA Calibrator | Ortho Clinical Diagnostics | 106 2306 |
| CEA Reagent Pack | Ortho Clinical Diagnostics | 192 0115 |
| Maintenance Pack | Fujirebio Diagnostics | 183 1312 |
| Signal Reagent | Ortho Clinical Diagnostics | 107 2693 |
| Tumor Marker Control: Level 1 and Level 2 | Fujirebio Diagnostics | 108-20 |
| Universal Wash Reagent | Fujirebio Diagnostics | 8389793 |

### Example 10: Assay Quality Control and Data Analysis

Raw data files generated from the four analytical methods (Luminex Method 1, Luminex Method 2, ELISA, and VITROS) for gp130, PECAM-1, C9, Myoglobin, IGFBP-6, ENO2, CEA (CEACAM), SAA, FGL1, CYFRA21-1, and CA125 were integrated into the laboratory management software workflow, where sample runs were analyzed and monitored, rerun handling (including QC failures at both batch and individual sample levels) managed, and assay QC evaluation, data analysis, and acceptance criteria were applied. Table 10 provides the quality control acceptance criteria for the Luminex and ELISA assays. Table 11 provides the QC acceptance criteria thresholds for standards, technical controls and process controls. Table 12 provides the quality control acceptance criteria for the VITROS assays. Table 13 provide the % CV tolerance accepted between duplicates.

TABLE 10
Plate QC Acceptance Criteria for Luminex and ELISA assays.

| Type | Metric | Criteria |
|---|---|---|
| Standard Curve | % Recovery | 5 standards pass |
| Standard Curve | % CV | 5 standards pass |
| Blank | MFI | Below upper limit cutoff |
| Tech Control | % Recovery | Within % cutoff, 2 pass |
| Tech Control | % CV | Below % cutoff, 2 pass |
| Process Control | % Recovery | Within % cutoff |
| Process Control | % CV | Below % cutoff |
| Any Well (For | Bead Count | ≥50 |
| Luminex Only) | | |

TABLE 11
QC Acceptance Criteria Thresholds for Standards, Controls and Process Controls

| | | | | | | | Blank | Process | |
|---|---|---|---|---|---|---|---|---|---|
| | | | | Std | Control | | (upper | Control | Process |
| | Luminex | | Std | % | % | Control | RAW | (% | Controls |
| Protein | Panel | Method | recovery | CV | recovery | % CV | cut off) | Recovery) | (% CV) |
| FGL1 | N/A | ELISA | 80-120 | 25 | 70-130 | 25 | 0.1 | 70-130 | 15 |
| gp130 | 15 | Method | 90-115 | 15 | 80-120 | 10 | 19.3 | 75-125 | 10 |
| | | 1 | | | | | | | |
| CD31/ | 16 | Method | 90-110 | 25 | 75-125 | 15 | 15 | 70-130 | 10 |
| PECAM-1 | | 2 | | | | | | | |
| C9 | 17 | Method | 90-115 | 25 | 70-130 | 20 | 19.6 | 70-130 | 15 |
| | | 1 | | | | | | | |
| Myoglobin | 17 | Method | 80-120 | 20 | 70-130 | 10 | 23.9 | 70-130 | 10 |
| | | 1 | | | | | | | |
| IGFBP-6 | 18 | Method | 90-110 | 20 | 85-115 | 10 | 21.2 | 85-115 | 10 |
| | | 1 | | | | | | | |
| CYFRA21-1 | 20 | Method | 80-120 | 15 | 70-130 | 15 | 13 | N/A | N/A |
| | | 1 | | | | | | | |
| Enolase2 | 20 | Method | 90-110 | 15 | 70-130 | 25 | 35.9 | 70-130 | 15 |
| | | 2 | | | | | | | |
| CEACAM- | 21 | Method | 90-110 | 15 | 75-125 | 10 | 40 | 75-125 | 10 |
| 5/CD66e | | 2 | | | | | | | |
| SAA | 22 | Method | 80-120 | 25 | 70-130 | 20 | 73.9 | 70-130 | 20 |
| | | 2 | | | | | | | |

TABLE 12
QC Acceptance criteria for Ortho Assays

| | Level 1 Acceptable | Level 2 Acceptable |
|---|---|---|
| Protein | Range | Range |
| CEA (ng/mL) | 2.83-4.67 | 37.5-61.5 |
| CA125 (U/mL) | 15.8-25.0 | 268-426 |

TABLE 13
Duplicate acceptable CV tolerance thresholds for each feature

| Feature | % CV Tolerance |
|---|---|
| gp130 | 10 |
| CD31 | 10 |
| Complement Component C9 | 15 |
| Myoglobin | 10 |
| IGFBP-6 | 10 |
| Enolase-2 | 15 |
| CYFRA21-1 | N/A |
| CEACAM | 10 |
| SAA | 20 |
| FGL1 | 15 |

Approved results were transferred to the classifier, where they were analyzed to generate probability risk calls based on QC-passed quantitative protein marker measurements from the four analytical methods.

Samples with failed QC results were flagged for reruns. Reruns were performed using the same four analytical methods. For a given subject, if any single marker assay failed either batch or sample QC, the classifier was not completed for that subject in the laboratory management software workflow.

### Example 11: Test Result Review and Reporting

When all proteins for a sample met quality control acceptance criteria, the classifier was triggered. The AUROC of the selected lung cancer classifier on the training and hold-out validation data in the lab-developed test described herein is shown in FIG. 48. The selected lung cancer classifier model met the acceptance criteria for validation. In particular, the AUROC on hold-out validation fell within the 95% confidence interval established from training. The training AUC was 0.83 (95% CI 0.78 to 0.88). The testing AUC was 0.82 (95% CI 0.77 to 0.87). The specificity at 85% sensitivity fell within the 95% confidence interval established from training. The training specificity was 61% (95% CI 53% to 74%). The testing specificity was 56% (95% CI 42% to 74%). Using the probability threshold at 85% sensitivity, the bootstrap estimates of the sensitivity per cancer stage on the testing partition were as follows. Stage I lung cancer was 81% sensitivity (95% CI 50% to 100%), Stage II lung cancer was 100% (95% CI 56% to 100%), Stage III lung cancer was 88% (95% CI 50% to 100%), and Stage IV cancer was 89% (95% CI 50% to 100%). The protein features used in the classifier model were: Complement Component C9 (C9), Cell adhesion molecule (CEA) from the Luminex assay, Cell adhesion molecule (CEA) from the VITROS assay, Keratin, type I cytoskeletal 19 (KRT19) (specifically CYFRA21-1), CA-125 Antigen (CA125), Myoglobin (MB), Gamma-enolase (ENO2), Glycoprotein 130 (GP130), Fibrinogen-like protein 1 (FGL1), Insulin-like growth factor-binding protein 6 (IGFBP-6), Platelet endothelial cell adhesion molecule (PECAM1), and Serum amyloid A protein (SAA).

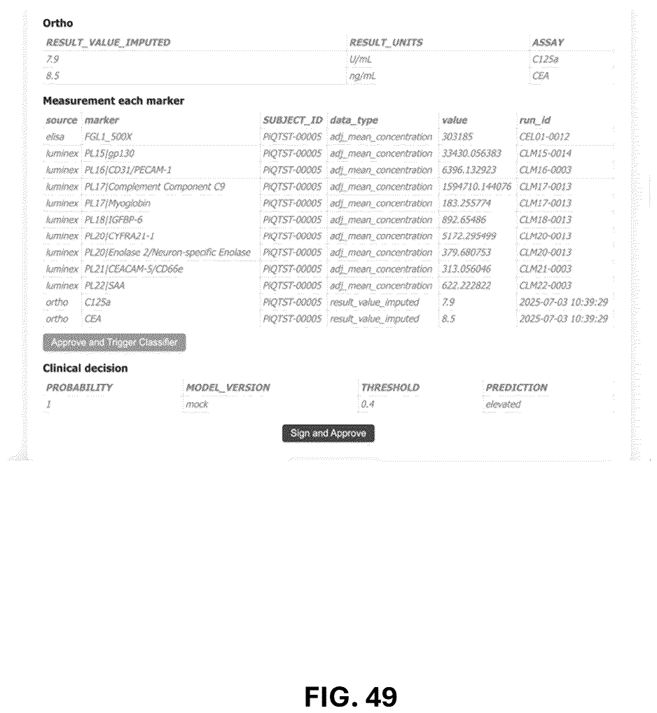

Following analysis in the classifier, a summary table of the clinical decision was generated for review (FIG. 49). Once the clinical decision was generated, the aggregated result data was reviewed, and the clinical decision parameters were verified for accuracy including confirming that the “prediction” output corresponds with the “probability” and “threshold” rules.

For example, when probability was greater than the threshold, the sample was classified as “Elevated”. An “Elevated” test result indicates that an individual has a higher risk for lung cancer than the baseline adults eligible for lung cancer screening. The adults eligible for lung cancer screening were adults over age 50 with at least a 20 pack-year smoking history. The baseline was determined from the model output based on the probability and threshold rules. The ‘Elevated’ test result corresponded to a likelihood of cancer based on the model output (e.g., baseline). An “Elevated” test result does not definitively indicate that the individual has lung cancer. When the probability was less than or equal to the threshold, the sample was classified as “Not Elevated”. A “Not Elevated” test result indicated that an individual has a risk of lung cancer similar to that of the baseline associated with adults eligible for lung cancer screening. The adults eligible for lung cancer screening were adults over age 50 with at least a 20 pack-year smoking history. The baseline was determined from the model output based on the probability and threshold rules. The ‘Not Elevated’ test result corresponded to a minimal likelihood of cancer or no likelihood of cancer based on the model output (e.g., baseline). A “Not Elevated” screening test result does not guarantee the absence of cancer or potential development of cancer in the future. Patients with a “Not Elevated” test result are advised to continue participating in a lung cancer screening program with a recommended screening method.

In this example, when an error was found unrelated to the analytical result, such as a clerical error or demographics entry error, the results were reevaluated. When the outcome was the same as original result, an updated result was reported as “Corrected” with the result (e.g., Elevated or Not Elevated). If the outcome was different from the original result, an updated report was reported as “Amended” with the result (e.g., Elevated or Not Elevated).

### Example 12: Analytical Validation

Analytical validation studies were performed for a laboratory test, as described herein, developed to detect protein biomarkers associated with the detection of lung cancer from plasma samples isolated from peripheral whole blood collected in K₂EDTA tubes. The collection and shipping methods were consistent with those described in Example 5. The laboratory test is intended for individuals who are eligible for lung cancer screening including adults over 50 years old who have at least a 20 pack-year smoking history. The laboratory test quantifies the concentration of 11 protein biomarkers using immunoassays as described in Examples 7, 8, and 9. The measured protein concentrations were analyzed through an algorithm to classify the individuals as either elevated or not elevated for lung cancer as described in Example 11.

The analytical validation studies were performed in accordance with guidelines established by the College of American Pathologists (CAP) and the Clinical Laboratory Improvement Amendments of 1988 (CLIA '88), appropriate for a laboratory-developed test (LDT). For the Ortho/VITROS immunoassay, which is based on an FDA-approved reagent kit, the analytical verification studies (e.g., accuracy, precision and reportable range) were conducted as required per CAP guidelines.

Quality control criteria for (1) standards and blanks (2) technical quality controls, and (3) process controls were established to qualify sample data for downstream analysis.

For standards, duplicate concentration measurements were completed for each standard and the concentration-based % CV was established for each standard. If a plate failed to meet the standard curve acceptance criteria listed in Table 10 (e.g., five standards in the curve passing the percent recovery and % CV for standards), the plate was excluded from analysis. A plate may have also been excluded if the average of all blanks on a plate were not at or below the Max MFI cutoff for each protein (Table 10). A minimum bead count of 50 beads for each protein was needed for the data from the protein to be included in analysis (Table 10).

For technical quality controls, duplicate concentration measurements were completed for each High QC (HQC), Medium QC (MQC) and Low QC (LQC) and the concentration-based % CV was established. The concentration-based % CV of each HQC, MQC and LQC was at or below the Max duplicate % CV cutoff for each protein. The percent recovery for each HQC, MQC and LQC was calculated. The percent recovery for each of these controls fell within the allowable percent recovery ranges (Table 11). The HQC, MQC and LQC's on Luminex assays met the requirement of a minimum bead count of 50 beads for each protein. If a technical quality control did not meet these requirements, the plate was failed and excluded from analysis.

For process controls or process quality controls, the percent recovery was calculated. The precent recovery for each process control fell within the allowable percent recovery ranges (Table 11). The concentration-based % CV of each process control was at or below the Max duplicate % CV cutoff for each protein. Process controls on Luminex assays met the requirement of a minimum bead count of 50 beads or the plate was failed. If an assay process control did not meet these requirements, the plate was failed and excluded from analysis.

No quality control criteria was established for CYFRA21 for the plasma process control because the level of CYFRA21 in the plasma process control was below the assay lower limit of quantitation (LLOQ). Since the primary purpose of the process control is to verify that there were no issues with sample dilution for a particular Luminex panel and enolase2 is in the same panel as CYFRA21 (Panel 20) and can be measured, it was decided that it was acceptable to not include CYFRA21 in the process control quality control.

### Analytical Precision

An analytical precision study was conducted to evaluate the degree of variation expected when the assay was performed under standard laboratory conditions over a period of time. The two components evaluated were repeatability (within-run precision) and intermediate precision (within-laboratory precision). The analytical precision study was run on all immunoassay platforms (Luminex, ELISA, Ortho/VITROS). Samples including clinical samples, commercial samples, and contrived samples were procured or prepared and processed through the immunoassay workflows for analytical precision.

A total of thirty-five plasma samples per batch were tested across four days (1 batch per day), using a single lot of assay reagents, with 2 operators for the first two days and 2 new operators for the next two days. This resulted in a total of 44 or 48 replicates per biological sample run in this study.

Luminex Method 1 utilized three contrived samples in each batch. One sample with 11 replicates and two samples with 12 replicates. Luminex Method 2 utilized three contrived samples in each batch that were different from the samples used in Luminex Method 1. One sample with 11 replicates and two samples with 12 replicates. Details of Luminex Method 1 and 2 are provided in Example 7. FGL1 ELISA utilized three contrived samples in each batch that were different from the samples used in Luminex Method 1 and Method 2. One sample with 11 replicates and two samples with 12 replicates. Details of the ELISA Method are provided in Example 8. The CEA and CA125 Ortho/VITROS assays utilized 9 contrived samples in each batch that were different from Luminex Method 1 and Method 2 and FGL1 ELISA Method. Details of the Ortho/VITROS Methos are provided in Example 9. Table 14 shows the acceptance criteria for the analytical precision study. Data that passed the inclusion criteria was utilized for two-way ANOVA analysis.

TABLE 14
Acceptance Criteria for Analytical Precision Study

| | | Within-lab & |
|---|---|---|
| | | Within-run CV |
| Immunoassay | Analyte | (%) |
| Luminex | gp130 | ≤20% |
| | CD31/PECAM-1 | ≤20% |
| | C9 | ≤20% |
| | Myoglobin | ≤20% |
| | IGFBP6 | ≤20% |
| | CYFRA21-1 | ≤20% |
| | Enolase-2 | ≤20% |
| | CEACAM-5/CD66e | ≤20% |
| | SAA | ≤20% |
| ELISA | FGL1 | ≤20% |
| Ortho | CEA | ≤20% |
| | CA125 | ≤20% |

A two-way nested ANOVA was used to detect variability from the experimental variables of interest. Within-lab precision was derived from the total variation in the ANOVA analysis. Repeatability (within-run precision) was derived from the error in two-way ANOVA analysis. Day-to-day variation within operators and operator-to-operator variation was also derived from the ANOVA analysis. Intra-day variation was determined by the CV values.

The % CV for within-run precision for each protein, except SAA, was less than 10%. The CV for SAA was 13% which is still well below the acceptance threshold of 20%. The CV for within-lab precision for all proteins, except SAA and C9, was less than or equal to 10% suggesting good within-lab precision. The within-lab precision values for SAA and C9 were around 11% and 13% respectively which was comfortably below the acceptance threshold of 20%. The repeatability of FGL1 ELISA assay is 5% and the within-lab precision is about 8%. The full results of the ANOVA analyses are provided in Table 15 and Table 16.

TABLE 15
Summary of two-way ANOVA results for proteins assayed on Luminex

| | Acceptable | | |
|---|---|---|---|
| | Within-run and | | Within-Lab |
| | Within-lab | Repeatability | Precision |
| Protein | % CV | (% CV) | (% CV) |
| PL15\|gp130 | ≤20% | 2% | 4% |
| PL17\|Complement | ≤20% | 8% | 11% |
| Component C9 | | | |
| PL17\|Myoglobin | ≤20% | 10% | 10% |
| PL18\|IGFBP-6 | ≤20% | 3% | 5% |
| PL20\|CYFRA21-1 | ≤20% | 3% | 7% |
| PL20\|Enolase | ≤20% | 6% | 7% |
| 2/Neuron-specific | | | |
| Enolase | | | |
| PL16\|CD31/PECAM-1 | ≤20% | 4% | 4% |
| PL21\|CEACAM-5/ | ≤20% | 3% | 13% |
| CD66e | | | |
| PL22\|SAA | ≤20% | 13% | 5% |

TABLE 16
Summary of two-way ANOVA results for FGLI ELISA assay

| | Acceptable | | |
|---|---|---|---|
| | Within-run and | Repeatability/ | Within-Lab |
| | Within-lab | Within-run | Precision |
| Protein | % CV | (% CV) | (% CV) |
| FGL1 | ≤20% | 5% | 8% |

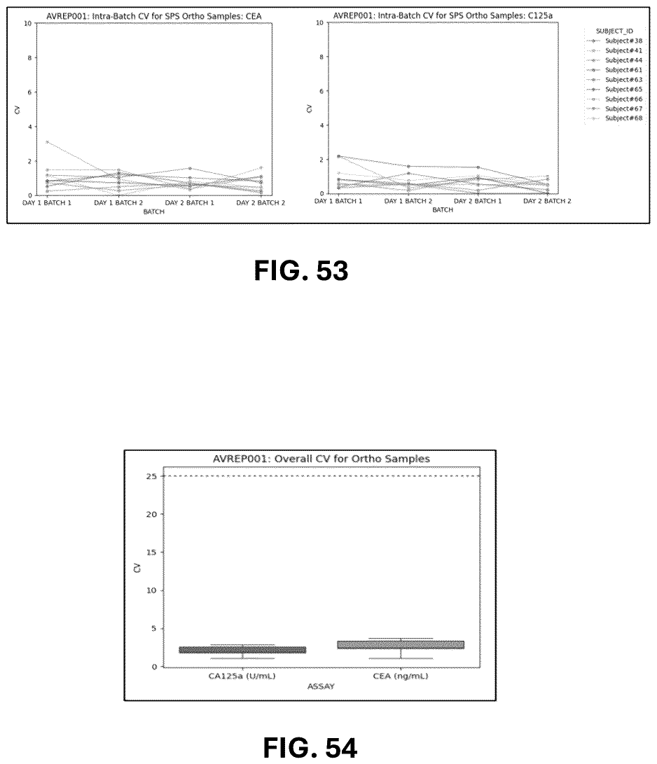

Intra-day CV values for both CA125 and CEA for all subjects were below 5% (FIG. 53). The results indicated robust intra-day precision for both ortho/VITROS assays. Inter-day CV values for CA125 and CEA for all subjects were also 5%. The overall CV was low indicating robust precision of the Ortho immunoassays (FIG. 54).

All 12 assays (Luminex, ELISA and Ortho) met the precision study acceptance criteria.

### Parallelism and Linearity

This study was conducted to confirm analyte parallelism for each of the target proteins, by determining concordance between the dilution factor corrected sample concentration measurements and the standard curve concentrations. This was done for measurements in the linear portion of the standard curve. Linearity of serially diluted samples was assessed using linear regression analysis between expected concentrations and measured concentrations to determine whether the proteins demonstrated proportionality across the dilution range. Samples including clinical samples, commercial samples, and contrived samples were procured or prepared and processed through the immunoassay workflows for parallelism and linearity. Five independent samples were chosen for Luminex Method 1, Luminex Method 2 and FGL1 ELISA in this experiment, and they were serially diluted seven times with 2-fold steps. The samples were processed through the Luminex and ELISA workflows for data collection. Luminex Method 1, Luminex Method 2, and ELISA workflows are described in Examples 7 and 8.

There are no Clinical and Laboratory Standards Institute (CLSI) guidelines around parallelism criteria and acceptance. The methodology adopted for this study was taken from Valentin et. al which is herein incorporated by reference in its entirety.

Linear regression and coefficient of determination were used at protein basis to validate parallelism for each target protein and linearity of the serially diluted samples. The data from a sample was included for linear regression analysis when a minimum of three data points from the sample fell within the Standard 1 to Standard 6/Standard 7 range. A linear regression analysis was performed on the measured concentrations for the diluted plasma samples versus 1/dilution factor. When the slope was between 0.8 to 1.2, the study was considered as passing acceptance criteria. A protein feature passed when a minimum of two out of the five samples tested for parallelism met the study acceptance criteria. Data that passed quality control was used for linear regression analysis at per protein and per sample basis.

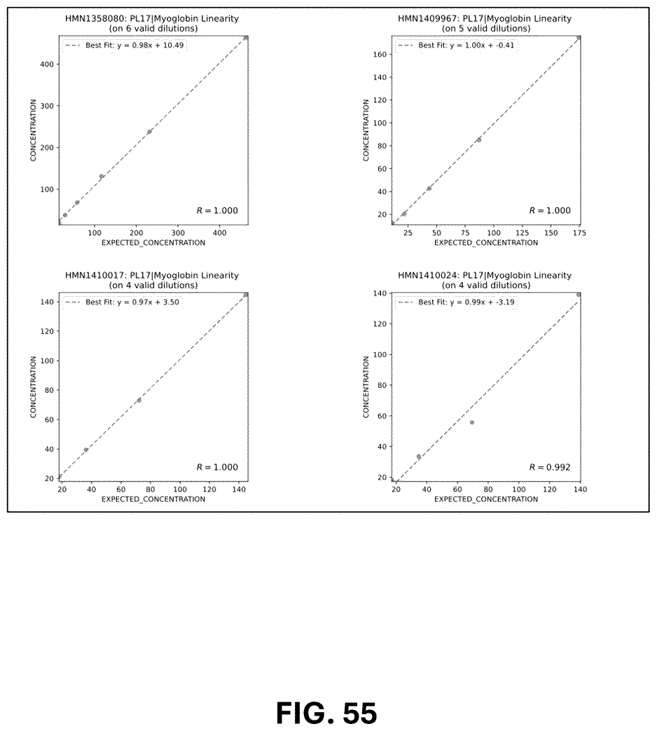

As mentioned previously, at least three data points from a sample had to fall in the range between Standard 1 to Standard 6 to qualify for linear regression analysis. Most of the proteins in each sample used 6 data points for linear regression. The fewest data points used for linear regression was 3 data points. For gp130, C9, Myoglobin, IGFBP-6, CYFRA21-1 and Enolase 2, all five samples passed the study acceptance criteria (Table 17). As an example, the plots for Myoglobin are included in FIG. 55. In the case of CD31/PECAM-1 and CEACAM-5/CD66e, all five samples met the study acceptance criteria while SAA had two samples meeting study acceptance criteria (Table 18). All five samples met study acceptance criteria for FGL1 (Table 19). All protein features passed the parallelism and linearity study acceptance criteria, indicating that there is no significant bias in concentration across the concentration ranges.

TABLE 17
Slopes from Linear Regression analysis for Luminex assays - part 1

| | Acceptable | | | | | |
|---|---|---|---|---|---|---|
| | range for | Sam- | Sam- | Sam- | Sam- | Sam- |
| Protein | slope | ple 1 | ple 2 | ple 3 | ple 4 | ple 5 |
| gp130 | 0.8-1.2 | 0.98 | 0.95 | 0.99 | 1.0 | 0.99 |
| Complement | 0.8-1.2 | 0.99 | 0.96 | 1.02 | 0.98 | 0.99 |
| Component C9 | | | | | | |
| Myoglobin | 0.8-1.2 | 0.97 | 0.99 | 1.00 | 0.98 | 0.99 |
| IGFBP-6 | 0.8-1.2 | 1.00 | 1.02 | 1.00 | 0.98 | 0.98 |
| CYFRA21-1 | 0.8-1.2 | 1.01 | 0.96 | 0.99 | 1.01 | 1.00 |
| Enolase | 0.8-1.2 | 1.00 | 0.95 | 0.97 | 1.01 | 0.99 |
| 2/Neuron- | | | | | | |
| specific | | | | | | |
| Enolase | | | | | | |

TABLE 18
Slopes from Linear Regression analysis for Luminex assays - part 2

| | Acceptable | | | | | |
|---|---|---|---|---|---|---|
| | range for | Sam- | Sam- | Sam- | Sam- | Sam- |
| Protein | slope | ple 1 | ple 2 | ple 3 | ple 4 | ple 5 |
| PL16\|CD31/ | 0.8-1.2 | 1.00 | 0.99 | 1.00 | 0.99 | 0.99 |
| PECAM-1 | | | | | | |
| PL21\|CEACAM- | 0.8-1.2 | 0.98 | 0.99 | 0.98 | 0.97 | 0.98 |
| 5/CD66e | | | | | | |
| PL22\|SAA1 | 0.8-1.2 | N/A | N/A | N/A | 1.04 | 1.04 |

TABLE 19
Slopes from Linear Regression analysis for FGL1 ELISA

| | Acceptable | | | | | |
|---|---|---|---|---|---|---|
| | range for | Sample | Sample | Sample | Sample | Sample |
| Protein | slope | 1 | 2 | 3 | 4 | 5 |
| FGL1 | 0.8-1.2 | 0.94 | 0.97 | 0.96 | 0.97 | 0.95 |

### Analytical Accuracy

This study was designed to test analytical accuracy which refers to the closeness of agreement between a measured value and the true or accepted reference value. Due to absence of orthogonal tests for individual components of the assay on comparable platforms (e.g., Luminex, ELISA and Ortho/VITROS platforms), accuracy was not assessed through inter-laboratory comparison. Instead, analytical accuracy of the protein concentration measurements was evaluated for each immunoassay method by determining the agreement of two separate measurements of samples that span the full analytical measurement range for each of the proteins.

Samples including clinical samples, commercial samples, and contrived samples were procured or prepared and processed through the immunoassay workflows for analytical accuracy. A total of 140 plasma samples (2 aliquots from 70 unique samples), spread across 4 batch runs, were processed through Luminex and ELISA workflows. Detailed methods for Luminex and ELISA workflows are provided in Examples 7 and 8. In the case of Ortho/VITROS, three contrived plasma samples with pre-determined CEA and CA-125 concentrations targeting the high, medium and low levels of the assay's analytical measurement range were utilized for accuracy study (Table 20). Details methods for the Ortho/VITROS workflow is provided in Example 9.

TABLE 20
Analytical Measurement range targeted by contrived samples

| | High | Medium | Low |
|---|---|---|---|
| Assay | (80-100%) | (40-60%) | (0-10%) |
| CEA assay | 320-400 ng/ml | 160-240 ng/ml | 0.31-40 ng/ml |
| CA125 assay | 800-1000 U/mL | 400-600 U/mL | 5.5-100 U/mL |

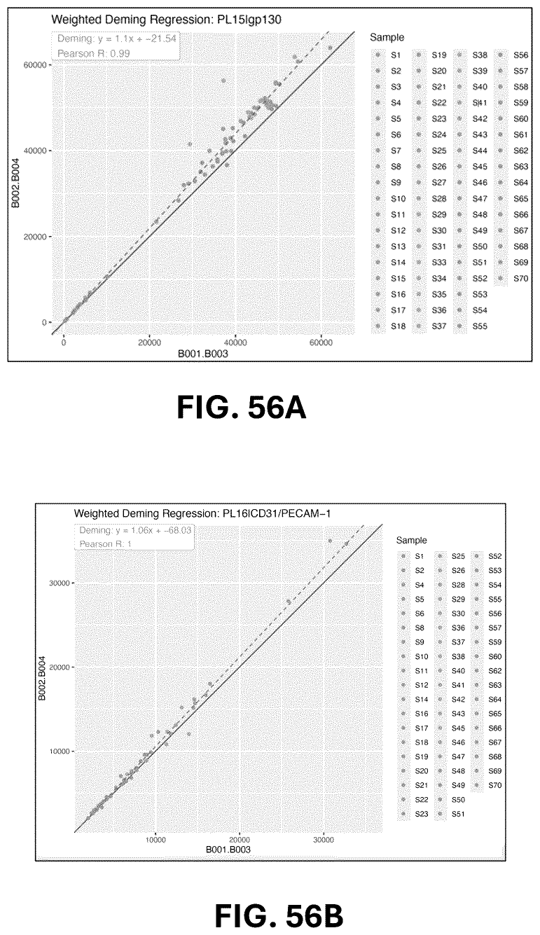

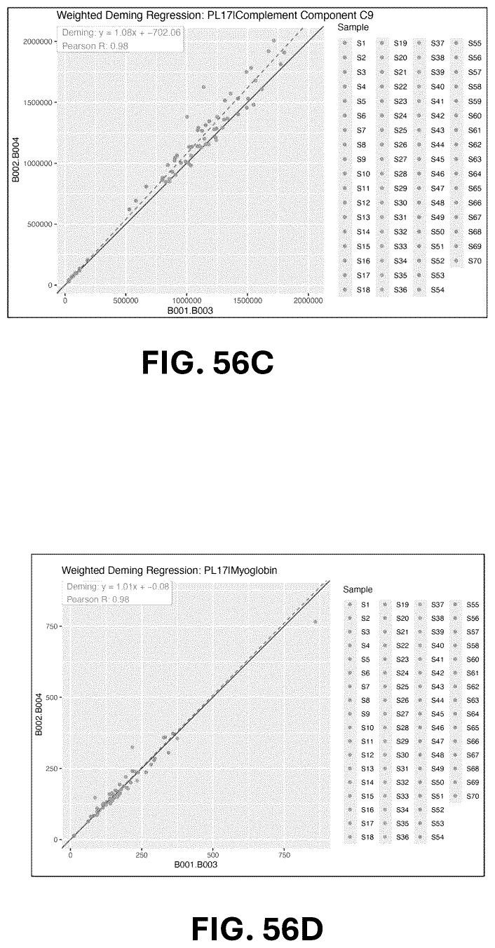

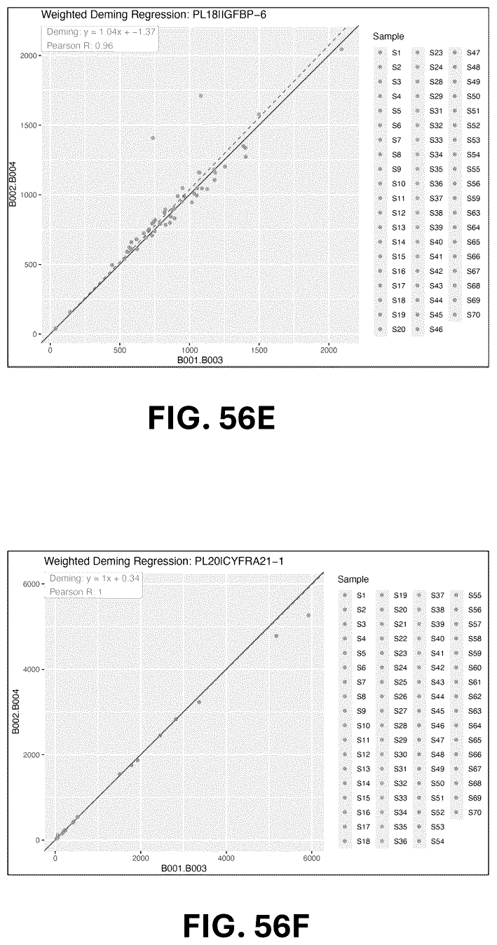

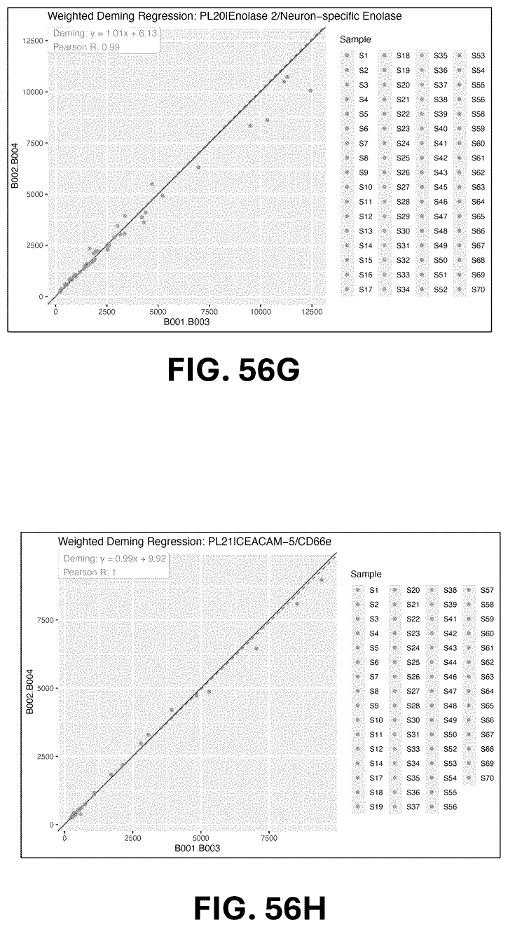

The Deming Regression and Pearson correlation was calculated between the two aliquots from the same subject and across all subjects. For the Deming Regression, the slope fell between 0.87 to 1.13 to be considered as passing study acceptance criteria. Similarly, for Pearson correlation, a coefficient >0.94, was considered as passing study criteria. The Deming slope and Pearson correlation was determined for proteins assessed using the Luminex and ELISA platforms and was found to meet accuracy acceptance criteria for all proteins except SAA1 (Table 21 and Table 22). FIGS. 56A-56I show the Deming regression plots for the proteins meeting the acceptance criteria. X-axis and Y-axis are concentrations in pg/mL.

TABLE 21
Deming Regression (slope) and Pearson correlation results
for proteins assayed by Luminex

| | Acceptable | Acceptable | | |
|---|---|---|---|---|
| | Deming | Pearson | Deming | Pearson |
| Protein | slope | correlation | Slope | R |
| gp130 | 0.87 to 1.13 | ≥0.94 | 1.10 | 0.99 |
| CD31/PECAM-1 | 0.87 to 1.13 | ≥0.94 | 1.06 | 1.00 |
| Complement | 0.87 to 1.13 | ≥0.94 | 1.08 | 0.98 |
| Component C9 | | | | |
| Myoglobin | 0.87 to 1.13 | ≥0.94 | 1.01 | 0.98 |
| IGFBP-6 | 0.87 to 1.13 | ≥0.94 | 1.04 | 0.96 |
| CYFRA21-1 | 0.87 to 1.13 | ≥0.94 | 1.00 | 1.00 |
| Enolase 2/Neuron- | 0.87 to 1.13 | ≥0.94 | 1.01 | 0.99 |
| specific Enolase | | | | |
| CEACAM-5/CD66e | 0.87 to 1.13 | ≥0.94 | 0.99 | 1.00 |
| SAA1 | 0.87 to 1.13 | ≥0.94 | Not | Not |
| | | | enough | enough |
| | | | data | data |

TABLE 22
Deming Regression (slope) and Pearson correlation results
for proteins assayed by ELISA

| | Acceptable | Acceptable | | |
|---|---|---|---|---|
| | Deming | Pearson | Deming | Pearson |
| Protein | slope | correlation | Slope | R |
| ELISA\|FGL1 | 0.87 to 1.13 | ≥0.94 | 1.04 | 0.99 |

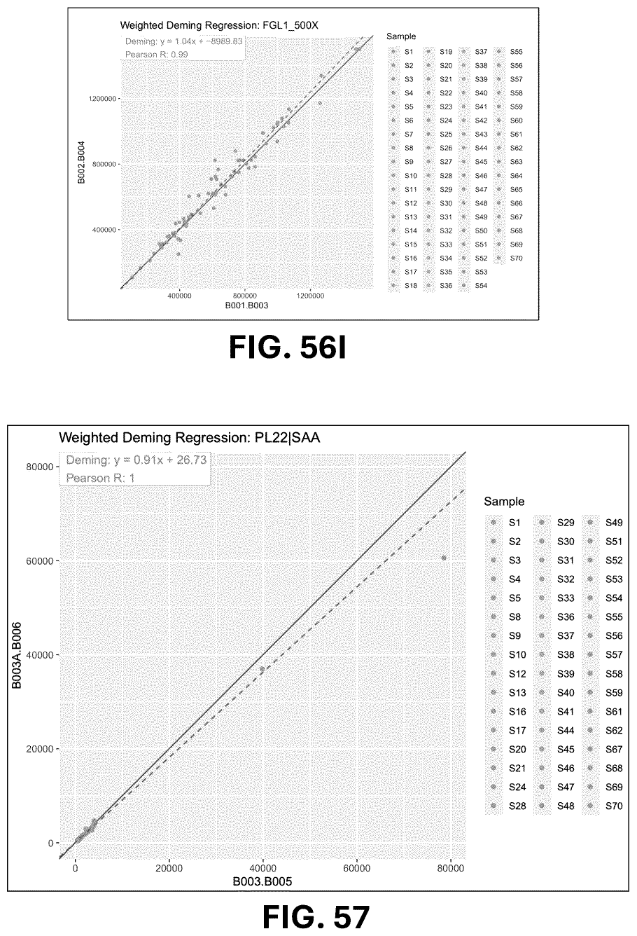

In the case of SAA1, 38 of the 70 samples passed QC which did not meet the minimum requirement of 40 samples to proceed with statistical analysis. To address this, a new set of samples were processed to quantify SAA1. The new batch runs were able to produce data that passed QC criteria, and the Deming slope and Pearson correlation were computed. The results obtained indicated that neither of these met the study acceptance criteria. It was determined there were inconsistencies between batches during processing of the plates particularly in the incubation steps, which may have impacted the results. Additionally, there were three samples at the high end of the SAA curve which may have skewed the regression line. Due to the unknown impact of the inconsistencies during data collection a third run was conducted utilizing samples with a better distribution across the dynamic range of the assay. The Deming regression and Pearson correlation were calculated, and both met the acceptance criteria (Table 23). FIG. 57 shows the Deming regression for SAA. X-axis and Y-axis are concentrations in pg/mL.

TABLE 23
Deming slope and Pearson R results for SAA

| | Deming | Pearson |
|---|---|---|
| Feature | Slope | R |
| PL22\|SAA | 0.91 | 1.00 |

For the Ortho/VITROS assays, accuracy was assessed by calculating the percent recovery of the low, medium, and high-level samples. The percent recovery for each sample fell between 87% and 113% to be considered as passing study acceptance criteria. All samples analyzed in the Ortho/VITROS assay at all concentration levels demonstrated percent recovery that passed study acceptance criteria (Table 24).

TABLE 24
Percent recovery for low, medium, high samples
processed through CEA and CA125 Ortho assays

| | | | | | % Recovery | % Inaccuracy | % Recovery |
|---|---|---|---|---|---|---|---|
| | Sample | | Avg | Previous | vs Previous | vs Previous | Acceptance |
| Feature | Description | Units | Conc | Value | Value | Value | Criteria |
| CA125 | Spiked | U/mL | 17.4 | 16.9 | 103.0% | 3.0% | 87-113% |
| | Low | | | | | | |
| | Spiked Mid | | 506 | 493 | 102.6% | 2.6% | |
| | Spiked | | 895 | 875 | 102.3% | 2.3% | |
| | High | | | | | | |
| CEA | Spiked | ng/mL | 12.3 | 12.6 | 97.6% | −2.4% | |
| | Low | | | | | | |
| | Spiked Mid | | 192 | 192 | 99.8% | −0.2% | |
| | Spiked | | 353 | 351 | 100.5% | 0.5% | |
| | High | | | | | | |

### Analytical Reportable Range

The Analytical Reportable Range study was designed to validate the measurable range of the assay by evaluating both Analytical Measuring Interval (AMI) and the Extended Measuring Interval (EMI). The goal was to confirm that the assay produces accurate, precise and linear results across the full range of the expected sample concentrations. Samples including clinical samples, commercial samples, and contrived samples were procured or prepared and processed through the immunoassay workflows for analytical reportable range.

For Luminex and ELISA assays, data collected from Analytical Precision and Parallelism & Linearity studies was utilized for analyses and establishing the reportable ranges for individual proteins. The outer limits of the AMI for each protein were fixed based on analysis completed as part of the Analytical Sensitivity, Parallelism & Linearity and Precision studies. The calculated LLOQ and ULOQ defined the outer limits of the corresponding AMI for each protein. Standard 6 was set as the LLOQ, and the highest standard (Standard 1) was set as ULOQ for each protein. The established AMI and EMI limits from the Analytical Sensitivity study and used for reportable range are listed in Table 25.

TABLE 25
AMI and EMI limits established as part of Analytical Sensitivity Study

| Feature | LOD | LLOQ | ULOQ | DF | ULOQ × DF |
|---|---|---|---|---|---|
| PL15\|gp130 | 12.5 | 301.8 | 73,333 | 2 | 146660 |
| PL16\|CD31/ | 165.8 | 754.7 | 183,390 | 2 | 366780 |
| PECAM-1 | | | | | |
| PL17\|Complement | 5078.7 | 30,509 | 7,413,650 | 50 | 37,0682,500 |
| Component C9 | | | | | |
| PL17\|Myoglobin | 3.1 | 11.7 | 2840 | 50 | 142,000 |
| PL18\|IGFBP-6 | 4.3 | 38.7 | 9400 | 200 | 1,880,000 |
| PL20\|CYFRA21-1 | 6.2 | 36.2 | 8800 | 2 | 17,600. |
| PL20\|Enolase 2/ | 66.4 | 222.2 | 54,000 | 2 | 108,000 |
| Neuron-specific | | | | | |
| Enolase | | | | | |
| PL21\|CEACAM-5/ | 3.9 | 57.6 | 14,000 | 8 | 112,000 |
| CD66e | | | | | |
| PL22\|SAA1 | 263.9 | 456.4 | 110,900 | 4000 | 443,600,000 |
| ELISA\|FGL1 500X | 21.5 | 93.8 | 3000 | 500 | 150,000 |

The dilution linearity within the AMI for each protein was demonstrated as part of the Parallelism and Linearity study. Details of the Parallelism and Linearity study can be found previously in this example. The reproducibility and repeatability were assessed for each protein within the AMI and all assays met study acceptance criteria. The HQC, MQC and LQC sample data from the Analytical Accuracy study was used to validate the AMI limits. The results demonstrate that the intra-plate CV was ≤20% which met the acceptance criteria for AMI validation. The AMI validation results are shown in Table 26.

TABLE 26
AMI Validation Results

| | C1-Low | | C2-Med | | C3-High | |
|---|---|---|---|---|---|---|
| | Average | | Average | C2 | Average | |
| Protein | concentration | C1 max | concentration | max | concentration | C3 max |
| Features | (pg/mL) | % CV | (pg/mL) | % CV | (pg/mL) | % CV |
| gp130 | 1020.8 | 1.5 | 3065.4 | 5.1 | 16,429.5 | 3.7 |
| CD31/ | 2507.5 | 5.5 | 7585.7 | 4.8 | 41,312.3 | 2.9 |
| PECAM-1 | | | | | | |
| Complement | 89,481.2 | 10.1 | 286,738.1 | 7.3 | 1,590,950.4 | 2.8 |
| Component C9 | | | | | | |
| Myoglobin | 60.0 | 4.1 | 171.6 | 2.6 | 844.3 | 1.6 |
| IGFBP-6 | 134.4 | 4.9 | 410.3 | 4.7 | 2290.3 | 2.4 |
| CYFRA21-1 | 149 | 3.0 | NA | NA | 1531.1 | 4.1 |
| Enolase | 512.1 | 6.0 | NA | NA | 4325.5 | 2.0 |
| 2/Neuron- | | | | | | |
| specific | | | | | | |
| Enolase | | | | | | |
| CEACAM- | 241.3 | 5.5 | NA | NA | 2566.9 | 5.4 |
| 5/CD66e | | | | | | |
| SAA | 21,993.3 | 3.2 | 21,993.3 | 5.7 | 82,878.3 | 19.5 |
| FGL1 | 147.6 | 5.2 | 492 | 1.9 | 1531.3 | 3.0 |

For Ortho/VITROS Immunoassays, the manufacturer specified upper and lower limits were verified by testing samples that overlapped these portions of the assay ranges. Three contrived plasma samples with pre-determined CEA and CA-125 concentrations, targeting the high and low levels of the assay's analytical measurement range were utilized to verify the reportable range (Table 20). The reportable range for the Ortho/VITROS assays were verified by calculating the percent recovery (e.g., the ratio of measured concentrations at each level (high and low) to the previously measured concentration value). The acceptance criteria were set for the study where percent recovery for each sample fell between 87% and 113%.

For the Ortho/VITROS assays (CEA and CA125), the reportable range was verified. The percent recovery was determined and fell within 87-113% which had been fixed as the study acceptance criteria (Table 27).

TABLE 27
Results from the Reportable Range verification studies for Ortho/VITROS assays

| | | | | | % | | |
|---|---|---|---|---|---|---|---|
| | | | | | Recovery | % | |
| | | | | | vs | Discrepancy | % Recovery |
| | Sample | | Avg | Previous | Previous | vs Previous | Acceptance |
| Feature | Description | Units | Conc | Value | Value | Value | Criteria |
| CA125 | Spiked | U/mL | 8.4 | 8.5 | 99.2% | −0.8% | 87-113% |
| | Low | | 6.7 | 6.3 | 106.3% | 6.3% | |
| | | | 28.9 | 29.0 | 99.8% | −0.2% | |
| | Spiked | | 839.7 | 802 | 104.7% | 4.7% | |
| | High | | 713.7 | 683 | 104.5% | 4.5% | |
| | | | 654.7 | 641 | 102.1% | 2.1% | |
| CEA | Spiked | ng/mL | 1.0 | 1.0 | 95.3% | −4.7% | 87-113% |
| | Low | | 3.2 | 3.3 | 96.2% | −3.8% | |
| | | | 5.8 | 5.9 | 98.2% | −1.8% | |
| | Spiked | | 321.7 | 321 | 100.2% | 0.2% | |
| | High | | 261.3 | 261 | 100.1% | 0.1% | |
| | | | 228.7 | 236 | 96.9% | −3.1% | |

### Analytical Specificity

The interference study was designed to evaluate the potential impact of common endogenous interferents (lipemia, bilirubin (both conjugated and unconjugated), and hemolysis) on the analytical performance of the immunoassays described herein (e.g., Luminex, ELISA, Ortho/VITROS. The objective was to determine whether the presence of these substances affects the accuracy of any of the protein marker measurements. This study was executed to test and define the tolerance thresholds of the various interfering substances. A variation of +/−13% was defined as the deviation threshold for protein measurements when comparing plasma samples with and without interferents. No study acceptance criteria were defined, and this study was completed as a characterization of tolerance for various interferents.

Triglycerides, hemolysate and bilirubin (conjugated and unconjugated) were the interference substances tested. Samples including clinical samples, commercial samples, and contrived samples were procured or prepared and processed through the immunoassay workflows for analytical specificity. Plasma samples were prepared by spiking in various concentrations of the interfering substances (Table 28). Unspiked plasma samples served as the controls. A single female plasma pooled sample was used for both Luminex methods and FGL1 ELISA, and different proteins for each Luminex method were spiked into the sample. The contrived sample for Luminex Method 1 was spiked with CYFRA21-1 and Enolase 2 to ensure a high enough concentration for measurement. The contrived sample for Luminex Method 2 was spiked with SAA and no additional protein was added to the FGL1 sample pool. Seven different concentration levels of the interfering substances were tested, and eight replicates of spiked plasma samples were processed for each level.

TABLE 28
Concentrations of different Interference substances being tested

| | Bilirubin | Bilirubin | | |
|---|---|---|---|---|
| Dilution | conjugated | unconjugated | Hemolysate | Triglycerides |
| Steps | (mg/dL) | (mg/dL) | (g/dL) | (mg/dL) |
| 1 | 30 | 30 | 1 | 1500 |
| 2 | 15 | 15 | 0.5 | 750 |
| 3 | 7.5 | 7.5 | 0.25 | 375 |
| 4 | 3.75 | 3.75 | 0.1 | 187.5 |
| 5 | 1.88 | 1.88 | 0.063 | 93.75 |
| 6 | 0.94 | 0.94 | 0.0313 | 46.88 |
| 7 | 0.47 | 0.47 | 0.0156 | 23.44 |

All the interfering substances tested caused more than +13% variation in protein measurements but at different concentration levels. Five protein assays exhibited interference due to the presence of triglycerides (TRG). This included gp130 (at triglyceride level>187.5 g/dL), CD31 (at triglyceride level>1500 g/dL), C9 (at triglyceride level>375 g/dL), CYFRA 21 (at triglyceride level>0 g/dL) and Enolase (at triglyceride level>0 g/dL). The p-values indicated that majority of the results are statistically significant, with values less than 0.05 (Table 29). % Differences that failed acceptance criteria are underlined in Table 29. FGL1 was not impacted by any of the concentration levels of TRG tested.

TABLE 29
% Difference in Measured Concentration of the Protein Markers
(Features) at Different Triglyceride (TRG) Levels.

| | 1500 | 750 | 375 | 187.5 | 93.75 | 46.88 | 23.44 | 0 |
|---|---|---|---|---|---|---|---|---|
| FEATURE | (mg/dL) | (mg/dL) | (mg/dL) | (mg/dL) | (mg/dL) | (mg/dL) | (mg/dL) | (mg/dL) |
| PL15\|gp130 | 11.2 | 14.6 | 14 | 9.8 | 8 | 5.5 | 2.8 | 0 |
| PL16\|CD31/ | 14.3 | 10.6 | 7 | 5.6 | 2.1 | −0.3 | −1.8 | 0 |
| PECAM-1 | | | | | | | | |
| PL17\|Complement | 18.7 | 16.3 | 9.3 | 7.5 | 3.9 | 5.1 | 3.6 | 0 |
| Component C9 | | | | | | | | |
| PL17\|Myoglobin | 7.7 | 11.9 | 8.7 | 10 | 6.4 | 5.9 | 4.1 | 0 |
| PL18\|IGFBP-6 | 3.3 | 5.4 | 5.6 | 6.4 | −0.1 | 3.4 | 2.6 | 0 |
| PL20\|CYFRA21-1 | −22.8 | −43.4 | −39.4 | −46.7 | −36.7 | −54.2 | −42 | 0 |
| PL20\|Enolase | −23.6 | −22 | −24.5 | −22.1 | −20.2 | −22.4 | −17.1 | 0 |
| 2/Neuron- | | | | | | | | |
| specific | | | | | | | | |
| Enolase | | | | | | | | |
| PL21\|CEACAM- | −0.4 | 2.1 | 2.1 | 3.4 | 2.5 | 2.6 | 2.6 | 0 |
| 5/CD66e | | | | | | | | |
| PL22\|SAA | 31 | 10.1 | 2.4 | −0.8 | 7.8 | −1 | 4.3 | 0 |
| FGL1_500X | −8.2 | −2.3 | 0.2 | 2.1 | 3.1 | 5.5 | 1.8 | 0 |

Three protein assays exhibited interference due to the presence of hemolysate (HEM). This included CD31 (at hemolysate level>0.5 g/dL), Myoglobin (at hemolysate level>0.25 g/dL), and Enolase (at hemolysate level>0 g/dL). The p-values indicated that most of the results are statistically significant, with values less than 0.05 (Table 30). % Differences that failed acceptance criteria are underlined in Table 30. FGL1 showed an interference higher than the tolerance thresholds at the highest concentration of hemolysate (1 g/dL).

TABLE 30
% Difference in Measured Concentration of the Protein Markers
(Features) at Different Hemolysate (HEM) Levels.

| | 1 | 0.5 | 0.25 | 0.1 | 0.063 | 0.0313 | 0.0156 | 0 |
|---|---|---|---|---|---|---|---|---|
| Feature | (g/dL) | (g/dL) | (g/dL) | (g/dL) | (g/dL) | (g/dL) | (g/dL) | (g/dL) |
| PL15\|gp130 | 3.1 | 4.3 | 5.2 | 5.1 | 3.6 | 4.3 | 2.7 | 0 |
| PL16\|CD31/ | 23.4 | 11.8 | 4.1 | 6.1 | 2.2 | 1.3 | −0.3 | 0 |
| PECAM-1 | | | | | | | | |
| PL17\|Complement | −2.8 | −1 | 0.7 | −2.3 | −2.9 | −1.8 | −2.9 | 0 |
| Component C9 | | | | | | | | |
| PL17\|Myoglobin | 12.5 | 13.6 | 10.2 | 2.1 | 1.1 | −0.3 | −0.6 | 0 |
| PL18\|IGFBP-6 | −9.3 | −5.7 | −3.9 | −4.7 | −6.2 | −5.4 | −5.6 | 0 |
| PL20\|CYFRA21-1 | −2.3 | −6 | 2.2 | 8.6 | −3.4 | −7.6 | −4.6 | 0 |
| PL20\|Enolase | 7855.2 | 6400.4 | 4496.6 | 2868.3 | 1648.5 | 923.9 | 543.2 | 0 |
| 2/Neuron-specific | | | | | | | | |
| Enolase | | | | | | | | |
| PL21\|CEACAM- | −2.6 | −3 | −0.8 | 0.9 | −2.2 | −1.2 | −2.4 | 0 |
| 5/CD66e | | | | | | | | |
| PL22\|SAA | −4 | 2 | 0.7 | 4.8 | 4.1 | 12.5 | 12.2 | 0 |
| FGL1_500X | −15.7 | −2.6 | 1.5 | −0.3 | −3.5 | −4.8 | 2 | 0 |

Two protein assays exhibited interference due to the presence of bilirubin unconjugated (BU). This included CYFRA-21 (at BU level=0.94 mg/dl), and Enolase (at BU level>0 mg/dL). The p-values indicated that the majority of the results are statistically significant, with values less than 0.05 (Table 31). % Differences that failed acceptance criteria are underlined in Table 31. None of the levels of BU impacted the protein measurements of FGL1 significantly.

TABLE 31
% Difference in Measured Concentration of the Protein Markers
(Features) at Different Bilirubin Unconjugated (BU) Levels

| | 30 | 15 | 7.5 | 3.75 | 1.88 | 0.94 | 0.47 | 0 |
|---|---|---|---|---|---|---|---|---|
| FEATURE | (mg/dL) | (mg/dL) | (mg/dL) | (mg/dL) | (mg/dL) | (mg/dL) | (mg/dL) | (mg/dL) |
| PL15\|gp130 | 0.1 | 2.1 | 1 | 2.4 | 3.3 | 2.9 | 1.5 | 0 |
| PL16\|CD31/ | −2.5 | −1.9 | −0.9 | 0.1 | 0.5 | 0.6 | −1.3 | 0 |
| PECAM-1 | | | | | | | | |
| PL17\|Complement | 7.3 | 3 | 4.8 | 3.3 | −1.3 | 1.3 | −1 | 0 |
| Component C9 | | | | | | | | |
| PL17\|Myoglobin | 0.8 | 0 | 2.1 | 1.2 | −0.7 | 0 | −1.5 | 0 |
| PL18\|IGFBP-6 | −4.4 | −1.5 | −0.8 | −0.8 | −2.6 | 2.1 | −4.9 | 0 |
| PL20\|CYFRA21-1 | −11.2 | −9.4 | 1.5 | −9 | 12.8 | −15.2 | −10.9 | 0 |
| PL20\|Enolase | 47.3 | 55.2 | 62.4 | 59.3 | 70 | 57.5 | 36.7 | 0 |
| 2/Neuron-specific | | | | | | | | |
| Enolase | | | | | | | | |
| PL21\|CEACAM- | −2.5 | −1.2 | −0.9 | −0.7 | 0.8 | −1.2 | −0.8 | 0 |
| 5/CD66e | | | | | | | | |
| PL22\|SAA | −1.9 | −2.7 | −9 | −6 | −3.9 | −6.8 | −6.8 | 0 |
| FGL1_500X | 2.3 | 3.2 | 4.3 | 5.9 | 4.8 | 3.2 | 2.3 | 0 |

Two protein assays exhibited interference due to the presence of bilirubin conjugated (B). This included CYFRA-21 (at B level>15 mg/dl), and Enolase (at B level>15 mg/dL). The p-values indicated that most of the results are statistically significant, with values less than 0.05 (Table 32). Percent differences that failed acceptance criteria are underlined in Table 32. Only at the highest concentration (B level=30 mg/dL), were the protein measurements of FGL1 significantly impacted.

TABLE 32
Percent Difference in Measured Concentration of the Protein Markers
(Features) at Different Bilirubin conjugated (B) Levels

| | 30 | 15 | 7.5 | 3.75 | 1.88 | 0.94 | 0.47 | 0 |
|---|---|---|---|---|---|---|---|---|
| FEATURE | (mg/dL) | (mg/dL) | (mg/dL) | (mg/dL) | (mg/dL) | (mg/dL) | (mg/dL) | (mg/dL) |
| PL15\|gp130 | 2.3 | 3.6 | 5.2 | 5.9 | 5.2 | 1.5 | 4.7 | 0 |
| PL16\|CD31/ | −1.7 | 2.3 | 2.2 | 1.3 | 2 | 2.1 | 1.5 | 0 |
| PECAM-1 | | | | | | | | |
| PL17\|Complement | 0.9 | 1.4 | 2.9 | 0.4 | −0.2 | −1.1 | −0.1 | 0 |
| Component C9 | | | | | | | | |
| PL17\|Myoglobin | 1 | 4.7 | 6.4 | 5.1 | 4 | 1.7 | 0.8 | 0 |
| PL18\|IGFBP-6 | −3.5 | −0.3 | 3 | −0.5 | −2.7 | −1.3 | −0.2 | 0 |
| PL20\|CYFRA21-1 | −18.5 | −8.8 | −6.8 | −3.5 | 6.7 | −1.9 | 11.1 | 0 |
| PL20\|Enolase | −16.2 | −7 | −6.4 | −1.7 | 2 | −1.9 | 0.3 | 0 |
| 2/Neuron- | | | | | | | | |
| specific | | | | | | | | |
| Enolase | | | | | | | | |
| PL21\|CEACAM- | −2.9 | 0 | −0.7 | 3.3 | −1.9 | −1 | 1.4 | 0 |
| 5/CD66e | | | | | | | | |
| PL22\|SAA | −3.8 | 0 | −4.3 | 2.4 | 5.1 | 2.6 | 2.7 | 0 |
| FGL1_500X | 267.9 | −7 | −2.6 | −2.2 | 4.7 | 3.7 | 3.3 | 0 |

In conclusion, several proteins assays exhibited interference beyond the accepted threshold of +/−13% for protein measurements. CYFRA21-1 displayed no tolerance for triglycerides while Enolase-2 displayed no tolerance for triglycerides, hemolysate and bilirubin unconjugated interference substance. A summary of the observed tolerance levels for each interference substance is presented in Table 33.

TABLE 33
Observed tolerance levels for the Interference Substances tested

| | | | Bilirubin | Bilirubin |
|---|---|---|---|---|
| Protein Assay | Triglyceride | Hemolysate | (Unconjugated) | (conjugate) |
| gp130 | 187.5 g/dL | 1 g/dL | 30 mg/dL | 30 mg/dL |
| Complement | 375 g/dL | | | |
| Component C9 | | | | |
| Myoglobin | 1500 g/dL | 0.25 g/dL | | |
| IGFBP-6 | 1500 g/dL | 1 g/dL | | |
| CYFRA21-1 | 0 g/dL | | 1.88 mg/dL | 15 mg/dL |
| Enolase 2/ | 0 g/dL | 0 g/dL | 0 mg/dL | 15 mg/dL |
| Neuron- | | | | |
| specific | | | | |
| Enolase | | | | |
| CD31/ | 750 g/dL | 0.5 g/dL | 30 mg/dL | 30 mg/dL |
| PECAM-1 | | | | |
| CEACAM-5/ | 1500 g/dL | 1 g/dL | | |
| CD66e | | | | |
| SAA | 750 g/dL | | | |

### Analytical Sensitivity

The Analytical Sensitivity study was designed to validate the detection capabilities of the immunoassay methods described herein (e.g., Luminex, ELISA, Ortho/VITROS, specifically assessing the following performance characteristics: Limit of Blank (LOB), Limit of Detection (LOD), Lower Limit of Quantitation (LLoQ), and Upper Limit of Quantitation (ULoQ). Data collected from prior studies and other analytical validation studies were used for in silico analysis of analytical sensitivity. The protein standards and blanks that were processed as part of a prior study (41 plate/batches of data) were used for the data analysis. The Standards (N=2 in each plate) were used to establish the LLOQ and ULOQ for each analyte. The Blank wells (N=4 to 6 per plate) were used to estimate the LOB and LOD for each analyte. The evaluation approaches outlined in CLSI EP17-A2: 2012 were applied to determine the above characteristics.

To determine the LOB, a parametric method was used for each protein by utilizing the mean and standard deviation of all the blank results for that protein in the dataset. To determine the LOD, a parametric analysis was performed using the LOB and standard deviation of all the blank results for that protein in the dataset. To determine the LLOQ, the percent of recovery (% Recovery) and concentration-based intra-batch percent Coefficient of Variation (% CV) were determined for each level of standard in each plate. Subsequently, the average values across the 41 plates were compared to the thresholds in Table 34. The lowest standard that satisfied both analyte-specific acceptance criteria outlined in Table 34, was designated as the LLOQ for the specific protein. To determine the ULoQ, the average values of % recovery and concentration-based intra-batch % CV across the plates were compared to the thresholds in Table 34. The highest standard that adheres to both analyte-specific acceptance criteria outlined in Table 34, was designated as the ULoQ for the specific protein.

TABLE 34
Specified percent recovery and intra-batch % CV thresholds
for standards defined for each protein

| | Luminex | Standard % | Standard % |
|---|---|---|---|
| Protein | Panel | recovery | CV |
| FGL1 ELISA | N/A | 80-120 | 25 |
| gp130 | 15 | 90-115 | 15 |
| CD31/PECAM-1 | 16 | 90-110 | 25 |
| C9 | 17 | 90-115 | 25 |
| Myoglobin | 17 | 80-120 | 20 |
| IGFBP-6 | 18 | 90-110 | 20 |
| CYFRA21-1 | 20 | 80-120 | 15 |
| Enolase2 | 20 | 90-110 | 15 |
| CEACAM-5/CD66e | 21 | 90-110 | 15 |
| SAA | 22 | 80-120 | 25 |

Standards (Std) 1 through 6 (N=2 in each plate, 82 measurements in total across the 41 batches) were used to evaluate the concentrations of LLoQ and ULoQ for each analyte. The average % Recovery and concentration-based intra plate % CV were calculated for each of the Luminex assays and ELISA. The results obtained from these metrics for the highest and lowest standard levels are presented in Table 35. Std 6, which is the lowest standard that passed % CV and % Recovery acceptance criteria, was defined as the LLOQ. Std 1, which is the highest standard that passed % CV and % Recovery acceptance criteria, was defined as the ULOQ.

TABLE 35
The average % Recovery and intra-plate % CV of the highest (#1) and lowest
(#6) standards in the 10 Luminex and ELISA assays based on standards data

| | | Avg. Intra- | Avg. % | | Avg. Intra- | Avg. % | % Recovery | % CV |
|---|---|---|---|---|---|---|---|---|
| FEATURE | Std | plate % CV | Recovery | Std | plate % CV | Recovery | cutoff | cutoff |
| ELISA\| | Std1 | 0.5 ± 0.0 | 97.1 ± 2.6 | Std6 | 8.5 ± 4.2 | 105.8 ± 13.6 | 80-120 | 25 |
| FGL1 | | | | | | | | |
| gp130 | Std1 | 2.2 ± 1.7 | 101.0 ± 3.2 | Std6 | 3.0 ± 1.5 | 100.1 ± 0.1 | 90-115 | 15 |
| CD31/ | Std1 | 5.6 ± 1.4 | 100.9 ± 0.5 | Std6 | 6.3 ± 5.1 | 101.3 ± 2.2 | 90-110 | 25 |
| PECAM-1 | | | | | | | | |
| C9 | Std1 | 9.7 ± 5.0 | 102.1 ± 3.7 | Std6 | 5.4 ± 3.5 | 100.7 ± 1.3 | 90-115 | 25 |
| Myoglobin | Std1 | 3.9 ± 2.3 | 95.1 ± 11.1 | Std6 | 3.6 ± 2.3 | 100.0 ± 1.1 | 80-120 | 20 |
| IGFBP-6 | Std1 | 2.5 ± 1.7 | 101.2 ± 0.8 | Std6 | 5.4 ± 3.9 | 100.4 ± 0.9 | 90-110 | 20 |
| CYFRA21-1 | Std1 | 2.8 ± 2.3 | 97.9 ± 6.9 | Std6 | 4.0 ± 2.9 | 99.9 ± 0.7 | 80-120 | 15 |
| Enolase 2 | Std1 | 2.2 ± 2.8 | 100.9 ± 1.7 | Std6 | 3.3 ± 2.2 | 100.4 ± 0.8 | 90-110 | 15 |
| CEACAM- | Std1 | 1.9 ± 1.3 | 100.9 ± 0.9 | Std6 | 3.9 ± 2.6 | 100.3 ± 0.4 | 90-110 | 15 |
| 5/CD66e | | | | | | | | |
| SAA | Std1 | 2.5 ± 1.7 | 106.1 ± 11.7 | Std6 | 4.6 ± 3.8 | 105.6 ± 3.1 | 80-120 | 25 |

*Data shown in mean ± standard deviation, % CV are concentration-based

Blank (BLK) wells (N=4 to 6 per plate, 164 to 246 measurements in total across the 41 batches) were used to estimate the LOB and LOD for each analyte. The computed LOB and LOD for the Luminex assays and ELISA are presented in Table 36.

TABLE 36
Computed LOB and LOD for the Luminex assays and ELISA
based on data from blanks

| | No. | LOB | LOB in | LOD | LOD in |
|---|---|---|---|---|---|
| FEATURE | blank | (pg/mL) | LLOQ | (pg/mL) | LLOQ |
| ELISA\|FGL1 | 82 | 10.8 | 11.5% | 21.5 | 22.9% |
| PL15\|gp130 | 246 | 6.1 | 2.0% | 12.2 | 4.1% |
| PL16\|CD31/PECAM-1 | 246 | 82.9 | 11.0% | 165.8 | 22.0% |
| PL18\|IGFBP-6 | 246 | 2.1 | 5.5% | 4.3 | 11.1% |
| PL20\|Enolase 2/Neuron- | 246 | 32.7 | 14.7% | 65.5 | 29.5% |
| specific Enolase | | | | | |
| PL20\|CYFRA21-1 | 246 | 3.1 | 8.6% | 6.2 | 17.2% |
| PL21\|CEACAM-5/CD66e | 246 | 1.9 | 3.4% | 3.9 | 6.7% |
| PL17\|Myoglobin | 246 | 1.6 | 13.5% | 3.2 | 27.1% |
| PL17\|Complement | 246 | 2574.0 | 8.4% | 5147.9 | 16.9% |
| Component C9 | | | | | |
| PL22\|SAA | 164 | 111.4 | 24.4% | 222.9 | 48.8% |

### Cross-Contamination

The cross-contamination study was designed to assess the potential for cross-contamination within the automated assay workflows described herein, specifically for the Luminex and ELISA platforms. Both workflows involve multiple steps such as reagent additions, plate shaking, and washing, which may introduce a low risk of carryover between wells. Data collected from prior studies and other analytical validation studies were used for analysis of cross-contamination. The evaluation was based on the analysis of signal outputs from designated blank wells across multiple plates processed. Elevated signals in these wells may indicate potential cross-contamination, providing insight into the integrity of the automated workflow.

Mean Fluorescence Intensity (MFI) was collated from blanks [wells G3, H1, H2 & H3] on Luminex batches and Absorbance (RAW) values were collated from blanks on ELISA batches [wells G3, H1, H2 & H3]. Paired t-tests were performed comparing wells H1 vs G3, H2 vs G3, H1 vs H3 and H2 vs H3. Based on results obtained it was concluded that cross-contamination does not appear to be a source of concern for either Luminex or ELISA assays.

### Equipment Equivalence

Equivalence between the two equipment systems for ELISA workflows was demonstrated.

### Freeze-Thaw

Sample stability has been evaluated under multiple freeze-thaw cycles, and it was determined that one freeze thaw cycle (post-aliquoting) does not adversely impact protein measurements in the immunoassay workflows described herein.

### CONCLUSION

All Analytical Validation studies were successfully designed and executed in accordance with the College of American Pathologists (CAP) checklist requirements and the Clinical Laboratory Improvement Amendments of 1988 (CLIA′88), as applicable to a laboratory-developed test (LDT). The validation data were reviewed and confirmed to meet predefined acceptance criteria. A summary of the analytical study results is provided in Table 37. The concentrations of assay LLoQ and ULoQ for the 10 Luminex and ELISA assays based on standard data are included in Table 38.

TABLE 37
Analytical Validation-Summary of Results

| Analytical | | | | |
|---|---|---|---|---|
| Validation | Immunoassay | | | Pass/ |
| Study | Workflow | Acceptance Criteria | Study Outcome | Fail |
| Analytical | Luminex | Repeatability and | Repeatability: less | Pass |
| | Method 1 | within-lab imprecision | than 10% for all | |
| | | should be less or equal | protein features. | |
| | | to 20% | Within-lab | |
| | | | imprecision: less than | |
| | | | 10% for all protein | |
| | | | features except C9 | |
| | | | (11%). | |
| Precision | Luminex | Repeatability and | Repeatability: less | Pass |
| | Method 2 | within-lab imprecision | than 5% for all | |
| | | should be less or equal | protein features | |
| | | to 20% | except SAA (13%). | |
| | | | Within-lab | |
| | | | imprecision: less than | |
| | | | 5% for all protein | |
| | | | features except SAA | |
| | | | (13%). | |
| | ELISA | Repeatability and | Repeatability: less | Pass |
| | | within-lab imprecision | than 5%. | |
| | | should be less or equal | Within-lab | |
| | | to 20% | imprecision: less than | |
| | | | 8.5%. | |
| | Ortho | Repeatability and | Intra-day CV: all | Pass |
| | | within-lab imprecision | below 5% | |
| | | should be less or equal | Inter-day CV: all | |
| | | to 20% | below 5% | |
| Analytical | Luminex | The Deming Regression | All proteins had | Pass |
| Accuracy | assays | slope should fall within | Deming slope between | |
| | | 0.87 and 1.13, and the | 0.87 to 1.13 and | |
| | | Pearson correlation | Pearson R value ≥0.94 | |
| | | coefficient >0.94 | | |
| | ELISA | The Deming Regression | FGL1 had Deming | Pass |
| | | slope should fall within | slope between 0.87 to | |
| | | 0.87 and 1.13, and the | 1.13 and Pearson R | |
| | | Pearson correlation | value ≥0.94 | |
| | | coefficient >0.94 | | |
| | Ortho | Percent recovery versus | Percent recovery at all | Pass |
| | | reference value for each | levels was between 87 | |
| | | sample level (Low, | to 113% | |
| | | Medium, and High) | | |
| | | should fall between 87% | | |
| | | and 113% | | |
| Parallelism and | Luminex | Acceptable slope falls | For all proteins, the | Pass |
| Linearity | assays | between 0.8 to 1.2 | slope from linear | |
| | | | regression analysis | |
| | | | were between 0.8 to 1.2 | |
| | ELISA | Acceptable slope falls | FGL1 had a slope | Pass |
| | | between 0.8 to 1.2 | between 0.8 to 1.2 | |
| Analytical | Luminex | Acceptable slope falls | Standard 6 was set as | Pass |
| Reportable | assays, ELISA, | between 0.8 to 1.2. | the LLOQ and | |
| range | Ortho | Repeatability and | Standard 1 was set as | |
| | | within-lab imprecision | ULOQ for each | |
| | | should be less or equal | protein; The AMI | |
| | | to 20% for Luminex & | established for | |
| | | FGL1 ELISA. | sensitivity study was | |
| | | Percent recovery versus | used as reportable | |
| | | reference value for each | range | |
| | | Ortho sample level | | |
| | | (Low and High) should | | |
| | | fall between 87% and | | |
| | | 113% | | |
| Analytical | Luminex | Concentrations of each | Several protein | Pass |
| Specificity | assays and | protein in a pooled | biomarkers were | |
| | ELISA | sample may not change | impacted by the | |
| | | by more than ±13% with | presence of Interfering | |
| | | the presence of | substances like | |
| | | triglycerides, bilirubin | triglycerides, | |
| | | or hemolysis. | hemolysate and | |
| | | | bilirubin (conjugated | |
| | | | and unconjugated) | |
| Analytical | Luminex | For Luminex and | Standard 6 was set as | Pass |
| Sensitivity | assays and | ELISA assays: | the LLOQ and | |
| | ELISA | LLOQ is the lowest | Standard 1 was set as | |
| | | standard that passed | ULOQ for each | |
| | | both %CV and | protein; LOB and LOD | |
| | | %Recovery. | values were | |
| | | ULOQ is the highest | computationally | |
| | | standard that passed | established based on | |
| | | both % CV and | data sets from studies | |
| | | % Recovery. | previously executed | |
| | | LOB (MFI) = 1.645 | | |
| | | *SD (Blank) | | |
| | | LOD (conc) = LOB | | |
| | | (conc) + (1.645 * SD | | |
| | | (blank))/slope near | | |
| | | blank | | |
| Cross | Luminex | The harmonic mean | Cross-contamination | Pass |
| Contamination | assays and | p-value should fall on | does not appear to be a | |
| | ELISA | or below 0.01 | source of concern for | |
| | | | either Luminex assays | |
| | | | or ELISA. | |

TABLE 38
Concentrations of assay LLOQ and ULOQ for the 10 Luminex and ELISA assays
in PiQ-308 based on standards data in LDT001

| | | | ULOQ.std | | LLOQ.std |
|---|---|---|---|---|---|
| FEATURE | Dilution | ULOQ.std# | (pg/mL) | LLOQ.std# | (pg/mL) |
| ELISA\|FGL1 | 1:500 | std1 | 3,000 | std6 | 94 |
| PL15\|gp130 | 1:2 | std1 | 73,330 | std6 | 302 |
| PL16\|CD31/PECAM-1 | 1:2 | std1 | 183,390 | std6 | 755 |
| PL17\|Complement | 1:50 | std1 | 7,413,650 | std6 | 30,509 |
| Component C9 | | | | | |
| PL17\|Myoglobin | 1:50 | std1 | 2,840 | std6 | 12 |
| PL18\|IGFBP-6 | 1:200 | std1 | 9,400 | std6 | 39 |
| PL20\|CYFRA21-1 | 1:2 | std1 | 8,800 | std6 | 36 |
| PL20\|Enolase 2/Neuron- | 1:2 | std1 | 54,000 | std6 | 222 |
| specific Enolase | | | | | |
| PL21\|CEACAM-5/CD66e | 1:8 | std1 | 14,000 | std6 | 58 |
| PL22\|SAA | 1:4000 | std1 | 110,900 | std6 | 456 |

### Example 13: Clinical Validation

The test involved quantification of 11 proteins resulting in data comprising 12 measurements (one protein was measured twice) and algorithmic analyses of these data resulted in an outcome of either an elevated or not-elevated risk for lung cancer. Protein quantification for the test was performed on the Luminex, ELISA and Ortho/VITROS immunoassays. This clinical validation study confirmed the performance and accuracy of the test using human plasma specimens against the known diagnoses. In this evaluation, the reference method was the pathology report, and the candidate method was the classification results generated from protein concentration measurements in the CV study. Concordance was assessed with subject samples that passed QC from a study size of 70 subject samples.

The results had to satisfy the criteria provided in Table 10 and Table 11 in order for the results to be accepted for analysis. The following summarizes some of the criteria and calculations in determining if results are to be accepted for analysis.

Each Standard had duplicate concentration measurements and the concentration-based % CV for each standard was rounded to the nearest integer. The equation for % CV is as follows: % CV=(Standard Deviation)/(Mean)×100%. Each standard had duplicate concentration measurements, and the percent recovery was defined by the following equation: Percentage of recovery=(Measured concentration)/(Theoretical concentration)×100%. The calculated percent recovery was rounded to the nearest integer. A plate was excluded from analysis if the plate failed to meet the standard curve acceptance criteria in Table 10 for 5 of 6 standards in the standard curve, which includes Percent Recovery and % CV for blanks and standards. The average of all blanks on a plate was at or below the Max MFI cutoff for each protein, or the plate was failed and excluded from the analysis. All standards had a minimum bead count of 50 beads for each protein, or the protein failed and was excluded from the analysis.

The HQC, MQC and LQC's on Luminex assays had a minimum bead count of 50 beads for each protein. If an assay had an HQC, MQC and LQC, then at least 2 of the QC's met the requirements in Table 10, or the plate was failed. If an assay had only a HQC and LQC, then both QC's met the above requirements, or the plate was failed and excluded from the analysis.

The percent recovery for each Process Quality Control (PQC) or process control (PC) was calculated and rounded to the nearest integer. The percent recovery for each PQC or PC was then compared to the allowable Percent Recovery ranges in Table 11. The concentration-based % CV of each PQC or PC was at or below the Max duplicate % CV cutoff for each protein. All PQCs or PCs on Luminex assays had a minimum bead count of 50 beads or the plate was failed. If an assay PQC or PC did not meet the requirements in Table 11, the plate was failed and excluded from the analysis.

For Ortho/VITROS assay quality control acceptance, two controls from tumor marker control kit (Lot #52016) per assay were tested and passed the criteria as in Table 12. The quality control criteria was reagent lot specific. The level 1 acceptable range uses Lot #51791 and the level 2 acceptable range uses Lot #51792.

The acceptance criterion for the overall percent agreement was set at 80%. Clinical accuracy was established for the test if the overall percent agreement is greater than or equal to 80%.

Clinical validation included running 70 biological samples in 2 batches (B001 and B002) through the four analytical methods, each with method-specific work instructions, and acceptance criteria as described elsewhere herein. The four methods were Method 1: Luminex (Panels 15, 17, 18, and 20); Method 2: Luminex (Panels 16, 21, and 22); Method 3: ELISA (FGL1); and Method 4: Vitros ECiQ (CEA and CA125). The output of the methods were concentrations of the plasma proteins of interest and in turn were the inputs to the classifier algorithm. The classifier output a clinical call of cancer risk of Elevated or Not Elevated.

For this validation, the new call of cancer and noncancer presented in Table 39 corresponded to “Elevated” and “Not Elevated” classification. There were 5 hemolyzed samples observed during processing and were excluded from the analysis: The sample IDs are as follows: 0001645, 0003083, 0002875, 0004074, and 0003409.

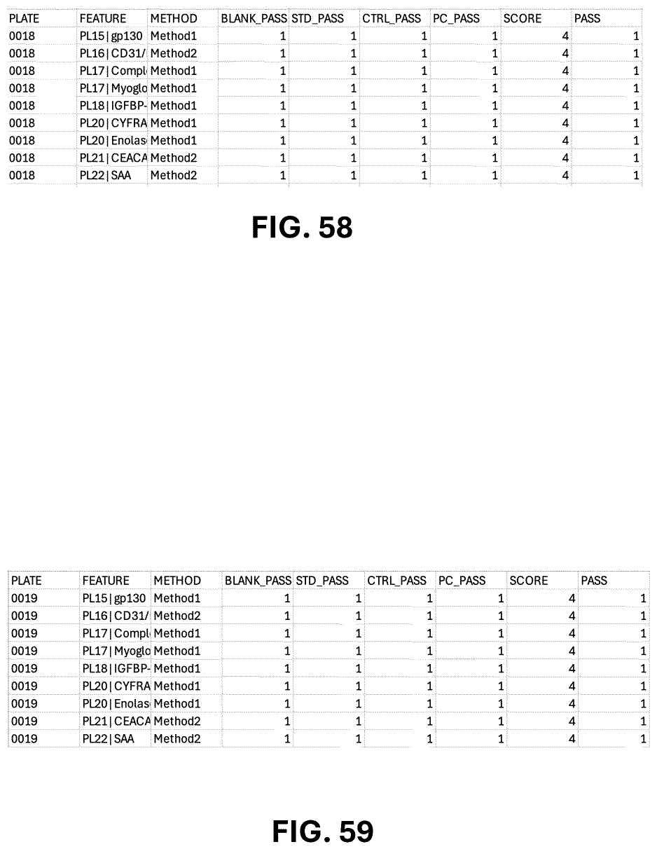

Quality control analysis for each batch/plate for each method was performed. FIG. 58 shows the batch level quality control for batch B001. FIG. 59 shows the batch level quality control for batch B002. In both FIG. 58 and FIG. 59, a designation of ‘1’ indicated a pass for the specified criteria. In this instance both batches passed the quality control analysis for the blank, the standard (STD), the control (CTRL), and the process control (PC). Additionally, a score of ‘4’ was needed for the marker to pass. If the score was less than ‘4’, the batch was re-run. In this instance, both batches had a score of ‘4’. Therefore, both B001 and B002 passed the batch level quality control criteria.

Sample-level quality control analysis for each sample in a batch for each method was performed. Twenty-one of 35 samples passed sample % CV for the sample-level quality control analysis of batch B001. Twenty-two of 35 samples passed sample % CV for the sample-level quality control analysis of batch B002. Although 43 of 70 samples passed the analytical quality control criteria for all 12 assays, 5 samples needed to be excluded due to the presence of hemolysis as noted above. Of the 65 total samples remaining, 41 passed quality control analysis for all proteins. 100% of these 41 samples (41/41) passed the percent agreement criterion. Table 39 show the concordance between historical call and the new call.

TABLE 39
Clinical Call Concordance with Confirmed Historical Call
for all Samples that Passed QC

| | | | Historical |
|---|---|---|---|
| | | | and New Call |
| | | | Concordant? |
| Sample ID | Historical Call | New Call | (Yes/No) |
| A001-0032 | CANCER | CANCER | Yes |
| A001-1044 | CANCER | CANCER | Yes |
| A001-2036 | NON-CANCER | NON-CANCER | Yes |
| A001-2078 | NON-CANCER | NON-CANCER | Yes |
| A002-0079 | NON-CANCER | NON-CANCER | Yes |
| A002-0092 | NON-CANCER | NON-CANCER | Yes |
| A003-0021 | CANCER | CANCER | Yes |
| A003-0026 | NON-CANCER | NON-CANCER | Yes |
| A003-0040 | CANCER | CANCER | Yes |
| A006-0025 | NON-CANCER | NON-CANCER | Yes |
| A007-0030 | NON-CANCER | NON-CANCER | Yes |
| A007-0031 | NON-CANCER | NON-CANCER | Yes |
| A007-0042 | NON-CANCER | NON-CANCER | Yes |
| A007-0150 | NON-CANCER | NON-CANCER | Yes |
| A008-0057 | CANCER | CANCER | Yes |
| A008-0067 | CANCER | CANCER | Yes |
| A008-0068 | CANCER | CANCER | Yes |
| A009-0203 | NON-CANCER | NON-CANCER | Yes |
| A015-0112 | NON-CANCER | NON-CANCER | Yes |
| A016-0115 | NON-CANCER | NON-CANCER | Yes |
| A017-0097 | CANCER | CANCER | Yes |
| A017-0241 | NON-CANCER | NON-CANCER | Yes |
| A017-0246 | NON-CANCER | NON-CANCER | Yes |
| A017-0290 | NON-CANCER | NON-CANCER | Yes |
| A017-0299 | CANCER | CANCER | Yes |
| A018-0056 | CANCER | CANCER | Yes |
| A018-0111 | CANCER | CANCER | Yes |
| A018-0226 | CANCER | CANCER | Yes |
| A018-0356 | NON-CANCER | NON-CANCER | Yes |
| A018-0372 | CANCER | CANCER | Yes |
| A019-0007 | NON-CANCER | NON-CANCER | Yes |
| A021-0167 | CANCER | CANCER | Yes |
| A021-3031 | NON-CANCER | NON-CANCER | Yes |
| A021-3052 | CANCER | CANCER | Yes |
| A021-3092 | CANCER | CANCER | Yes |
| A021-3096 | CANCER | CANCER | Yes |
| A022-0041 | NON-CANCER | NON-CANCER | Yes |
| A022-0044 | NON-CANCER | NON-CANCER | Yes |
| A027-0170 | NON-CANCER | NON-CANCER | Yes |
| A027-0288 | NON-CANCER | NON-CANCER | Yes |
| A028-0036 | NON-CANCER | NON-CANCER | Yes |
| A028-0128 | NON-CANCER | NON-CANCER | Yes |
| A028-0183 | NON-CANCER | NON-CANCER | Yes |

The Clinical Validation study for the test methods was successfully designed and executed in accordance with the College of American Pathologists (CAP) checklist requirements and the Clinical Laboratory Improvement Amendments of 1988 (CLIA′88), as applicable to a laboratory-developed test (LDT). The clinical validation data was reviewed and confirmed to meet predefined acceptance criteria.

### Example 14: Treating a Subject with an Elevated Test Result

A subject is a current smoker over age 50 with a 20 pack-year smoking history. The blood of the subject is drawn and processed using the methods disclosed herein, generating a plasma sample. The plasma sample is sent to a laboratory with the appropriate documentation for use in the lung cancer test disclosed herein. The plasma sample is run on a Lumiex platform, ELISA platform, and an Ortho/VITROS assay as disclosed herein. The laboratory receives data on the plasma sample related to proteins. The data is related to proteins FGL1, gp130, C9, myoglobin, ENO2, KRT19 (CYFRA21-1), SAA, CEACAM-5, IGFBP-6, PECAM-1, and CA-125. The data is run through an algorithm and the results reviewed by the laboratory. The test result for the subject is “Elevated”. The report including the test result is reported to the subject. Based on the “Elevated” result, the subject begins a therapeutic regimen to treat lung cancer. The treatment regimen consists of treatment using a therapeutic agent, such as one or more therapeutic agents provided herein, for example as provided in Table 40 that target the one or more proteins data is received on including FGL1, gp130, C9, myoglobin, ENO2, KRT19 (CYFRA21-1), SAA, CEACAM-5, IGFBP-6, PECAM-1, or CA-125.

While preferred embodiments of the present inventive concepts have been shown and described herein, it will be obvious to those skilled in the art that such embodiments are provided by way of example only. Numerous variations, changes, and substitutions will now occur to those skilled in the art without departing from the inventive concepts. It should be understood that various alternatives to the embodiments of the inventive concepts described herein may be employed in practicing the inventive concepts. It is intended that the following claims define the scope of the inventive concepts and that methods and structures within the scope of these claims and their equivalents be covered thereby.

1. A method, comprising:
(a) extracting lung cancer associated proteomic markers from a biofluid sample obtained from a subject or a processed sample therefrom, wherein the lung cancer associated proteomic markers comprise seven or more of: Myoglobin (MB), Complement Component C9 (C9), Cell adhesion molecule (CEA), CA-125 Antigen (CA125), Fragment of Cytokeratin 19 (CYFRA21-1), Glycoprotein 130 (GP130), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, or a proxy proteomic marker thereof as determined using a proteomic association analysis;
(b) analyzing the lung cancer associated proteomic markers in (a) by a method comprising:
(1) selectively binding at least, a subset of the lung cancer associated proteomic markers to one or more detection reagents to form one or more detectable complexes; and
(2) detecting one or more signals obtained from the one or more detectable complexes corresponding to a concentration or an amount of the lung cancer associated proteomic markers in the one or more detectable complexes to produce a plurality of proteomic measurements;
(c) generating a data set comprising the plurality of proteomic measurements; and
(d) analyzing the data set from (c).

2. The method of claim 1, wherein the lung cancer associated proteomic markers are predictive of lung cancer when the plurality of proteomic measurements is analyzed with a classifier that is trained to distinguish lung cancer samples from non-cancer samples with a performance characteristic that comprises a sensitivity of at least 80% and a specificity of at least 55%.

3. The method of claim 1, wherein the lung cancer associated proteomic markers further comprise ten or more of: Myoglobin (MB), Complement Component C9 (C9), Cell adhesion molecule (CEA), CA-125 Antigen (CA125), Fragment of Cytokeratin 19 (CYFRA21-1), Glycoprotein 130 (GP130), Gamma-enolase (ENO2), Fibrinogen-like protein 1 (FGL1), Insulin-like growth factor-binding protein 6 (IGFBP-6), Platelet endothelial cell adhesion molecule (PECAM1), Serum amyloid A protein (SAA), any fragment thereof, or a proxy proteomic marker thereof.

4. The method of claim 1, wherein the plurality of proteomic measurements is obtained from fewer than or equal to about 20 of the lung cancer associated proteomic markers.

5. The method of claim 1, wherein the plurality of proteomic measurements is obtained from fewer than or equal to about 11 of the lung cancer associated proteomic markers.

6. The method of claim 1, wherein the biofluid sample is a blood sample and the processed sample is a plasma sample or a serum sample comprising plasma or serum isolated from the blood sample.

7. The method of claim 2, wherein the lung cancer is stage 1 non-small cell lung cancer.

8. The method of claim 7, wherein the sensitivity is greater than or equal to about 87%.

9. The method of claim 2, wherein the lung cancer is stage 2 non-small cell lung cancer.

10. The method of claim 9, wherein the sensitivity is greater than or equal to about 88%.

11. The method of claim 2, wherein the lung cancer is stage 3 or 4 non-small cell lung cancer.

12. The method of claim 11, wherein the sensitivity is about 100%.

13. The method of claim 1, wherein the classifier is trained to distinguish the lung cancer samples from the non-cancer samples using a training cohort comprising no more than 20% of subjects with the lung cancer.

14. The method of claim 1, wherein the classifier is trained to distinguish the lung cancer samples from the non-cancer samples with an area under the curve (AUC) that is greater than or equal to about 0.80.

15. The method of claim 1, wherein the at least the subset of the one or more lung cancer associated proteomic markers is immobilized to a solid support directly or indirectly.

16. The method of claim 15, wherein the solid support is a bead, a welled plate, a lateral flow membrane, a planar surface, a flow cell, or any combination thereof.

17. The method of claim 1, wherein the one or more detection reagents comprises an antibody or an antigen-binding fragment coupled directly or indirectly to a detectable label.

18. The method of claim 17, wherein the detectable label comprises a fluorescent label, an enzymatic label, a radioactive label, an affinity label, or any combination thereof.

19. The method of claim 18, wherein the affinity label comprises streptavidin-biotin.

20. The method of claim 18, wherein the fluorescent label comprises a fluorescent molecule or a fluorescence resonance energy transfer pair.

21. The method of claim 18, wherein the enzymatic label comprises horse radish peroxidase.

22. The method of claim 1, wherein the method for analyzing the lung cancer associated proteomic markers in (b) comprises performing an immunoassay that comprises an enzyme-linked immunosorbent assay (ELISA), a particle-based immunoassay, a proximity extension assay, a lateral flow assay, or any combination thereof.

23. The method of claim 22, wherein the immunoassay comprises a fluorescence or bioluminescence readout.

24. The method of claim 22, wherein the particle-based immunoassay comprises forming a detectable complex of the one or more detectable complexes, wherein the detectable complex comprises:
(i) a receptor immobilized to a particle, wherein the receptor specifically binds to a lung cancer associated proteomic marker of the at least the subset of the lung cancer associated proteomic markers; and
(ii) the one or more detection reagents comprising a binding moiety coupled to a detectable label, wherein the binding moiety specifically binds to the lung cancer associated proteomic marker or a molecular tag directly or indirectly coupled thereto.

25. The method of claim 24, wherein the receptor comprises an antibody or an antigen-binding fragment.

26. The method of claim 24, wherein a detection reagent of the one or more detection reagents comprises an antibody or an antigen-binding fragment coupled to the detectable label.

27. The method of claim 26, wherein the detectable label is a fluorescent, enzymatic, radioactive, and affinity label.

28. The method of claim 24, wherein the particle is a bead, a nanoparticle, or a surface plasmon resonance (SPR) particle.

29. The method of claim 28, wherein the bead comprises a coating layer coupled to a surface of the bead, wherein the coating layer comprises carboxyl (—COOH) groups, streptavidin, or avidin.

30. The method of claim 1, further comprising administering a therapeutic agent for the treatment of the lung cancer to the subject, wherein the therapeutic agent is provided in Table 2 or Table 40.

31. The method of claim 1, wherein the method for analyzing the lung cancer associated proteomic markers in (b) comprises performing at least two different immunoassays.

32. The method of claim 31, wherein the at least two different immunoassays comprise a first immunoassay, a second immunoassay, and a third immunoassay, wherein:
(i) Myoglobin (MB), Complement Component C9 (C9), Fragment of Cytokeratin 19 (CYFRA21-1), Glycoprotein 130 (GP130), Gamma-enolase (ENO2), Insulin-like growth factor-binding protein 6 (IGFBP-6), Platelet endothelial cell adhesion molecule (PECAM1), Serum amyloid A protein (SAA), or any fragment thereof, or the proxy proteomic marker thereof, is analyzed using the first immunoassay;
(ii) Cell adhesion molecule (CEA), CA-125 Antigen (CA125), or any fragment thereof, or the proxy proteomic marker thereof, is analyzed using the second immunoassay; and
(iii) Fibrinogen-like protein 1 (FGL1), a fragment thereof, or the proxy proteomic marker thereof, is analyzed using the third immunoassay.


SEQUENCE LISTING (86 sequences)

SEQ ID NO: 1 - protein, 312 aa, synthetic construct
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 2 - protein, 49 aa, synthetic construct
TPKFELKNTSGLMFQILQRVILPSGHLTLLQGTILIQKIKCIQMAISLM

SEQ ID NO: 3 - protein, 559 aa, synthetic construct
MSACRSFAVAICILEISILTAQYTTSYDPELTESSGSASHIDCRMSPWSEWSQCDPCLRQMFRSRSIEVFGQFNGKRCTDAVGDRRQCVPTEPCEDAEDDCGNDFQCSTGRCIKMRLRCNGDNDCGDFSDEDDCESEPRPPCRDRVVEESELARTAGYGINILGMDPLSTPFDNEFYNGLCNRDRDGNTLTYYRRPWNVASLIYETKGEKNFRTEHYEEQIEAFKSIIQEKTSNFNAAISLKFTPTETNKAEQCCEETASSISLHGKGSFRFSYSKNETYQLFLSYSSKKEKMFLHVKGEIHLGRFVMRNRDVVLTTTFVDDIKALPTTYEKGEYFAFLETYGTHYSSSGSLGGLYELIYVLDKASMKRKGVELKDIKRCLGYHLDVSLAFSEISVGAEFNKDDCVKRGEGRAVNITSENLIDDVVSLIRGGTRKYAFELKEKLLRGTVIDVTDFVNWASSINDAPVLISQKLSPIYNLVPVKMKNAHLKKQNLERAIEDYINEFSVRKCHTCQNGGTVILMDGKCLCACPFKFEGIACEISKQKISEGLPALEFPNEK

SEQ ID NO: 4 - protein, 738 aa, synthetic construct
MQPRWAQGATMWLGVLLTLLLCSSLEGQENSFTINSVDMKSLPDWTVQNGKNLTLQCFADVSTTSHVKPQHQMLFYKDDVLFYNISSMKSTESYFIPEVRIYDSGTYKCTVIVNNKEKTTAEYQVLVEGVPSPRVTLDKKEAIQGGIVRVNCSVPEEKAPIHFTIEKLELNEKMVKLKREKNSRDQNFVILEFPVEEQDRVLSFRCQARIISGIHMQTSESTKSELVTVTESFSTPKFHISPTGMIMEGAQLHIKCTIQVTHLAQEFPEIIIQKDKAIVAHNRHGNKAVYSVMAMVEHSGNYTCKVESSRISKVSSIVVNITELFSKPELESSFTHLDQGERLNLSCSIPGAPPANFTIQKEDTIVSQTQDFTKIASKSDSGTYICTAGIDKVVKKSNTVQIVVCEMLSQPRISYDAQFEVIKGQTIEVRCESISGTLPISYQLLKTSKVLENSTKNSNDPAVFKDNPTEDVEYQCVADNCHSHAKMLSEVLRVKVIAPVDEVQISILSSKVVESGEDIVLQCAVNEGSGPITYKFYREKEGKPFYQMTSNATQAFWTKQKASKEQEGEYYCTAFNRANHASSVPRSKILTVRVILAPWKKGLIAVVIIGVIIALLIIAAKCYFLRKAKAKQMPVEMSRPAVPLLNSNNEKMSDPNMEANSHYGHNDDVRNHAMKPINDNKEPLNSDVQYTEVQVSSAESHKDLGKKDTETVYSEVRKAVPDAVESRYSRTEGSLDGT

SEQ ID NO: 5 - protein, 154 aa, synthetic construct
MGLSDGEWQLVLNVWGKVEADIPGHGQEVLIRLFKGHPETLEKFDKFKHLKSEDEMKASEDLKKHGATVLTALGGILKKKGHHEAEIKPLAQSHATKHKIPVKYLEFISECIIQVLQSKHPGDFGADAQGAMNKALELFRKDMASNYKELGFQG

SEQ ID NO: 6 - protein, 240 aa, synthetic construct
MTPHRLLPPLLLLLALLLAASPGGALARCPGCGQGVQAGCPGGCVEEEDGGSPAEGCAEAEGCLRREGQECGVYTPNCAPGLQCHPPKDDEAPLRALLLGRGRCLPARAPAVAEENPKESKPQAGTARPQDVNRRDQQRNPGTSTTPSQPNSAGVQDTEMGPCRRHLDSVLQQLQTEVYRGAQTLYVPNCDHRGFYRKRQCRSSQGQRRGPCWCVDRMGKSLPGSPDGNGSSSCPTGSSG

SEQ ID NO: 7 - protein, 400 aa, synthetic construct
MTSYSYRQSSATSSFGGLGGGSVRFGPGVAFRAPSIHGGSGGRGVSVSSARFVSSSSSGAYGGGYGGVLTASDGLLAGNEKLTMQNLNDRLASYLDKVRALEAANGELEVKIRDWYQKQGPGPSRDYSHYYTTIQDLRDKILGATIENSRIVLQIDNARLAADDFRTKFETEQALRMSVEADINGLRRVLDELTLARTDLEMQIEGLKEELAYLKKNHEEEISTLRGQVGGQVSVEVDSAPGTDLAKILSDMRSQYEVMAEQNRKDAEAWFTSRTEELNREVAGHTEQLQMSRSEVTDLRRTLQGLEIELQSQLSMKAALEDTLAETEARFGAQLAHIQALISGIEAQLGDVRADSERQNQEYQRLMDIKSRLEQEIATYRSLLEGQEDHYNNLSASKVL

SEQ ID NO: 8 - protein, 434 aa, synthetic construct
MSIEKIWAREILDSRGNPTVEVDLYTAKGLFRAAVPSGASTGIYEALELRDGDKQRYLGKGVLKAVDHINSTIAPALISSGLSVVEQEKLDNLMLELDGTENKSKFGANAILGVSLAVCKAGAAERELPLYRHIAQLAGNSDLILPVPAFNVINGGSHAGNKLAMQEFMILPVGAESFRDAMRLGAEVYHTLKGVIKDKYGKDATNVGDEGGFAPNILENSEALELVKEAIDKAGYTEKIVIGMDVAASEFYRDGKYDLDFKSPTDPSRYITGDQLGALYQDFVRDYPVVSIEDPFDQDDWAAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 9 - protein, 702 aa, synthetic construct
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

SEQ ID NO: 10 - protein, 122 aa, synthetic construct
MKLLTGLVFCSLVLGVSSRSFFSFLGEAFDGARDMWRAYSDMREANYIGSDKYFHARGNYDAAKRGPGGAWAAEVITDARENIQRFFGHGAEDSLADQAANEWGRSGKDPNHFRPAGLPEKY

SEQ ID NO: 11 - protein, 122 aa, synthetic construct
MKLLTGLVFCSLVLSVSSRSFFSFLGEAFDGARDMWRAYSDMREANYIGSDKYFHARGNYDAAKRGPGGAWAAEVISNARENIQRLTGRGAEDSLADQAANKWGRSGRDPNHFRPAGLPEKY

SEQ ID NO: 12 - protein, 130 aa, synthetic construct
MRLFTGIVFCSLVMGVTSESWRSFFKEALQGVGDMGRAYWDIMISNHQNSNRYLYARGNYDAAQRGPGGVWAAKLISRSRVYLQGLIDCYLFGNSSTVLEDSKSNEKAEEWGRSGKDPDRFRPDGLPKKY

SEQ ID NO: 13 - protein, 14507 aa, synthetic construct
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTYTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWTSVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGSSPAGTSPGSPEMSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLRTLNTLDINLESGTTSSPSWKSSPYERIAPSESTTDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVSWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPEPVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAVANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSLAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHEILTTDEDTTAIEAMHPSTSTAATNVETTSSGHGSQSSVLADSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSSGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSLLTPGQVKTTEVLDASAEPETSSPPSLSSTSVEILATSEVTTDTEKIHPFSNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLDTSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLNPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSTLPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISTEVITRLSTSPIKTESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILAITEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLAASKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMPSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRIHIPDSDQSTMSPDIITEVITRLSSSSMMSESTQMTITTQKSSPGATAQSTLTLATTTAPLARTHSTVPPRFLHSEMTTLMSRSPENPSWKSSLFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSSPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASMQMASTIPTEMMITTPYVFPDVPETTSSLATSLGAETSTALPRTTPSVFNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPRTTPKFSHSKSDTTLPVAITSPGPEASSAVSTTTISPDMSDLVTSLVPSSGTDTSTTFPTLSETPYEPETTATWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTETSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSATAVDAICTHRPDPEDLGLDRERLYWELSNLTNGIQELGPYTLDRNSLYVNGFTHRSSMPTTSTPGTSTVDVGTSGTPSSSPSPTTAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTSSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTTGPLLVPFTLNFTITNLQYEENMGHPGSRKFNITESVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMRRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPMTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGPYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGCRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMQHLLSPLFQRSSLGARYTGCRVIALRSVKNGAETRVDLLCTYLQPLSGPGLPIKQVFHELSQQTHGITRLGPYSLDKDSLYLNGYNEPGPDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGVITCLICGVLVTTRRRKKEGEYNVQQQCPGYYQSHLDLEDLQ

SEQ ID NO: 14 - protein, 14 aa, synthetic construct
ALEWLAHIFSNDEK

SEQ ID NO: 15 - protein, 12 aa, synthetic construct
KAEEEELARPPR

SEQ ID NO: 16 - protein, 22 aa, synthetic construct
TIEDAIAVLSVAEEAADRHPER

SEQ ID NO: 17 - protein, 13 aa, synthetic construct
FFGLPITGMLNSR

SEQ ID NO: 18 - protein, 19 aa, synthetic construct
MAHGYGEESEEERGLEPGK

SEQ ID NO: 19 - protein, 12 aa, synthetic construct
VDPLVSLMMVEK

SEQ ID NO: 20 - protein, 17 aa, synthetic construct
FIVTEAPQHGYLLNLDK

SEQ ID NO: 21 - protein, 16 aa, synthetic construct
AMPNLVSLHLQHCQIR

SEQ ID NO: 22 - protein, 15 aa, synthetic construct
VDGSVDFYRDWATYK

SEQ ID NO: 23 - protein, 17 aa, synthetic construct
CGVPDVAQFVLTEGNPR

SEQ ID NO: 24 - protein, 14 aa, synthetic construct
DPNHFRPAGLPEKY

SEQ ID NO: 25 - protein, 18 aa, synthetic construct
VSPHSGVVALTKPVPEPR

SEQ ID NO: 26 - protein, 13 aa, synthetic construct
LTSALDELLQATR

SEQ ID NO: 27 - protein, 13 aa, synthetic construct
RVQLYDLGLQIHK

SEQ ID NO: 28 - protein, 16 aa, synthetic construct
YAPISGGDHAEVDVPK

SEQ ID NO: 29 - protein, 20 aa, synthetic construct
ALPVFCDMDTEGGGWLVFQR

SEQ ID NO: 30 - protein, 9 aa, synthetic construct
VFAILENKK

SEQ ID NO: 31 - protein, 15 aa, synthetic construct
EIAEDELVAAEALLK

SEQ ID NO: 32 - protein, 19 aa, synthetic construct
FGVPSPSTLEPHPRPLLQR

SEQ ID NO: 33 - protein, 20 aa, synthetic construct
GPGGVWAAEAISDARENIQR

SEQ ID NO: 34 - protein, 15 aa, synthetic construct
GPGGAWAAEVISNAR

SEQ ID NO: 35 - protein, 32 aa, synthetic construct
IEPSSSQVAEGQTLDLNCVVPGQSHAQVTWHK

SEQ ID NO: 36 - protein, 7 aa, synthetic construct
RLVVCEF

SEQ ID NO: 37 - protein, 19 aa, synthetic construct
TLPFHSVIYHEDDINYPHK

SEQ ID NO: 38 - protein, 14 aa, synthetic construct
EANYIGSDKYFHAR

SEQ ID NO: 39 - protein, 14 aa, synthetic construct
VYYREDLSPSITQR

SEQ ID NO: 40 - protein, 14 aa, synthetic construct
EQMVDASRPELLYR

SEQ ID NO: 41 - protein, 13 aa, synthetic construct
REEFWLGLDNLNK

SEQ ID NO: 42 - protein, 17 aa, synthetic construct
LAMQEFMILPVGASSFK

SEQ ID NO: 43 - protein, 17 aa, synthetic construct
QLIKAIQLTYNPDESSK

SEQ ID NO: 44 - protein, 16 aa, synthetic construct
SGRDPNHFRPAGLPEK

SEQ ID NO: 45 - protein, 17 aa, synthetic construct
SIHLFIDSLLNEENPSK

SEQ ID NO: 46 - protein, 12 aa, synthetic construct
DGSFSVVITGLR

SEQ ID NO: 47 - protein, 10 aa, synthetic construct
KYVLPNFEVK

SEQ ID NO: 48 - protein, 18 aa, synthetic construct
SPVTLLAAVMSLPEEHNK

SEQ ID NO: 49 - protein, 12 aa, synthetic construct
ADLHAVQGWAAR

SEQ ID NO: 50 - protein, 10 aa, synthetic construct
DLLLPQPDLR

SEQ ID NO: 51 - protein, 16 aa, synthetic construct
VHFLPVVISDNGMPSR

SEQ ID NO: 52 - protein, 22 aa, synthetic construct
SLSNVIAHEISHSWTGNLVTNK

SEQ ID NO: 53 - protein, 19 aa, synthetic construct
CHCSPGYVAEAGPPHCTAK

SEQ ID NO: 54 - protein, 19 aa, synthetic construct
HVFWGSGSHTLPALLENLK

SEQ ID NO: 55 - protein, 11 aa, synthetic construct
NIDVFNVEDQK

SEQ ID NO: 56 - protein, 14 aa, synthetic construct
SFVQGLGVASDVVR

SEQ ID NO: 57 - protein, 16 aa, synthetic construct
GLEPGQEYNVLLTAEK

SEQ ID NO: 58 - protein, 19 aa, synthetic construct
MSAQTHGLLGQFFQPFDFK

SEQ ID NO: 59 - protein, 11 aa, synthetic construct
VTNDVCSEPLR

SEQ ID NO: 60 - protein, 9 aa, synthetic construct
ILLLEAGPK

SEQ ID NO: 61 - protein, 11 aa, synthetic construct
LGFINWDAINK

SEQ ID NO: 62 - protein, 10 aa, synthetic construct
FLLYGLLGGK

SEQ ID NO: 63 - protein, 11 aa, synthetic construct
AISMLQGLCYR

SEQ ID NO: 64 - protein, 12 aa, synthetic construct
GWPEVWAGSVGR

SEQ ID NO: 65 - protein, 18 aa, synthetic construct
PQYMVLVPSLLHTETTEK

SEQ ID NO: 66 - protein, 12 aa, synthetic construct
GFEFSVPFTEMK

SEQ ID NO: 67 - protein, 22 aa, synthetic construct
GQSGSCVDVNECLTPGVCAHGK

SEQ ID NO: 68 - protein, 18 aa, synthetic construct
EAALSYGFYGCHCGVGGR

SEQ ID NO: 69 - protein, 15 aa, synthetic construct
LSQLSVTDVTTSSLR

SEQ ID NO: 70 - protein, 15 aa, synthetic construct
ILFIFIDSDHTDNQR

SEQ ID NO: 71 - protein, 11 aa, synthetic construct
FLQDILWEEAK

SEQ ID NO: 72 - protein, 12 aa, synthetic construct
EALLTFMEQVHR

SEQ ID NO: 73 - protein, 10 aa, synthetic construct
IENYTPDLPR

SEQ ID NO: 74 - protein, 16 aa, synthetic construct
FNGAPTANFQQDVGTK

SEQ ID NO: 75 - protein, 17 aa, synthetic construct
HVSSPLASYFLSFPYAR

SEQ ID NO: 76 - protein, 13 aa, synthetic construct
VGPVSAVGVTAPK

SEQ ID NO: 77 - protein, 11 aa, synthetic construct
CTWLIEGQPNR

SEQ ID NO: 78 - protein, 15 aa, synthetic construct
SGYPDIGFPLFPLSK

SEQ ID NO: 79 - protein, 13 aa, synthetic construct
NLFYLPHGLSIDK

SEQ ID NO: 80 - protein, 12 aa, synthetic construct
KLVPFATELHER

SEQ ID NO: 81 - protein, 20 aa, synthetic construct
SPVGVQPILNEHTFCAGMSK

SEQ ID NO: 82 - protein, 12 aa, synthetic construct
VPEESVLPLVQK

SEQ ID NO: 83 - protein, 14 aa, synthetic construct
NNFESEDYCMAVCK

SEQ ID NO: 84 - protein, 15 aa, synthetic construct
AMGIMNSFVNDIFER

SEQ ID NO: 85 - protein, 12 aa, synthetic construct
FIVLSNNYLQIR

SEQ ID NO: 86 - protein, 17 aa, synthetic construct
IQVLAVDNSVPQIAVNK